SEQUENCE LISTING (758 sequences)

SEQ ID NO: 1 - DNA, 351 bp, synthetic construct
  misc_feature 1..351: note = Description of Artificial Sequence: Synthetic polynucleotide
  source 1..351: mol_type = other DNA
caggtccagctggtgcagtctgggggaggcttggtacagcctggggggtccctgagactctcctgtgcagcctctggatttactctaagtagttacgacatgcactgggtccgccaatctacaggaaaaggtctggagtgggtctcagctattggttctaccggtgacacatactatccaggctccgtgaagggccgattcaccatcaccagagaaaaagccaagaactccgtgtatcttcaaatgaacagcctgagagccggggacacggctgtgtattactgtgtaagagaggggtgggaggtaccctttgactactggggccagggaaccctggtcactgtctcctca

SEQ ID NO: 2 - protein, 117 aa, synthetic construct
  REGION 1..117: note = Description of Artificial Sequence: Synthetic polypeptide
QVQLVQSGGGLVQPGGSLRLSCAASGFTLSSYDMHWVRQSTGKGLEWVSAIGSTGDTYYPGSVKGRFTITREKAKNSVYLQMNSLRAGDTAVYYCVREGWEVPFDYWGQGTLVTVSS

SEQ ID NO: 3 - DNA, 24 bp, synthetic construct
  misc_feature 1..24: note = Description of Artificial Sequence: Synthetic oligonucleotide
  source 1..24: mol_type = other DNA
ggatttactctaagtagttacgac

SEQ ID NO: 4 - protein, 8 aa, synthetic construct
  REGION 1..8: note = Description of Artificial Sequence: Synthetic peptide
GFTLSSYD

SEQ ID NO: 5 - DNA, 21 bp, synthetic construct
  misc_feature 1..21: note = Description of Artificial Sequence: Synthetic oligonucleotide
  source 1..21: mol_type = other DNA
attggttctaccggtgacaca

SEQ ID NO: 6 - protein, 7 aa, synthetic construct
  REGION 1..7: note = Description of Artificial Sequence: Synthetic peptide
IGSTGDT

SEQ ID NO: 7 - DNA, 33 bp, synthetic construct
  misc_feature 1..33: note = Description of Artificial Sequence: Synthetic oligonucleotide
  source 1..33: mol_type = other DNA
gtaagagaggggtgggaggtaccctttgactac

SEQ ID NO: 8 - protein, 11 aa, synthetic construct
  REGION 1..11: note = Description of Artificial Sequence: Synthetic peptide
VREGWEVPFDY

SEQ ID NO: 9 - DNA, 327 bp, synthetic construct
  misc_feature 1..327: note = Description of Artificial Sequence: Synthetic polynucleotide
  source 1..327: mol_type = other DNA
gacatccagatgacccagtctccagccaccctgtctgtgtctccaggggaaagagccgccctctcctgcagggccagtcagagtgttagcagcaacttagcctggtaccaccagaaacctggccaggctcccaggctcctcatctatggtgcatccaccagggccactggtatcccagccaggttcagtggcattgggtctgggacagagttcactctcattatcagcagcctgcagtctgaagattttgcattttatttctgtcagcagtataataactggcctccattcactttcggccctgggaccaaggtggagatcaaacga

SEQ ID NO: 10 - protein, 109 aa, synthetic construct
  REGION 1..109: note = Description of Artificial Sequence: Synthetic polypeptide
DIQMTQSPATLSVSPGERAALSCRASQSVSSNLAWYHQKPGQAPRLLIYGASTRATGIPARFSGIGSGTEFTLIISSLQSEDFAFYFCQQYNNWPPFTFGPGTKVEIKR

SEQ ID NO: 11 - DNA, 18 bp, synthetic construct
  misc_feature 1..18: note = Description of Artificial Sequence: Synthetic oligonucleotide
  source 1..18: mol_type = other DNA
cagagtgttagcagcaac

SEQ ID NO: 12 - protein, 6 aa, synthetic construct
  REGION 1..6: note = Description of Artificial Sequence: Synthetic peptide
QSVSSN

SEQ ID NO: 13-14 - skipped (2 entries)

SEQ ID NO: 15 - DNA, 30 bp, synthetic construct
  misc_feature 1..30: note = Description of Artificial Sequence: Synthetic oligonucleotide
  source 1..30: mol_type = other DNA
cagcagtataataactggcctccattcact

SEQ ID NO: 16 - protein, 10 aa, synthetic construct
  REGION 1..10: note = Description of Artificial Sequence: Synthetic peptide
QQYNNWPPFT

SEQ ID NO: 17 - DNA, 351 bp, synthetic construct
  misc_feature 1..351: note = Description of Artificial Sequence: Synthetic polynucleotide
  source 1..351: mol_type = other DNA
gaggtgcagctggtggagtctgggggaggcttggtacagcctggggggtccctgagactctcctgtgcagcctctggatttactctaagtagttacgacatgcactgggtccgccaatctacaggaaaaggtctggagtgggtctcagctattggttctaccggtgacacatactatccaggctccgtgaagggccgattcaccatcaccagagaaaaagccaagaactccgtgtatcttcaaatgaacagcctgagagccggggacacggctgtgtattactgtgtaagagaggggtgggaggtaccctttgactactggggccagggaaccctggtcaccgtctcctca

SEQ ID NO: 18 - protein, 117 aa, synthetic construct
  REGION 1..117: note = Description of Artificial Sequence: Synthetic polypeptide
EVQLVESGGGLVQPGGSLRLSCAASGFTLSSYDMHWVRQSTGKGLEWVSAIGSTGDTYYPGSVKGRFTITREKAKNSVYLQMNSLRAGDTAVYYCVREGWEVPFDYWGQGTLVTVSS

SEQ ID NO: 19 - DNA, 324 bp, synthetic construct
  misc_feature 1..324: note = Description of Artificial Sequence: Synthetic polynucleotide
  source 1..324: mol_type = other DNA
gaaatagtgatgacgcagtctccagccaccctgtctgtgtctccaggggaaagagccgccctctcctgcagggccagtcagagtgttagcagcaacttagcctggtaccaccagaaacctggccaggctcccaggctcctcatctatggtgcatccaccagggccactggtatcccagccaggttcagtggcattgggtctgggacagagttcactctcattatcagcagcctgcagtctgaagattttgcattttatttctgtcagcagtataataactggcctccattcactttcggccctgggaccaaagtggatatcaaa

SEQ ID NO: 20 - protein, 108 aa, synthetic construct
  REGION 1..108: note = Description of Artificial Sequence: Synthetic polypeptide
EIVMTQSPATLSVSPGERAALSCRASQSVSSNLAWYHQKPGQAPRLLIYGASTRATGIPARFSGIGSGTEFTLIISSLQSEDFAFYFCQQYNNWPPFTFGPGTKVDIK

SEQ ID NO: 21 - DNA, 351 bp, synthetic construct
  misc_feature 1..351: note = Description of Artificial Sequence: Synthetic polynucleotide
  source 1..351: mol_type = other DNA
gaggtgcagctggtggagtctgggggaggcttggtacagcctggggggtccctgagactctcctgtgcagcctctggatttactctaagtagttacgacatgcactgggtccgccaagctacaggaaaaggtctggagtgggtctcagctattggttctaccggtgacacatactatccaggctccgtgaagggccgattcaccatctccagagaaaatgccaagaactccttgtatcttcaaatgaacagcctgagagccggggacacggctgtgtattactgtgtaagagaggggtgggaggtaccctttgactactggggccagggaaccctggtcaccgtctcctca

SEQ ID NO: 22 - protein, 117 aa, synthetic construct
  REGION 1..117: note = Description of Artificial Sequence: Synthetic polypeptide
EVQLVESGGGLVQPGGSLRLSCAASGFTLSSYDMHWVRQATGKGLEWVSAIGSTGDTYYPGSVKGRFTISRENAKNSLYLQMNSLRAGDTAVYYCVREGWEVPFDYWGQGTLVTVSS

SEQ ID NO: 23 - DNA, 324 bp, synthetic construct
  misc_feature 1..324: note = Description of Artificial Sequence: Synthetic polynucleotide
  source 1..324: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccattcactttcggccctgggaccaaagtggatatcaaa

SEQ ID NO: 24 - protein, 108 aa, synthetic construct
  REGION 1..108: note = Description of Artificial Sequence: Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYNNWPPFTFGPGTKVDIK

SEQ ID NO: 25 - DNA, 342 bp, synthetic construct
  misc_feature 1..342: note = Description of Artificial Sequence: Synthetic polynucleotide
  source 1..342: mol_type = other DNA
caggtgcagctggtgcagtctgggggaggcgtggtccagcctgggaggtccctgagactctcctgtgcagcgtctggattcaccttcagtagctatggcatgcactgggtccgccaggctccaggcaaggggctggagtgggtggcgtttataggatttgatggaagtaatatacattatggagactccgtgaggggccgaatcatcatatccagagacaattccgagaacacgttgtatctggaaatgaacagcctgagagccgaggacacggcaatgtactattgtgcgagagagaagggtttagactggggccagggaaccacggtcaccgtctcctca

SEQ ID NO: 26 - protein, 114 aa, synthetic construct
  REGION 1..114: note = Description of Artificial Sequence: Synthetic polypeptide
QVQLVQSGGGVVQPGRSLRLSCAASGFTFSSYGMHWVRQAPGKGLEWVAFIGFDGSNIHYGDSVRGRIIISRDNSENTLYLEMNSLRAEDTAMYYCAREKGLDWGQGTTVTVSS

SEQ ID NO: 27 - DNA, 24 bp, synthetic construct
  misc_feature 1..24: note = Description of Artificial Sequence: Synthetic oligonucleotide
  source 1..24: mol_type = other DNA
ggattcaccttcagtagctatggc

SEQ ID NO: 28 - protein, 8 aa, synthetic construct
  REGION 1..8: note = Description of Artificial Sequence: Synthetic peptide
GFTFSSYG

SEQ ID NO: 29 - DNA, 24 bp, synthetic construct
  misc_feature 1..24: note = Description of Artificial Sequence: Synthetic oligonucleotide
  source 1..24: mol_type = other DNA
ataggatttgatggaagtaatata

SEQ ID NO: 30 - protein, 8 aa, synthetic construct
  REGION 1..8: note = Description of Artificial Sequence: Synthetic peptide
IGFDGSNI

SEQ ID NO: 31 - DNA, 21 bp, synthetic construct
  misc_feature 1..21: note = Description of Artificial Sequence: Synthetic oligonucleotide
  source 1..21: mol_type = other DNA
gcgagagagaagggtttagac

SEQ ID NO: 32 - protein, 7 aa, synthetic construct
  REGION 1..7: note = Description of Artificial Sequence: Synthetic peptide
AREKGLD

SEQ ID NO: 33 - DNA, 321 bp, synthetic construct
  misc_feature 1..321: note = Description of Artificial Sequence: Synthetic polynucleotide
  source 1..321: mol_type = other DNA
gccatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattacacttttggccaggggaccaaggtggaaatcaaacga

SEQ ID NO: 34 - protein, 107 aa, synthetic construct
  REGION 1..107: note = Description of Artificial Sequence: Synthetic polypeptide
AIQMTQSPSTLSASVGDRVTITCRASQSISSWLAWYQQKPGKAPKLLIYKASSLESGVPSRFSGSGSGTEFTLTISSLQPDDFATYYCQQYNSYYTFGQGTKVEIKR

SEQ ID NO: 35 - DNA, 18 bp, synthetic construct
  misc_feature 1..18: note = Description of Artificial Sequence: Synthetic oligonucleotide
  source 1..18: mol_type = other DNA
cagagtattagtagctgg

SEQ ID NO: 36 - protein, 6 aa, synthetic construct
  REGION 1..6: note = Description of Artificial Sequence: Synthetic peptide
QSISSW

SEQ ID NO: 37-38 - skipped (2 entries)

SEQ ID NO: 39 - DNA, 24 bp, synthetic construct
  misc_feature 1..24: note = Description of Artificial Sequence: Synthetic oligonucleotide
  source 1..24: mol_type = other DNA
caacagtataatagttattacact

SEQ ID NO: 40 - protein, 8 aa, synthetic construct
  REGION 1..8: note = Description of Artificial Sequence: Synthetic peptide
QQYNSYYT

SEQ ID NO: 41 - DNA, 342 bp, synthetic construct
  misc_feature 1..342: note = Description of Artificial Sequence: Synthetic polynucleotide
  source 1..342: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcgtttataggatttgatggaagtaatatacattatggagactccgtgaggggccgaatcatcatatccagagacaattccgagaacacgttgtatctggaaatgaacagcctgagagccgaggacacggcaatgtactattgtgcgagagagaagggtttagactggggccagggaaccctggtcaccgtctcctca

SEQ ID NO: 42 - protein, 114 aa, synthetic construct
  REGION 1..114: note = Description of Artificial Sequence: Synthetic polypeptide
QVQLVESGGGVVQPGRSLRLSCAASGFTFSSYGMHWVRQAPGKGLEWVAFIGFDGSNIHYGDSVRGRIIISRDNSENTLYLEMNSLRAEDTAMYYCAREKGLDWGQGTLVTVSS

SEQ ID NO: 43 - DNA, 318 bp, synthetic construct
  misc_feature 1..318: note = Description of Artificial Sequence: Synthetic polynucleotide
  source 1..318: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattacacttttggccaggggaccaagctggagatcaaa

SEQ ID NO: 44 - protein, 106 aa, synthetic construct
  REGION 1..106: note = Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYYTFGQGTKLEIK

SEQ ID NO: 45 - DNA, 342 bp, synthetic construct
  misc_feature 1..342: note = Description of Artificial Sequence: Synthetic polynucleotide
  source 1..342: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttataggatttgatggaagtaatatatactatgcagactccgtgaagggccgattcaccatctccagagacaattccaagaacacgctgtatctgcaaatgaacagcctgagagctgaggacacggctgtgtattactgtgcgagagagaagggtttagactggggccagggaaccctggtcaccgtctcctca

SEQ ID NO: 46 - protein, 114 aa, synthetic construct
  REGION 1..114: note = Description of Artificial Sequence: Synthetic polypeptide
QVQLVESGGGVVQPGRSLRLSCAASGFTFSSYGMHWVRQAPGKGLEWVAVIGFDGSNIYYADSVKGRFTISRDNSKNTLYLQMNSLRAEDTAVYYCAREKGLDWGQGTLVTVSS

SEQ ID NO: 47 - DNA, 319 bp, synthetic construct
  misc_feature 1..319: note = Description of Artificial Sequence: Synthetic polynucleotide
  source 1..319: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattacacttttggccaggggaccaagctggagatcaaac

SEQ ID NO: 48 - protein, 106 aa, synthetic construct; identical to SEQ ID NO: 44
  REGION 1..106: note = Description of Artificial Sequence: Synthetic polypeptide

SEQ ID NO: 49 - DNA, 342 bp, synthetic construct
  misc_feature 1..342: note = Description of Artificial Sequence: Synthetic polynucleotide
  source 1..342: mol_type = other DNA
caggtgcagctgcaggagtctgggggaggcgtggtccagcctgggaggtccctgagactctcctgtgcagcgtctggattcaccttcagtagctatggcatgcactgggtccgccaggctccaggcaaggggctggagtgggtggcgtttataggatttgatggaagtaatatatattatggagactccgtgaggggccgaatcatcatatccagagacaattccgagaacacgttgtatctggaaatgaacagcctgagagccgaggacacggcagtgtattattgtgcgagagagaagggtttagactggggccagggaaccctggtcactgtctcctca

SEQ ID NO: 50 - protein, 114 aa, synthetic construct
  REGION 1..114: note = Description of Artificial Sequence: Synthetic polypeptide
QVQLQESGGGVVQPGRSLRLSCAASGFTFSSYGMHWVRQAPGKGLEWVAFIGFDGSNIYYGDSVRGRIIISRDNSENTLYLEMNSLRAEDTAVYYCAREKGLDWGQGTLVTVSS

SEQ ID NO: 51 - DNA, 24 bp, synthetic construct
  misc_feature 1..24: note = Description of Artificial Sequence: Synthetic oligonucleotide
  source 1..24: mol_type = other DNA
ggattcaccttcagtagctatggc

SEQ ID NO: 52 - protein, 8 aa, synthetic construct
  REGION 1..8: note = Description of Artificial Sequence: Synthetic peptide
GFTFSSYG

SEQ ID NO: 53 - DNA, 24 bp, synthetic construct
  misc_feature 1..24: note = Description of Artificial Sequence: Synthetic oligonucleotide
  source 1..24: mol_type = other DNA
ataggatttgatggaagtaatata

SEQ ID NO: 54 - protein, 8 aa, synthetic construct
  REGION 1..8: note = Description of Artificial Sequence: Synthetic peptide
IGFDGSNI

SEQ ID NO: 55 - DNA, 21 bp, synthetic construct
  misc_feature 1..21: note = Description of Artificial Sequence: Synthetic oligonucleotide
  source 1..21: mol_type = other DNA
gcgagagagaagggtttagac

SEQ ID NO: 56 - protein, 7 aa, synthetic construct
  REGION 1..7: note = Description of Artificial Sequence: Synthetic peptide
AREKGLD

SEQ ID NO: 57 - DNA, 342 bp, synthetic construct
  misc_feature 1..342: note = Description of Artificial Sequence: Synthetic polynucleotide
  source 1..342: mol_type = other DNA
gccatccagatgacccagtctccagactccctggctgtgtctctgggcgagagggccaccatcaactgcaagtccagccagagtgtttttcacacctccaacaataagaactacttagtttggtatcagcagaaaccaggacagcctcctaagttgctcctttactgggcctctacccgggaatccggggtccctgaccgattcagtggcagcgggtctgggacagatttcactctcaccatcagcagcctgcaggctgaagatgtggcaaattattactgtcaccaatattacagtattccgtggacgttcggccaagggaccaaggtggagatcaaacga

SEQ ID NO: 58 - protein, 114 aa, synthetic construct
  REGION 1..114: note = Description of Artificial Sequence: Synthetic polypeptide
AIQMTQSPDSLAVSLGERATINCKSSQSVFHTSNNKNYLVWYQQKPGQPPKLLLYWASTRESGVPDRFSGSGSGTDFTLTISSLQAEDVANYYCHQYYSIPWTFGQGTKVEIKR

SEQ ID NO: 59 - DNA, 36 bp, synthetic construct
  misc_feature 1..36: note = Description of Artificial Sequence: Synthetic oligonucleotide
  source 1..36: mol_type = other DNA
cagagtgtttttcacacctccaacaataagaactac

SEQ ID NO: 60 - protein, 12 aa, synthetic construct
  REGION 1..12: note = Description of Artificial Sequence: Synthetic peptide
QSVFHTSNNKNY

SEQ ID NO: 61-62 - skipped (2 entries)

SEQ ID NO: 63 - DNA, 27 bp, synthetic construct
  misc_feature 1..27: note = Description of Artificial Sequence: Synthetic oligonucleotide
  source 1..27: mol_type = other DNA
caccaatattacagtattccgtggacg

SEQ ID NO: 64 - protein, 9 aa, synthetic construct
  REGION 1..9: note = Description of Artificial Sequence: Synthetic peptide
HQYYSIPWT

SEQ ID NO: 65 - DNA, 342 bp, synthetic construct
  misc_feature 1..342: note = Description of Artificial Sequence: Synthetic polynucleotide
  source 1..342: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcgtttataggatttgatggaagtaatatatattatggagactccgtgaggggccgaatcatcatatccagagacaattccgagaacacgttgtatctggaaatgaacagcctgagagccgaggacacggcagtgtattattgtgcgagagagaagggtttagactggggccagggaaccctggtcaccgtctcctca

SEQ ID NO: 66 - protein, 114 aa, synthetic construct
  REGION 1..114: note = Description of Artificial Sequence: Synthetic polypeptide
QVQLVESGGGVVQPGRSLRLSCAASGFTFSSYGMHWVRQAPGKGLEWVAFIGFDGSNIYYGDSVRGRIIISRDNSENTLYLEMNSLRAEDTAVYYCAREKGLDWGQGTLVTVSS

SEQ ID NO: 67 - DNA, 339 bp, synthetic construct
  misc_feature 1..339: note = Description of Artificial Sequence: Synthetic polynucleotide
  source 1..339: mol_type = other DNA
gacatcgtgatgacccagtctccagactccctggctgtgtctctgggcgagagggccaccatcaactgcaagtccagccagagtgtttttcacacctccaacaataagaactacttagtttggtatcagcagaaaccaggacagcctcctaagttgctcctttactgggcctctacccgggaatccggggtccctgaccgattcagtggcagcgggtctgggacagatttcactctcaccatcagcagcctgcaggctgaagatgtggcaaattattactgtcaccaatattacagtattccgtggacgttcggccaagggaccaaggtggaaatcaaa

SEQ ID NO: 68 - protein, 113 aa, synthetic construct
  REGION 1..113: note = Description of Artificial Sequence: Synthetic polypeptide
DIVMTQSPDSLAVSLGERATINCKSSQSVFHTSNNKNYLVWYQQKPGQPPKLLLYWASTRESGVPDRFSGSGSGTDFTLTISSLQAEDVANYYCHQYYSIPWTFGQGTKVEIK

SEQ ID NO: 69 - DNA, 342 bp, synthetic construct; identical to SEQ ID NO: 45
  misc_feature 1..342: note = Description of Artificial Sequence: Synthetic polynucleotide
  source 1..342: mol_type = other DNA

SEQ ID NO: 70 - protein, 114 aa, synthetic construct; identical to SEQ ID NO: 46
  REGION 1..114: note = Description of Artificial Sequence: Synthetic polypeptide

SEQ ID NO: 71 - DNA, 339 bp, synthetic construct
  misc_feature 1..339: note = Description of Artificial Sequence: Synthetic polynucleotide
  source 1..339: mol_type = other DNA
gacatcgtgatgacccagtctccagactccctggctgtgtctctgggcgagagggccaccatcaactgcaagtccagccagagtgtttttcacacctccaacaataagaactacttagcttggtaccagcagaaaccaggacagcctcctaagctgctcatttactgggcctctacccgggaatccggggtccctgaccgattcagtggcagcgggtctgggacagatttcactctcaccatcagcagcctgcaggctgaagatgtggcagtttattactgtcaccaatattacagtattccgtggacgttcggccaagggaccaaggtggaaatcaaa

SEQ ID NO: 72 - protein, 113 aa, synthetic construct
  REGION 1..113: note = Description of Artificial Sequence: Synthetic polypeptide
DIVMTQSPDSLAVSLGERATINCKSSQSVFHTSNNKNYLAWYQQKPGQPPKLLIYWASTRESGVPDRFSGSGSGTDFTLTISSLQAEDVAVYYCHQYYSIPWTFGQGTKVEIK

SEQ ID NO: 73 - DNA, 354 bp, synthetic construct
  misc_feature 1..354: note = Description of Artificial Sequence: Synthetic polynucleotide
  source 1..354: mol_type = other DNA
gaagtgcagctggtgcagtctgggggaggcttggtacagcctggggggtccctgagactctcctgtgcagcctctggattcacctttaacaactatgccatgaactgggtccgccaggctccaggaaagggactggactgggtctcaactattagtggtagcggtggtactacaaactacgcagactccgtgaagggccgtttcattatttcccgagacagttccaaacacacgctgtatctgcaaatgaacagcctgagagccgaggacacggccgtatattactgtgcgaaagattctaactggggaaatttcgatctctggggccgtggcaccacggtcactgtctcctca

SEQ ID NO: 74 - protein, 118 aa, synthetic construct
  REGION 1..118: note = Description of Artificial Sequence: Synthetic polypeptide
EVQLVQSGGGLVQPGGSLRLSCAASGFTFNNYAMNWVRQAPGKGLDWVSTISGSGGTTNYADSVKGRFIISRDSSKHTLYLQMNSLRAEDTAVYYCAKDSNWGNFDLWGRGTTVTVSS

SEQ ID NO: 75 - DNA, 24 bp, synthetic construct
  misc_feature 1..24: note = Description of Artificial Sequence: Synthetic oligonucleotide
  source 1..24: mol_type = other DNA
ggattcacctttaacaactatgcc

SEQ ID NO: 76 - protein, 8 aa, synthetic construct
  REGION 1..8: note = Description of Artificial Sequence: Synthetic peptide
GFTFNNYA

SEQ ID NO: 77 - DNA, 24 bp, synthetic construct
  misc_feature 1..24: note = Description of Artificial Sequence: Synthetic oligonucleotide
  source 1..24: mol_type = other DNA
attagtggtagcggtggtactaca

SEQ ID NO: 78 - protein, 8 aa, synthetic construct
  REGION 1..8: note = Description of Artificial Sequence: Synthetic peptide
ISGSGGTT

SEQ ID NO: 79 - DNA, 33 bp, synthetic construct
  misc_feature 1..33: note = Description of Artificial Sequence: Synthetic oligonucleotide
  source 1..33: mol_type = other DNA
gcgaaagattctaactggggaaatttcgatctc

SEQ ID NO: 80 - protein, 11 aa, synthetic construct
  REGION 1..11: note = Description of Artificial Sequence: Synthetic peptide
AKDSNWGNFDL

SEQ ID NO: 81 - DNA, 342 bp, synthetic construct
  misc_feature 1..342: note = Description of Artificial Sequence: Synthetic polynucleotide
  source 1..342: mol_type = other DNA
gacatccagatgacccagtctccagactccctggctgtgtctctgggcgagagggccaccatcaactgcaagtccagccagagtgttttatacaggtccaacaataggaacttcttaggttggtaccagcagaaaccagggcagcctcctaatctactcatttactgggcatctacccgggaatccggggtccctgaccgattcagtggcagcgggtctgggacagatttcactctcaccatcagcagcctgcaggctgaagatgtggcagtttattactgtcaacaatattatactactccgtacacttttggccaggggaccaaggtggaaatcaaacga

SEQ ID NO: 82 - protein, 114 aa, synthetic construct
  REGION 1..114: note = Description of Artificial Sequence: Synthetic polypeptide
DIQMTQSPDSLAVSLGERATINCKSSQSVLYRSNNRNFLGWYQQKPGQPPNLLIYWASTRESGVPDRFSGSGSGTDFTLTISSLQAEDVAVYYCQQYYTTPYTFGQGTKVEIKR

SEQ ID NO: 83 - DNA, 36 bp, synthetic construct
  misc_feature 1..36: note = Description of Artificial Sequence: Synthetic oligonucleotide
  source 1..36: mol_type = other DNA
cagagtgttttatacaggtccaacaataggaacttc

SEQ ID NO: 84 - protein, 12 aa, synthetic construct
  REGION 1..12: note = Description of Artificial Sequence: Synthetic peptide
QSVLYRSNNRNF

SEQ ID NO: 85-86 - skipped (2 entries)

SEQ ID NO: 87 - DNA, 27 bp, synthetic construct
  misc_feature 1..27: note = Description of Artificial Sequence: Synthetic oligonucleotide
  source 1..27: mol_type = other DNA
caacaatattatactactccgtacact

SEQ ID NO: 88 - protein, 9 aa, synthetic construct
  REGION 1..9: note = Description of Artificial Sequence: Synthetic peptide
QQYYTTPYT

SEQ ID NO: 89 - DNA, 354 bp, synthetic construct
  misc_feature 1..354: note = Description of Artificial Sequence: Synthetic polynucleotide
  source 1..354: mol_type = other DNA
gaggtgcagctggtggagtctgggggaggcttggtacagcctggggggtccctgagactctcctgtgcagcctctggattcacctttaacaactatgccatgaactgggtccgccaggctccaggaaagggactggactgggtctcaactattagtggtagcggtggtactacaaactacgcagactccgtgaagggccgtttcattatttcccgagacagttccaaacacacgctgtatctgcaaatgaacagcctgagagccgaggacacggccgtatattactgtgcgaaagattctaactggggaaatttcgatctctggggccgtggcaccctggtcactgtctcctca

SEQ ID NO: 90 - protein, 118 aa, synthetic construct [sequence omitted]

SEQ ID NO: 91 - DNA, 339 bp, synthetic construct
  misc_feature 1..339: note = Description of Artificial Sequence: Synthetic polynucleotide
  source 1..339: mol_type = other DNA
gacatcgtgatgacccagtctccagactccctggctgtgtctctgggcgagagggccaccatcaactgcaagtccagccagagtgttttatacaggtccaacaataggaacttcttaggttggtaccagcagaaaccagggcagcctcctaatctactcatttactgggcatctacccgggaatccggggtccctgaccgattcagtggcagcgggtctgggacagatttcactctcaccatcagcagcctgcaggctgaagatgtggcagtttattactgtcaacaatattatactactccgtacacttttggccaggggaccaagctggagatcaaa

SEQ ID NO: 92 - protein, 113 aa, synthetic construct [sequence omitted]

SEQ ID NO: 93 - DNA, 354 bp, synthetic construct
  misc_feature 1..354: note = Description of Artificial Sequence: Synthetic polynucleotide
  source 1..354: mol_type = other DNA
gaggtgcagctggtggagtctgggggaggcttggtacagcctggggggtccctgagactctcctgtgcagcctctggattcacctttaacaactatgccatgagctgggtccgccaggctccagggaaggggctggagtgggtctcagctattagtggtagcggtggtactacatactacgcagactccgtgaagggccggttcaccatctccagagacaattccaagaacacgctgtatctgcaaatgaacagcctgagagccgaggacacggccgtatattactgtgcgaaagattctaactggggaaatttcgatctctggggccgtggcaccctggtcactgtctcctca

SEQ ID NO: 94 - protein, 118 aa, synthetic construct
  REGION 1..118: note = Description of Artificial Sequence: Synthetic polypeptide
EVQLVESGGGLVQPGGSLRLSCAASGFTFNNYAMSWVRQAPGKGLEWVSAISGSGGTTYYADSVKGRFTISRDNSKNTLYLQMNSLRAEDTAVYYCAKDSNWGNFDLWGRGTLVTVSS

SEQ ID NO: 95 - DNA, 339 bp, synthetic construct
  misc_feature 1..339: note = Description of Artificial Sequence: Synthetic polynucleotide
  source 1..339: mol_type = other DNA
gacatcgtgatgacccagtctccagactccctggctgtgtctctgggcgagagggccaccatcaactgcaagtccagccagagtgttttatacaggtccaacaataggaacttcttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcaacaatattatactactccgtacacttttggccaggggaccaagctggagatcaaa

SEQ ID NO: 96 - protein, 113 aa, synthetic construct
  REGION 1..113: note = Description of Artificial Sequence: Synthetic polypeptide
DIVMTQSPDSLAVSLGERATINCKSSQSVLYRSNNRNFLAWYQQKPGQPPKLLIYWASTRESGVPDRFSGSGSGTDFTLTISSLQAEDVAVYYCQQYYTTPYTFGQGTKLEIK

SEQ ID NO: 97 - DNA, 351 bp, synthetic construct
  misc_feature 1..351: note = Description of Artificial Sequence: Synthetic polynucleotide
  source 1..351: mol_type = other DNA
caggtgcagctggtgcagtctgggggaggcttggtacagcctggggggtccctgagactctcctgtgcagtctctggattcaccctcagtagctacgatatgcactgggtccgccaacctacaggaaaaggtctggagtgggtctcagctattggttctactggtgacacatactatccaggctccgtgaagggccgattcaccatctccagagaaaatgccaagaactccttgtatcttcaaatgaacagcctgagagccggggacacggctgtgtattactgtgcaagagagggatgggacgtaccctttgacttctggggccagggaaccctggtcaccgtctcctca

SEQ ID NO: 98 - protein, 117 aa, synthetic construct
  REGION 1..117: note = Description of Artificial Sequence: Synthetic polypeptide
QVQLVQSGGGLVQPGGSLRLSCAVSGFTLSSYDMHWVRQPTGKGLEWVSAIGSTGDTYYPGSVKGRFTISRENAKNSLYLQMNSLRAGDTAVYYCAREGWDVPFDFWGQGTLVTVSS

SEQ ID NO: 99 - DNA, 24 bp, synthetic construct
  misc_feature 1..24: note = Description of Artificial Sequence: Synthetic oligonucleotide
  source 1..24: mol_type = other DNA
ggattcaccctcagtagctacgat

SEQ ID NO: 100 - protein, 8 aa, synthetic construct
  REGION 1..8: note = Description of Artificial Sequence: Synthetic peptide
GFTLSSYD

SEQ ID NO: 101 - DNA, 21 bp, synthetic construct
  misc_feature 1..21: note = Description of Artificial Sequence: Synthetic oligonucleotide
  source 1..21: mol_type = other DNA
attggttctactggtgacaca

SEQ ID NO: 102 - protein, 7 aa, synthetic construct
  REGION 1..7: note = Description of Artificial Sequence: Synthetic peptide
IGSTGDT

SEQ ID NO: 103 - DNA, 33 bp, synthetic construct
  misc_feature 1..33: note = Description of Artificial Sequence: Synthetic oligonucleotide
  source 1..33: mol_type = other DNA
gcaagagagggatgggacgtaccctttgacttc

SEQ ID NO: 104 - protein, 11 aa, synthetic construct
  REGION 1..11: note = Description of Artificial Sequence: Synthetic peptide
AREGWDVPFDF

SEQ ID NO: 105 - DNA, 324 bp, synthetic construct
  misc_feature 1..324: note = Description of Artificial Sequence: Synthetic polynucleotide
  source 1..324: mol_type = other DNA
gccatccagttgacccagtctccatcctccctgtctgcatctgtaggagacagagtcaccatcacttgccgggcaagtcaggacattagaaatgatttaggctggtatcagcagaaaccagggaaagcccctaagctcctgatctatgctgcatccagtttacaaagtggggtcccatcacggttcagcggcagtggatctggcacagatttcactctcaccatcagcagcctgcagcctgaagattttgcaacttattactgtctacaagattacaattacccgtggacgttcggccaagggaccaaggtggagatcaaacga

SEQ ID NO: 106 - protein, 108 aa, synthetic construct
  REGION 1..108: note = Description of Artificial Sequence: Synthetic polypeptide
AIQLTQSPSSLSASVGDRVTITCRASQDIRNDLGWYQQKPGKAPKLLIYAASSLQSGVPSRFSGSGSGTDFTLTISSLQPEDFATYYCLQDYNYPWTFGQGTKVEIKR

SEQ ID NO: 107 - DNA, 18 bp, synthetic construct
  misc_feature 1..18: note = Description of Artificial Sequence: Synthetic oligonucleotide
  source 1..18: mol_type = other DNA
caggacattagaaatgat

SEQ ID NO: 108 - protein, 6 aa, synthetic construct
  REGION 1..6: note = Description of Artificial Sequence: Synthetic peptide
QDIRND

SEQ ID NO: 109-110 - skipped (2 entries)

SEQ ID NO: 111 - DNA, 27 bp, synthetic construct
  misc_feature 1..27: note = Description of Artificial Sequence: Synthetic oligonucleotide
  source 1..27: mol_type = other DNA
ctacaagattacaattacccgtggacg

SEQ ID NO: 112 - protein, 9 aa, synthetic construct
  REGION 1..9: note = Description of Artificial Sequence: Synthetic peptide
LQDYNYPWT

SEQ ID NO: 113 - DNA, 351 bp, synthetic construct
  misc_feature 1..351: note = Description of Artificial Sequence: Synthetic polynucleotide
  source 1..351: mol_type = other DNA
gaggtgcagctggtggagtctgggggaggcttggtacagcctggggggtccctgagactctcctgtgcagtctctggattcaccctcagtagctacgatatgcactgggtccgccaacctacaggaaaaggtctggagtgggtctcagctattggttctactggtgacacatactatccaggctccgtgaagggccgattcaccatctccagagaaaatgccaagaactccttgtatcttcaaatgaacagcctgagagccggggacacggctgtgtattactgtgcaagagagggatgggacgtaccctttgacttctggggccagggaaccctggtcaccgtctcctca

SEQ ID NO: 114 - protein, 117 aa, synthetic construct
  REGION 1..117: note = Description of Artificial Sequence: Synthetic polypeptide
EVQLVESGGGLVQPGGSLRLSCAVSGFTLSSYDMHWVRQPTGKGLEWVSAIGSTGDTYYPGSVKGRFTISRENAKNSLYLQMNSLRAGDTAVYYCAREGWDVPFDFWGQGTLVTVSS

SEQ ID NO: 115 - DNA, 321 bp, synthetic construct
  misc_feature 1..321: note = Description of Artificial Sequence: Synthetic polynucleotide
  source 1..321: mol_type = other DNA
gccatccagatgacccagtctccatcctccctgtctgcatctgtaggagacagagtcaccatcacttgccgggcaagtcaggacattagaaatgatttaggctggtatcagcagaaaccagggaaagcccctaagctcctgatctatgctgcatccagtttacaaagtggggtcccatcacggttcagcggcagtggatctggcacagatttcactctcaccatcagcagcctgcagcctgaagattttgcaacttattactgtctacaagattacaattacccgtggacgttcggccaagggaccaaggtggaaatcaaa

SEQ ID NO: 116 - protein, 107 aa, synthetic construct
  REGION 1..107: note = Description of Artificial Sequence: Synthetic polypeptide
AIQMTQSPSSLSASVGDRVTITCRASQDIRNDLGWYQQKPGKAPKLLIYAASSLQSGVPSRFSGSGSGTDFTLTISSLQPEDFATYYCLQDYNYPWTFGQGTKVEIK

SEQ ID NO: 117 - DNA, 351 bp, synthetic construct
  misc_feature 1..351: note = Description of Artificial Sequence: Synthetic polynucleotide
  source 1..351: mol_type = other DNA
gaggtgcagctggtggagtctgggggaggcttggtacagcctggggggtccctgagactctcctgtgcagcctctggattcaccctcagtagctacgatatgcactgggtccgccaagctacaggaaaaggtctggagtgggtctcagctattggttctactggtgacacatactatccaggctccgtgaagggccgattcaccatctccagagaaaatgccaagaactccttgtatcttcaaatgaacagcctgagagccggggacacggctgtgtattactgtgcaagagagggatgggacgtaccctttgacttctggggccagggaaccctggtcaccgtctcctca

SEQ ID NO: 118 - protein, 117 aa, synthetic construct
  REGION 1..117: note = Description of Artificial Sequence: Synthetic polypeptide
EVQLVESGGGLVQPGGSLRLSCAASGFTLSSYDMHWVRQATGKGLEWVSAIGSTGDTYYPGSVKGRFTISRENAKNSLYLQMNSLRAGDTAVYYCAREGWDVPFDFWGQGTLVTVSS

SEQ ID NO: 119 - DNA, 321 bp, synthetic construct
  misc_feature 1..321: note = Description of Artificial Sequence: Synthetic polynucleotide
  source 1..321: mol_type = other DNA
gccatccagatgacccagtctccatcctccctgtctgcatctgtaggagacagagtcaccatcacttgccgggcaagtcaggacattagaaatgatttaggctggtatcagcagaaaccagggaaagcccctaagctcctgatctatgctgcatccagtttacaaagtggggtcccatcaaggttcagcggcagtggatctggcacagatttcactctcaccatcagcagcctgcagcctgaagattttgcaacttattactgtctacaagattacaattacccgtggacgttcggccaagggaccaaggtggaaatcaaa

SEQ ID NO: 120 - protein, 107 aa, synthetic construct; identical to SEQ ID NO: 116
  REGION 1..107: note = Description of Artificial Sequence: Synthetic polypeptide

SEQ ID NO: 121 - DNA, 384 bp, synthetic construct
  misc_feature 1..384: note = Description of Artificial Sequence: Synthetic polynucleotide
  source 1..384: mol_type = other DNA
caggtgcagctgcaggagtcggggccaggactggtgaagccttcggagaccctgtccctcacctgcactgtctctggggactccatcaatacttactactggagctggttccggcagcccccagggaagggactggagtggattgggtatatctattatagtggaaccaccaactacaacccctccctcaagagtcgagtcaccatatcaatagacacgcccaggaaccagttctccctgaagctgatctctgtgaccgcagcggacacggccgtgtattactgtgcgagagagaggattactatgattcggggagttaccctctactattactcctacggtatggacgtctggggccaagggaccacggtcaccgtctcctca

SEQ ID NO: 122 - protein, 128 aa, synthetic construct
  REGION 1..128: note = Description of Artificial Sequence: Synthetic polypeptide
QVQLQESGPGLVKPSETLSLTCTVSGDSINTYYWSWFRQPPGKGLEWIGYIYYSGTTNYNPSLKSRVTISIDTPRNQFSLKLISVTAADTAVYYCARERITMIRGVTLYYYSYGMDVWGQGTTVTVSS

SEQ ID NO: 123 - DNA, 24 bp, synthetic construct
  misc_feature 1..24: note = Description of Artificial Sequence: Synthetic oligonucleotide
  source 1..24: mol_type = other DNA
ggggactccatcaatacttactac

SEQ ID NO: 124 - protein, 8 aa, synthetic construct
  REGION 1..8: note = Description of Artificial Sequence: Synthetic peptide
GDSINTYY

SEQ ID NO: 125 - DNA, 21 bp, synthetic construct
  misc_feature 1..21: note = Description of Artificial Sequence: Synthetic oligonucleotide
  source 1..21: mol_type = other DNA
atctattatagtggaaccacc

SEQ ID NO: 126 - protein, 7 aa, synthetic construct
  REGION 1..7: note = Description of Artificial Sequence: Synthetic peptide
IYYSGTT

SEQ ID NO: 127 - DNA, 66 bp, synthetic construct
  misc_feature 1..66: note = Description of Artificial Sequence: Synthetic oligonucleotide
  source 1..66: mol_type = other DNA
gcgagagagaggattactatgattcggggagttaccctctactattactcctacggtatggacgtc

SEQ ID NO: 128 - protein, 22 aa, synthetic construct
  REGION 1..22: note = Description of Artificial Sequence: Synthetic peptide
ARERITMIRGVTLYYYSYGMDV

SEQ ID NO: 129 - DNA, 324 bp, synthetic construct
  misc_feature 1..324: note = Description of Artificial Sequence: Synthetic polynucleotide
  source 1..324: mol_type = other DNA
gacatccagatgacccagtctccatccttcctgtctgcatctgtaggagacagagtcaccatcacttgctgggccagtcaggacattagcagttatttagcctggtatcagcaaaaaccagggatagcccctaagctcctgatctatgctgcatccactttgcaaagtggggtcccatcaaggttcggcggcagtggatctgggacagaattcactctcacaatcagcagcctgcagcctgaagattttgcaacttattactgtcaacagcttaatagttaccctcggacgttcggccaagggaccaaggtggaaatcaaacga

SEQ ID NO: 130 - protein, 108 aa, synthetic construct
  REGION 1..108: note = Description of Artificial Sequence: Synthetic polypeptide
DIQMTQSPSFLSASVGDRVTITCWASQDISSYLAWYQQKPGIAPKLLIYAASTLQSGVPSRFGGSGSGTEFTLTISSLQPEDFATYYCQQLNSYPRTFGQGTKVEIKR

SEQ ID NO: 131 - DNA, 18 bp, synthetic construct
  misc_feature 1..18: note = Description of Artificial Sequence: Synthetic oligonucleotide
  source 1..18: mol_type = other DNA
caggacattagcagttat

SEQ ID NO: 132 - protein, 6 aa, synthetic construct
  REGION 1..6: note = Description of Artificial Sequence: Synthetic peptide
QDISSY

SEQ ID NO: 133-134 - skipped (2 entries)

SEQ ID NO: 135 - DNA, 27 bp, synthetic construct
  misc_feature 1..27: note = Description of Artificial Sequence: Synthetic oligonucleotide
  source 1..27: mol_type = other DNA
caacagcttaatagttaccctcggacg

SEQ ID NO: 136 - protein, 9 aa, synthetic construct
  REGION 1..9: note = Description of Artificial Sequence: Synthetic peptide
QQLNSYPRT

SEQ ID NO: 137 - DNA, 384 bp, synthetic construct; identical to SEQ ID NO: 121
  misc_feature 1..384: note = Description of Artificial Sequence: Synthetic polynucleotide
  source 1..384: mol_type = other DNA

SEQ ID NO: 138 - protein, 128 aa, synthetic construct; identical to SEQ ID NO: 122
  REGION 1..128: note = Description of Artificial Sequence: Synthetic polypeptide

SEQ ID NO: 139 - DNA, 321 bp, synthetic construct
  misc_feature 1..321: note = Description of Artificial Sequence: Synthetic polynucleotide
  source 1..321: mol_type = other DNA
gacatccagatgacccagtctccatccttcctgtctgcatctgtaggagacagagtcaccatcacttgctgggccagtcaggacattagcagttatttagcctggtatcagcaaaaaccagggatagcccctaagctcctgatctatgctgcatccactttgcaaagtggggtcccatcaaggttcggcggcagtggatctgggacagaattcactctcacaatcagcagcctgcagcctgaagattttgcaacttattactgtcaacagcttaatagttaccctcggacgttcggccaagggaccaaggtggaaatcaaa

SEQ ID NO: 140 - protein, 107 aa, synthetic construct
  REGION 1..107: note = Description of Artificial Sequence: Synthetic polypeptide
DIQMTQSPSFLSASVGDRVTITCWASQDISSYLAWYQQKPGIAPKLLIYAASTLQSGVPSRFGGSGSGTEFTLTISSLQPEDFATYYCQQLNSYPRTFGQGTKVEIK

SEQ ID NO: 141 - DNA, 384 bp, synthetic construct
  misc_feature 1..384: note = Description of Artificial Sequence: Synthetic polynucleotide
  source 1..384: mol_type = other DNA
caggtgcagctgcaggagtcgggcccaggactggtgaagccttcggagaccctgtccctcacctgcactgtctctggggactccatcaatacttactactggagctggatccggcagcccccagggaagggactggagtggattgggtatatctattatagtggaaccaccaactacaacccctccctcaagagtcgagtcaccatatcagtagacacgtccaagaaccagttctccctgaagctgagctctgtgaccgctgcggacacggccgtgtattactgtgcgagagagaggattactatgattcggggagttaccctctactattactcctacggtatggacgtctggggccaagggaccacggtcaccgtctcctca

SEQ ID NO: 142 - protein, 128 aa, synthetic construct
  REGION 1..128: note = Description of Artificial Sequence: Synthetic polypeptide
QVQLQESGPGLVKPSETLSLTCTVSGDSINTYYWSWIRQPPGKGLEWIGYIYYSGTTNYNPSLKSRVTISVDTSKNQFSLKLSSVTAADTAVYYCARERITMIRGVTLYYYSYGMDVWGQGTTVTVSS

SEQ ID NO: 143 - DNA, 321 bp, synthetic construct
  misc_feature 1..321: note = Description of Artificial Sequence: Synthetic polynucleotide
  source 1..321: mol_type = other DNA
gacatccagatgacccagtctccatcctccctgtctgcatctgtaggagacagagtcaccatcacttgccgggcaagtcaggacattagcagttatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtcaacagcttaatagttaccctcggacgttcggccaagggaccaaggtggaaatcaaa

SEQ ID NO: 144 - protein, 107 aa, synthetic construct
  REGION 1..107: note = Description of Artificial Sequence: Synthetic polypeptide
DIQMTQSPSSLSASVGDRVTITCRASQDISSYLGWYQQKPGKAPKRLIYAASSLQSGVPSRFSGSGSGTEFTLTISSLQPEDFATYYCQQLNSYPRTFGQGTKVEIK

SEQ ID NO: 145 - DNA, 378 bp, synthetic construct
  misc_feature 1..378: note = Description of Artificial Sequence: Synthetic polynucleotide
  source 1..378: mol_type = other DNA
caggtgcagctggtgcagtctggagctgaggtgaagaagcctggggcctcagtgaaggtctcctgcaaggcttctggttacacctttaccaactatggtatcagctgggtgcgacaggcccctggacaaggacttgagttaatgggatggattagtggttacaatggtaacacaaactatgcacaagaactccaggccagagtcaccatgaccacagacacatccacgagcacagcctacatggagctgaggaacctgagatctgacgacacggccgtatattactgtgcgagagatagagtcgttgtagcagctgctaattactacttttattctatggacgtctggggccaagggaccacggtcaccgtctcctca

SEQ ID NO: 146 - protein, 126 aa, synthetic construct
  REGION 1..126: note = Description of Artificial Sequence: Synthetic polypeptide
QVQLVQSGAEVKKPGASVKVSCKASGYTFTNYGISWVRQAPGQGLELMGWISGYNGNTNYAQELQARVTMTTDTSTSTAYMELRNLRSDDTAVYYCARDRVVVAAANYYFYSMDVWGQGTTVTVSS

SEQ ID NO: 147 - DNA, 24 bp, synthetic construct
  misc_feature 1..24: note = Description of Artificial Sequence: Synthetic oligonucleotide
  source 1..24: mol_type = other DNA
ggttacacctttaccaactatggt

SEQ ID NO: 148 - protein, 8 aa, synthetic construct
  REGION 1..8: note = Description of Artificial Sequence: Synthetic peptide
GYTFTNYG

SEQ ID NO: 149 - DNA, 24 bp, synthetic construct
  misc_feature 1..24: note = Description of Artificial Sequence: Synthetic oligonucleotide
  source 1..24: mol_type = other DNA
attagtggttacaatggtaacaca

SEQ ID NO: 150 - protein, 8 aa, synthetic construct
  REGION 1..8: note = Description of Artificial Sequence: Synthetic peptide
ISGYNGNT

SEQ ID NO: 151 - DNA, 57 bp, synthetic construct
  misc_feature 1..57: note = Description of Artificial Sequence: Synthetic oligonucleotide
  source 1..57: mol_type = other DNA
gcgagagatagagtcgttgtagcagctgctaattactacttttattctatggacgtc

SEQ ID NO: 152 - protein, 19 aa, synthetic construct
  REGION 1..19: note = Description of Artificial Sequence: Synthetic peptide
ARDRVVVAAANYYFYSMDV

SEQ ID NO: 153 - DNA, 339 bp, synthetic construct
  misc_feature 1..339: note = Description of Artificial Sequence: Synthetic polynucleotide
  source 1..339: mol_type = other DNA
gccatccagatgacccagtctccactctccctgtccgtcacccttggacagccggcctccatctcctgcaggtctagtcaaagcctcgtatacagtgatggagacacctacttgaattggtttcagcagaggccaggccaatctccaaggcgcctaatttataaggtttctaaccgggactctggggtcccagacagattcagcggcagtgggtcaggcactgctttcacactgaaaatcagcggggtggaggccgaggatgttggggtttactactgcatgcaagctacacactggcctcggacgttcggccaagggaccaaggtggaaatcaaacga

SEQ ID NO: 154 - protein, 113 aa, synthetic construct
  REGION 1..113: note = Description of Artificial Sequence: Synthetic polypeptide
AIQMTQSPLSLSVTLGQPASISCRSSQSLVYSDGDTYLNWFQQRPGQSPRRLIYKVSNRDSGVPDRFSGSGSGTAFTLKISGVEAEDVGVYYCMQATHWPRTFGQGTKVEIKR

SEQ ID NO: 155 - DNA, 33 bp, synthetic construct
  misc_feature 1..33: note = Description of Artificial Sequence: Synthetic oligonucleotide
  source 1..33: mol_type = other DNA
caaagcctcgtatacagtgatggagacacctac

SEQ ID NO: 156 - protein, 11 aa, synthetic construct
  REGION 1..11: note = Description of Artificial Sequence: Synthetic peptide
QSLVYSDGDTY

SEQ ID NO: 157-158 - skipped (2 entries)

SEQ ID NO: 159 - DNA, 27 bp, synthetic construct
  misc_feature 1..27: note = Description of Artificial Sequence: Synthetic oligonucleotide
  source 1..27: mol_type = other DNA
atgcaagctacacactggcctcggacg

SEQ ID NO: 160 - protein, 9 aa, synthetic construct
  REGION 1..9: note = Description of Artificial Sequence: Synthetic peptide
MQATHWPRT

SEQ ID NO: 161 - DNA, 378 bp, synthetic construct
  misc_feature 1..378: note = Description of Artificial Sequence: Synthetic polynucleotide
  source 1..378: mol_type = other DNA
caggttcagctggtgcagtctggagctgaggtgaagaagcctggggcctcagtgaaggtctcctgcaaggcttctggttacacctttaccaactatggtatcagctgggtgcgacaggcccctggacaaggacttgagttaatgggatggattagtggttacaatggtaacacaaactatgcacaagaactccaggccagagtcaccatgaccacagacacatccacgagcacagcctacatggagctgaggaacctgagatctgacgacacggccgtatattactgtgcgagagatagagtcgttgtagcagctgctaattactacttttattctatggacgtctggggccaagggaccacggtcaccgtctcctca

SEQ ID NO: 162 - protein, 126 aa, synthetic construct; identical to SEQ ID NO: 146
  REGION 1..126: note = Description of Artificial Sequence: Synthetic polypeptide

SEQ ID NO: 163 - DNA, 336 bp, synthetic construct
  misc_feature 1..336: note = Description of Artificial Sequence: Synthetic polynucleotide
  source 1..336: mol_type = other DNA
gatgttgtgatgactcagtctccactctccctgtccgtcacccttggacagccggcctccatctcctgcaggtctagtcaaagcctcgtatacagtgatggagacacctacttgaattggtttcagcagaggccaggccaatctccaaggcgcctaatttataaggtttctaaccgggactctggggtcccagacagattcagcggcagtgggtcaggcactgctttcacactgaaaatcagcggggtggaggccgaggatgttggggtttactactgcatgcaagctacacactggcctcggacgttcggccaagggaccaaggtggaaatcaaa

SEQ ID NO: 164 - protein, 112 aa, synthetic construct
  REGION 1..112: note = Description of Artificial Sequence: Synthetic polypeptide
DVVMTQSPLSLSVTLGQPASISCRSSQSLVYSDGDTYLNWFQQRPGQSPRRLIYKVSNRDSGVPDRFSGSGSGTAFTLKISGVEAEDVGVYYCMQATHWPRTFGQGTKVEIK

SEQ ID NO: 165 - DNA, 378 bp, synthetic construct
  misc_feature 1..378: note = Description of Artificial Sequence: Synthetic polynucleotide
  source 1..378: mol_type = other DNA
caggttcagctggtgcagtctggagctgaggtgaagaagcctggggcctcagtgaaggtctcctgcaaggcttctggttacacctttaccaactatggtatcagctgggtgcgacaggcccctggacaagggcttgagtggatgggatggattagtggttacaatggtaacacaaactatgcacagaagctccagggcagagtcaccatgaccacagacacatccacgagcacagcctacatggagctgaggagcctgagatctgacgacacggccgtgtattactgtgcgagagatagagtcgttgtagcagctgctaattactacttttattctatggacgtctggggccaagggaccacggtcaccgtctcctca

SEQ ID NO: 166 - protein, 126 aa, synthetic construct
  REGION 1..126: note = Description of Artificial Sequence: Synthetic polypeptide
QVQLVQSGAEVKKPGASVKVSCKASGYTFTNYGISWVRQAPGQGLEWMGWISGYNGNTNYAQKLQGRVTMTTDTSTSTAYMELRSLRSDDTAVYYCARDRVVVAAANYYFYSMDVWGQGTTVTVSS

SEQ ID NO: 167 - DNA, 336 bp, synthetic construct
  misc_feature 1..336: note = Description of Artificial Sequence: Synthetic polynucleotide
  source 1..336: mol_type = other DNA
gatgttgtgatgactcagtctccactctccctgcccgtcacccttggacagccggcctccatctcctgcaggtctagtcaaagcctcgtatacagtgatggagacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaagctacacactggcctcggacgttcggccaagggaccaaggtggaaatcaaa

SEQ ID NO: 168 - protein, 112 aa, synthetic construct
  REGION 1..112: note = Description of Artificial Sequence: Synthetic polypeptide
DVVMTQSPLSLPVTLGQPASISCRSSQSLVYSDGDTYLNWFQQRPGQSPRRLIYKVSNRDSGVPDRFSGSGSGTDFTLKISRVEAEDVGVYYCMQATHWPRTFGQGTKVEIK

SEQ ID NO: 169 - DNA, 375 bp, synthetic construct
  misc_feature 1..375: note = Description of Artificial Sequence: Synthetic polynucleotide
  source 1..375: mol_type = other DNA
caggtccacttgaaggagtctggtcctacgctggtgaaacccacacagaccctcacgctgacctgcaccttctctggattctcactcatcactagtggagtgggtgtgggctggattcgtcagccccccggaaaggccctggagtggcttgcactcatttattggaatggtgataagcgctacagcccatctctgaagagcaggctcaccatcaccaaggacacctccaaaaaccaggtggtccttacaatgaccaacatggaccctgtggacacagccacatattactgtgcacacaggataactgaaactagttactacttctactacggtatggacgtctggggccaagggaccacggtcaccgtctcctca

SEQ ID NO: 170 - protein, 125 aa, synthetic construct
  REGION 1..125: note = Description of Artificial Sequence: Synthetic polypeptide
QVHLKESGPTLVKPTQTLTLTCTFSGFSLITSGVGVGWIRQPPGKALEWLALIYWNGDKRYSPSLKSRLTITKDTSKNQVVLTMTNMDPVDTATYYCAHRITETSYYFYYGMDVWGQGTTVTVSS

SEQ ID NO: 171 - DNA, 30 bp, synthetic construct
  misc_feature 1..30: note = Description of Artificial Sequence: Synthetic oligonucleotide
  source 1..30: mol_type = other DNA
ggattctcactcatcactagtggagtgggt

SEQ ID NO: 172 - protein, 10 aa, synthetic construct
  REGION 1..10: note = Description of Artificial Sequence: Synthetic peptide
GFSLITSGVG

SEQ ID NO: 173 - DNA, 21 bp, synthetic construct
  misc_feature 1..21: note = Description of Artificial Sequence: Synthetic oligonucleotide
  source 1..21: mol_type = other DNA
atttattggaatggtgataag

SEQ ID NO: 174 - protein, 7 aa, synthetic construct
  REGION 1..7: note = Description of Artificial Sequence: Synthetic peptide
IYWNGDK

SEQ ID NO: 175 - DNA, 51 bp, synthetic construct
  misc_feature 1..51: note = Description of Artificial Sequence: Synthetic oligonucleotide
  source 1..51: mol_type = other DNA
gcacacaggataactgaaactagttactacttctactacggtatggacgtc

SEQ ID NO: 176 - protein, 17 aa, synthetic construct
  REGION 1..17: note = Description of Artificial Sequence: Synthetic peptide
AHRITETSYYFYYGMDV

SEQ ID NO: 177 - DNA, 339 bp, synthetic construct
  misc_feature 1..339: note = Description of Artificial Sequence: Synthetic polynucleotide
  source 1..339: mol_type = other DNA
gacatccagatgacccagtctccactctccctgcccgtcacccctggagagccggcctccatctcctgcaggtctagtcagagcctcctgcatagtcatggatacgactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgctcactttcggcggagggaccaaggtggaaatcaaacga

SEQ ID NO: 178 - protein, 113 aa, synthetic construct
  REGION 1..113: note = Description of Artificial Sequence: Synthetic polypeptide
DIQMTQSPLSLPVTPGEPASISCRSSQSLLHSHGYDYLDWYLQKPGQSPQLLIYLGSNRASGVPDRFSGSGSGTDFTLKISRVEAEDVGVYYCMQALQTPLTFGGGTKVEIKR

SEQ ID NO: 179 - DNA, 33 bp, synthetic construct
  misc_feature 1..33: note = Description of Artificial Sequence: Synthetic oligonucleotide
  source 1..33: mol_type = other DNA
cagagcctcctgcatagtcatggatacgactat

SEQ ID NO: 180 - protein, 11 aa, synthetic construct
  REGION 1..11: note = Description of Artificial Sequence: Synthetic peptide
QSLLHSHGYDY

SEQ ID NO: 181-182 - skipped (2 entries)

SEQ ID NO: 183 - DNA, 27 bp, synthetic construct
  misc_feature 1..27: note = Description of Artificial Sequence: Synthetic oligonucleotide
  source 1..27: mol_type = other DNA
atgcaagctctacaaactccgctcact

SEQ ID NO: 184 - protein, 9 aa, synthetic construct
  REGION 1..9: note = Description of Artificial Sequence: Synthetic peptide
MQALQTPLT

SEQ ID NO: 185 - DNA, 375 bp, synthetic construct
  misc_feature 1..375: note = Description of Artificial Sequence: Synthetic polynucleotide
  source 1..375: mol_type = other DNA
cagatcaccttgaaggagtctggtcctacgctggtgaaacccacacagaccctcacgctgacctgcaccttctctggattctcactcatcactagtggagtgggtgtgggctggattcgtcagccccccggaaaggccctggagtggcttgcactcatttattggaatggtgataagcgctacagcccatctctgaagagcaggctcaccatcaccaaggacacctccaaaaaccaggtggtccttacaatgaccaacatggaccctgtggacacagccacatattactgtgcacacaggataactgaaactagttactacttctactacggtatggacgtctggggccaagggaccacggtcaccgtctcctca

SEQ ID NO: 186 - protein, 125 aa, synthetic construct
  REGION 1..125: note = Description of Artificial Sequence: Synthetic polypeptide
QITLKESGPTLVKPTQTLTLTCTFSGFSLITSGVGVGWIRQPPGKALEWLALIYWNGDKRYSPSLKSRLTITKDTSKNQVVLTMTNMDPVDTATYYCAHRITETSYYFYYGMDVWGQGTTVTVSS

SEQ ID NO: 187 - DNA, 336 bp, synthetic construct
  misc_feature 1..336: note = Description of Artificial Sequence: Synthetic polynucleotide
  source 1..336: mol_type = other DNA
gatattgtgatgactcagtctccactctccctgcccgtcacccctggagagccggcctccatctcctgcaggtctagtcagagcctcctgcatagtcatggatacgactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgctcactttcggcggagggaccaaggtggagatcaaa

SEQ ID NO: 188 - protein, 112 aa, synthetic construct
  REGION 1..112: note = Description of Artificial Sequence: Synthetic polypeptide
DIVMTQSPLSLPVTPGEPASISCRSSQSLLHSHGYDYLDWYLQKPGQSPQLLIYLGSNRASGVPDRFSGSGSGTDFTLKISRVEAEDVGVYYCMQALQTPLTFGGGTKVEIK

SEQ ID NO: 189 - DNA, 375 bp, synthetic construct
  misc_feature 1..375: note = Description of Artificial Sequence: Synthetic polynucleotide
  source 1..375: mol_type = other DNA
cagatcaccttgaaggagtctggtcctacgctggtgaaacccacacagaccctcacgctgacctgcaccttctctggattctcactcatcactagtggagtgggtgtgggctggatccgtcagcccccaggaaaggccctggagtggcttgcactcatttattggaatggtgataagcgctacagcccatctctgaagagcaggctcaccatcaccaaggacacctccaaaaaccaggtggtccttacaatgaccaacatggaccctgtggacacagccacatattactgtgcacacaggataactgaaactagttactacttctactacggtatggacgtctggggccaagggaccacggtcaccgtctcctca

SEQ ID NO: 190 - protein, 125 aa, synthetic construct; identical to SEQ ID NO: 186
  REGION 1..125: note = Description of Artificial Sequence: Synthetic polypeptide

SEQ ID NO: 191 - DNA, 336 bp, synthetic construct; identical to SEQ ID NO: 187
  misc_feature 1..336: note = Description of Artificial Sequence: Synthetic polynucleotide
  source 1..336: mol_type = other DNA

SEQ ID NO: 192 - protein, 112 aa, synthetic construct; identical to SEQ ID NO: 188
  REGION 1..112: note = Description of Artificial Sequence: Synthetic polypeptide

SEQ ID NO: 193 - DNA, 375 bp, synthetic construct
  misc_feature 1..375: note = Description of Artificial Sequence: Synthetic polynucleotide
  source 1..375: mol_type = other DNA
cagatcaccttgaaggagtctggtcctactctggtgaaaccctcacagaccctcacgctgacctgcaccttctctgggttctcactcagcactagtggagtgggtgtgggctggatccgtcagcccccaggaaaggccctggagtggcttgcactcatttattggaattctgataagcgctacagcccatctctgaagagcaggctcaccatcaccaaggacacctccaaaaaccaggtagtccttacaatgaccaacatggaccctgtggacacagccacatattactgtgcacacagacatgacagctcgtcctactacttctactacggtatggacgtctggggccaagggatcacggtcaccgtctcctca

SEQ ID NO: 194 - protein, 125 aa, synthetic construct
  REGION 1..125: note = Description of Artificial Sequence: Synthetic polypeptide
QITLKESGPTLVKPSQTLTLTCTFSGFSLSTSGVGVGWIRQPPGKALEWLALIYWNSDKRYSPSLKSRLTITKDTSKNQVVLTMTNMDPVDTATYYCAHRHDSSSYYFYYGMDVWGQGITVTVSS

SEQ ID NO: 195 - DNA, 30 bp, synthetic construct
  misc_feature 1..30: note = Description of Artificial Sequence: Synthetic oligonucleotide
  source 1..30: mol_type = other DNA
gggttctcactcagcactagtggagtgggt

SEQ ID NO: 196 - protein, 10 aa, synthetic construct
  REGION 1..10: note = Description of Artificial Sequence: Synthetic peptide
GFSLSTSGVG

SEQ ID NO: 197 - DNA, 21 bp, synthetic construct
  misc_feature 1..21: note = Description of Artificial Sequence: Synthetic oligonucleotide
  source 1..21: mol_type = other DNA
atttattggaattctgataag

SEQ ID NO: 198 - protein, 7 aa, synthetic construct
  REGION 1..7: note = Description of Artificial Sequence: Synthetic peptide
IYWNSDK

SEQ ID NO: 199 - DNA, 51 bp, synthetic construct
  misc_feature 1..51: note = Description of Artificial Sequence: Synthetic oligonucleotide
  source 1..51: mol_type = other DNA
gcacacagacatgacagctcgtcctactacttctactacggtatggacgtc

SEQ ID NO: 200 - protein, 17 aa, synthetic construct
  REGION 1..17: note = Description of Artificial Sequence: Synthetic peptide
AHRHDSSSYYFYYGMDV

SEQ ID NO: 201 - DNA, 339 bp, synthetic construct
  misc_feature 1..339: note = Description of Artificial Sequence: Synthetic polynucleotide
  source 1..339: mol_type = other DNA
gacatccagatgacccagtctccgctctccctgcccgtcacccctggagagccggcctccatctcctgcaggtctagtcagagcctcctccatagtcatggatacaactatttggattggtacctgcagaagccagggcagtctccacaactcctgatctatttgggttctaatcgggcctccggggtccctgacaggttcagtggcggtggatcaggcacagattttacactgaaaatcagcagagtggaggctgaggatgttgggatttattactgcatgcaagctctacagactcctctcactttcggcggagggaccaaggtggagatcaaacga

SEQ ID NO: 202 - protein, 113 aa, synthetic construct
  REGION 1..113: note = Description of Artificial Sequence: Synthetic polypeptide
DIQMTQSPLSLPVTPGEPASISCRSSQSLLHSHGYNYLDWYLQKPGQSPQLLIYLGSNRASGVPDRFSGGGSGTDFTLKISRVEAEDVGIYYCMQALQTPLTFGGGTKVEIKR

SEQ ID NO: 203 - DNA, 33 bp, synthetic construct
  misc_feature 1..33: note = Description of Artificial Sequence: Synthetic oligonucleotide
  source 1..33: mol_type = other DNA
cagagcctcctccatagtcatggatacaactat

SEQ ID NO: 204 - protein, 11 aa, synthetic construct
  REGION 1..11: note = Description of Artificial Sequence: Synthetic peptide
QSLLHSHGYNY

SEQ ID NO: 205-206 - skipped (2 entries)

SEQ ID NO: 207 - DNA, 27 bp, synthetic construct
  misc_feature 1..27: note = Description of Artificial Sequence: Synthetic oligonucleotide
  source 1..27: mol_type = other DNA
atgcaagctctacagactcctctcact

SEQ ID NO: 208 - protein, 9 aa, synthetic construct
  REGION 1..9: note = Description of Artificial Sequence: Synthetic peptide
MQALQTPLT

SEQ ID NO: 209 - DNA, 375 bp, synthetic construct
  misc_feature 1..375: note = Description of Artificial Sequence: Synthetic polynucleotide
  source 1..375: mol_type = other DNA
cagatcaccttgaaggagtctggtcctactctggtgaaaccctcacagaccctcacgctgacctgcaccttctctgggttctcactcagcactagtggagtgggtgtgggctggatccgtcagcccccaggaaaggccctggagtggcttgcactcatttattggaattctgataagcgctacagcccatctctgaagagcaggctcaccatcaccaaggacacctccaaaaaccaggtagtccttacaatgaccaacatggaccctgtggacacagccacatattactgtgcacacagacatgacagctcgtcctactacttctactacggtatggacgtctggggccaagggaccacggtcaccgtctcctca

SEQ ID NO: 210 - protein, 125 aa, synthetic construct
  REGION 1..125: note = Description of Artificial Sequence: Synthetic polypeptide
QITLKESGPTLVKPSQTLTLTCTFSGFSLSTSGVGVGWIRQPPGKALEWLALIYWNSDKRYSPSLKSRLTITKDTSKNQVVLTMTNMDPVDTATYYCAHRHDSSSYYFYYGMDVWGQGTTVTVSS

SEQ ID NO: 211 - DNA, 336 bp, synthetic construct
  misc_feature 1..336: note = Description of Artificial Sequence: Synthetic polynucleotide
  source 1..336: mol_type = other DNA
gatattgtgatgactcagtctccgctctccctgcccgtcacccctggagagccggcctccatctcctgcaggtctagtcagagcctcctccatagtcatggatacaactatttggattggtacctgcagaagccagggcagtctccacaactcctgatctatttgggttctaatcgggcctccggggtccctgacaggttcagtggcggtggatcaggcacagattttacactgaaaatcagcagagtggaggctgaggatgttgggatttattactgcatgcaagctctacagactcctctcactttcggcggagggaccaaggtggagatcaaa

SEQ ID NO: 212 - protein, 112 aa, synthetic construct
  REGION 1..112: note = Description of Artificial Sequence: Synthetic polypeptide
DIVMTQSPLSLPVTPGEPASISCRSSQSLLHSHGYNYLDWYLQKPGQSPQLLIYLGSNRASGVPDRFSGGGSGTDFTLKISRVEAEDVGIYYCMQALQTPLTFGGGTKVEIK

SEQ ID NO: 213 - DNA, 375 bp, synthetic construct
  misc_feature 1..375: note = Description of Artificial Sequence: Synthetic polynucleotide
  source 1..375: mol_type = other DNA
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattggaattctgataagcgctacagcccatctctgaagagcaggctcaccatcaccaaggacacctccaaaaaccaggtggtccttacaatgaccaacatggaccctgtggacacagccacatattactgtgcacacagacatgacagctcgtcctactacttctactacggtatggacgtctggggccaagggaccacggtcaccgtctcctca

SEQ ID NO: 214 - protein, 125 aa, synthetic construct
  REGION 1..125: note = Description of Artificial Sequence: Synthetic polypeptide
QITLKESGPTLVKPTQTLTLTCTFSGFSLSTSGVGVGWIRQPPGKALEWLALIYWNSDKRYSPSLKSRLTITKDTSKNQVVLTMTNMDPVDTATYYCAHRHDSSSYYFYYGMDVWGQGTTVTVSS

SEQ ID NO: 215 - DNA, 336 bp, synthetic construct
  misc_feature 1..336: note = Description of Artificial Sequence: Synthetic polynucleotide
  source 1..336: mol_type = other DNA
gatattgtgatgactcagtctccactctccctgcccgtcacccctggagagccggcctccatctcctgcaggtctagtcagagcctcctccatagtcatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacagactcctctcactttcggcggagggaccaaggtggagatcaaa

SEQ ID NO: 216 - protein, 112 aa, synthetic construct
  REGION 1..112: note = Description of Artificial Sequence: Synthetic polypeptide
DIVMTQSPLSLPVTPGEPASISCRSSQSLLHSHGYNYLDWYLQKPGQSPQLLIYLGSNRASGVPDRFSGSGSGTDFTLKISRVEAEDVGVYYCMQALQTPLTFGGGTKVEIK

SEQ ID NO: 217 - DNA, 381 bp, synthetic construct
  misc_feature 1..381: note = Description of Artificial Sequence: Synthetic polynucleotide
  source 1..381: mol_type = other DNA
gagatgcaactggtggagtctgggggaggcttggtccagcctggggggtccctgagactctcctgtgcagcctctggattcacctttagtagtcactggatgaagtgggtccgccaggctccagggaaggggctggagtgggtggccaacataaaccaagatggaagtgagaaatactatgtggactctgtgaagggccgattcaccatctccagagacaacgccaagaactcactgtttctgcaaatgaacagcctgagagccgaggacacggctgtgtattactgtgcgagagatattgtactaatggtctatgatatggactactactactacggtatggacgtctggggccaagggaccacggtcaccgtctcctca

SEQ ID NO: 218 - protein, 127 aa, synthetic construct
  REGION 1..127: note = Description of Artificial Sequence: Synthetic polypeptide
EMQLVESGGGLVQPGGSLRLSCAASGFTFSSHWMKWVRQAPGKGLEWVANINQDGSEKYYVDSVKGRFTISRDNAKNSLFLQMNSLRAEDTAVYYCARDIVLMVYDMDYYYYGMDVWGQGTTVTVSS

SEQ ID NO: 219 - DNA, 24 bp, synthetic construct
  misc_feature 1..24: note = Description of Artificial Sequence: Synthetic oligonucleotide
  source 1..24: mol_type = other DNA
ggattcacctttagtagtcactgg

SEQ ID NO: 220 - protein, 8 aa, synthetic construct
  REGION 1..8: note = Description of Artificial Sequence: Synthetic peptide
GFTFSSHW

SEQ ID NO: 221 - DNA, 24 bp, synthetic construct
  misc_feature 1..24: note = Description of Artificial Sequence: Synthetic oligonucleotide
  source 1..24: mol_type = other DNA
ataaaccaagatggaagtgagaaa

SEQ ID NO: 222 - protein, 8 aa, synthetic construct
  REGION 1..8: note = Description of Artificial Sequence: Synthetic peptide
INQDGSEK

SEQ ID NO: 223 - DNA, 60 bp, synthetic construct
  misc_feature 1..60: note = Description of Artificial Sequence: Synthetic oligonucleotide
  source 1..60: mol_type = other DNA
gcgagagatattgtactaatggtctatgatatggactactactactacggtatggacgtc

SEQ ID NO: 224 - protein, 20 aa, synthetic construct
  REGION 1..20: note = Description of Artificial Sequence: Synthetic peptide
ARDIVLMVYDMDYYYYGMDV

SEQ ID NO: 225 - DNA, 336 bp, synthetic construct
  misc_feature 1..336: note = Description of Artificial Sequence: Synthetic polynucleotide
  source 1..336: mol_type = other DNA
gatattgtgatgactcagtctccactctccctgcccgtcacccctggagagccggcctccatctcctgcaggtctagtcagagcctcctgcatagtaatggaaacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaaactctacaaactccgctcactttcggcggagggaccaaggtggagatcaaa

SEQ ID NO: 226 - protein, 112 aa, synthetic construct
  REGION 1..112: note = Description of Artificial Sequence: Synthetic polypeptide
DIVMTQSPLSLPVTPGEPASISCRSSQSLLHSNGNNYLDWYLQKPGQSPQLLIYLGSNRASGVPDRFSGSGSGTDFTLKISRVEAEDVGVYYCMQTLQTPLTFGGGTKVEIK

SEQ ID NO: 227 - DNA, 33 bp, synthetic construct
  misc_feature 1..33: note = Description of Artificial Sequence: Synthetic oligonucleotide
  source 1..33: mol_type = other DNA
cagagcctcctgcatagtaatggaaacaactat

SEQ ID NO: 228 - protein, 11 aa, synthetic construct
  REGION 1..11: note = Description of Artificial Sequence: Synthetic peptide
SSQSLLHSNGN

SEQ ID NO: 229-230 - skipped (2 entries)

SEQ ID NO: 231 - DNA, 27 bp, synthetic construct
  misc_feature 1..27: note = Description of Artificial Sequence: Synthetic oligonucleotide
  source 1..27: mol_type = other DNA
atgcaaactctacaaactccgctcact

SEQ ID NO: 232 - protein, 9 aa, synthetic construct
  REGION 1..9: note = Description of Artificial Sequence: Synthetic peptide
MQTLQTPLT

SEQ ID NO: 233 - DNA, 381 bp, synthetic construct
  misc_feature 1..381: note = Description of Artificial Sequence: Synthetic polynucleotide
  source 1..381: mol_type = other DNA
gaggtgcagctggtggagtctgggggaggcttggtccagcctggggggtccctgagactctcctgtgcagcctctggattcacctttagtagtcactggatgaagtgggtccgccaggctccagggaaggggctggagtgggtggccaacataaaccaagatggaagtgagaaatactatgtggactctgtgaagggccgattcaccatctccagagacaacgccaagaactcactgtttctgcaaatgaacagcctgagagccgaggacacggctgtgtattactgtgcgagagatattgtactaatggtctatgatatggactactactactacggtatggacgtctggggccaagggaccacggtcaccgtctcctca

SEQ ID NO: 234 - protein, 127 aa, synthetic construct
  REGION 1..127: note = Description of Artificial Sequence: Synthetic polypeptide
EVQLVESGGGLVQPGGSLRLSCAASGFTFSSHWMKWVRQAPGKGLEWVANINQDGSEKYYVDSVKGRFTISRDNAKNSLFLQMNSLRAEDTAVYYCARDIVLMVYDMDYYYYGMDVWGQGTTVTVSS

SEQ ID NO: 235 - DNA, 336 bp, synthetic construct; identical to SEQ ID NO: 225
  misc_feature 1..336: note = Description of Artificial Sequence: Synthetic polynucleotide
  source 1..336: mol_type = other DNA

SEQ ID NO: 236 - protein, 112 aa, synthetic construct; identical to SEQ ID NO: 226
  REGION 1..112: note = Description of Artificial Sequence: Synthetic polypeptide

SEQ ID NO: 237 - DNA, 381 bp, synthetic construct
  misc_feature 1..381: note = Description of Artificial Sequence: Synthetic polynucleotide
  source 1..381: mol_type = other DNA
gaggtgcagctggtggagtctgggggaggcttggtccagcctggggggtccctgagactctcctgtgcagcctctggattcacctttagtagtcactggatgagctgggtccgccaggctccagggaaggggctggagtgggtggccaacataaaccaagatggaagtgagaaatactatgtggactctgtgaagggccgattcaccatctccagagacaacgccaagaactcactgtatctgcaaatgaacagcctgagagccgaggacacggctgtgtattactgtgcgagagatattgtactaatggtctatgatatggactactactactacggtatggacgtctgggggcaagggaccacggtcaccgtctcctca

SEQ ID NO: 238 - protein, 127 aa, synthetic construct
  REGION 1..127: note = Description of Artificial Sequence: Synthetic polypeptide
EVQLVESGGGLVQPGGSLRLSCAASGFTFSSHWMSWVRQAPGKGLEWVANINQDGSEKYYVDSVKGRFTISRDNAKNSLYLQMNSLRAEDTAVYYCARDIVLMVYDMDYYYYGMDVWGQGTTVTVSS

SEQ ID NO: 239 - DNA, 336 bp, synthetic construct; identical to SEQ ID NO: 225
  misc_feature 1..336: note = Description of Artificial Sequence: Synthetic polynucleotide
  source 1..336: mol_type = other DNA

SEQ ID NO: 240 - protein, 112 aa, synthetic construct; identical to SEQ ID NO: 226
  REGION 1..112: note = Description of Artificial Sequence: Synthetic polypeptide

SEQ ID NO: 241 - DNA, 381 bp, synthetic construct
  misc_feature 1..381: note = Description of Artificial Sequence: Synthetic polynucleotide
  source 1..381: mol_type = other DNA
caggtgcagctggtggagtctgggggaggcgtggtccagcctgggaggtccctgagactctcctgtgcagtctctggattcaccttcagtagctatggcatgcactgggtccgccaggctccaggcaaggggctggagtgggtggcagctatatcatatgatggaagtaataaatactatgtagactccgtgaagggccgattcaccatctccagagacaattccaagaaaacgctgtatctgcaaatgaacagcctgagagctgaggacacggctgtgtataattgtgcgaaaaatattgtactagtgatgtatgatatagactatcactactatgggatggacgtctggggccaagggaccacggtcaccgtctcctca

SEQ ID NO: 242 - protein, 127 aa, synthetic construct
  REGION 1..127: note = Description of Artificial Sequence: Synthetic polypeptide
QVQLVESGGGVVQPGRSLRLSCAVSGFTFSSYGMHWVRQAPGKGLEWVAAISYDGSNKYYVDSVKGRFTISRDNSKKTLYLQMNSLRAEDTAVYNCAKNIVLVMYDIDYHYYGMDVWGQGTTVTVSS

SEQ ID NO: 243 - DNA, 24 bp, synthetic construct
  misc_feature 1..24: note = Description of Artificial Sequence: Synthetic oligonucleotide
  source 1..24: mol_type = other DNA
ggattcaccttcagtagctatggc

SEQ ID NO: 244 - protein, 8 aa, synthetic construct
  REGION 1..8: note = Description of Artificial Sequence: Synthetic peptide
GFTFSSYG

SEQ ID NO: 245 - DNA, 24 bp, synthetic construct
  misc_feature 1..24: note = Description of Artificial Sequence: Synthetic oligonucleotide
  source 1..24: mol_type = other DNA
atatcatatgatggaagtaataaa

SEQ ID NO: 246 - protein, 8 aa, synthetic construct
  REGION 1..8: note = Description of Artificial Sequence: Synthetic peptide
ISYDGSNK

SEQ ID NO: 247 - DNA, 60 bp, synthetic construct
  misc_feature 1..60: note = Description of Artificial Sequence: Synthetic oligonucleotide
  source 1..60: mol_type = other DNA
gcgaaaaatattgtactagtgatgtatgatatagactatcactactatgggatggacgtc

SEQ ID NO: 248 - protein, 20 aa, synthetic construct
  REGION 1..20: note = Description of Artificial Sequence: Synthetic peptide
AKNIVLVMYDIDYHYYGMDV

SEQ ID NO: 249 - DNA, 336 bp, synthetic construct
  misc_feature 1..336: note = Description of Artificial Sequence: Synthetic polynucleotide
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacaactcctgatctatttgggttttaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaga

SEQ ID NO: 250 - protein, 112 aa, synthetic construct
  REGION 1..112: note = Description of Artificial Sequence: Synthetic polypeptide
DIVMTQSPLSLPVTPGEPASISCRSSQSLLHSNGYNYLDWYLQKPGQSPQLLIYLGFNRASGVPDRFSGSGSGTDFTLKISRVEAEDVGVYYCMQALQTPLTFGGGTKVEIR

SEQ ID NO: 251 - DNA, 33 bp, synthetic construct
  misc_feature 1..33: note = Description of Artificial Sequence: Synthetic oligonucleotide
  source 1..33: mol_type = other DNA
cagagcctcctgcatagtaatggatacaactat

SEQ ID NO: 252 - protein, 11 aa, synthetic construct
  REGION 1..11: note = Description of Artificial Sequence: Synthetic peptide
QSLLHSNGYNY

SEQ ID NO: 253-254 - skipped (2 entries)

SEQ ID NO: 255 - DNA, 27 bp, synthetic construct
  misc_feature 1..27: note = Description of Artificial Sequence: Synthetic oligonucleotide
  source 1..27: mol_type = other DNA
atgcaagctctacaaactcctctcact

SEQ ID NO: 256 - protein, 9 aa, synthetic construct
  REGION 1..9: note = Description of Artificial Sequence: Synthetic peptide
MQALQTPLT

SEQ ID NO: 257 - DNA, 381 bp, synthetic construct; identical to SEQ ID NO: 241
  misc_feature 1..381: note = Description of Artificial Sequence: Synthetic polynucleotide
  source 1..381: mol_type = other DNA

SEQ ID NO: 258 - protein, 127 aa, synthetic construct; identical to SEQ ID NO: 242
  REGION 1..127: note = Description of Artificial Sequence: Synthetic polypeptide

SEQ ID NO: 259 - DNA, 336 bp, synthetic construct
  misc_feature 1..336: note = Description of Artificial Sequence: Synthetic polynucleotide
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacaactcctgatctatttgggttttaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaaa

SEQ ID NO: 260 - protein, 112 aa, synthetic construct
  REGION 1..112: note = Description of Artificial Sequence: Synthetic polypeptide
DIVMTQSPLSLPVTPGEPASISCRSSQSLLHSNGYNYLDWYLQKPGQSPQLLIYLGFNRASGVPDRFSGSGSGTDFTLKISRVEAEDVGVYYCMQALQTPLTFGGGTKVEIK

SEQ ID NO: 261 - DNA, 381 bp, synthetic construct
  misc_feature 1..381: note = Description of Artificial Sequence: Synthetic polynucleotide
  source 1..381: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaaaatattgtactagtgatgtatgatatagactatcactactatgggatggacgtctgggggcaagggaccacggtcaccgtctcctca

SEQ ID NO: 262 - protein, 127 aa, synthetic construct
  REGION 1..127: note = Description of Artificial Sequence: Synthetic polypeptide
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKNIVLVMYDIDYHYYGMDVWGQGTTVTVSS

SEQ ID NO: 263 - DNA, 336 bp, synthetic construct
  misc_feature 1..336: note = Description of Artificial Sequence: Synthetic polynucleotide
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttttaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaaa

SEQ ID NO: 264 - protein, 112 aa, synthetic construct; identical to SEQ ID NO: 260
  REGION 1..112: note = Description of Artificial Sequence: Synthetic polypeptide

SEQ ID NO: 265 - DNA, 381 bp, synthetic construct; identical to SEQ ID NO: 241
  misc_feature 1..381: note = Description of Artificial Sequence: Synthetic polynucleotide
  source 1..381: mol_type = other DNA

SEQ ID NO: 266 - protein, 127 aa, synthetic construct; identical to SEQ ID NO: 242
  REGION 1..127: note = Description of Artificial Sequence: Synthetic polypeptide

SEQ ID NO: 267 - DNA, 24 bp, synthetic construct
  misc_feature 1..24: note = Description of Artificial Sequence: Synthetic oligonucleotide
  source 1..24: mol_type = other DNA
ggattcaccttcagtagctatggc

SEQ ID NO: 268 - protein, 8 aa, synthetic construct
  REGION 1..8: note = Description of Artificial Sequence: Synthetic peptide
GFTFSSYG

SEQ ID NO: 269 - DNA, 24 bp, synthetic construct
  misc_feature 1..24: note = Description of Artificial Sequence: Synthetic oligonucleotide
  source 1..24: mol_type = other DNA
atatcatatgatggaagtaataaa

SEQ ID NO: 270 - protein, 8 aa, synthetic construct
  REGION 1..8: note = Description of Artificial Sequence: Synthetic peptide
ISYDGSNK

SEQ ID NO: 271 - DNA, 60 bp, synthetic construct; identical to SEQ ID NO: 247
  misc_feature 1..60: note = Description of Artificial Sequence: Synthetic oligonucleotide
  source 1..60: mol_type = other DNA

SEQ ID NO: 272 - protein, 20 aa, synthetic construct
  REGION 1..20: note = Description of Artificial Sequence: Synthetic peptide
AKNIVLVMYDIDYHYYGMDV

SEQ ID NO: 273 - DNA, 336 bp, synthetic construct; identical to SEQ ID NO: 249
  misc_feature 1..336: note = Description of Artificial Sequence: Synthetic polynucleotide
  source 1..336: mol_type = other DNA

SEQ ID NO: 274 - protein, 112 aa, synthetic construct; identical to SEQ ID NO: 250
  REGION 1..112: note = Description of Artificial Sequence: Synthetic polypeptide

SEQ ID NO: 275 - DNA, 33 bp, synthetic construct; identical to SEQ ID NO: 251
  misc_feature 1..33: note = Description of Artificial Sequence: Synthetic oligonucleotide
  source 1..33: mol_type = other DNA

SEQ ID NO: 276 - protein, 11 aa, synthetic construct
  REGION 1..11: note = Description of Artificial Sequence: Synthetic peptide
QSLLHSNGYNY

SEQ ID NO: 277-278 - skipped (2 entries)

SEQ ID NO: 279 - DNA, 27 bp, synthetic construct
  misc_feature 1..27: note = Description of Artificial Sequence: Synthetic oligonucleotide
  source 1..27: mol_type = other DNA
atgcaagctctacaaactcctctcact

SEQ ID NO: 280 - protein, 9 aa, synthetic construct
  REGION 1..9: note = Description of Artificial Sequence: Synthetic peptide
MQALQTPLT

SEQ ID NO: 281 - DNA, 381 bp, synthetic construct; identical to SEQ ID NO: 241
  misc_feature 1..381: note = Description of Artificial Sequence: Synthetic polynucleotide
  source 1..381: mol_type = other DNA

SEQ ID NO: 282 - protein, 127 aa, synthetic construct; identical to SEQ ID NO: 242
  REGION 1..127: note = Description of Artificial Sequence: Synthetic polypeptide

SEQ ID NO: 283 - DNA, 336 bp, synthetic construct; identical to SEQ ID NO: 259
  misc_feature 1..336: note = Description of Artificial Sequence: Synthetic polynucleotide
  source 1..336: mol_type = other DNA

SEQ ID NO: 284 - protein, 112 aa, synthetic construct; identical to SEQ ID NO: 260
  REGION 1..112: note = Description of Artificial Sequence: Synthetic polypeptide

SEQ ID NO: 285 - DNA, 381 bp, synthetic construct; identical to SEQ ID NO: 261
  misc_feature 1..381: note = Description of Artificial Sequence: Synthetic polynucleotide
  source 1..381: mol_type = other DNA

SEQ ID NO: 286 - protein, 127 aa, synthetic construct; identical to SEQ ID NO: 262
  REGION 1..127: note = Description of Artificial Sequence: Synthetic polypeptide

SEQ ID NO: 287 - DNA, 336 bp, synthetic construct; identical to SEQ ID NO: 263
  misc_feature 1..336: note = Description of Artificial Sequence: Synthetic polynucleotide
  source 1..336: mol_type = other DNA

SEQ ID NO: 288 - protein, 112 aa, synthetic construct; identical to SEQ ID NO: 260
  REGION 1..112: note = Description of Artificial Sequence: Synthetic polypeptide

SEQ ID NO: 289 - DNA, 372 bp, synthetic construct
  misc_feature 1..372: note = Description of Artificial Sequence: Synthetic polynucleotide
  source 1..372: mol_type = other DNA
cagatcaccttgaaggagtctggtcctacgctggtaaaacccacacagaccctcacgctgacctgcaccttctctgggttctcactcagcgctagtggagtgggtgtgggctggttccgtcagcccccaggaaaggccctggagtggcttgcactcatttattggaatgatgataagcgttacagcccatctctaaagaacagcctcaccatcaccaaggacacctccaaaaaccaggtggtccttacaatgaccaacatggaccctgtggacacagccacatattactgtgcacacagaatacatctatggtcctacttctactacggtatggacgtctggggccaagggaccacggtcaccgtctcctca

SEQ ID NO: 290 - protein, 124 aa, synthetic construct
  REGION 1..124: note = Description of Artificial Sequence: Synthetic polypeptide
QITLKESGPTLVKPTQTLTLTCTFSGFSLSASGVGVGWFRQPPGKALEWLALIYWNDDKRYSPSLKNSLTITKDTSKNQVVLTMTNMDPVDTATYYCAHRIHLWSYFYYGMDVWGQGTTVTVSS

SEQ ID NO: 291 - DNA, 30 bp, synthetic construct
  misc_feature 1..30: note = Description of Artificial Sequence: Synthetic oligonucleotide
  source 1..30: mol_type = other DNA
gggttctcactcagcgctagtggagtgggt

SEQ ID NO: 292 - protein, 10 aa, synthetic construct
  REGION 1..10: note = Description of Artificial Sequence: Synthetic peptide
GFSLSASGVG

SEQ ID NO: 293 - DNA, 21 bp, synthetic construct
  misc_feature 1..21: note = Description of Artificial Sequence: Synthetic oligonucleotide
  source 1..21: mol_type = other DNA
atttattggaatgatgataag

SEQ ID NO: 294 - protein, 7 aa, synthetic construct
  REGION 1..7: note = Description of Artificial Sequence: Synthetic peptide
IYWNDDK

SEQ ID NO: 295 - DNA, 48 bp, synthetic construct
  misc_feature 1..48: note = Description of Artificial Sequence: Synthetic oligonucleotide
  source 1..48: mol_type = other DNA
gcacacagaatacatctatggtcctacttctactacggtatggacgtc

SEQ ID NO: 296 - protein, 16 aa, synthetic construct
  REGION 1..16: note = Description of Artificial Sequence: Synthetic peptide
AHRIHLWSYFYYGMDV

SEQ ID NO: 297 - DNA, 336 bp, synthetic construct
  misc_feature 1..336: note = Description of Artificial Sequence: Synthetic polynucleotide
  source 1..336: mol_type = other DNA
gatattgtgatgactcagtctccactctccctgcccgtcacccctggagagccggcctccatctcctgcaggtctagtcagactctcctgcatagtaatggatacaactatttcgattggtacctgcagaagccagggcagtctccacagctcctgatctatttgggttctaatcgggcctccggggtccctgacagattcagtggcagtggatcaggcacagattttacactgaaaatcagcagagtggaggctgaggatgttggaatttattactgcatgcaagctctacaaactcctctcactttcggcggagggaccaaggtggagatcaga

SEQ ID NO: 298 - protein, 112 aa, synthetic construct
  REGION 1..112: note = Description of Artificial Sequence: Synthetic polypeptide
DIVMTQSPLSLPVTPGEPASISCRSSQTLLHSNGYNYFDWYLQKPGQSPQLLIYLGSNRASGVPDRFSGSGSGTDFTLKISRVEAEDVGIYYCMQALQTPLTFGGGTKVEIR

SEQ ID NO: 299 - DNA, 33 bp, synthetic construct
  misc_feature 1..33: note = Description of Artificial Sequence: Synthetic oligonucleotide
  source 1..33: mol_type = other DNA
cagactctcctgcatagtaatggatacaactat

SEQ ID NO: 300 - protein, 11 aa, synthetic construct
  REGION 1..11: note = Description of Artificial Sequence: Synthetic peptide
QTLLHSNGYNY

SEQ ID NO: 301-302 - skipped (2 entries)

SEQ ID NO: 303 - DNA, 27 bp, synthetic construct
  misc_feature 1..27: note = Description of Artificial Sequence: Synthetic oligonucleotide
  source 1..27: mol_type = other DNA
atgcaagctctacaaactcctctcact

SEQ ID NO: 304 - protein, 9 aa, synthetic construct
  REGION 1..9: note = Description of Artificial Sequence: Synthetic peptide
MQALQTPLT

SEQ ID NO: 305 - DNA, 372 bp, synthetic construct; identical to SEQ ID NO: 289
  misc_feature 1..372: note = Description of Artificial Sequence: Synthetic polynucleotide
  source 1..372: mol_type = other DNA

SEQ ID NO: 306 - protein, 124 aa, synthetic construct; identical to SEQ ID NO: 290
  REGION 1..124: note = Description of Artificial Sequence: Synthetic polypeptide

SEQ ID NO: 307 - DNA, 336 bp, synthetic construct
  misc_feature 1..336: note = Description of Artificial Sequence: Synthetic polynucleotide
  source 1..336: mol_type = other DNA
gatattgtgatgactcagtctccactctccctgcccgtcacccctggagagccggcctccatctcctgcaggtctagtcagactctcctgcatagtaatggatacaactatttcgattggtacctgcagaagccagggcagtctccacagctcctgatctatttgggttctaatcgggcctccggggtccctgacagattcagtggcagtggatcaggcacagattttacactgaaaatcagcagagtggaggctgaggatgttggaatttattactgcatgcaagctctacaaactcctctcactttcggcggagggaccaaggtggagatcaaa

SEQ ID NO: 308 - protein, 112 aa, synthetic construct
  REGION 1..112: note = Description of Artificial Sequence: Synthetic polypeptide
DIVMTQSPLSLPVTPGEPASISCRSSQTLLHSNGYNYFDWYLQKPGQSPQLLIYLGSNRASGVPDRFSGSGSGTDFTLKISRVEAEDVGIYYCMQALQTPLTFGGGTKVEIK

SEQ ID NO: 309 - DNA, 372 bp, synthetic construct
  misc_feature 1..372: note = Description of Artificial Sequence: Synthetic polynucleotide
  source 1..372: mol_type = other DNA
cagatcaccttgaaggagtctggtcctacgctggtgaaacccacacagaccctcacgctgacctgcaccttctctgggttctcactcagcgctagtggagtgggtgtgggctggatccgtcagcccccaggaaaggccctggagtggcttgcactcatttattggaatgatgataagcgctacagcccatctctgaagagcaggctcaccatcaccaaggacacctccaaaaaccaggtggtccttacaatgaccaacatggaccctgtggacacagccacatattactgtgcacacagaatacatctatggtcctacttctactacggtatggacgtctgggggcaagggaccacggtcaccgtctcctca

SEQ ID NO: 310 - protein, 124 aa, synthetic construct
  REGION 1..124: note = Description of Artificial Sequence: Synthetic polypeptide
QITLKESGPTLVKPTQTLTLTCTFSGFSLSASGVGVGWIRQPPGKALEWLALIYWNDDKRYSPSLKSRLTITKDTSKNQVVLTMTNMDPVDTATYYCAHRIHLWSYFYYGMDVWGQGTTVTVSS

SEQ ID NO: 311 - DNA, 336 bp, synthetic construct
  misc_feature 1..336: note = Description of Artificial Sequence: Synthetic polynucleotide
  source 1..336: mol_type = other DNA
gatattgtgatgactcagtctccactctccctgcccgtcacccctggagagccggcctccatctcctgcaggtctagtcagactctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaaa

SEQ ID NO: 312 - protein, 112 aa, synthetic construct
  REGION 1..112: note = Description of Artificial Sequence: Synthetic polypeptide
DIVMTQSPLSLPVTPGEPASISCRSSQTLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 313 - DNA, 381 bp, synthetic construct
  misc_feature 1..381: note = Description of Artificial Sequence: Synthetic polynucleotide
  source 1..381: mol_type = other DNA
caggttcagctggtgcagtctggacctgaggtgaagaaccctggggcctcagtgaaggtctcctgcaaggcttctggttacacctttaccacctatggtatcagttgggtacgacaggcccctggacaagggcttgagtggatgggatggatcagcggttacaatggtaaaacaaacgatgcacagaagttccaggacagagtcgccatgaccacagacacatccacgagcacagcctacatggagctgaggagcctgagatctgacgacacggccatttattactgttcgagagatcgtttagtagtaccacctgcccttaattattcctactacgttatggacgtctggggccaagggaccacggtcaccgtctcctca

SEQ ID NO: 314 - protein, 127 aa, synthetic construct
  REGION 1..127: note = Description of Artificial Sequence: Synthetic polypeptide
QVQLVQSGPEVKNPGASVKVSCKASGYTFTTYGISWVRQAPGQGLEWMGWISGYNGKTNDAQKFQDRVAMTTDTSTSTAYMELRSLRSDDTAIYYCSRDRLVVPPALNYSYYVMDVWGQGTTVTVSS

SEQ ID NO: 315 - DNA, 24 bp, synthetic construct
  misc_feature 1..24: note = Description of Artificial Sequence: Synthetic oligonucleotide
  source 1..24: mol_type = other DNA
ggttacacctttaccacctatggt

SEQ ID NO: 316 - protein, 8 aa, synthetic construct
  REGION 1..8: note = Description of Artificial Sequence: Synthetic peptide
GYTFTTYG

SEQ ID NO: 317 - DNA, 24 bp, synthetic construct
  misc_feature 1..24: note = Description of Artificial Sequence: Synthetic oligonucleotide
  source 1..24: mol_type = other DNA
atcagcggttacaatggtaaaaca

SEQ ID NO: 318 - protein, 8 aa, synthetic construct
  REGION 1..8: note = Description of Artificial Sequence: Synthetic peptide
ISGYNGKT

SEQ ID NO: 319 - DNA, 60 bp, synthetic construct
  misc_feature 1..60: note = Description of Artificial Sequence: Synthetic oligonucleotide
  source 1..60: mol_type = other DNA
tcgagagatcgtttagtagtaccacctgcccttaattattcctactacgttatggacgtc

SEQ ID NO: 320 - protein, 20 aa, synthetic construct
  REGION 1..20: note = Description of Artificial Sequence: Synthetic peptide
SRDRLVVPPALNYSYYVMDV

SEQ ID NO: 321 - DNA, 336 bp, synthetic construct
  misc_feature 1..336: note = Description of Artificial Sequence: Synthetic polynucleotide
  source 1..336: mol_type = other DNA
gatgttgtgatgactcagtctccactctccctgcccgtcacccttggacagccggcctccatctcctgcaggtctagtcaaagcctcgtatacagtgatggaaacacctacttgaattggtctcagcagaggccaggtcaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgtacacttttggccaggggaccaagctggagatcaaa

SEQ ID NO: 322 - protein, 112 aa, synthetic construct
  REGION 1..112: note = Description of Artificial Sequence: Synthetic polypeptide
DVVMTQSPLSLPVTLGQPASISCRSSQSLVYSDGNTYLNWSQQRPGQSPRRLIYKVSNRDSGVPDRFSGSGSGTDFTLKISRVEAEDVGVYYCMQGTHWPYTFGQGTKLEIK

SEQ ID NO: 323 - DNA, 33 bp, synthetic construct
  misc_feature 1..33: note = Description of Artificial Sequence: Synthetic oligonucleotide
  source 1..33: mol_type = other DNA
caaagcctcgtatacagtgatggaaacacctac

SEQ ID NO: 324 - protein, 11 aa, synthetic construct
  REGION 1..11: note = Description of Artificial Sequence: Synthetic peptide
QSLVYSDGNTY

SEQ ID NO: 325-326 - skipped (2 entries)

SEQ ID NO: 327 - DNA, 27 bp, synthetic construct
  misc_feature 1..27: note = Description of Artificial Sequence: Synthetic oligonucleotide
  source 1..27: mol_type = other DNA
atgcaaggtacacactggccgtacact

SEQ ID NO: 328 - protein, 9 aa, synthetic construct
  REGION 1..9: note = Description of Artificial Sequence: Synthetic peptide
MQGTHWPYT

SEQ ID NO: 329 - DNA, 381 bp, synthetic construct; identical to SEQ ID NO: 313
  misc_feature 1..381: note = Description of Artificial Sequence: Synthetic polynucleotide
  source 1..381: mol_type = other DNA

SEQ ID NO: 330 - protein, 127 aa, synthetic construct; identical to SEQ ID NO: 314
  REGION 1..127: note = Description of Artificial Sequence: Synthetic polypeptide

SEQ ID NO: 331 - DNA, 336 bp, synthetic construct; identical to SEQ ID NO: 321
  misc_feature 1..336: note = Description of Artificial Sequence: Synthetic polynucleotide
  source 1..336: mol_type = other DNA

SEQ ID NO: 332 - protein, 112 aa, synthetic construct; identical to SEQ ID NO: 322
  REGION 1..112: note = Description of Artificial Sequence: Synthetic polypeptide

SEQ ID NO: 333 - DNA, 381 bp, synthetic construct
  misc_feature 1..381: note = Description of Artificial Sequence: Synthetic polynucleotide
  source 1..381: mol_type = other DNA
caggttcagctggtgcagtctggagctgaggtgaagaagcctggggcctcagtgaaggtctcctgcaaggcttctggttacacctttaccacctatggtatcagctgggtgcgacaggcccctggacaagggcttgagtggatgggatggatcagcggttacaatggtaaaacaaactatgcacagaagctccagggcagagtcaccatgaccacagacacatccacgagcacagcctacatggagctgaggagcctgagatctgacgacacggccgtgtattactgttcgagagatcgtttagtagtaccacctgcccttaattattcctactacgttatggacgtctgggggcaagggaccacggtcaccgtctcctca

SEQ ID NO: 334 - protein, 127 aa, synthetic construct
  REGION 1..127: note = Description of Artificial Sequence: Synthetic polypeptide
QVQLVQSGAEVKKPGASVKVSCKASGYTFTTYGISWVRQAPGQGLEWMGWISGYNGKTNYAQKLQGRVTMTTDTSTSTAYMELRSLRSDDTAVYYCSRDRLVVPPALNYSYYVMDVWGQGTTVTVSS

SEQ ID NO: 335 - DNA, 336 bp, synthetic construct
  misc_feature 1..336: note = Description of Artificial Sequence: Synthetic polynucleotide
  source 1..336: mol_type = other DNA
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgtacacttttggccaggggaccaagctggagatcaaa

SEQ ID NO: 336 - protein, 112 aa, synthetic construct
  REGION 1..112: note = Description of Artificial Sequence: Synthetic polypeptide
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPYTFGQGTKLEIK

SEQ ID NO: 337 - DNA, 354 bp, synthetic construct
  misc_feature 1..354: note = Description of Artificial Sequence: Synthetic polynucleotide
  source 1..354: mol_type = other DNA
gaggtgcagctggtggagtctgggggaggcctggtcaagcctggggggtccctgagactctcctgtgcagcctctggattcaccttcagtagctatagcatggactgggtccgccaggctccagggaaggggctggagtgggtctcatccattagtagtagtagtagttacatatactacgcagactctgtgaagggccgattcaccatctccagagacaccgccaagaactcactgtatctgcaaatgaacagcctgagagacgaggacacggctgtttattactgtgcgagagagggcagtagcagactttttgactactggggccagggaaccctggtcaccgtctcctca

SEQ ID NO: 338 - protein, 118 aa, synthetic construct
  REGION 1..118: note = Description of Artificial Sequence: Synthetic polypeptide
EVQLVESGGGLVKPGGSLRLSCAASGFTFSSYSMDWVRQAPGKGLEWVSSISSSSSYIYYADSVKGRFTISRDTAKNSLYLQMNSLRDEDTAVYYCAREGSSRLFDYWGQGTLVTVSS

SEQ ID NO: 339 - DNA, 24 bp, synthetic construct
  misc_feature 1..24: note = Description of Artificial Sequence: Synthetic oligonucleotide
  source 1..24: mol_type = other DNA
ggattcaccttcagtagctatagc

SEQ ID NO: 340 - protein, 8 aa, synthetic construct
  REGION 1..8: note = Description of Artificial Sequence: Synthetic peptide
GFTFSSYS

SEQ ID NO: 341 - DNA, 24 bp, synthetic construct
  misc_feature 1..24: note = Description of Artificial Sequence: Synthetic oligonucleotide
  source 1..24: mol_type = other DNA
attagtagtagtagtagttacata

SEQ ID NO: 342 - protein, 8 aa, synthetic construct
  REGION 1..8: note = Description of Artificial Sequence: Synthetic peptide
ISSSSSYI

SEQ ID NO: 343 - DNA, 33 bp, synthetic construct
  misc_feature 1..33: note = Description of Artificial Sequence: Synthetic oligonucleotide
  source 1..33: mol_type = other DNA
gcgagagagggcagtagcagactttttgactac

SEQ ID NO: 344 - protein, 11 aa, synthetic construct
  REGION 1..11: note = Description of Artificial Sequence: Synthetic peptide
AREGSSRLFDY

SEQ ID NO: 345 - DNA, 321 bp, synthetic construct
  misc_feature 1..321: note = Description of Artificial Sequence: Synthetic polynucleotide
  source 1..321: mol_type = other DNA
gacatccagatgacccagtctccttccaccctgtctgcatctgtaggagacagagtcaccatcacttgccgggccagtcagagtattagtagctggttggcctggtatcagcagagaccagggaaagcccctaagctcctgatctataaggcgtctagtttagaaggtggagtcccatcaaggttcagcggcagtggatctgggacagaattcactctcaccatcagcagcctgcagcctgaggattttgcaacttattactgccaacagtataatagttattggtacacttttggccaggggaccaagctggagatcaaa

SEQ ID NO: 346 - protein, 107 aa, synthetic construct
  REGION 1..107: note = Description of Artificial Sequence: Synthetic polypeptide
DIQMTQSPSTLSASVGDRVTITCRASQSISSWLAWYQQRPGKAPKLLIYKASSLEGGVPSRFSGSGSGTEFTLTISSLQPEDFATYYCQQYNSYWYTFGQGTKLEIK

SEQ ID NO: 347 - DNA, 18 bp, synthetic construct
  misc_feature 1..18: note = Description of Artificial Sequence: Synthetic oligonucleotide
  source 1..18: mol_type = other DNA
cagagtattagtagctgg

SEQ ID NO: 348 - protein, 6 aa, synthetic construct
  REGION 1..6: note = Description of Artificial Sequence: Synthetic peptide
QSISSW

SEQ ID NO: 349-350 - skipped (2 entries)

SEQ ID NO: 351 - DNA, 27 bp, synthetic construct
  misc_feature 1..27: note = Description of Artificial Sequence: Synthetic oligonucleotide
  source 1..27: mol_type = other DNA
caacagtataatagttattggtacact

SEQ ID NO: 352 - protein, 9 aa, synthetic construct
  REGION 1..9: note = Description of Artificial Sequence: Synthetic peptide
QQYNSYWYT

SEQ ID NO: 353 - DNA, 354 bp, synthetic construct; identical to SEQ ID NO: 337
  misc_feature 1..354: note = Description of Artificial Sequence: Synthetic polynucleotide
  source 1..354: mol_type = other DNA

SEQ ID NO: 354 - protein, 118 aa, synthetic construct; identical to SEQ ID NO: 338
  REGION 1..118: note = Description of Artificial Sequence: Synthetic polypeptide

SEQ ID NO: 355 - DNA, 321 bp, synthetic construct; identical to SEQ ID NO: 345
  misc_feature 1..321: note = Description of Artificial Sequence: Synthetic polynucleotide
  source 1..321: mol_type = other DNA

SEQ ID NO: 356 - protein, 107 aa, synthetic construct; identical to SEQ ID NO: 346
  REGION 1..107: note = Description of Artificial Sequence: Synthetic polypeptide

SEQ ID NO: 357 - DNA, 354 bp, synthetic construct
  misc_feature 1..354: note = Description of Artificial Sequence: Synthetic polynucleotide
  source 1..354: mol_type = other DNA
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagagggcagtagcagactttttgactactggggccaaggaaccctggtcaccgtctcctca

SEQ ID NO: 358 - protein, 118 aa, synthetic construct
  REGION 1..118: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAREGSSRLFDYWGQGTLVTVSS

SEQ ID NO: 359 - DNA, 321 bp, synthetic construct
  misc_feature 1..321: note = Description of Artificial Sequence: Synthetic polynucleotide
  source 1..321: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattggtacacttttggccaggggaccaagctggagatcaaa

SEQ ID NO: 360 - protein, 107 aa, synthetic construct
  REGION 1..107: note = Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYWYTFGQGTKLEIK

SEQ ID NO: 361 - DNA, 384 bp, synthetic construct
  misc_feature 1..384: note = Description of Artificial Sequence: Synthetic polynucleotide
  source 1..384: mol_type = other DNA
caggtgcacctggtggagtctgggggaggcttggtcaagcctggagggtccctgagactctcctgtgcagcctctggattcaccttcagtgaccactacatgagctggatccgccaggctccagggaaggggctggagtggatttcatacattagtaatgatggtggtaccaaatactatgtggactctgtggagggccgattcatcatttccagggacaacgccaagaactcattgtatctacatatgaacagcctcagagccgacgacacggccgtgtattactgtgcgagagatcagggatatattggctacgactcgtattattactattcctacggtatggacgtctggggccaagggaccacggtcaccgtcgcctca

SEQ ID NO: 362 - protein, 128 aa, synthetic construct
  REGION 1..128: note = Description of Artificial Sequence: Synthetic polypeptide
QVHLVESGGGLVKPGGSLRLSCAASGFTFSDHYMSWIRQAPGKGLEWISYISNDGGTKYYVDSVEGRFIISRDNAKNSLYLHMNSLRADDTAVYYCARDQGYIGYDSYYYYSYGMDVWGQGTTVTVAS

SEQ ID NO: 363 - DNA, 24 bp, synthetic construct
  misc_feature 1..24: note = Description of Artificial Sequence: Synthetic oligonucleotide
  source 1..24: mol_type = other DNA
ggattcaccttcagtgaccactac

SEQ ID NO: 364 - protein, 8 aa, synthetic construct
  REGION 1..8: note = Description of Artificial Sequence: Synthetic peptide
GFTFSDHY

SEQ ID NO: 365 - DNA, 24 bp, synthetic construct
  misc_feature 1..24: note = Description of Artificial Sequence: Synthetic oligonucleotide
  source 1..24: mol_type = other DNA
attagtaatgatggtggtaccaaa

SEQ ID NO: 366 - protein, 8 aa, synthetic construct
  REGION 1..8: note = Description of Artificial Sequence: Synthetic peptide
ISNDGGTK

SEQ ID NO: 367 - DNA, 63 bp, synthetic construct
  misc_feature 1..63: note = Description of Artificial Sequence: Synthetic oligonucleotide
  source 1..63: mol_type = other DNA
gcgagagatcagggatatattggctacgactcgtattattactattcctacggtatggacgtc

SEQ ID NO: 368 - protein, 21 aa, synthetic construct
  REGION 1..21: note = Description of Artificial Sequence: Synthetic peptide
ARDQGYIGYDSYYYYSYGMDV

SEQ ID NO: 369 - DNA, 321 bp, synthetic construct
  misc_feature 1..321: note = Description of Artificial Sequence: Synthetic polynucleotide
  source 1..321: mol_type = other DNA
aaaattgtgttgacgcagtctccaggcaccctgcctttgtttccaggggaaagagccaccctctcctgtagggccagtcagagtgttaacaacaaattcttagcctggtaccagcagaaatctggccaggctcccaggctcctcatctatggtgcatccagcagggccactggcatcccagacaggttcagtggcagtgggtctgggaccgacttcactctcaccatcagcggactggagcctgaagattttgaagtgtattattgtcaagtatatggtaactcactcactctcggcggagggaccaaggtggagatcaag

SEQ ID NO: 370 - protein, 107 aa, synthetic construct
  REGION 1..107: note = Description of Artificial Sequence: Synthetic polypeptide
KIVLTQSPGTLPLFPGERATLSCRASQSVNNKFLAWYQQKSGQAPRLLIYGASSRATGIPDRFSGSGSGTDFTLTISGLEPEDFEVYYCQVYGNSLTLGGGTKVEIK

SEQ ID NO: 371 - DNA, 21 bp, synthetic construct
  misc_feature 1..21: note = Description of Artificial Sequence: Synthetic oligonucleotide
  source 1..21: mol_type = other DNA
cagagtgttaacaacaaattc

SEQ ID NO: 372 - protein, 7 aa, synthetic construct
  REGION 1..7: note = Description of Artificial Sequence: Synthetic peptide
QSVNNKF

SEQ ID NO: 373-374 - skipped (2 entries)

SEQ ID NO: 375 - DNA, 24 bp, synthetic construct
  misc_feature 1..24: note = Description of Artificial Sequence: Synthetic oligonucleotide
  source 1..24: mol_type = other DNA
caagtatatggtaactcactcact

SEQ ID NO: 376 - protein, 8 aa, synthetic construct
  REGION 1..8: note = Description of Artificial Sequence: Synthetic peptide
QVYGNSLT

SEQ ID NO: 377 - DNA, 384 bp, synthetic construct
  misc_feature 1..384: note = Description of Artificial Sequence: Synthetic polynucleotide
  source 1..384: mol_type = other DNA
caggtgcagctggtggagtctgggggaggcttggtcaagcctggagggtccctgagactctcctgtgcagcctctggattcaccttcagtgaccactacatgagctggatccgccaggctccagggaaggggctggagtggatttcatacattagtaatgatggtggtaccaaatactatgtggactctgtggagggccgattcatcatttccagggacaacgccaagaactcattgtatctacatatgaacagcctcagagccgacgacacggccgtgtattactgtgcgagagatcagggatatattggctacgactcgtattattactattcctacggtatggacgtctggggccaagggaccacggtcaccgtctcctca

SEQ ID NO: 378 - protein, 128 aa, synthetic construct
  REGION 1..128: note = Description of Artificial Sequence: Synthetic polypeptide
QVQLVESGGGLVKPGGSLRLSCAASGFTFSDHYMSWIRQAPGKGLEWISYISNDGGTKYYVDSVEGRFIISRDNAKNSLYLHMNSLRADDTAVYYCARDQGYIGYDSYYYYSYGMDVWGQGTTVTVSS

SEQ ID NO: 379 - DNA, 321 bp, synthetic construct
  misc_feature 1..321: note = Description of Artificial Sequence: Synthetic polynucleotide
  source 1..321: mol_type = other DNA
gaaattgtgttgacgcagtctccaggcaccctgcctttgtttccaggggaaagagccaccctctcctgtagggccagtcagagtgttaacaacaaattcttagcctggtaccagcagaaatctggccaggctcccaggctcctcatctatggtgcatccagcagggccactggcatcccagacaggttcagtggcagtgggtctgggaccgacttcactctcaccatcagcggactggagcctgaagattttgaagtgtattattgtcaagtatatggtaactcactcactctcggcggagggaccaaggtggagatcaaa

SEQ ID NO: 380 - protein, 107 aa, synthetic construct
  REGION 1..107: note = Description of Artificial Sequence: Synthetic polypeptide
EIVLTQSPGTLPLFPGERATLSCRASQSVNNKFLAWYQQKSGQAPRLLIYGASSRATGIPDRFSGSGSGTDFTLTISGLEPEDFEVYYCQVYGNSLTLGGGTKVEIK

SEQ ID NO: 381 - DNA, 384 bp, synthetic construct
  misc_feature 1..384: note = Description of Artificial Sequence: Synthetic polynucleotide
  source 1..384: mol_type = other DNA
caggtgcagctggtggagtctgggggaggcttggtcaagcctggagggtccctgagactctcctgtgcagcctctggattcaccttcagtgaccactacatgagctggatccgccaggctccagggaaggggctggagtgggtttcatacattagtaatgatggtggtaccaaatactacgcagactctgtgaagggccgattcaccatctccagggacaacgccaagaactcactgtatctgcaaatgaacagcctgagagccgaggacacggccgtgtattactgtgcgagagatcagggatatattggctacgactcgtattattactattcctacggtatggacgtctgggggcaagggaccacggtcaccgtctcctca

SEQ ID NO: 382 - protein, 128 aa, synthetic construct
  REGION 1..128: note = Description of Artificial Sequence: Synthetic polypeptide
QVQLVESGGGLVKPGGSLRLSCAASGFTFSDHYMSWIRQAPGKGLEWVSYISNDGGTKYYADSVKGRFTISRDNAKNSLYLQMNSLRAEDTAVYYCARDQGYIGYDSYYYYSYGMDVWGQGTTVTVSS

SEQ ID NO: 383 - DNA, 321 bp, synthetic construct
  misc_feature 1..321: note = Description of Artificial Sequence: Synthetic polynucleotide
  source 1..321: mol_type = other DNA
gaaattgtgttgacgcagtctccaggcaccctgtctttgtctccaggggaaagagccaccctctcctgcagggccagtcagagtgttaacaacaaattcttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcaagtatatggtaactcactcactttcggcggagggaccaaggtggagatcaaa

SEQ ID NO: 384 - protein, 107 aa, synthetic construct
  REGION 1..107: note = Description of Artificial Sequence: Synthetic polypeptide
EIVLTQSPGTLSLSPGERATLSCRASQSVNNKFLAWYQQKPGQAPRLLIYGASSRATGIPDRFSGSGSGTDFTLTISRLEPEDFAVYYCQVYGNSLTFGGGTKVEIK

SEQ ID NO: 385 - DNA, 360 bp, synthetic construct
  misc_feature 1..360: note = Description of Artificial Sequence: Synthetic polynucleotide
  source 1..360: mol_type = other DNA
gaggtgcagaaggtggagtctgggggaggcctggtcaagccgggggggtccctgagactctcctgtacagcctctggattcaccttcagtacttataacatgaattgggtccgccaggctccagggaagggactggagtgggtctcatccattaggagtagtagtaattacatatactacgcagactcagtgaagggccgattcaccatctccagagacaacgccaagaattcactgtatctgcaaatgaacagcctgagagccgatgacacggctgtgtattactgtgcgagagatggcagcagttggtacgactactctgactactggggccagggaaccctggtcaccgtctcctca

SEQ ID NO: 386 - protein, 120 aa, synthetic construct
  REGION 1..120: note = Description of Artificial Sequence: Synthetic polypeptide
EVQKVESGGGLVKPGGSLRLSCTASGFTFSTYNMNWVRQAPGKGLEWVSSIRSSSNYIYYADSVKGRFTISRDNAKNSLYLQMNSLRADDTAVYYCARDGSSWYDYSDYWGQGTLVTVSS

SEQ ID NO: 387 - DNA, 24 bp, synthetic construct
  misc_feature 1..24: note = Description of Artificial Sequence: Synthetic oligonucleotide
  source 1..24: mol_type = other DNA
ggattcaccttcagtacttataac

SEQ ID NO: 388 - protein, 8 aa, synthetic construct
  REGION 1..8: note = Description of Artificial Sequence: Synthetic peptide
GFTFSTYN

SEQ ID NO: 389 - DNA, 24 bp, synthetic construct
  misc_feature 1..24: note = Description of Artificial Sequence: Synthetic oligonucleotide
  source 1..24: mol_type = other DNA
attaggagtagtagtaattacata

SEQ ID NO: 390 - protein, 8 aa, synthetic construct
  REGION 1..8: note = Description of Artificial Sequence: Synthetic peptide
IRSSSNYI

SEQ ID NO: 391 - DNA, 39 bp, synthetic construct
  misc_feature 1..39: note = Description of Artificial Sequence: Synthetic oligonucleotide
  source 1..39: mol_type = other DNA
gcgagagatggcagcagttggtacgactactctgactac

SEQ ID NO: 392 - protein, 13 aa, synthetic construct
  REGION 1..13: note = Description of Artificial Sequence: Synthetic peptide
ARDGSSWYDYSDY

SEQ ID NO: 393 - DNA, 321 bp, synthetic construct
  misc_feature 1..321: note = Description of Artificial Sequence: Synthetic polynucleotide
  source 1..321: mol_type = other DNA
gacatccagatgacccagtctccttccaccctgtctgcatctgtaggagacagagtcaccatcacttgccgggccagtcagagtattagtagctggttggcctggtatcaacagataccagggaaagcccctaaactcctgatctataaggcgtctagtttagaaaatggggtcccatcaaggttcagcggcagtggatctgggacagaattcactctcatcatcagcagcctgcagcctgatgattttgcaacttattactgccaacagtatattagttattctcggacgttcggccaagggaccaaggtggaaatcaaa

SEQ ID NO: 394 - protein, 107 aa, synthetic construct
  REGION 1..107: note = Description of Artificial Sequence: Synthetic polypeptide
DIQMTQSPSTLSASVGDRVTITCRASQSISSWLAWYQQIPGKAPKLLIYKASSLENGVPSRFSGSGSGTEFTLIISSLQPDDFATYYCQQYISYSRTFGQGTKVEIK

SEQ ID NO: 395 - DNA, 18 bp, synthetic construct
  misc_feature 1..18: note = Description of Artificial Sequence: Synthetic oligonucleotide
  source 1..18: mol_type = other DNA
cagagtattagtagctgg

SEQ ID NO: 396 - protein, 6 aa, synthetic construct
  REGION 1..6: note = Description of Artificial Sequence: Synthetic peptide
QSISSW

SEQ ID NO: 397-398 - skipped (2 entries)

SEQ ID NO: 399 - DNA, 27 bp, synthetic construct
  misc_feature 1..27: note = Description of Artificial Sequence: Synthetic oligonucleotide
  source 1..27: mol_type = other DNA
caacagtatattagttattctcggacg

SEQ ID NO: 400 - protein, 9 aa, synthetic construct
  REGION 1..9: note = Description of Artificial Sequence: Synthetic peptide
QQYISYSRT

SEQ ID NO: 401 - DNA, 360 bp, synthetic construct
  misc_feature 1..360: note = Description of Artificial Sequence: Synthetic polynucleotide
  source 1..360: mol_type = other DNA
gaggtgcagctggtggagtctgggggaggcctggtcaagccgggggggtccctgagactctcctgtacagcctctggattcaccttcagtacttataacatgaattgggtccgccaggctccagggaagggactggagtgggtctcatccattaggagtagtagtaattacatatactacgcagactcagtgaagggccgattcaccatctccagagacaacgccaagaattcactgtatctgcaaatgaacagcctgagagccgatgacacggctgtgtattactgtgcgagagatggcagcagttggtacgactactctgactactggggccagggaaccctggtcaccgtctcctca

SEQ ID NO: 402 - protein, 120 aa, synthetic construct
  REGION 1..120: note = Description of Artificial Sequence: Synthetic polypeptide
EVQLVESGGGLVKPGGSLRLSCTASGFTFSTYNMNWVRQAPGKGLEWVSSIRSSSNYIYYADSVKGRFTISRDNAKNSLYLQMNSLRADDTAVYYCARDGSSWYDYSDYWGQGTLVTVSS

SEQ ID NO: 403 - DNA, 321 bp, synthetic construct; identical to SEQ ID NO: 393
  misc_feature 1..321: note = Description of Artificial Sequence: Synthetic polynucleotide
  source 1..321: mol_type = other DNA

SEQ ID NO: 404 - protein, 107 aa, synthetic construct; identical to SEQ ID NO: 394
  REGION 1..107: note = Description of Artificial Sequence: Synthetic polypeptide

SEQ ID NO: 405 - DNA, 360 bp, synthetic construct
  misc_feature 1..360: note = Description of Artificial Sequence: Synthetic polynucleotide
  source 1..360: mol_type = other DNA
gaggtgcagctggtggagtctgggggaggcctggtcaagcctggggggtccctgagactctcctgtgcagcctctggattcaccttcagtacttataacatgaactgggtccgccaggctccagggaaggggctggagtgggtctcatccattaggagtagtagtaattacatatactacgcagactcagtgaagggccgattcaccatctccagagacaacgccaagaactcactgtatctgcaaatgaacagcctgagagccgaggacacggctgtgtattactgtgcgagagatggcagcagttggtacgactactctgactactggggccaaggaaccctggtcaccgtctcctca

SEQ ID NO: 406 - protein, 120 aa, synthetic construct
  REGION 1..120: note = Description of Artificial Sequence: Synthetic polypeptide
EVQLVESGGGLVKPGGSLRLSCAASGFTFSTYNMNWVRQAPGKGLEWVSSIRSSSNYIYYADSVKGRFTISRDNAKNSLYLQMNSLRAEDTAVYYCARDGSSWYDYSDYWGQGTLVTVSS

SEQ ID NO: 407 - DNA, 321 bp, synthetic construct
  misc_feature 1..321: note = Description of Artificial Sequence: Synthetic polynucleotide
  source 1..321: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtatattagttattctcggacgttcggccaagggaccaaggtggaaatcaaa

SEQ ID NO: 408 - protein, 107 aa, synthetic construct
  REGION 1..107: note = Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYISYSRTFGQGTKVEIK

SEQ ID NO: 409 - DNA, 360 bp, synthetic construct; identical to SEQ ID NO: 401
  misc_feature 1..360: note = Description of Artificial Sequence: Synthetic polynucleotide
  source 1..360: mol_type = other DNA

SEQ ID NO: 410 - protein, 120 aa, synthetic construct; identical to SEQ ID NO: 402
  REGION 1..120: note = Description of Artificial Sequence: Synthetic polypeptide

SEQ ID NO: 411 - DNA, 24 bp, synthetic construct
  misc_feature 1..24: note = Description of Artificial Sequence: Synthetic oligonucleotide
  source 1..24: mol_type = other DNA
ggattcaccttcagtacttataac

SEQ ID NO: 412 - protein, 8 aa, synthetic construct
  REGION 1..8: note = Description of Artificial Sequence: Synthetic peptide
GFTFSTYN

SEQ ID NO: 413 - DNA, 24 bp, synthetic construct
  misc_feature 1..24: note = Description of Artificial Sequence: Synthetic oligonucleotide
  source 1..24: mol_type = other DNA
attaggagtagtagtaattacata

SEQ ID NO: 414 - protein, 8 aa, synthetic construct
  REGION 1..8: note = Description of Artificial Sequence: Synthetic peptide
IRSSSNYI

SEQ ID NO: 415 - DNA, 39 bp, synthetic construct; identical to SEQ ID NO: 391
  misc_feature 1..39: note = Description of Artificial Sequence: Synthetic oligonucleotide
  source 1..39: mol_type = other DNA

SEQ ID NO: 416 - protein, 13 aa, synthetic construct
  REGION 1..13: note = Description of Artificial Sequence: Synthetic peptide
ARDGSSWYDYSDY

SEQ ID NO: 417 - DNA, 321 bp, synthetic construct; identical to SEQ ID NO: 393
  misc_feature 1..321: note = Description of Artificial Sequence: Synthetic polynucleotide
  source 1..321: mol_type = other DNA

SEQ ID NO: 418 - protein, 107 aa, synthetic construct; identical to SEQ ID NO: 394
  REGION 1..107: note = Description of Artificial Sequence: Synthetic polypeptide

SEQ ID NO: 419 - DNA, 18 bp, synthetic construct
  misc_feature 1..18: note = Description of Artificial Sequence: Synthetic oligonucleotide
  source 1..18: mol_type = other DNA
cagagtattagtagctgg

SEQ ID NO: 420 - protein, 6 aa, synthetic construct
  REGION 1..6: note = Description of Artificial Sequence: Synthetic peptide
QSISSW

SEQ ID NO: 421-422 - skipped (2 entries)

SEQ ID NO: 423 - DNA, 27 bp, synthetic construct
  misc_feature 1..27: note = Description of Artificial Sequence: Synthetic oligonucleotide
  source 1..27: mol_type = other DNA
caacagtatattagttattctcggacg

SEQ ID NO: 424 - protein, 9 aa, synthetic construct
  REGION 1..9: note = Description of Artificial Sequence: Synthetic peptide
QQYISYSRT

SEQ ID NO: 425 - DNA, 360 bp, synthetic construct; identical to SEQ ID NO: 401
  misc_feature 1..360: note = Description of Artificial Sequence: Synthetic polynucleotide
  source 1..360: mol_type = other DNA

SEQ ID NO: 426 - protein, 120 aa, synthetic construct; identical to SEQ ID NO: 402
  REGION 1..120: note = Description of Artificial Sequence: Synthetic polypeptide

SEQ ID NO: 427 - DNA, 321 bp, synthetic construct; identical to SEQ ID NO: 393
  misc_feature 1..321: note = Description of Artificial Sequence: Synthetic polynucleotide
  source 1..321: mol_type = other DNA

SEQ ID NO: 428 - protein, 107 aa, synthetic construct; identical to SEQ ID NO: 394
  REGION 1..107: note = Description of Artificial Sequence: Synthetic polypeptide

SEQ ID NO: 429 - DNA, 360 bp, synthetic construct; identical to SEQ ID NO: 405
  misc_feature 1..360: note = Description of Artificial Sequence: Synthetic polynucleotide
  source 1..360: mol_type = other DNA

SEQ ID NO: 430 - protein, 120 aa, synthetic construct; identical to SEQ ID NO: 406
  REGION 1..120: note = Description of Artificial Sequence: Synthetic polypeptide

SEQ ID NO: 431 - DNA, 321 bp, synthetic construct; identical to SEQ ID NO: 407
  misc_feature 1..321: note = Description of Artificial Sequence: Synthetic polynucleotide
  source 1..321: mol_type = other DNA

SEQ ID NO: 432 - protein, 107 aa, synthetic construct; identical to SEQ ID NO: 408
  REGION 1..107: note = Description of Artificial Sequence: Synthetic polypeptide

SEQ ID NO: 433 - DNA, 360 bp, synthetic construct
  misc_feature 1..360: note = Description of Artificial Sequence: Synthetic polynucleotide
  source 1..360: mol_type = other DNA
gaggtgcagctggtggagtctgggggaggcctggtcaagccgggggggtccctgagactctcctgtacagcctctggattcaccttcagtacttataacatgaattgggtccgccaggctccagggaagggactggagtgggtctcatccattaggagtagtagtaattacatatactacgcagactcagtgaagggccgattcaccatctccagagacaacgccaagagttcactgtatctgcaaatgaacagcctgagagccgaggacacggctgtgtattactgtgcgagagatggcagcagttggtacgactactctgactactggggccagggaaccctggtcaccgtctcctca

SEQ ID NO: 434 - protein, 120 aa, synthetic construct
  REGION 1..120: note = Description of Artificial Sequence: Synthetic polypeptide
EVQLVESGGGLVKPGGSLRLSCTASGFTFSTYNMNWVRQAPGKGLEWVSSIRSSSNYIYYADSVKGRFTISRDNAKSSLYLQMNSLRAEDTAVYYCARDGSSWYDYSDYWGQGTLVTVSS

SEQ ID NO: 435 - DNA, 24 bp, synthetic construct
  misc_feature 1..24: note = Description of Artificial Sequence: Synthetic oligonucleotide
  source 1..24: mol_type = other DNA
ggattcaccttcagtacttataac

SEQ ID NO: 436 - protein, 8 aa, synthetic construct
  REGION 1..8: note = Description of Artificial Sequence: Synthetic peptide
GFTFSTYN

SEQ ID NO: 437 - DNA, 24 bp, synthetic construct
  misc_feature 1..24: note = Description of Artificial Sequence: Synthetic oligonucleotide
  source 1..24: mol_type = other DNA
attaggagtagtagtaattacata

SEQ ID NO: 438 - protein, 8 aa, synthetic construct
  REGION 1..8: note = Description of Artificial Sequence: Synthetic peptide
IRSSSNYI

SEQ ID NO: 439 - DNA, 39 bp, synthetic construct; identical to SEQ ID NO: 391
  misc_feature 1..39: note = Description of Artificial Sequence: Synthetic oligonucleotide
  source 1..39: mol_type = other DNA

SEQ ID NO: 440 - protein, 13 aa, synthetic construct
  REGION 1..13: note = Description of Artificial Sequence: Synthetic peptide
ARDGSSWYDYSDY

SEQ ID NO: 441 - DNA, 321 bp, synthetic construct
  misc_feature 1..321: note = Description of Artificial Sequence: Synthetic polynucleotide
  source 1..321: mol_type = other DNA
gacatccagatgacccagtctccttccaccctgtctgcatctgtaggagacagagtcaccatcacttgccgggccagtcagagtattagtagctggttggcctggtatcaacaggtaccagggaaagcccctaaactcctgatctataaggcgtctagtttagaaaatggggtcccatcaaggttcagcggcagtggatctgggacagaattcactctcatcatcagcagcctgcagcctgatgattttgcaacttattactgccaacagtatattagttattctcggacgttcggccaagggaccaaggtggaaatcaaa

SEQ ID NO: 442 - protein, 107 aa, synthetic construct
  REGION 1..107: note = Description of Artificial Sequence: Synthetic polypeptide
DIQMTQSPSTLSASVGDRVTITCRASQSISSWLAWYQQVPGKAPKLLIYKASSLENGVPSRFSGSGSGTEFTLIISSLQPDDFATYYCQQYISYSRTFGQGTKVEIK

SEQ ID NO: 443 - DNA, 18 bp, synthetic construct
  misc_feature 1..18: note = Description of Artificial Sequence: Synthetic oligonucleotide
  source 1..18: mol_type = other DNA
cagagtattagtagctgg

SEQ ID NO: 444 - protein, 6 aa, synthetic construct
  REGION 1..6: note = Description of Artificial Sequence: Synthetic peptide
QSISSW

SEQ ID NO: 445-446 - skipped (2 entries)

SEQ ID NO: 447 - DNA, 27 bp, synthetic construct
  misc_feature 1..27: note = Description of Artificial Sequence: Synthetic oligonucleotide
  source 1..27: mol_type = other DNA
caacagtatattagttattctcggacg

SEQ ID NO: 448 - protein, 9 aa, synthetic construct
  REGION 1..9: note = Description of Artificial Sequence: Synthetic peptide
QQYISYSRT

SEQ ID NO: 449 - DNA, 360 bp, synthetic construct; identical to SEQ ID NO: 433
  misc_feature 1..360: note = Description of Artificial Sequence: Synthetic polynucleotide
  source 1..360: mol_type = other DNA

SEQ ID NO: 450 - protein, 120 aa, synthetic construct; identical to SEQ ID NO: 434
  REGION 1..120: note = Description of Artificial Sequence: Synthetic polypeptide

SEQ ID NO: 451 - DNA, 321 bp, synthetic construct; identical to SEQ ID NO: 441
  misc_feature 1..321: note = Description of Artificial Sequence: Synthetic polynucleotide
  source 1..321: mol_type = other DNA

SEQ ID NO: 452 - protein, 107 aa, synthetic construct; identical to SEQ ID NO: 442
  REGION 1..107: note = Description of Artificial Sequence: Synthetic polypeptide

SEQ ID NO: 453 - DNA, 360 bp, synthetic construct
  misc_feature 1..360: note = Description of Artificial Sequence: Synthetic polynucleotide
  source 1..360: mol_type = other DNA
gaggtgcagctggtggagtctgggggaggcctggtcaagcctggggggtccctgagactctcctgtgcagcctctggattcaccttcagtacttataacatgaactgggtccgccaggctccagggaaggggctggagtgggtctcatccattaggagtagtagtaattacatatactacgcagactcagtgaagggccgattcaccatctccagagacaacgccaagaactcactgtatctgcaaatgaacagcctgagagccgaggacacggctgtgtattactgtgcgagagatggcagcagttggtacgactactctgactactggggccagggaaccctggtcaccgtctcctca

SEQ ID NO: 454 - protein, 120 aa, synthetic construct; identical to SEQ ID NO: 406
  REGION 1..120: note = Description of Artificial Sequence: Synthetic polypeptide

SEQ ID NO: 455 - DNA, 321 bp, synthetic construct; identical to SEQ ID NO: 407
  misc_feature 1..321: note = Description of Artificial Sequence: Synthetic polynucleotide
  source 1..321: mol_type = other DNA

SEQ ID NO: 456 - protein, 107 aa, synthetic construct; identical to SEQ ID NO: 408
  REGION 1..107: note = Description of Artificial Sequence: Synthetic polypeptide

SEQ ID NO: 457 - DNA, 360 bp, synthetic construct; identical to SEQ ID NO: 401
  misc_feature 1..360: note = Description of Artificial Sequence: Synthetic polynucleotide
  source 1..360: mol_type = other DNA

SEQ ID NO: 458 - protein, 120 aa, synthetic construct; identical to SEQ ID NO: 402
  REGION 1..120: note = Description of Artificial Sequence: Synthetic polypeptide

SEQ ID NO: 459 - DNA, 24 bp, synthetic construct
  misc_feature 1..24: note = Description of Artificial Sequence: Synthetic oligonucleotide
  source 1..24: mol_type = other DNA
ggattcaccttcagtacttataac

SEQ ID NO: 460 - protein, 8 aa, synthetic construct
  REGION 1..8: note = Description of Artificial Sequence: Synthetic peptide
GFTFSTYN

SEQ ID NO: 461 - DNA, 24 bp, synthetic construct
  misc_feature 1..24: note = Description of Artificial Sequence: Synthetic oligonucleotide
  source 1..24: mol_type = other DNA
attaggagtagtagtaattacata

SEQ ID NO: 462 - protein, 8 aa, synthetic construct
  REGION 1..8: note = Description of Artificial Sequence: Synthetic peptide
IRSSSNYI

SEQ ID NO: 463 - DNA, 39 bp, synthetic construct; identical to SEQ ID NO: 391
  misc_feature 1..39: note = Description of Artificial Sequence: Synthetic oligonucleotide
  source 1..39: mol_type = other DNA

SEQ ID NO: 464 - protein, 13 aa, synthetic construct
  REGION 1..13: note = Description of Artificial Sequence: Synthetic peptide
ARDGSSWYDYSDY

SEQ ID NO: 465 - DNA, 321 bp, synthetic construct; identical to SEQ ID NO: 393
  misc_feature 1..321: note = Description of Artificial Sequence: Synthetic polynucleotide
  source 1..321: mol_type = other DNA

SEQ ID NO: 466 - protein, 107 aa, synthetic construct; identical to SEQ ID NO: 394
  REGION 1..107: note = Description of Artificial Sequence: Synthetic polypeptide

SEQ ID NO: 467 - DNA, 18 bp, synthetic construct
  misc_feature 1..18: note = Description of Artificial Sequence: Synthetic oligonucleotide
  source 1..18: mol_type = other DNA
cagagtattagtagctgg

SEQ ID NO: 468 - protein, 6 aa, synthetic construct
  REGION 1..6: note = Description of Artificial Sequence: Synthetic peptide
QSISSW

SEQ ID NO: 469-470 - skipped (2 entries)

SEQ ID NO: 471 - DNA, 27 bp, synthetic construct
  misc_feature 1..27: note = Description of Artificial Sequence: Synthetic oligonucleotide
  source 1..27: mol_type = other DNA
caacagtatattagttattctcggacg

SEQ ID NO: 472 - protein, 9 aa, synthetic construct
  REGION 1..9: note = Description of Artificial Sequence: Synthetic peptide
QQYISYSRT

SEQ ID NO: 473 - DNA, 360 bp, synthetic construct; identical to SEQ ID NO: 401
  misc_feature 1..360: note = Description of Artificial Sequence: Synthetic polynucleotide
  source 1..360: mol_type = other DNA

SEQ ID NO: 474 - protein, 120 aa, synthetic construct; identical to SEQ ID NO: 402
  REGION 1..120: note = Description of Artificial Sequence: Synthetic polypeptide

SEQ ID NO: 475 - DNA, 321 bp, synthetic construct; identical to SEQ ID NO: 393
  misc_feature 1..321: note = Description of Artificial Sequence: Synthetic polynucleotide
  source 1..321: mol_type = other DNA

SEQ ID NO: 476 - protein, 107 aa, synthetic construct; identical to SEQ ID NO: 394
  REGION 1..107: note = Description of Artificial Sequence: Synthetic polypeptide

SEQ ID NO: 477 - DNA, 360 bp, synthetic construct; identical to SEQ ID NO: 405
  misc_feature 1..360: note = Description of Artificial Sequence: Synthetic polynucleotide
  source 1..360: mol_type = other DNA

SEQ ID NO: 478 - protein, 120 aa, synthetic construct; identical to SEQ ID NO: 406
  REGION 1..120: note = Description of Artificial Sequence: Synthetic polypeptide

SEQ ID NO: 479 - DNA, 321 bp, synthetic construct; identical to SEQ ID NO: 407
  misc_feature 1..321: note = Description of Artificial Sequence: Synthetic polynucleotide
  source 1..321: mol_type = other DNA

SEQ ID NO: 480 - protein, 107 aa, synthetic construct; identical to SEQ ID NO: 408
  REGION 1..107: note = Description of Artificial Sequence: Synthetic polypeptide

SEQ ID NO: 481 - DNA, 354 bp, synthetic construct
  misc_feature 1..354: note = Description of Artificial Sequence: Synthetic polynucleotide
  source 1..354: mol_type = other DNA
gaggtgcaactagtggagtctgggggaggcttggtacagcctggggggtccctgagactctcctgtgtagtctctggattcaccttcggtgactacgacatgcactgggtccgtcaagctacaggaagaggtctggagtgggtctcaggtattgctcctgctggtgacacatcctatacaggctccgtgaagggccgattcaccatctccagagagaatgccaagaactccttgcatcttcaaatgaacagcctgacaaccggggacacggctatatattattgtgctagagaggatatagcagtgcctggttttgattactggggccagggaaccctggtcaccgtctcctca

SEQ ID NO: 482 - protein, 118 aa, synthetic construct
  REGION 1..118: note = Description of Artificial Sequence: Synthetic polypeptide
EVQLVESGGGLVQPGGSLRLSCVVSGFTFGDYDMHWVRQATGRGLEWVSGIAPAGDTSYTGSVKGRFTISRENAKNSLHLQMNSLTTGDTAIYYCAREDIAVPGFDYWGQGTLVTVSS

SEQ ID NO: 483 - DNA, 24 bp, synthetic construct
  misc_feature 1..24: note = Description of Artificial Sequence: Synthetic oligonucleotide
  source 1..24: mol_type = other DNA
ggattcaccttcggtgactacgac

SEQ ID NO: 484 - protein, 8 aa, synthetic construct
  REGION 1..8: note = Description of Artificial Sequence: Synthetic peptide
GFTFGDYD

SEQ ID NO: 485 - DNA, 21 bp, synthetic construct
  misc_feature 1..21: note = Description of Artificial Sequence: Synthetic oligonucleotide
  source 1..21: mol_type = other DNA
attgctcctgctggtgacaca

SEQ ID NO: 486 - protein, 7 aa, synthetic construct
  REGION 1..7: note = Description of Artificial Sequence: Synthetic peptide
IAPAGDT

SEQ ID NO: 487 - DNA, 36 bp, synthetic construct
  misc_feature 1..36: note = Description of Artificial Sequence: Synthetic oligonucleotide
  source 1..36: mol_type = other DNA
gctagagaggatatagcagtgcctggttttgattac

SEQ ID NO: 488 - protein, 12 aa, synthetic construct
  REGION 1..12: note = Description of Artificial Sequence: Synthetic peptide
AREDIAVPGFDY

SEQ ID NO: 489 - DNA, 324 bp, synthetic construct
  misc_feature 1..324: note = Description of Artificial Sequence: Synthetic polynucleotide
  source 1..324: mol_type = other DNA
gaaatagtgatgacgcagtctccagccaccctgtctgtgtctccaggggaacgaggcaccctctcctgcagggccagtcagagtgttagcagcaacttagcctggtaccagcagaaacctggccaggctcccagactcctcatctatggtgcatccacgagggccactggcttcccagccaggttcagtggcagtgggtctgggacagagttcactctcaccatcagcagcctgcagtctgaagattttgcagtttattactgtcagcagtataataagtggcctccgttcactttcggccctgggaccaaagtggatttcaaa

SEQ ID NO: 490 - protein, 108 aa, synthetic construct
  REGION 1..108: note = Description of Artificial Sequence: Synthetic polypeptide
EIVMTQSPATLSVSPGERGTLSCRASQSVSSNLAWYQQKPGQAPRLLIYGASTRATGFPARFSGSGSGTEFTLTISSLQSEDFAVYYCQQYNKWPPFTFGPGTKVDFK

SEQ ID NO: 491 - DNA, 18 bp, synthetic construct
  misc_feature 1..18: note = Description of Artificial Sequence: Synthetic oligonucleotide
  source 1..18: mol_type = other DNA
cagagtgttagcagcaac

SEQ ID NO: 492 - protein, 6 aa, synthetic construct
  REGION 1..6: note = Description of Artificial Sequence: Synthetic peptide
QSVSSN

SEQ ID NO: 493-494 - skipped (2 entries)

SEQ ID NO: 495 - DNA, 30 bp, synthetic construct
  misc_feature 1..30: note = Description of Artificial Sequence: Synthetic oligonucleotide
  source 1..30: mol_type = other DNA
cagcagtataataagtggcctccgttcact

SEQ ID NO: 496 - protein, 10 aa, synthetic construct
  REGION 1..10: note = Description of Artificial Sequence: Synthetic peptide
QQYNKWPPFT

SEQ ID NO: 497 - DNA, 354 bp, synthetic construct; identical to SEQ ID NO: 481
  misc_feature 1..354: note = Description of Artificial Sequence: Synthetic polynucleotide
  source 1..354: mol_type = other DNA

SEQ ID NO: 498 - protein, 118 aa, synthetic construct; identical to SEQ ID NO: 482
  REGION 1..118: note = Description of Artificial Sequence: Synthetic polypeptide

SEQ ID NO: 499 - DNA, 324 bp, synthetic construct
  misc_feature 1..324: note = Description of Artificial Sequence: Synthetic polynucleotide
  source 1..324: mol_type = other DNA
gaaatagtgatgacgcagtctccagccaccctgtctgtgtctccaggggaacgaggcaccctctcctgcagggccagtcagagtgttagcagcaacttagcctggtaccagcagaaacctggccaggctcccagactcctcatctatggtgcatccacgagggccactggcttcccagccaggttcagtggcagtgggtctgggacagagttcactctcaccatcagcagcctgcagtctgaagattttgcagtttattactgtcagcagtataataagtggcctccgttcactttcggccctgggaccaaagtggatatcaaa

SEQ ID NO: 500 - protein, 108 aa, synthetic construct
  REGION 1..108: note = Description of Artificial Sequence: Synthetic polypeptide
EIVMTQSPATLSVSPGERGTLSCRASQSVSSNLAWYQQKPGQAPRLLIYGASTRATGFPARFSGSGSGTEFTLTISSLQSEDFAVYYCQQYNKWPPFTFGPGTKVDIK

SEQ ID NO: 501 - DNA, 354 bp, synthetic construct
  misc_feature 1..354: note = Description of Artificial Sequence: Synthetic polynucleotide
  source 1..354: mol_type = other DNA
gaggtgcagctggtggagtctgggggaggcttggtacagcctggggggtccctgagactctcctgtgcagcctctggattcaccttcggtgactacgacatgcactgggtccgccaagctacaggaaaaggtctggagtgggtctcagctattgctcctgctggtgacacatactatccaggctccgtgaagggccgattcaccatctccagagaaaatgccaagaactccttgtatcttcaaatgaacagcctgagagccggggacacggctgtgtattactgtgctagagaggatatagcagtgcctggttttgattactggggccaaggaaccctggtcaccgtctcctca

SEQ ID NO: 502 - protein, 118 aa, synthetic construct
  REGION 1..118: note = Description of Artificial Sequence: Synthetic polypeptide
EVQLVESGGGLVQPGGSLRLSCAASGFTFGDYDMHWVRQATGKGLEWVSAIAPAGDTYYPGSVKGRFTISRENAKNSLYLQMNSLRAGDTAVYYCAREDIAVPGFDYWGQGTLVTVSS

SEQ ID NO: 503 - DNA, 324 bp, synthetic construct
  misc_feature 1..324: note = Description of Artificial Sequence: Synthetic polynucleotide
  source 1..324: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataagtggcctccgttcactttcggccctgggaccaaagtggatatcaaa

SEQ ID NO: 504 - protein, 108 aa, synthetic construct
  REGION 1..108: note = Description of Artificial Sequence: Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYNKWPPFTFGPGTKVDIK

SEQ ID NO: 505 - DNA, 378 bp, synthetic construct
  misc_feature 1..378: note = Description of Artificial Sequence: Synthetic polynucleotide
  source 1..378: mol_type = other DNA
caaattctgctggtgcaatctggacctgaggtgaaggagcctggggcctcagtgaaggtctcctgcaaggcttctggttacacctttaccaactacgctatcagctgggtgcgacaggtccctggacaagggcttgagtggatgggatgggtcagcgcttacaatggtcacacaaactatgcacatgaagtccagggcagagtcaccatgaccacagacacatccacgaccacagcctacatggagctgaggagcctgagatctgacgacacggccatgtattactgtgcgagagggggtgtagtcgtgccagttgctccccacttctacaacggtatggacgtctggggccaagggaccacggtcaccgtctcctca

SEQ ID NO: 506 - protein, 126 aa, synthetic construct
  REGION 1..126: note = Description of Artificial Sequence: Synthetic polypeptide
QILLVQSGPEVKEPGASVKVSCKASGYTFTNYAISWVRQVPGQGLEWMGWVSAYNGHTNYAHEVQGRVTMTTDTSTTTAYMELRSLRSDDTAMYYCARGGVVVPVAPHFYNGMDVWGQGTTVTVSS

SEQ ID NO: 507 - DNA, 24 bp, synthetic construct
  misc_feature 1..24: note = Description of Artificial Sequence: Synthetic oligonucleotide
  source 1..24: mol_type = other DNA
ggttacacctttaccaactacgct

SEQ ID NO: 508 - protein, 8 aa, synthetic construct
  REGION 1..8: note = Description of Artificial Sequence: Synthetic peptide
GYTFTNYA

SEQ ID NO: 509 - DNA, 24 bp, synthetic construct
  misc_feature 1..24: note = Description of Artificial Sequence: Synthetic oligonucleotide
  source 1..24: mol_type = other DNA
gtcagcgcttacaatggtcacaca

SEQ ID NO: 510 - protein, 8 aa, synthetic construct
  REGION 1..8: note = Description of Artificial Sequence: Synthetic peptide
VSAYNGHT

SEQ ID NO: 511 - DNA, 57 bp, synthetic construct
  misc_feature 1..57: note = Description of Artificial Sequence: Synthetic oligonucleotide
  source 1..57: mol_type = other DNA
gcgagagggggtgtagtcgtgccagttgctccccacttctacaacggtatggacgtc

SEQ ID NO: 512 - protein, 19 aa, synthetic construct
  REGION 1..19: note = Description of Artificial Sequence: Synthetic peptide
ARGGVVVPVAPHFYNGMDV

SEQ ID NO: 513 - DNA, 336 bp, synthetic construct
  misc_feature 1..336: note = Description of Artificial Sequence: Synthetic polynucleotide
  source 1..336: mol_type = other DNA
gatattgtgatgactcagtttccactctccctgcccgtcacccctggagagccggcctccatctcctgcaggtctagtcagagcctcctgcatattaatgaatacaactatttggattggtacctaaagaagccagggcagtctccacagctcctgatctatttgggttttaatcgggcctccggggtccctgacaggttcagtggcagtggatcaggcacagattttacactgaaaatcagcagagtggaggctgaggatgttggggtctattactgcatgcaagctcttcaaactccgtggacgttaggccaagggaccaaggtggaaatcaaa

SEQ ID NO: 514 - protein, 112 aa, synthetic construct
  REGION 1..112: note = Description of Artificial Sequence: Synthetic polypeptide
DIVMTQFPLSLPVTPGEPASISCRSSQSLLHINEYNYLDWYLKKPGQSPQLLIYLGFNRASGVPDRFSGSGSGTDFTLKISRVEAEDVGVYYCMQALQTPWTLGQGTKVEIK

SEQ ID NO: 515 - DNA, 33 bp, synthetic construct
  misc_feature 1..33: note = Description of Artificial Sequence: Synthetic oligonucleotide
  source 1..33: mol_type = other DNA
cagagcctcctgcatattaatgaatacaactat

SEQ ID NO: 516 - protein, 11 aa, synthetic construct
  REGION 1..11: note = Description of Artificial Sequence: Synthetic peptide
QSLLHINEYNY

SEQ ID NO: 517-518 - skipped (2 entries)

SEQ ID NO: 519 - DNA, 27 bp, synthetic construct
  misc_feature 1..27: note = Description of Artificial Sequence: Synthetic oligonucleotide
  source 1..27: mol_type = other DNA
atgcaagctcttcaaactccgtggacg

SEQ ID NO: 520 - protein, 9 aa, synthetic construct
  REGION 1..9: note = Description of Artificial Sequence: Synthetic peptide
MQALQTPWT

SEQ ID NO: 521 - DNA, 378 bp, synthetic construct
  misc_feature 1..378: note = Description of Artificial Sequence: Synthetic polynucleotide
  source 1..378: mol_type = other DNA
caggttcagctggtgcagtctggacctgaggtgaaggagcctggggcctcagtgaaggtctcctgcaaggcttctggttacacctttaccaactacgctatcagctgggtgcgacaggtccctggacaagggcttgagtggatgggatgggtcagcgcttacaatggtcacacaaactatgcacatgaagtccagggcagagtcaccatgaccacagacacatccacgaccacagcctacatggagctgaggagcctgagatctgacgacacggccatgtattactgtgcgagagggggtgtagtcgtgccagttgctccccacttctacaacggtatggacgtctggggccaagggaccacggtcaccgtctcctca

SEQ ID NO: 522 - protein, 126 aa, synthetic construct
  REGION 1..126: note = Description of Artificial Sequence: Synthetic polypeptide
QVQLVQSGPEVKEPGASVKVSCKASGYTFTNYAISWVRQVPGQGLEWMGWVSAYNGHTNYAHEVQGRVTMTTDTSTTTAYMELRSLRSDDTAMYYCARGGVVVPVAPHFYNGMDVWGQGTTVTVSS

SEQ ID NO: 523 - DNA, 336 bp, synthetic construct
  misc_feature 1..336: note = Description of Artificial Sequence: Synthetic polynucleotide
  source 1..336: mol_type = other DNA
gatattgtgatgactcagtctccactctccctgcccgtcacccctggagagccggcctccatctcctgcaggtctagtcagagcctcctgcatattaatgaatacaactatttggattggtacctaaagaagccagggcagtctccacagctcctgatctatttgggttttaatcgggcctccggggtccctgacaggttcagtggcagtggatcaggcacagattttacactgaaaatcagcagagtggaggctgaggatgttggggtctattactgcatgcaagctcttcaaactccgtggacgttaggccaagggaccaaggtggaaatcaaa

SEQ ID NO: 524 - protein, 112 aa, synthetic construct
  REGION 1..112: note = Description of Artificial Sequence: Synthetic polypeptide
DIVMTQSPLSLPVTPGEPASISCRSSQSLLHINEYNYLDWYLKKPGQSPQLLIYLGFNRASGVPDRFSGSGSGTDFTLKISRVEAEDVGVYYCMQALQTPWTLGQGTKVEIK

SEQ ID NO: 525 - DNA, 378 bp, synthetic construct
  misc_feature 1..378: note = Description of Artificial Sequence: Synthetic polynucleotide
  source 1..378: mol_type = other DNA
caggttcagctggtgcagtctggagctgaggtgaagaagcctggggcctcagtgaaggtctcctgcaaggcttctggttacacctttaccaactacgctatcagctgggtgcgacaggcccctggacaagggcttgagtggatgggatgggtcagcgcttacaatggtcacacaaactatgcacagaagctccagggcagagtcaccatgaccacagacacatccacgagcacagcctacatggagctgaggagcctgagatctgacgacacggccgtgtattactgtgcgagagggggtgtagtcgtgccagttgctccccacttctacaacggtatggacgtctgggggcaagggaccacggtcaccgtctcctca

SEQ ID NO: 526 - protein, 126 aa, synthetic construct
  REGION 1..126: note = Description of Artificial Sequence: Synthetic polypeptide
QVQLVQSGAEVKKPGASVKVSCKASGYTFTNYAISWVRQAPGQGLEWMGWVSAYNGHTNYAQKLQGRVTMTTDTSTSTAYMELRSLRSDDTAVYYCARGGVVVPVAPHFYNGMDVWGQGTTVTVSS

SEQ ID NO: 527 - DNA, 336 bp, synthetic construct
  misc_feature 1..336: note = Description of Artificial Sequence: Synthetic polynucleotide
  source 1..336: mol_type = other DNA
gatattgtgatgactcagtctccactctccctgcccgtcacccctggagagccggcctccatctcctgcaggtctagtcagagcctcctgcatattaatgaatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctcttcaaactccgtggacgttcggccaagggaccaaggtggaaatcaaa

SEQ ID NO: 528 - protein, 112 aa, synthetic construct
  REGION 1..112: note = Description of Artificial Sequence: Synthetic polypeptide
DIVMTQSPLSLPVTPGEPASISCRSSQSLLHINEYNYLDWYLQKPGQSPQLLIYLGSNRASGVPDRFSGSGSGTDFTLKISRVEAEDVGVYYCMQALQTPWTFGQGTKVEIK

SEQ ID NO: 529 - DNA, 351 bp, synthetic construct
  misc_feature 1..351: note = Description of Artificial Sequence: Synthetic polynucleotide
  source 1..351: mol_type = other DNA
gaggtgcagctggtggagtctgggggaggcttggtacagcctggggggtccctgagactctcctgtgcagcctctggattcaccctaagtagctacgacatgcactgggtccgccaagcaacaggaaaaggtctggagtgggtctcagctattggcagtactggtgacacatactatacaggctccgtgatgggccgattcaccatctccagagacgctgccaaaaactccttctatcttgaaatgaacagcctgagagtcggggacacggctgtatattactgtgcaagagagggaataagaacaccctatgattattggggccagggagcccgggtcaccgtctcctca

SEQ ID NO: 530 - protein, 117 aa, synthetic construct
  REGION 1..117: note = Description of Artificial Sequence: Synthetic polypeptide
EVQLVESGGGLVQPGGSLRLSCAASGFTLSSYDMHWVRQATGKGLEWVSAIGSTGDTYYTGSVMGRFTISRDAAKNSFYLEMNSLRVGDTAVYYCAREGIRTPYDYWGQGARVTVSS

SEQ ID NO: 531 - DNA, 24 bp, synthetic construct
  misc_feature 1..24: note = Description of Artificial Sequence: Synthetic oligonucleotide
  source 1..24: mol_type = other DNA
ggattcaccctaagtagctacgac

SEQ ID NO: 532 - protein, 8 aa, synthetic construct
  REGION 1..8: note = Description of Artificial Sequence: Synthetic peptide
GFTLSSYD

SEQ ID NO: 533 - DNA, 21 bp, synthetic construct
  misc_feature 1..21: note = Description of Artificial Sequence: Synthetic oligonucleotide
  source 1..21: mol_type = other DNA
attggcagtactggtgacaca

SEQ ID NO: 534 - protein, 7 aa, synthetic construct
  REGION 1..7: note = Description of Artificial Sequence: Synthetic peptide
IGSTGDT

SEQ ID NO: 535 - DNA, 33 bp, synthetic construct
  misc_feature 1..33: note = Description of Artificial Sequence: Synthetic oligonucleotide
  source 1..33: mol_type = other DNA
gcaagagagggaataagaacaccctatgattat

SEQ ID NO: 536 - protein, 11 aa, synthetic construct
  REGION 1..11: note = Description of Artificial Sequence: Synthetic peptide
AREGIRTPYDY

SEQ ID NO: 537 - DNA, 324 bp, synthetic construct
  misc_feature 1..324: note = Description of Artificial Sequence: Synthetic polynucleotide
  source 1..324: mol_type = other DNA
gaaatagtgatgacgcagtctccagccaccctgtctgtgtctccaggggaaagagccaccctctcctgcagggccagtcagagtgttagcagcaatgtagcctggtaccagcagaaacctggccaggctcccaggctcctcatctatggtgcatccaccagggccactggtatcccagccaggttcagtggcagtgggtctgggacagaattcactctcaccatcagcagcctgcagtctgaagattttgcagtttattactgtcagcagtataataattggcctccattcactttcggccctgggaccaaagtggatatcaaa

SEQ ID NO: 538 - protein, 108 aa, synthetic construct
  REGION 1..108: note = Description of Artificial Sequence: Synthetic polypeptide
EIVMTQSPATLSVSPGERATLSCRASQSVSSNVAWYQQKPGQAPRLLIYGASTRATGIPARFSGSGSGTEFTLTISSLQSEDFAVYYCQQYNNWPPFTFGPGTKVDIK

SEQ ID NO: 539 - DNA, 18 bp, synthetic construct
  misc_feature 1..18: note = Description of Artificial Sequence: Synthetic oligonucleotide
  source 1..18: mol_type = other DNA
cagagtgttagcagcaat

SEQ ID NO: 540 - protein, 6 aa, synthetic construct
  REGION 1..6: note = Description of Artificial Sequence: Synthetic peptide
QSVSSN

SEQ ID NO: 541-542 - skipped (2 entries)

SEQ ID NO: 543 - DNA, 30 bp, synthetic construct
  misc_feature 1..30: note = Description of Artificial Sequence: Synthetic oligonucleotide
  source 1..30: mol_type = other DNA
cagcagtataataattggcctccattcact

SEQ ID NO: 544 - protein, 10 aa, synthetic construct
  REGION 1..10: note = Description of Artificial Sequence: Synthetic peptide
QQYNNWPPFT

SEQ ID NO: 545 - DNA, 351 bp, synthetic construct
  misc_feature 1..351: note = Description of Artificial Sequence: Synthetic polynucleotide
  source 1..351: mol_type = other DNA
gaggtgcagctggtggagtctgggggaggcttggtacagcctggggggtccctgagactctcctgtgcagcctctggattcaccctaagtagctacgacatgcactgggtccgccaagcaacaggaaaaggtctggagtgggtctcagctattggcagtactggtgacacatactatacaggctccgtgatgggccgattcaccatctccagagacgctgccaaaaactccttctatcttgaaatgaacagcctgagagtcggggacacggctgtatattactgtgcaagagagggaataagaacaccctatgattattggggccagggaaccctggtcaccgtctcctca

SEQ ID NO: 546 - protein, 117 aa, synthetic construct
  REGION 1..117: note = Description of Artificial Sequence: Synthetic polypeptide
EVQLVESGGGLVQPGGSLRLSCAASGFTLSSYDMHWVRQATGKGLEWVSAIGSTGDTYYTGSVMGRFTISRDAAKNSFYLEMNSLRVGDTAVYYCAREGIRTPYDYWGQGTLVTVSS

SEQ ID NO: 547 - DNA, 324 bp, synthetic construct; identical to SEQ ID NO: 537
  misc_feature 1..324: note = Description of Artificial Sequence: Synthetic polynucleotide
  source 1..324: mol_type = other DNA

SEQ ID NO: 548 - protein, 108 aa, synthetic construct; identical to SEQ ID NO: 538
  REGION 1..108: note = Description of Artificial Sequence: Synthetic polypeptide

SEQ ID NO: 549 - DNA, 351 bp, synthetic construct
  misc_feature 1..351: note = Description of Artificial Sequence: Synthetic polynucleotide
  source 1..351: mol_type = other DNA
gaggtgcagctggtggagtctgggggaggcttggtacagcctggggggtccctgagactctcctgtgcagcctctggattcaccctaagtagctacgacatgcactgggtccgccaagctacaggaaaaggtctggagtgggtctcagctattggcagtactggtgacacatactatccaggctccgtgaagggccgattcaccatctccagagaaaatgccaagaactccttgtatcttcaaatgaacagcctgagagccggggacacggctgtgtattactgtgcaagagagggaataagaacaccctatgattattggggccaaggaaccctggtcaccgtctcctca

SEQ ID NO: 550 - protein, 117 aa, synthetic construct
  REGION 1..117: note = Description of Artificial Sequence: Synthetic polypeptide
EVQLVESGGGLVQPGGSLRLSCAASGFTLSSYDMHWVRQATGKGLEWVSAIGSTGDTYYPGSVKGRFTISRENAKNSLYLQMNSLRAGDTAVYYCAREGIRTPYDYWGQGTLVTVSS

SEQ ID NO: 551 - DNA, 324 bp, synthetic construct
  misc_feature 1..324: note = Description of Artificial Sequence: Synthetic polynucleotide
  source 1..324: mol_type = other DNA
gaaatagtgatgacgcagtctccagccaccctgtctgtgtctccaggggaaagagccaccctctcctgcagggccagtcagagtgttagcagcaatttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataattggcctccattcactttcggccctgggaccaaagtggatatcaaa

SEQ ID NO: 552 - protein, 108 aa, synthetic construct; identical to SEQ ID NO: 24
  REGION 1..108: note = Description of Artificial Sequence: Synthetic polypeptide

SEQ ID NO: 553 - DNA, 351 bp, synthetic construct; identical to SEQ ID NO: 529
  misc_feature 1..351: note = Description of Artificial Sequence: Synthetic polynucleotide
  source 1..351: mol_type = other DNA

SEQ ID NO: 554 - protein, 117 aa, synthetic construct; identical to SEQ ID NO: 530
  REGION 1..117: note = Description of Artificial Sequence: Synthetic polypeptide

SEQ ID NO: 555 - DNA, 24 bp, synthetic construct
  misc_feature 1..24: note = Description of Artificial Sequence: Synthetic oligonucleotide
  source 1..24: mol_type = other DNA
ggattcaccctaagtagctacgac

SEQ ID NO: 556 - protein, 8 aa, synthetic construct
  REGION 1..8: note = Description of Artificial Sequence: Synthetic peptide
GFTLSSYD

SEQ ID NO: 557 - DNA, 21 bp, synthetic construct
  misc_feature 1..21: note = Description of Artificial Sequence: Synthetic oligonucleotide
  source 1..21: mol_type = other DNA
attggcagtactggtgacaca

SEQ ID NO: 558 - protein, 7 aa, synthetic construct
  REGION 1..7: note = Description of Artificial Sequence: Synthetic peptide
IGSTGDT

SEQ ID NO: 559 - DNA, 33 bp, synthetic construct; identical to SEQ ID NO: 535
  misc_feature 1..33: note = Description of Artificial Sequence: Synthetic oligonucleotide
  source 1..33: mol_type = other DNA

SEQ ID NO: 560 - protein, 11 aa, synthetic construct
  REGION 1..11: note = Description of Artificial Sequence: Synthetic peptide
AREGIRTPYDY

SEQ ID NO: 561 - DNA, 324 bp, synthetic construct; identical to SEQ ID NO: 537
  misc_feature 1..324: note = Description of Artificial Sequence: Synthetic polynucleotide
  source 1..324: mol_type = other DNA

SEQ ID NO: 562 - protein, 108 aa, synthetic construct; identical to SEQ ID NO: 538
  REGION 1..108: note = Description of Artificial Sequence: Synthetic polypeptide

SEQ ID NO: 563 - DNA, 18 bp, synthetic construct
  misc_feature 1..18: note = Description of Artificial Sequence: Synthetic oligonucleotide
  source 1..18: mol_type = other DNA
cagagtgttagcagcaat

SEQ ID NO: 564 - protein, 6 aa, synthetic construct
  REGION 1..6: note = Description of Artificial Sequence: Synthetic peptide
QSVSSN

SEQ ID NO: 565-566 - skipped (2 entries)

SEQ ID NO: 567 - DNA, 30 bp, synthetic construct; identical to SEQ ID NO: 543
  misc_feature 1..30: note = Description of Artificial Sequence: Synthetic oligonucleotide
  source 1..30: mol_type = other DNA

SEQ ID NO: 568 - protein, 10 aa, synthetic construct
  REGION 1..10: note = Description of Artificial Sequence: Synthetic peptide
QQYNNWPPFT

SEQ ID NO: 569 - DNA, 351 bp, synthetic construct; identical to SEQ ID NO: 545
  misc_feature 1..351: note = Description of Artificial Sequence: Synthetic polynucleotide
  source 1..351: mol_type = other DNA

SEQ ID NO: 570 - protein, 117 aa, synthetic construct; identical to SEQ ID NO: 546
  REGION 1..117: note = Description of Artificial Sequence: Synthetic polypeptide

SEQ ID NO: 571 - DNA, 324 bp, synthetic construct; identical to SEQ ID NO: 537
  misc_feature 1..324: note = Description of Artificial Sequence: Synthetic polynucleotide
  source 1..324: mol_type = other DNA

SEQ ID NO: 572 - protein, 108 aa, synthetic construct; identical to SEQ ID NO: 538
  REGION 1..108: note = Description of Artificial Sequence: Synthetic polypeptide

SEQ ID NO: 573 - DNA, 351 bp, synthetic construct; identical to SEQ ID NO: 549
  misc_feature 1..351: note = Description of Artificial Sequence: Synthetic polynucleotide
  source 1..351: mol_type = other DNA

SEQ ID NO: 574 - protein, 117 aa, synthetic construct; identical to SEQ ID NO: 550
  REGION 1..117: note = Description of Artificial Sequence: Synthetic polypeptide

SEQ ID NO: 575 - DNA, 324 bp, synthetic construct; identical to SEQ ID NO: 551
  misc_feature 1..324: note = Description of Artificial Sequence: Synthetic polynucleotide
  source 1..324: mol_type = other DNA

SEQ ID NO: 576 - protein, 108 aa, synthetic construct; identical to SEQ ID NO: 24
  REGION 1..108: note = Description of Artificial Sequence: Synthetic polypeptide

SEQ ID NO: 577 - DNA, 363 bp, synthetic construct
  misc_feature 1..363: note = Description of Artificial Sequence: Synthetic polynucleotide
  source 1..363: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattaattggaacagtggtagcataggctatgcggactctgtgaagggccgattcaccatctccagagacaacgccaagcactccctgtatctgcaaatgaacagtctgagacctgaggacacggccttgtattactgtgtaaaagaggtgactacgggatactactacggtatggacgtctggggccaagggaccacggtcaccgtctcctca

SEQ ID NO: 578 - protein, 121 aa, synthetic construct
  REGION 1..121: note = Description of Artificial Sequence: Synthetic polypeptide
EVQLVESGGGLVQPGRSLRLSCAASGFTFDDYAMHWVRQAPGKGLEWVSGINWNSGSIGYADSVKGRFTISRDNAKHSLYLQMNSLRPEDTALYYCVKEVTTGYYYGMDVWGQGTTVTVSS

SEQ ID NO: 579 - DNA, 24 bp, synthetic construct
  misc_feature 1..24: note = Description of Artificial Sequence: Synthetic oligonucleotide
  source 1..24: mol_type = other DNA
ggattcacctttgatgattatgcc

SEQ ID NO: 580 - protein, 8 aa, synthetic construct
  REGION 1..8: note = Description of Artificial Sequence: Synthetic peptide
GFTFDDYA

SEQ ID NO: 581 - DNA, 24 bp, synthetic construct
  misc_feature 1..24: note = Description of Artificial Sequence: Synthetic oligonucleotide
  source 1..24: mol_type = other DNA
attaattggaacagtggtagcata

SEQ ID NO: 582 - protein, 8 aa, synthetic construct
  REGION 1..8: note = Description of Artificial Sequence: Synthetic peptide
INWNSGSI

SEQ ID NO: 583 - DNA, 42 bp, synthetic construct
  misc_feature 1..42: note = Description of Artificial Sequence: Synthetic oligonucleotide
  source 1..42: mol_type = other DNA
gtaaaagaggtgactacgggatactactacggtatggacgtc

SEQ ID NO: 584 - protein, 14 aa, synthetic construct
  REGION 1..14: note = Description of Artificial Sequence: Synthetic peptide
VKEVTTGYYYGMDV

SEQ ID NO: 585 - DNA, 321 bp, synthetic construct
  misc_feature 1..321: note = Description of Artificial Sequence: Synthetic polynucleotide
  source 1..321: mol_type = other DNA
gacatccagttgacccagtctccatccttcctgtctgcatctgtaggagacagagtcaccatcacttgctgggccagtcagggcattagcagttatttagcctggtatcagaaaaaaccagggaaagcccctaacctcctgatctatgatgcatccactttgcaaagtggggtcccatcaaggttcagcggcagtggatctgggacagaattcactctcacactcagcagcctgcagcctgaagattttgcaacttattactgtcaacagcttaatatttacccattcactttcggccctgggaccaaagtggatatcaaa

SEQ ID NO: 586 - protein, 107 aa, synthetic construct
  REGION 1..107: note = Description of Artificial Sequence: Synthetic polypeptide
DIQLTQSPSFLSASVGDRVTITCWASQGISSYLAWYQKKPGKAPNLLIYDASTLQSGVPSRFSGSGSGTEFTLTLSSLQPEDFATYYCQQLNIYPFTFGPGTKVDIK

SEQ ID NO: 587 - DNA, 18 bp, synthetic construct
  misc_feature 1..18: note = Description of Artificial Sequence: Synthetic oligonucleotide
  source 1..18: mol_type = other DNA
cagggcattagcagttat

SEQ ID NO: 588 - protein, 6 aa, synthetic construct
  REGION 1..6: note = Description of Artificial Sequence: Synthetic peptide
QGISSY

SEQ ID NO: 589-590 - skipped (2 entries)

SEQ ID NO: 591 - DNA, 27 bp, synthetic construct
  misc_feature 1..27: note = Description of Artificial Sequence: Synthetic oligonucleotide
  source 1..27: mol_type = other DNA
caacagcttaatatttacccattcact

SEQ ID NO: 592 - protein, 9 aa, synthetic construct
  REGION 1..9: note = Description of Artificial Sequence: Synthetic peptide
QQLNIYPFT

SEQ ID NO: 593 - DNA, 363 bp, synthetic construct; identical to SEQ ID NO: 577
  misc_feature 1..363: note = Description of Artificial Sequence: Synthetic polynucleotide
  source 1..363: mol_type = other DNA

SEQ ID NO: 594 - protein, 121 aa, synthetic construct; identical to SEQ ID NO: 578
  REGION 1..121: note = Description of Artificial Sequence: Synthetic polypeptide

SEQ ID NO: 595 - DNA, 321 bp, synthetic construct; identical to SEQ ID NO: 585
  misc_feature 1..321: note = Description of Artificial Sequence: Synthetic polynucleotide
  source 1..321: mol_type = other DNA

SEQ ID NO: 596 - protein, 107 aa, synthetic construct; identical to SEQ ID NO: 586
  REGION 1..107: note = Description of Artificial Sequence: Synthetic polypeptide

SEQ ID NO: 597 - DNA, 363 bp, synthetic construct
  misc_feature 1..363: note = Description of Artificial Sequence: Synthetic polynucleotide
  source 1..363: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattaattggaacagtggtagcataggctatgcggactctgtgaagggccgattcaccatctccagagacaacgccaagaactccctgtatctgcaaatgaacagtctgagagctgaggacacggccttgtattactgtgtaaaagaggtgactacgggatactactacggtatggacgtctgggggcaagggaccacggtcaccgtctcctca

SEQ ID NO: 598 - protein, 121 aa, synthetic construct
  REGION 1..121: note = Description of Artificial Sequence: Synthetic polypeptide
EVQLVESGGGLVQPGRSLRLSCAASGFTFDDYAMHWVRQAPGKGLEWVSGINWNSGSIGYADSVKGRFTISRDNAKNSLYLQMNSLRAEDTALYYCVKEVTTGYYYGMDVWGQGTTVTVSS

SEQ ID NO: 599 - DNA, 321 bp, synthetic construct
  misc_feature 1..321: note = Description of Artificial Sequence: Synthetic polynucleotide
  source 1..321: mol_type = other DNA
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatctatgatgcatccactttgcaaagtggggtcccatcaaggttcagcggcagtggatctgggacagaattcactctcacaatcagcagcctgcagcctgaagattttgcaacttattactgtcaacagcttaatatttacccattcactttcggccctgggaccaaagtggatatcaaa

SEQ ID NO: 600 - protein, 107 aa, synthetic construct
  REGION 1..107: note = Description of Artificial Sequence: Synthetic polypeptide
DIQLTQSPSFLSASVGDRVTITCRASQGISSYLAWYQQKPGKAPKLLIYDASTLQSGVPSRFSGSGSGTEFTLTISSLQPEDFATYYCQQLNIYPFTFGPGTKVDIK

SEQ ID NO: 601 - DNA, 366 bp, synthetic construct
  misc_feature 1..366: note = Description of Artificial Sequence: Synthetic polynucleotide
  source 1..366: mol_type = other DNA
gaggtgcagttgttggagtctgggggaggcttggtacagcctggggggtccctgagactctcctgtgcagcctctggattcacgtttagtagctatgccatgaactgggtccgccaggctccagggaaggggctggattgggtctcaggtatcagtggtaatggtggtagcacctactacgcagactccgtgaagggccggttcaccatctccagagacatttccaagaacacgctgtatgtgcaaatgcacagcctgagagtcgaggacacggccgtttactactgtgcgaaagcccgttattacgatttttggggggggaatttcgatctctggggccgtggcacccaggtcactgtctcctca

SEQ ID NO: 602 - protein, 122 aa, synthetic construct
  REGION 1..122: note = Description of Artificial Sequence: Synthetic polypeptide
EVQLLESGGGLVQPGGSLRLSCAASGFTFSSYAMNWVRQAPGKGLDWVSGISGNGGSTYYADSVKGRFTISRDISKNTLYVQMHSLRVEDTAVYYCAKARYYDFWGGNFDLWGRGTQVTVSS

SEQ ID NO: 603 - DNA, 24 bp, synthetic construct
  misc_feature 1..24: note = Description of Artificial Sequence: Synthetic oligonucleotide
  source 1..24: mol_type = other DNA
ggattcacgtttagtagctatgcc

SEQ ID NO: 604 - protein, 8 aa, synthetic construct
  REGION 1..8: note = Description of Artificial Sequence: Synthetic peptide
GFTFSSYA

SEQ ID NO: 605 - DNA, 24 bp, synthetic construct
  misc_feature 1..24: note = Description of Artificial Sequence: Synthetic oligonucleotide
  source 1..24: mol_type = other DNA
atcagtggtaatggtggtagcacc

SEQ ID NO: 606 - protein, 8 aa, synthetic construct
  REGION 1..8: note = Description of Artificial Sequence: Synthetic peptide
ISGNGGST

SEQ ID NO: 607 - DNA, 45 bp, synthetic construct
  misc_feature 1..45: note = Description of Artificial Sequence: Synthetic oligonucleotide
  source 1..45: mol_type = other DNA
gcgaaagcccgttattacgatttttggggggggaatttcgatctc

SEQ ID NO: 608 - protein, 15 aa, synthetic construct
  REGION 1..15: note = Description of Artificial Sequence: Synthetic peptide
AKARYYDFWGGNFDL

SEQ ID NO: 609 - DNA, 324 bp, synthetic construct
  misc_feature 1..324: note = Description of Artificial Sequence: Synthetic polynucleotide
  source 1..324: mol_type = other DNA
gaaattgtgttgacgcagtctccaggcaccctgtctttgtctccaggggaaagagccaccctctcctgcagggccagtcagagtgttagcatcaggtacttagcctggtatcagcagaaacctggccaggctcccaggctcctcatctatggtgcatccagcagggccactggcatcccagacaggttcagtgtcagtgtgtctgggacagacttcactctcaccatcactagactggagcctgaagattttgcagtctattactgtcagcaatatggtagttcaccgctcactttcggcggagggaccaaggtggagatcaaa

SEQ ID NO: 610 - protein, 108 aa, synthetic construct
  REGION 1..108: note = Description of Artificial Sequence: Synthetic polypeptide
EIVLTQSPGTLSLSPGERATLSCRASQSVSIRYLAWYQQKPGQAPRLLIYGASSRATGIPDRFSVSVSGTDFTLTITRLEPEDFAVYYCQQYGSSPLTFGGGTKVEIK

SEQ ID NO: 611 - DNA, 21 bp, synthetic construct
  misc_feature 1..21: note = Description of Artificial Sequence: Synthetic oligonucleotide
  source 1..21: mol_type = other DNA
cagagtgttagcatcaggtac

SEQ ID NO: 612 - protein, 7 aa, synthetic construct
  REGION 1..7: note = Description of Artificial Sequence: Synthetic peptide
QSVSIRY

SEQ ID NO: 613-614 - skipped (2 entries)

SEQ ID NO: 615 - DNA, 27 bp, synthetic construct
  misc_feature 1..27: note = Description of Artificial Sequence: Synthetic oligonucleotide
  source 1..27: mol_type = other DNA
cagcaatatggtagttcaccgctcact

SEQ ID NO: 616 - protein, 9 aa, synthetic construct
  REGION 1..9: note = Description of Artificial Sequence: Synthetic peptide
QQYGSSPLT

SEQ ID NO: 617 - DNA, 366 bp, synthetic construct
  misc_feature 1..366: note = Description of Artificial Sequence: Synthetic polynucleotide
  source 1..366: mol_type = other DNA
gaggtgcagttgttggagtctgggggaggcttggtacagcctggggggtccctgagactctcctgtgcagcctctggattcacgtttagtagctatgccatgaactgggtccgccaggctccagggaaggggctggattgggtctcaggtatcagtggtaatggtggtagcacctactacgcagactccgtgaagggccggttcaccatctccagagacatttccaagaacacgctgtatgtgcaaatgcacagcctgagagtcgaggacacggccgtttactactgtgcgaaagcccgttattacgatttttggggggggaatttcgatctctggggccgtggcaccctggtcactgtctcctca

SEQ ID NO: 618 - protein, 122 aa, synthetic construct
  REGION 1..122: note = Description of Artificial Sequence: Synthetic polypeptide
EVQLLESGGGLVQPGGSLRLSCAASGFTFSSYAMNWVRQAPGKGLDWVSGISGNGGSTYYADSVKGRFTISRDISKNTLYVQMHSLRVEDTAVYYCAKARYYDFWGGNFDLWGRGTLVTVSS

SEQ ID NO: 619 - DNA, 324 bp, synthetic construct; identical to SEQ ID NO: 609
  misc_feature 1..324: note = Description of Artificial Sequence: Synthetic polynucleotide
  source 1..324: mol_type = other DNA

SEQ ID NO: 620 - protein, 108 aa, synthetic construct; identical to SEQ ID NO: 610
  REGION 1..108: note = Description of Artificial Sequence: Synthetic polypeptide

SEQ ID NO: 621 - DNA, 366 bp, synthetic construct
  misc_feature 1..366: note = Description of Artificial Sequence: Synthetic polynucleotide
  source 1..366: mol_type = other DNA
gaggtgcagctgttggagtctgggggaggcttggtacagcctggggggtccctgagactctcctgtgcagcctctggattcacgtttagtagctatgccatgagctgggtccgccaggctccagggaaggggctggagtgggtctcagctatcagtggtaatggtggtagcacctactacgcagactccgtgaagggccggttcaccatctccagagacaattccaagaacacgctgtatctgcaaatgaacagcctgagagccgaggacacggccgtatattactgtgcgaaagcccgttattacgatttttggggggggaatttcgatctctggggccgtggcaccctggtcactgtctcctca

SEQ ID NO: 622 - protein, 122 aa, synthetic construct
  REGION 1..122: note = Description of Artificial Sequence: Synthetic polypeptide
EVQLLESGGGLVQPGGSLRLSCAASGFTFSSYAMSWVRQAPGKGLEWVSAISGNGGSTYYADSVKGRFTISRDNSKNTLYLQMNSLRAEDTAVYYCAKARYYDFWGGNFDLWGRGTLVTVSS

SEQ ID NO: 623 - DNA, 324 bp, synthetic construct
  misc_feature 1..324: note = Description of Artificial Sequence: Synthetic polynucleotide
  source 1..324: mol_type = other DNA
gaaattgtgttgacgcagtctccaggcaccctgtctttgtctccaggggaaagagccaccctctcctgcagggccagtcagagtgttagcatcaggtacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcaatatggtagttcaccgctcactttcggcggagggaccaaggtggagatcaaa

SEQ ID NO: 624 - protein, 108 aa, synthetic construct
  REGION 1..108: note = Description of Artificial Sequence: Synthetic polypeptide
EIVLTQSPGTLSLSPGERATLSCRASQSVSIRYLAWYQQKPGQAPRLLIYGASSRATGIPDRFSGSGSGTDFTLTISRLEPEDFAVYYCQQYGSSPLTFGGGTKVEIK

SEQ ID NO: 625 - DNA, 381 bp, synthetic construct
  misc_feature 1..381: note = Description of Artificial Sequence: Synthetic polynucleotide
  source 1..381: mol_type = other DNA
caggttcagctggtgcagtctggacctgaggtgaagaaccctggggcctcagtgaaggtctcctgcaaggcttctggttacacctttaccacctatggtatcagttgggtacgacaggcccctggacaagggcttgagtggatgggatggatcagcggttacaatggtaaaacaaacgatgcacagaagttccaggacagagtcgccatgaccacagacacatccacgagcacagcctacatggagctgaggagcctgagatctgacgacacggccatttattactgttcgagagatcgtttagtagtaccacctgccctttattattcctactacgttatggacgtctggggccaagggaccacggtcaccgtctcctca

SEQ ID NO: 626 - protein, 127 aa, synthetic construct
  REGION 1..127: note = Description of Artificial Sequence: Synthetic polypeptide
QVQLVQSGPEVKNPGASVKVSCKASGYTFTTYGISWVRQAPGQGLEWMGWISGYNGKTNDAQKFQDRVAMTTDTSTSTAYMELRSLRSDDTAIYYCSRDRLVVPPALYYSYYVMDVWGQGTTVTVSS

SEQ ID NO: 627 - DNA, 24 bp, synthetic construct
  misc_feature 1..24: note = Description of Artificial Sequence: Synthetic oligonucleotide
  source 1..24: mol_type = other DNA
ggttacacctttaccacctatggt

SEQ ID NO: 628 - protein, 8 aa, synthetic construct
  REGION 1..8: note = Description of Artificial Sequence: Synthetic peptide
GYTFTTYG

SEQ ID NO: 629 - DNA, 24 bp, synthetic construct
  misc_feature 1..24: note = Description of Artificial Sequence: Synthetic oligonucleotide
  source 1..24: mol_type = other DNA
atcagcggttacaatggtaaaaca

SEQ ID NO: 630 - protein, 8 aa, synthetic construct
  REGION 1..8: note = Description of Artificial Sequence: Synthetic peptide
ISGYNGKT

SEQ ID NO: 631 - DNA, 60 bp, synthetic construct
  misc_feature 1..60: note = Description of Artificial Sequence: Synthetic oligonucleotide
  source 1..60: mol_type = other DNA
tcgagagatcgtttagtagtaccacctgccctttattattcctactacgttatggacgtc

SEQ ID NO: 632 - protein, 20 aa, synthetic construct
  REGION 1..20: note = Description of Artificial Sequence: Synthetic peptide
SRDRLVVPPALYYSYYVMDV

SEQ ID NO: 633 - DNA, 336 bp, synthetic construct
  misc_feature 1..336: note = Description of Artificial Sequence: Synthetic polynucleotide
  source 1..336: mol_type = other DNA
gatgttgtgatgactcagtctccactctccctgcccgtcacccttggacagccggcctccatctcctgcaggtctagtcaaagcctcgtatacagtgatggaaacacctacttgaattggtttcagcagaggccaggtcaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgtacacttttggccaggggaccaagctggagatcaaa

SEQ ID NO: 634 - protein, 112 aa, synthetic construct; identical to SEQ ID NO: 336
  REGION 1..112: note = Description of Artificial Sequence: Synthetic polypeptide

SEQ ID NO: 635 - DNA, 33 bp, synthetic construct; identical to SEQ ID NO: 323
  misc_feature 1..33: note = Description of Artificial Sequence: Synthetic oligonucleotide
  source 1..33: mol_type = other DNA

SEQ ID NO: 636 - protein, 11 aa, synthetic construct
  REGION 1..11: note = Description of Artificial Sequence: Synthetic peptide
QSLVYSDGNTY

SEQ ID NO: 637-638 - skipped (2 entries)

SEQ ID NO: 639 - DNA, 27 bp, synthetic construct
  misc_feature 1..27: note = Description of Artificial Sequence: Synthetic oligonucleotide
  source 1..27: mol_type = other DNA
atgcaaggtacacactggccgtacact

SEQ ID NO: 640 - protein, 9 aa, synthetic construct
  REGION 1..9: note = Description of Artificial Sequence: Synthetic peptide
MQGTHWPYT

SEQ ID NO: 641 - DNA, 381 bp, synthetic construct; identical to SEQ ID NO: 625
  misc_feature 1..381: note = Description of Artificial Sequence: Synthetic polynucleotide
  source 1..381: mol_type = other DNA

SEQ ID NO: 642 - protein, 127 aa, synthetic construct; identical to SEQ ID NO: 626
  REGION 1..127: note = Description of Artificial Sequence: Synthetic polypeptide

SEQ ID NO: 643 - DNA, 336 bp, synthetic construct; identical to SEQ ID NO: 633
  misc_feature 1..336: note = Description of Artificial Sequence: Synthetic polynucleotide
  source 1..336: mol_type = other DNA

SEQ ID NO: 644 - protein, 112 aa, synthetic construct; identical to SEQ ID NO: 336
  REGION 1..112: note = Description of Artificial Sequence: Synthetic polypeptide

SEQ ID NO: 645 - DNA, 381 bp, synthetic construct
  misc_feature 1..381: note = Description of Artificial Sequence: Synthetic polynucleotide
  source 1..381: mol_type = other DNA
caggttcagctggtgcagtctggagctgaggtgaagaagcctggggcctcagtgaaggtctcctgcaaggcttctggttacacctttaccacctatggtatcagctgggtgcgacaggcccctggacaagggcttgagtggatgggatggatcagcggttacaatggtaaaacaaactatgcacagaagctccagggcagagtcaccatgaccacagacacatccacgagcacagcctacatggagctgaggagcctgagatctgacgacacggccgtgtattactgttcgagagatcgtttagtagtaccacctgccctttattattcctactacgttatggacgtctgggggcaagggaccacggtcaccgtctcctca

SEQ ID NO: 646 - protein, 127 aa, synthetic construct
  REGION 1..127: note = Description of Artificial Sequence: Synthetic polypeptide
QVQLVQSGAEVKKPGASVKVSCKASGYTFTTYGISWVRQAPGQGLEWMGWISGYNGKTNYAQKLQGRVTMTTDTSTSTAYMELRSLRSDDTAVYYCSRDRLVVPPALYYSYYVMDVWGQGTTVTVSS

SEQ ID NO: 647 - DNA, 336 bp, synthetic construct; identical to SEQ ID NO: 335
  misc_feature 1..336: note = Description of Artificial Sequence: Synthetic polynucleotide
  source 1..336: mol_type = other DNA

SEQ ID NO: 648 - protein, 112 aa, synthetic construct; identical to SEQ ID NO: 336
  REGION 1..112: note = Description of Artificial Sequence: Synthetic polypeptide

SEQ ID NO: 649 - DNA, 381 bp, synthetic construct
  misc_feature 1..381: note = Description of Artificial Sequence: Synthetic polynucleotide
  source 1..381: mol_type = other DNA
caggttcagctggtgcagtctggacctgaggtgaagaaccctggggcctcagtgaaggtctcctgcaaggcttctggttacacctttaccacctatggtatcagttgggtacgacaggcccctggacaagggcttgagtggatgggatggatcagcggttacaatggtaaaacaaacgatgcacagaagttccaggacagagtcgccatgaccacagacacatccacgagcacagcctacatggagctgaggagcctgagatctgacgacacggccatttattactgttcgagagatcgtttagtagtaccacctgcccttaattattactactacgttatggacgtctggggccaagggaccacggtcaccgtctcctca

SEQ ID NO: 650 - protein, 127 aa, synthetic construct
  REGION 1..127: note = Description of Artificial Sequence: Synthetic polypeptide
QVQLVQSGPEVKNPGASVKVSCKASGYTFTTYGISWVRQAPGQGLEWMGWISGYNGKTNDAQKFQDRVAMTTDTSTSTAYMELRSLRSDDTAIYYCSRDRLVVPPALNYYYYVMDVWGQGTTVTVSS

SEQ ID NO: 651 - DNA, 24 bp, synthetic construct
  misc_feature 1..24: note = Description of Artificial Sequence: Synthetic oligonucleotide
  source 1..24: mol_type = other DNA
ggttacacctttaccacctatggt

SEQ ID NO: 652 - protein, 8 aa, synthetic construct
  REGION 1..8: note = Description of Artificial Sequence: Synthetic peptide
GYTFTTYG

SEQ ID NO: 653 - DNA, 24 bp, synthetic construct
  misc_feature 1..24: note = Description of Artificial Sequence: Synthetic oligonucleotide
  source 1..24: mol_type = other DNA
atcagcggttacaatggtaaaaca

SEQ ID NO: 654 - protein, 8 aa, synthetic construct
  REGION 1..8: note = Description of Artificial Sequence: Synthetic peptide
ISGYNGKT

SEQ ID NO: 655 - DNA, 60 bp, synthetic construct
  misc_feature 1..60: note = Description of Artificial Sequence: Synthetic oligonucleotide
  source 1..60: mol_type = other DNA
tcgagagatcgtttagtagtaccacctgcccttaattattactactacgttatggacgtc

SEQ ID NO: 656 - protein, 20 aa, synthetic construct
  REGION 1..20: note = Description of Artificial Sequence: Synthetic peptide
SRDRLVVPPALNYYYYVMDV

SEQ ID NO: 657 - DNA, 336 bp, synthetic construct; identical to SEQ ID NO: 633
  misc_feature 1..336: note = Description of Artificial Sequence: Synthetic polynucleotide
  source 1..336: mol_type = other DNA

SEQ ID NO: 658 - protein, 112 aa, synthetic construct; identical to SEQ ID NO: 336
  REGION 1..112: note = Description of Artificial Sequence: Synthetic polypeptide

SEQ ID NO: 659 - DNA, 33 bp, synthetic construct; identical to SEQ ID NO: 323
  misc_feature 1..33: note = Description of Artificial Sequence: Synthetic oligonucleotide
  source 1..33: mol_type = other DNA

SEQ ID NO: 660 - protein, 11 aa, synthetic construct
  REGION 1..11: note = Description of Artificial Sequence: Synthetic peptide
QSLVYSDGNTY

SEQ ID NO: 661-662 - skipped (2 entries)

SEQ ID NO: 663 - DNA, 27 bp, synthetic construct
  misc_feature 1..27: note = Description of Artificial Sequence: Synthetic oligonucleotide
  source 1..27: mol_type = other DNA
atgcaaggtacacactggccgtacact

SEQ ID NO: 664 - protein, 9 aa, synthetic construct
  REGION 1..9: note = Description of Artificial Sequence: Synthetic peptide
MQGTHWPYT

SEQ ID NO: 665 - DNA, 381 bp, synthetic construct; identical to SEQ ID NO: 649
  misc_feature 1..381: note = Description of Artificial Sequence: Synthetic polynucleotide
  source 1..381: mol_type = other DNA

SEQ ID NO: 666 - protein, 127 aa, synthetic construct; identical to SEQ ID NO: 650
  REGION 1..127: note = Description of Artificial Sequence: Synthetic polypeptide

SEQ ID NO: 667 - DNA, 336 bp, synthetic construct; identical to SEQ ID NO: 633
  misc_feature 1..336: note = Description of Artificial Sequence: Synthetic polynucleotide
  source 1..336: mol_type = other DNA

SEQ ID NO: 668 - protein, 112 aa, synthetic construct; identical to SEQ ID NO: 336
  REGION 1..112: note = Description of Artificial Sequence: Synthetic polypeptide

SEQ ID NO: 669 - DNA, 381 bp, synthetic construct
  misc_feature 1..381: note = Description of Artificial Sequence: Synthetic polynucleotide
  source 1..381: mol_type = other DNA
caggttcagctggtgcagtctggagctgaggtgaagaagcctggggcctcagtgaaggtctcctgcaaggcttctggttacacctttaccacctatggtatcagctgggtgcgacaggcccctggacaagggcttgagtggatgggatggatcagcggttacaatggtaaaacaaactatgcacagaagctccagggcagagtcaccatgaccacagacacatccacgagcacagcctacatggagctgaggagcctgagatctgacgacacggccgtgtattactgttcgagagatcgtttagtagtaccacctgcccttaattattactactacgttatggacgtctgggggcaagggaccacggtcaccgtctcctca

SEQ ID NO: 670 - protein, 127 aa, synthetic construct
  REGION 1..127: note = Description of Artificial Sequence: Synthetic polypeptide
QVQLVQSGAEVKKPGASVKVSCKASGYTFTTYGISWVRQAPGQGLEWMGWISGYNGKTNYAQKLQGRVTMTTDTSTSTAYMELRSLRSDDTAVYYCSRDRLVVPPALNYYYYVMDVWGQGTTVTVSS

SEQ ID NO: 671 - DNA, 336 bp, synthetic construct; identical to SEQ ID NO: 335
  misc_feature 1..336: note = Description of Artificial Sequence: Synthetic polynucleotide
  source 1..336: mol_type = other DNA

SEQ ID NO: 672 - protein, 112 aa, synthetic construct; identical to SEQ ID NO: 336
  REGION 1..112: note = Description of Artificial Sequence: Synthetic polypeptide

SEQ ID NO: 673 - DNA, 381 bp, synthetic construct
  misc_feature 1..381: note = Description of Artificial Sequence: Synthetic polynucleotide
  source 1..381: mol_type = other DNA
caggttcagctggtgcagtctggacctgaggtgaagaaccctggggcctcagtgaaggtctcctgcaaggcttctggttacacctttaccacctatggtatcagttgggtacgacaggcccctggacaagggcttgagtggatgggatggatcagcggttacaatggtaaaacaaacgatgcacagaagttccaggacagagtcgccatgaccacagacacatccacgagcacagcctacatggagctgaggagcctgagatctgacgacacggccatttattactgttcgagagatcgtttagtagtaccacctgccctttattattactactacgttatggacgtctggggccaagggaccacggtcaccgtctcctca

SEQ ID NO: 674 - protein, 127 aa, synthetic construct
  REGION 1..127: note = Description of Artificial Sequence: Synthetic polypeptide
QVQLVQSGPEVKNPGASVKVSCKASGYTFTTYGISWVRQAPGQGLEWMGWISGYNGKTNDAQKFQDRVAMTTDTSTSTAYMELRSLRSDDTAIYYCSRDRLVVPPALYYYYYVMDVWGQGTTVTVSS

SEQ ID NO: 675 - DNA, 24 bp, synthetic construct
  misc_feature 1..24: note = Description of Artificial Sequence: Synthetic oligonucleotide
  source 1..24: mol_type = other DNA
ggttacacctttaccacctatggt

SEQ ID NO: 676 - protein, 8 aa, synthetic construct
  REGION 1..8: note = Description of Artificial Sequence: Synthetic peptide
GYTFTTYG

SEQ ID NO: 677 - DNA, 24 bp, synthetic construct
  misc_feature 1..24: note = Description of Artificial Sequence: Synthetic oligonucleotide
  source 1..24: mol_type = other DNA
atcagcggttacaatggtaaaaca

SEQ ID NO: 678 - protein, 8 aa, synthetic construct
  REGION 1..8: note = Description of Artificial Sequence: Synthetic peptide
ISGYNGKT

SEQ ID NO: 679 - DNA, 60 bp, synthetic construct
  misc_feature 1..60: note = Description of Artificial Sequence: Synthetic oligonucleotide
  source 1..60: mol_type = other DNA
tcgagagatcgtttagtagtaccacctgccctttattattactactacgttatggacgtc

SEQ ID NO: 680 - protein, 20 aa, synthetic construct
  REGION 1..20: note = Description of Artificial Sequence: Synthetic peptide
SRDRLVVPPALYYYYYVMDV

SEQ ID NO: 681 - DNA, 336 bp, synthetic construct; identical to SEQ ID NO: 633
  misc_feature 1..336: note = Description of Artificial Sequence: Synthetic polynucleotide
  source 1..336: mol_type = other DNA

SEQ ID NO: 682 - protein, 112 aa, synthetic construct; identical to SEQ ID NO: 336
  REGION 1..112: note = Description of Artificial Sequence: Synthetic polypeptide

SEQ ID NO: 683 - DNA, 33 bp, synthetic construct; identical to SEQ ID NO: 323
  misc_feature 1..33: note = Description of Artificial Sequence: Synthetic oligonucleotide
  source 1..33: mol_type = other DNA

SEQ ID NO: 684 - protein, 11 aa, synthetic construct
  REGION 1..11: note = Description of Artificial Sequence: Synthetic peptide
QSLVYSDGNTY

SEQ ID NO: 685-686 - skipped (2 entries)

SEQ ID NO: 687 - DNA, 27 bp, synthetic construct
  misc_feature 1..27: note = Description of Artificial Sequence: Synthetic oligonucleotide
  source 1..27: mol_type = other DNA
atgcaaggtacacactggccgtacact

SEQ ID NO: 688 - protein, 9 aa, synthetic construct
  REGION 1..9: note = Description of Artificial Sequence: Synthetic peptide
MQGTHWPYT

SEQ ID NO: 689 - DNA, 381 bp, synthetic construct; identical to SEQ ID NO: 673
  misc_feature 1..381: note = Description of Artificial Sequence: Synthetic polynucleotide
  source 1..381: mol_type = other DNA

SEQ ID NO: 690 - protein, 127 aa, synthetic construct; identical to SEQ ID NO: 674
  REGION 1..127: note = Description of Artificial Sequence: Synthetic polypeptide

SEQ ID NO: 691 - DNA, 336 bp, synthetic construct; identical to SEQ ID NO: 633
  misc_feature 1..336: note = Description of Artificial Sequence: Synthetic polynucleotide
  source 1..336: mol_type = other DNA

SEQ ID NO: 692 - protein, 112 aa, synthetic construct; identical to SEQ ID NO: 336
  REGION 1..112: note = Description of Artificial Sequence: Synthetic polypeptide

SEQ ID NO: 693 - DNA, 381 bp, synthetic construct
  misc_feature 1..381: note = Description of Artificial Sequence: Synthetic polynucleotide
  source 1..381: mol_type = other DNA
caggttcagctggtgcagtctggagctgaggtgaagaagcctggggcctcagtgaaggtctcctgcaaggcttctggttacacctttaccacctatggtatcagctgggtgcgacaggcccctggacaagggcttgagtggatgggatggatcagcggttacaatggtaaaacaaactatgcacagaagctccagggcagagtcaccatgaccacagacacatccacgagcacagcctacatggagctgaggagcctgagatctgacgacacggccgtgtattactgttcgagagatcgtttagtagtaccacctgccctttattattactactacgttatggacgtctgggggcaagggaccacggtcaccgtctcctca

SEQ ID NO: 694 - protein, 127 aa, synthetic construct
  REGION 1..127: note = Description of Artificial Sequence: Synthetic polypeptide
QVQLVQSGAEVKKPGASVKVSCKASGYTFTTYGISWVRQAPGQGLEWMGWISGYNGKTNYAQKLQGRVTMTTDTSTSTAYMELRSLRSDDTAVYYCSRDRLVVPPALYYYYYVMDVWGQGTTVTVSS

SEQ ID NO: 695 - DNA, 336 bp, synthetic construct; identical to SEQ ID NO: 335
  misc_feature 1..336: note = Description of Artificial Sequence: Synthetic polynucleotide
  source 1..336: mol_type = other DNA

SEQ ID NO: 696 - protein, 112 aa, synthetic construct; identical to SEQ ID NO: 336
  REGION 1..112: note = Description of Artificial Sequence: Synthetic polypeptide

SEQ ID NO: 697 - DNA, 384 bp, synthetic construct; identical to SEQ ID NO: 361
  misc_feature 1..384: note = Description of Artificial Sequence: Synthetic polynucleotide
  source 1..384: mol_type = other DNA

SEQ ID NO: 698 - protein, 128 aa, synthetic construct; identical to SEQ ID NO: 362
  REGION 1..128: note = Description of Artificial Sequence: Synthetic polypeptide

SEQ ID NO: 699 - DNA, 24 bp, synthetic construct
  misc_feature 1..24: note = Description of Artificial Sequence: Synthetic oligonucleotide
  source 1..24: mol_type = other DNA
ggattcaccttcagtgaccactac

SEQ ID NO: 700 - protein, 8 aa, synthetic construct
  REGION 1..8: note = Description of Artificial Sequence: Synthetic peptide
GFTFSDHY

SEQ ID NO: 701 - DNA, 24 bp, synthetic construct
  misc_feature 1..24: note = Description of Artificial Sequence: Synthetic oligonucleotide
  source 1..24: mol_type = other DNA
attagtaatgatggtggtaccaaa

SEQ ID NO: 702 - protein, 8 aa, synthetic construct
  REGION 1..8: note = Description of Artificial Sequence: Synthetic peptide
ISNDGGTK

SEQ ID NO: 703 - DNA, 63 bp, synthetic construct; identical to SEQ ID NO: 367
  misc_feature 1..63: note = Description of Artificial Sequence: Synthetic oligonucleotide
  source 1..63: mol_type = other DNA

SEQ ID NO: 704 - protein, 21 aa, synthetic construct
  REGION 1..21: note = Description of Artificial Sequence: Synthetic peptide
ARDQGYIGYDSYYYYSYGMDV

SEQ ID NO: 705 - DNA, 321 bp, synthetic construct
  misc_feature 1..321: note = Description of Artificial Sequence: Synthetic polynucleotide
  source 1..321: mol_type = other DNA
aaaattgtgttgacgcagtctccaggcaccctgcctttgtttccaggggaaagagccaccctctcctgtagggccagtcagagtgttaacaacaaattcttagcctggtaccagcagaaatctggccaggctcccaggctcctcatctatggtgcatccagcagggccactggcatcccagacaggttcagtggcagtgggtctgggaccgacttcactctcaccatcagcggactggagcctgaagattttgaagtgtattattgtcaagtatatggtaactcactcactttcggcggagggaccaaggtggagatcaag

SEQ ID NO: 706 - protein, 107 aa, synthetic construct
  REGION 1..107: note = Description of Artificial Sequence: Synthetic polypeptide
KIVLTQSPGTLPLFPGERATLSCRASQSVNNKFLAWYQQKSGQAPRLLIYGASSRATGIPDRFSGSGSGTDFTLTISGLEPEDFEVYYCQVYGNSLTFGGGTKVEIK

SEQ ID NO: 707 - DNA, 21 bp, synthetic construct
  misc_feature 1..21: note = Description of Artificial Sequence: Synthetic oligonucleotide
  source 1..21: mol_type = other DNA
cagagtgttaacaacaaattc

SEQ ID NO: 708 - protein, 7 aa, synthetic construct
  REGION 1..7: note = Description of Artificial Sequence: Synthetic peptide
QSVNNKF

SEQ ID NO: 709-710 - skipped (2 entries)

SEQ ID NO: 711 - DNA, 24 bp, synthetic construct
  misc_feature 1..24: note = Description of Artificial Sequence: Synthetic oligonucleotide
  source 1..24: mol_type = other DNA
caagtatatggtaactcactcact

SEQ ID NO: 712 - protein, 8 aa, synthetic construct
  REGION 1..8: note = Description of Artificial Sequence: Synthetic peptide
QVYGNSLT

SEQ ID NO: 713 - DNA, 384 bp, synthetic construct; identical to SEQ ID NO: 377
  misc_feature 1..384: note = Description of Artificial Sequence: Synthetic polynucleotide
  source 1..384: mol_type = other DNA

SEQ ID NO: 714 - protein, 128 aa, synthetic construct; identical to SEQ ID NO: 378
  REGION 1..128: note = Description of Artificial Sequence: Synthetic polypeptide

SEQ ID NO: 715 - DNA, 321 bp, synthetic construct
  misc_feature 1..321: note = Description of Artificial Sequence: Synthetic polynucleotide
  source 1..321: mol_type = other DNA
gaaattgtgttgacgcagtctccaggcaccctgcctttgtttccaggggaaagagccaccctctcctgtagggccagtcagagtgttaacaacaaattcttagcctggtaccagcagaaatctggccaggctcccaggctcctcatctatggtgcatccagcagggccactggcatcccagacaggttcagtggcagtgggtctgggaccgacttcactctcaccatcagcggactggagcctgaagattttgaagtgtattattgtcaagtatatggtaactcactcactttcggcggagggaccaaggtggagatcaaa

SEQ ID NO: 716 - protein, 107 aa, synthetic construct
  REGION 1..107: note = Description of Artificial Sequence: Synthetic polypeptide
EIVLTQSPGTLPLFPGERATLSCRASQSVNNKFLAWYQQKSGQAPRLLIYGASSRATGIPDRFSGSGSGTDFTLTISGLEPEDFEVYYCQVYGNSLTFGGGTKVEIK

SEQ ID NO: 717 - DNA, 384 bp, synthetic construct; identical to SEQ ID NO: 381
  misc_feature 1..384: note = Description of Artificial Sequence: Synthetic polynucleotide
  source 1..384: mol_type = other DNA

SEQ ID NO: 718 - protein, 128 aa, synthetic construct; identical to SEQ ID NO: 382
  REGION 1..128: note = Description of Artificial Sequence: Synthetic polypeptide

SEQ ID NO: 719 - DNA, 321 bp, synthetic construct; identical to SEQ ID NO: 383
  misc_feature 1..321: note = Description of Artificial Sequence: Synthetic polynucleotide
  source 1..321: mol_type = other DNA

SEQ ID NO: 720 - protein, 107 aa, synthetic construct; identical to SEQ ID NO: 384
  REGION 1..107: note = Description of Artificial Sequence: Synthetic polypeptide

SEQ ID NO: 721 - DNA, 378 bp, synthetic construct; identical to SEQ ID NO: 505
  misc_feature 1..378: note = Description of Artificial Sequence: Synthetic polynucleotide
  source 1..378: mol_type = other DNA

SEQ ID NO: 722 - protein, 126 aa, synthetic construct; identical to SEQ ID NO: 506
  REGION 1..126: note = Description of Artificial Sequence: Synthetic polypeptide

SEQ ID NO: 723 - DNA, 24 bp, synthetic construct
  misc_feature 1..24: note = Description of Artificial Sequence: Synthetic oligonucleotide
  source 1..24: mol_type = other DNA
ggttacacctttaccaactacgct

SEQ ID NO: 724 - protein, 8 aa, synthetic construct
  REGION 1..8: note = Description of Artificial Sequence: Synthetic peptide
GYTFTNYA

SEQ ID NO: 725 - DNA, 24 bp, synthetic construct
  misc_feature 1..24: note = Description of Artificial Sequence: Synthetic oligonucleotide
  source 1..24: mol_type = other DNA
gtcagcgcttacaatggtcacaca

SEQ ID NO: 726 - protein, 8 aa, synthetic construct
  REGION 1..8: note = Description of Artificial Sequence: Synthetic peptide
VSAYNGHT

SEQ ID NO: 727 - DNA, 57 bp, synthetic construct; identical to SEQ ID NO: 511
  misc_feature 1..57: note = Description of Artificial Sequence: Synthetic oligonucleotide
  source 1..57: mol_type = other DNA

SEQ ID NO: 728 - protein, 19 aa, synthetic construct
  REGION 1..19: note = Description of Artificial Sequence: Synthetic peptide
ARGGVVVPVAPHFYNGMDV

SEQ ID NO: 729 - DNA, 336 bp, synthetic construct
  misc_feature 1..336: note = Description of Artificial Sequence: Synthetic polynucleotide
  source 1..336: mol_type = other DNA
gatattgtgatgactcagtttccactctccctgcccgtcacccctggagagccggcctccatctcctgcaggtctagtcagagcctcctgcatattaatgaatacaactatttggattggtacctaaagaagccagggcagtctccacagctcctgatctatttgggttttaatcgggcctccggggtccctgacaggttcagtggcagtggatcaggcacagattttacactgaaaatcagcagagtggaggctgaggatgttggggtctattactgcatgcaagctcttcaaactccgtggacgttcggccaagggaccaaggtggaaatcaaa

SEQ ID NO: 730 - protein, 112 aa, synthetic construct
  REGION 1..112: note = Description of Artificial Sequence: Synthetic polypeptide
DIVMTQFPLSLPVTPGEPASISCRSSQSLLHINEYNYLDWYLKKPGQSPQLLIYLGFNRASGVPDRFSGSGSGTDFTLKISRVEAEDVGVYYCMQALQTPWTFGQGTKVEIK

SEQ ID NO: 731 - DNA, 33 bp, synthetic construct; identical to SEQ ID NO: 515
  misc_feature 1..33: note = Description of Artificial Sequence: Synthetic oligonucleotide
  source 1..33: mol_type = other DNA

SEQ ID NO: 732 - protein, 11 aa, synthetic construct
  REGION 1..11: note = Description of Artificial Sequence: Synthetic peptide
QSLLHINEYNY

SEQ ID NO: 733-734 - skipped (2 entries)

SEQ ID NO: 735 - DNA, 27 bp, synthetic construct
  misc_feature 1..27: note = Description of Artificial Sequence: Synthetic oligonucleotide
  source 1..27: mol_type = other DNA
atgcaagctcttcaaactccgtggacg

SEQ ID NO: 736 - protein, 9 aa, synthetic construct
  REGION 1..9: note = Description of Artificial Sequence: Synthetic peptide
MQALQTPWT

SEQ ID NO: 737 - DNA, 378 bp, synthetic construct; identical to SEQ ID NO: 521
  misc_feature 1..378: note = Description of Artificial Sequence: Synthetic polynucleotide
  source 1..378: mol_type = other DNA

SEQ ID NO: 738 - protein, 126 aa, synthetic construct; identical to SEQ ID NO: 522
  REGION 1..126: note = Description of Artificial Sequence: Synthetic polypeptide

SEQ ID NO: 739 - DNA, 336 bp, synthetic construct
  misc_feature 1..336: note = Description of Artificial Sequence: Synthetic polynucleotide
  source 1..336: mol_type = other DNA
gatattgtgatgactcagtctccactctccctgcccgtcacccctggagagccggcctccatctcctgcaggtctagtcagagcctcctgcatattaatgaatacaactatttggattggtacctaaagaagccagggcagtctccacagctcctgatctatttgggttttaatcgggcctccggggtccctgacaggttcagtggcagtggatcaggcacagattttacactgaaaatcagcagagtggaggctgaggatgttggggtctattactgcatgcaagctcttcaaactccgtggacgttcggccaagggaccaaggtggaaatcaaa

SEQ ID NO: 740 - protein, 112 aa, synthetic construct
  REGION 1..112: note = Description of Artificial Sequence: Synthetic polypeptide
DIVMTQSPLSLPVTPGEPASISCRSSQSLLHINEYNYLDWYLKKPGQSPQLLIYLGFNRASGVPDRFSGSGSGTDFTLKISRVEAEDVGVYYCMQALQTPWTFGQGTKVEIK

SEQ ID NO: 741 - DNA, 378 bp, synthetic construct; identical to SEQ ID NO: 525
  misc_feature 1..378: note = Description of Artificial Sequence: Synthetic polynucleotide
  source 1..378: mol_type = other DNA

SEQ ID NO: 742 - protein, 126 aa, synthetic construct; identical to SEQ ID NO: 526
  REGION 1..126: note = Description of Artificial Sequence: Synthetic polypeptide

SEQ ID NO: 743 - DNA, 336 bp, synthetic construct; identical to SEQ ID NO: 527
  misc_feature 1..336: note = Description of Artificial Sequence: Synthetic polynucleotide
  source 1..336: mol_type = other DNA

SEQ ID NO: 744 - protein, 112 aa, synthetic construct; identical to SEQ ID NO: 528
  REGION 1..112: note = Description of Artificial Sequence: Synthetic polypeptide

SEQ ID NO: 745-750 - skipped (6 entries)

SEQ ID NO: 751 - protein, 330 aa, synthetic construct [sequence omitted]

SEQ ID NO: 752 - protein, 327 aa, synthetic construct
  REGION 1..327: note = Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 753 - protein, 327 aa, synthetic construct
  REGION 1..327: note = Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 754 - DNA, 2076 bp, Homo sapiens
  source 1..2076: mol_type = genomic DNA
atgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccag

SEQ ID NO: 755 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 756 - protein, 692 aa, Macaca mulatta
MGTVSSRRSWWPLPLPLLLLLLLGPAGARAQEDEDGDYEELVLALRSEEDGLADAPEHGATATFHRCAKDPWRLPGTYVVVLKEETHRSQSERTARRLQAQAARRGYLTKILHVFHHLLPGFLVKMSGDLLELALKLPHVDYIEEDSSVFAQSIPWNLERITPARYRADEYQPPKGGSLVEVYLLDTSIQSDHREIEGRVMVTDFESVPEEDGTRFHRQASKCDSHGTHLAGVVSGRDAGVAKGAGLRSLRVLNCQGKGTVSGTLIGLEFIRKSQLVQPVGPLVVLLPLAGGYSRVFNAACQRLARAGVVLVTAAGNFRDDACLYSPASAPEVITVGATNAQDQPVTLGTLGTNFGRCVDLFAPGEDIIGASSDCSTCFVSRSGTSQAAAHVAGIAAMMLSAEPELTLAELRQRLIHFSAKDVINEAWFPEDQRVLTPNLVAALPPSTHRAGWQLFCRTVWSAHSGPTRMATAVARCAQDEELLSCSSFSRSGKRRGERIEAQGGKRVCRAHNAFGGEGVYAIARCCLLPQVNCSVHTAPPAGASMGTRVHCHQQGHVLTGCSSHWEVEDLGTHKPPVLRPRGQPNQCVGHREASIHASCCHAPGLECKVKEHGIPAPQEQVIVACEDGWTLTGCSPLPGTSHVLGAYAVDNTCVVRSRDVSTTGSTSKEAVAAVAICCRSRHLVQASQELQ

SEQ ID NO: 757 - protein, 694 aa, Mus musculus
MGTHCSAWLRWPLLPLLPPLLLLLLLLCPTGAGAQDEDGDYEELMLALPSQEDGLADEAAHVATATFRRCSKEAWRLPGTYIVVLMEETQRLQIEQTAHRLQTRAARRGYVIKVLHIFYDLFPGFLVKMSSDLLGLALKLPHVEYIEEDSFVFAQSIPWNLERIIPAWHQTEEDRSPDGSSQVEVYLLDTSIQGAHREIEGRVTITDFNSVPEEDGTRFHRQASKCDSHGTHLAGVVSGRDAGVAKGTSLHSLRVLNCQGKGTVSGTLIGLEFIRKSQLIQPSGPLVVLLPLAGGYSRILNAACRHLARTGVVLVAAAGNFRDDACLYSPASAPEVITVGATNAQDQPVTLGTLGTNFGRCVDLFAPGKDIIGASSDCSTCFMSQSGTSQAAAHVAGIVARMLSREPTLTLAELRQRLIHFSTKDVINMAWFPEDQQVLTPNLVATLPPSTHETGGQLLCRTVWSAHSGPTRTATATARCAPEEELLSCSSFSRSGRRRGDWIEAIGGQQVCKALNAFGGEGVYAVARCCLVPRANCSIHNTPAARAGLETHVHCHQKDHVLTGCSFHWEVEDLSVRRQPALRSRRQPGQCVGHQAASVYASCCHAPGLECKIKEHGISGPSEQVTVACEAGWTLTGCNVLPGASLTLGAYSVDNLCVARVHDTARADRTSGEATVAAAICCRSRPSAKASWVQ

SEQ ID NO: 758 - protein, 653 aa, Homo sapiens
EFRCHDGKCISRQFVCDSDRDCLDGSDEASCPVLTCGPASFQCNSSTCIPQLWACDNDPDCEDGSDEWPQRCRGLYVFQGDSSPCSAFEFHCLSGECIHSSWRCDGGPDCKDKSDEENCAVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTEAEAAVATQETSTVRLKVSSTAVRTQHTTTRPVPDTSRLPGATPGLTTVEIV

(19) United States
(12) Patent Application Publication
(10) Pub. No.: US 2025/0064926 A1
(43) Pub. Date: Feb. 27, 2025

(54) HUMAN ANTIBODIES TO PCSK9 FOR USE IN METHODS OF TREATING PARTICULAR GROUPS OF SUBJECTS

(71) Applicant: Sanofi Biotechnology, Gentilly (FR)

(72) Inventors: Corinne Hanotin, Paris (FR); Laurence Bessac, Paris (FR); Umesh Chaudhari, Cambridge, MA (US)

(21) Appl. No.: 18/790,032

(22) Filed: Jul. 31, 2024

Related U.S. Application Data
(63) Continuation of application No. 17/560,402, filed on Dec. 23, 2021, now Pat. No. 12,083,176.
Application No. 17/560,402 is a continuation of application No. 16/365,317, filed on Mar. 26, 2019, now Pat. No. 11,246,925.
Application No. 16/365,317 is a continuation of application No. 15/389,130, filed on Dec. 22, 2016, now abandoned.
Application No. 15/389,130 is a division of application No. 13/982,373, filed on Jul. 29, 2013, now Pat. No. 9,561,155.

(30) Foreign Application Priority Data
Jan. 28, 2011 (EP) 11305088.4
Jan. 28, 2011 (EP) 11305089.2
Apr. 29, 2011 (EP) 11305513.1
Apr. 29, 2011 (EP) 11305514.9
Aug. 12, 2011 (EP) 11306039.6
Aug. 12, 2011 (EP) 11306040.4
Sep. 22, 2011 (EP) 11306201.2
Sep. 22, 2011 (EP) 11306202.0
Nov. 8, 2011 (EP) 11306449.7
Nov. 8, 2011 (EP) 11306450.5

(51) Int. Cl. A61K 39/395; A61J 1/05; A61K 31/215; A61K 31/366; A61K 31/404; A61K 31/435; A61K 31/47; A61K 31/505; A61K 39/00; A61K 45/06; A61P 3/06; C07K 14/47; C07K 16/40

(52) U.S. Cl. CPC A61K 39/3955; A61P 3/06; C07K 16/40; A61J 1/05; A61K 31/215; A61K 31/366; A61K 31/404; A61K 31/435; A61K 31/47; A61K 31/505; A61K 39/395; A61K 2039/505; A61K 2039/545; A61K 45/06; C07K 14/4703; C07K 2317/21; C07K 2317/76

(57) ABSTRACT

The present invention relates to methods for treating diseases or conditions in which proprotein convertase subtilisin/kexin type 9 (PCSK9) expression or activity causes an impact by administration of PCSK9-specific antibodies or antigen-binding fragments thereof and preferably by additional administration of an inhibitor of 3-hydroxy-3-methyl-glutaryl-CoA reductase (HMG-COA reductase). The present invention further relates to PCSK9-specific antibodies or antigen-binding fragments thereof for use in the treatment of diseases or conditions in which PCSK9 expression or activity causes an impact.

The present invention also relates to articles of manufacture comprising packaging material, PCSK9-specific antibodies or antigen-binding fragments thereof, and a label or packaging insert indicating which groups of patients can be treated with said antibodies or fragments, which groups of patients must not be treated with said antibodies or fragments, and which dosage regimen should be used.

The present invention further relates to methods of testing the efficacy of PCSK9-specific antibodies or antigen-binding fragments thereof for the treatment of certain diseases or conditions and for the treatment of specific sub-groups of patients.

### CROSS-REFERENCE TO RELATED APPLICATIONS

This application is a continuation of U.S. patent application Ser. No. 17/560,402, filed Dec. 23, 2021, which is a continuation of U.S. patent application Ser. No. 16/365,317, filed Mar. 26, 2019, now U.S. Pat. No. 11,246,925, which is a continuation of U.S. patent application Ser. No. 15/389,130, filed Dec. 22, 2016, which is a division of U.S. patent application Ser. No. 13/982,373, filed Jul. 29, 2013, now U.S. Pat. No. 9,561,155, which is a National Stage filing under 35 U.S.C. § 371 of International Patent Application No. PCT/EP2012/051320, filed Jan. 27, 2012, which claims the benefit of priority of European Patent Application Nos. 11305088.4, filed Jan. 28, 2011, 11305089.2, filed Jan. 28, 2011, 11305513.1, filed Apr. 29, 2011, 11305514.9, filed Apr. 29, 2011, 11306039.6, filed Aug. 12, 2011, 11306040.4, filed Aug. 12, 2011, 11306201.2 filed Sep. 22, 2011, 11306202.0, filed Sep. 22, 2011, 11306449.7, filed Nov. 8, 2011, and 11306450.5, filed Nov. 8, 2011, the entireties of which are hereby incorporated herein by reference.

### SEQUENCE LISTING

The instant application contains a Sequence Listing which has been submitted electronically in XML format and is hereby incorporated by reference in its entirety. Said XML file, created on Jul. 30, 2024, is named 756595_SA9-129USDVCON3_ST26.xml and is 995,230 bytes in size.

### DESCRIPTION

The present invention relates to methods for treating diseases or conditions in which proprotein convertase subtilisin/kexin type 9 (PCSK9) expression or activity causes an impact by administration of PCSK9-specific antibodies or antigen-binding fragments thereof and preferably by additional administration of an inhibitor of 3-hydroxy-3-methyl-glutaryl-CoA reductase (HMG-COA reductase). The present invention further relates to PCSK9-specific antibodies or antigen-binding fragments thereof (preferably in combination with HMG-COA reductase inhibitors) for use in the treatment of diseases or conditions in which PCSK9 expression or activity causes an impact.

The present invention also relates to articles of manufacture comprising packaging material, PCSK9-specific antibodies or antigen-binding fragments thereof, and a label or packaging insert indicating which groups of patients can be treated with said antibodies or fragments, which groups of patients must not be treated with said antibodies or fragments, and which dosage regimen should be used.

The present invention further relates to methods of testing the efficacy of PCSK9-specific antibodies or antigen-binding fragments thereof for the treatment of certain diseases or conditions and for the treatment of specific sub-groups of patients.

### BACKGROUND OF THE INVENTION

Proprotein convertase subtilisin/kexin type 9 (PCSK9) is a proprotein convertase belonging to the proteinase K subfamily of the secretory subtilase family. The encoded protein is synthesized as a soluble zymogen that undergoes autocatalytic intramolecular processing in the endoplasmic reticulum. Evidence suggest that PCSK9 increases plasma LDL cholesterol by promoting degradation of the LDL receptor, which mediates LDL endocytosis in the liver, the major route of LDL clearance from circulation. The structure of PCSK9 protein shows that it has a signal sequence, followed by a prodomain, a catalytic domain that contains a conserved triad of residues (D186, H226 and S386), and a C-terminal domain. It is synthesized as a soluble 74-kDa precursor that undergoes autocatalytic cleavage in the ER, generating a 14-kDa prodomain and 60-kDa catalytic fragment. The autocatalytic activity has been shown to be required for secretion. After cleavage the prodomain remains tightly associated with the catalytic domain.

Antibodies to PCSK9 are described in, for example, WO 2008/057457, WO 2008/057458, WO 2008/057459, WO 2008/063382, WO 2008/125623, and US 2008/0008697. Anti-PCSK9 antibodies that are particularly well-suited for practicing the present invention are disclosed in US 2010/0166768 A1, the content of which is hereby incorporated by reference in its entirety.

### TECHNICAL PROBLEMS UNDERLYING THE PRESENT INVENTION

Statins are among the most widely used drugs in the world. Although statins generally exhibit an excellent safety profile, it is desirable to further optimize the safety profile by reducing the already low rate of unwanted side-effects (such as myopathies).

Despite the widespread availability of lipid-lowering agents such as statins, approximately 30% of all adult patients treated for hypercholesterolemia in the United States between 1999 and 2006 failed to achieve their recommended LDL-C targets. Reasons for this include poor adherence to therapy, drug-resistance/intolerance and the positive relationship between adverse event rates and increasing dosage. Moreover, since the most effective lipid-lowering agents can only reduce LDL-C levels by up to 55%, target attainment rates in patients that require substantial reductions in LDL-C, such as those with familial hypercholesterolemia, are often significantly lower than might be expected. More effective lipid-lowering agents and treatment regimes are therefore required to improve target attainment rates in these patients.

Quite surprisingly, the inventors of the present invention found that the administration of anti-PCSK9 antibodies or fragments thereof increases the LDL-cholesterol lowering activity of statins, when administered in particular dosage regimens and/or to particular groups of patient.

Thus, the co-administration of anti-PCSK9 antibodies or fragments thereof enhances the efficacy of a statin therapy and allows a reduction in the dosage of statins, thereby reducing unwanted side-effects.

Furthermore, the inventors of the present invention found out that particular dosage regimens of anti-PCSK9 antibodies and/or statins are better suited for reducing LDL-cholesterol levels than others. The inventors also found out that some sub-groups of patients benefit more than others from a treatment with anti-PCSK9 antibodies or fragments thereof and/or statins. The inventors further found out that treatment with anti-PCSK9 antibodies or fragments thereof and/or statins is contraindicated for some sub-groups of patients.

The above overview does not necessarily describe all problems solved by the present invention.

### SUMMARY OF THE INVENTION

In a first aspect the present invention relates to a method for treating a disease or condition in which PCSK9 expression or activity causes an impact, comprising:
administering a therapeutic amount of an antibody or an antigen-binding fragment thereof which specifically binds hPCSK9 (human proprotein convertase subtilisin/kexin type 9) to a subject in need thereof, wherein the antibody or antigen-binding fragment thereof is administered in a dosage amount ranging from 5 mg to 500 mg, and
administering a therapeutic amount of an HMG-COA reductase inhibitor to said subject, wherein the HMG-COA reductase inhibitor is administered in a dosage amount ranging from 0.05 mg to 100 mg.

In a second aspect the present invention relates to an antibody or an antigen-binding fragment thereof which specifically binds hPCSK9 (human proprotein convertase subtilisin/kexin type 9) for use in the treatment of a disease or condition in which PCSK9 expression or activity causes an impact,
wherein the antibody or antigen-binding fragment thereof is for administration in a dosage amount ranging from 5 mg to 500 mg,
wherein the antibody or antigen-binding fragment thereof is further for administration in combination with an HMG-CoA reductase inhibitor at a dosage amount ranging from 0.05 mg to 100 mg.

In a third aspect the present invention relates to an article of manufacture comprising: (a) a packaging material; (b) an antibody or an antigen-binding fragment thereof which specifically binds hPCSK9; and (c) a label or packaging insert contained within the packaging material indicating that patients receiving treatment with said antibody or antigen-binding fragment can be treated for a disease or condition selected from the group consisting of hypercholesterolemia, hyperlipidemia, dyslipidemia, atherosclerosis and cardiovascular diseases.

In a fourth aspect the present invention relates to an article of manufacture comprising: (a) a packaging material; (b) an antibody or an antigen-binding fragment thereof which specifically binds hPCSK9; and (c) a label or packaging insert contained within the packaging material indicating the treatment of patients with said antibody or antigen-binding fragment thereof together with the application of a statin.

In a fifth aspect the present invention relates to an article of manufacture comprising (a) a packaging material; (b) an antibody or an antigen-binding fragment thereof which specifically binds hPCSK9; and (c) a label or packaging insert indicating that the treatment of patients with said antibody or antigen-binding fragment thereof together with a statin is contraindicated for patients belonging to one or more of the following groups: (i) smokers; (ii) persons being 70 years old or older; (iii) persons suffering from hypertension; (iv) women who are pregnant; (v) women who are trying to become pregnant; (vi) women who are breast-feeding; (vii) persons who have or ever had a disease affecting the liver; (viii) persons who had any unexplained abnormal blood tests for liver function; (ix) persons who drink excessive amounts of alcohol; (x) persons having kidney problems; (xi) persons suffering from hypothyroidism; (xii) persons suffering from muscle disorders; (xiii) persons having encountered previous muscular problems during treatment with lipid-lowering medicine; (xiv) persons having serious problems with their breathing; (xv) persons taking one or more of the following medicines: medicines altering the way the immune systems works (e.g. ciclosporin or antihistamines), antibiotics or antifungal medicines (e.g. crythromycin, clarithromycin, ketoconazole, itraconazole, rifampicin, fusidic acid), medicines regulating lipid levels (e.g. gemfibrozil, colestipol), calcium channel blockers (e.g. verapamil, diltiazem), medicines regulating the heart rhythm (digoxin, amiodarone), protease inhibitors used in the treatment of HIV (e.g. nelfinavir), warfarin, oral contraceptives, antacids or St. John's Wort; or (xvi) persons drinking more than 0.1 L of grapefruit juice per day; (xvii) persons having a body mass index (BMI) of more than 40; (xviii) persons having a body mass index (BMI) of less than 18; (xix) persons suffering from type 1 diabetes or type 2 diabetes; (xx) persons positive for hepatitis B or hepatitis C; or (xxi) persons having a known sensitivity to monoclonal antibody therapeutics.

In a sixth aspect the present invention relates to a method of testing the efficacy of an antibody or an antigen-binding fragment thereof which specifically binds hPCSK9 for the treatment of a disease or condition selected from the group consisting of hypercholesterolemia, hyperlipidemia, dyslipidemia, atherosclerosis and cardiovascular diseases, said method comprising:
treating a selected patient population with said antibody or antigen-binding fragment thereof, wherein each patient in said population has an LDL cholesterol (LDL-C) level of more than 100 mg/dL; and
determining the efficacy of said antibody or antigen-binding fragment thereof by determining the LDL-C level in the patient population before and after administration of said antibody or antigen-binding fragment thereof, wherein a reduction of the LDL-C level by at least 25% relative to a predose level in at least 75% of the patient population indicates that said antibody or antigen-binding fragment thereof is efficacious for the treatment of said disease or condition in said patient population.

In a seventh aspect the present invention relates to a method of testing the efficacy of an antibody or an antigen-binding fragment thereof which specifically binds hPCSK9 for the treatment of a disease or condition selected from the group consisting of hypercholesterolemia, hyperlipidemia, dyslipidemia, atherosclerosis and cardiovascular diseases, said method comprising:

determining the efficacy of an antibody or antigen-binding fragment thereof that has been used for the treatment of a selected patient population with said antibody or antigen-binding fragment thereof, wherein each patient in said population has an LDL cholesterol (LDL-C) level of more than 100 mg/dL by determining the LDL-C level in the patient population before and after administration of said antibody or antigen-binding fragment thereof, wherein a reduction of the LDL-C level by at least 25% relative to a predose level in at least 75% of the patient population indicates that said antibody or antigen-binding fragment thereof is efficacious for the treatment of said disease or condition in said patient population.

In an eighth aspect the present invention relates to a package comprising an antibody or antigen-binding fragment thereof which specifically binds hPCSK9 (see section “Preferred Antibodies for Practicing the Present Invention”) and a label, said label comprising a printed statement which informs the patient that the treatment of the antibody together with a statin is indicated in one or more of the indications selected from the group consisting of hypercholesterolemia, hyperlipidemia, dyslipidemia, atherosclerosis and cardiovascular diseases.

In a ninth aspect the present invention relates to a package comprising an antibody or antigen-binding fragment thereof which specifically binds hPCSK9 (see section “Preferred Antibodies for Practicing the Present Invention”) and a label, said label comprising a printed statement which informs the patient that the treatment of the antibody together with a statin is contraindicated for patients belonging to one or more of the following groups: (i) smokers; (ii) persons being 70 years old or older; (iii) persons suffering from hypertension; (iv) women who are pregnant; (v) women who are trying to become pregnant; (vi) women who are breast-feeding; (vii) persons who have or ever had a disease affecting the liver; (viii) persons who had any unexplained abnormal blood tests for liver function; (ix) persons who drink excessive amounts of alcohol; (x) persons having kidney problems; (xi) persons suffering from hypothyroidism; (xii) persons suffering from muscle disorders; (xiii) persons having encountered previous muscular problems during treatment with lipid-lowering medicine; (xiv) persons having serious problems with their breathing; (xv) persons taking one or more of the following medicines: medicines altering the way the immune systems works (e.g. ciclosporin or antihistamines), antibiotics or antifungal medicines (e.g. erythromycin, clarithromycin, ketoconazole, itraconazole, rifampicin, fusidic acid), medicines regulating lipid levels (e.g. gemfibrozil, colestipol), calcium channel blockers (e.g. verapamil, diltiazem), medicines regulating the heart rhythm (digoxin, amiodarone), protease inhibitors used in the treatment of HIV (e.g. nelfinavir), warfarin, oral contraceptives, antacids or St. John's Wort; or (xvi) persons drinking more than 0.1 L of grapefruit juice per day; (xvii) persons having a body mass index (BMI) of more than 40; (xviii) persons having a body mass index (BMI) of less than 18; (xix) persons suffering from type 1 diabetes or type 2 diabetes; (xx) persons positive for hepatitis B or hepatitis C; or (xxi) persons having a known sensitivity to monoclonal antibody therapeutics.

In a tenth aspect the present invention relates to a method of regulating the LDL level in the blood comprising:
administering a therapeutic amount of an antibody or an antigen-binding fragment thereof which specifically binds hPCSK9 (human proprotein convertase subtilisin/kexin type 9) to a subject in need thereof, wherein the antibody or antigen-binding fragment thereof is administered in a dosage amount ranging from 5 mg to 500 mg, and
administering a therapeutic amount of an HMG-COA reductase inhibitor to said subject, wherein the HMG-COA reductase inhibitor is administered in a dosage amount ranging from 0.05 mg to 100 mg.

In an eleventh aspect the present invention relates to a method of preventing effects of a (persistently) increased LDL level in the blood comprising:
administering a therapeutic amount of an antibody or an antigen-binding fragment thereof which specifically binds hPCSK9 (human proprotein convertase subtilisin/kexin type 9) to a subject in need thereof, wherein the antibody or antigen-binding fragment thereof is administered in a dosage amount ranging from 5 mg to 500 mg, and
administering a therapeutic amount of an HMG-COA reductase inhibitor to said subject, wherein the HMG-COA reductase inhibitor is administered in a dosage amount ranging from 0.05 mg to 100 mg.

In a twelfth aspect the present invention relates to a method of determining whether a pharmaceutical compound is utilizable for ameliorating, improving, inhibiting or preventing a disease or condition in which PCSK9 activity or expression has an impact comprising:
(a) administering to a subject a compound that specifically binds to PCSK9, preferably an antibody or antigen-binding fragment thereof specifically binding to PCSK9, and
(b) determining what fraction of PCSK9 in the blood is attached to the compound from (a).

In a thirteenth aspect the present invention relates to a method for treating a disease or condition in which PCSK9 expression or activity causes an impact comprising
administering a therapeutic amount of an antibody or an antigen-binding fragment thereof which specifically binds hPCSK9 (human proprotein convertase subtilisin/kexin type 9) to a subject in need thereof,
wherein the subject in need thereof falls into one or more of the following groups of subjects: (i) subjects having a serum LDL cholesterol (LDL-C) level of at least 100 mg/dL; (ii) subjects having a serum HDL-C level of less than 40 mg/dL; (iii) subjects having a serum cholesterol level of at least 200 mg/dL; (iv) subjects having a serum triacylglycerol level of at least 150 mg/dL, wherein said triacylglycerol level is determined after fasting for at least 8 hours; (v) subjects being at least 35 years old; (vi) subjects younger than 75 years; (vii) subjects having a BMI of 25 or more; (viii) male subjects; (ix) female subjects; (x) subjects in which the administration of said antibody or antigen-binding fragment thereof leads to a reduction in the serum LDL-C level by at least 30 mg/dL relative to predose level; or (xi) subjects in which the administration of said antibody or antigen-binding fragment thereof leads to a reduction in the serum LDL-C level by at least 20% relative to predose level.

In a fourteenth aspect the present invention relates to a method for treating a disease or condition in which PCSK9 expression or activity causes an impact comprising
administering a therapeutic amount of an antibody or an antigen-binding fragment thereof which specifically binds hPCSK9 (human proprotein convertase subtilisin/kexin type 9) to a subject in need thereof,
wherein the subject in need thereof does not fall into one or more of the following groups of subjects: (i) smokers; (ii) persons being 70 years old or older; (iii) persons suffering from hypertension; (iv) women who are pregnant; (v) women who are trying to become pregnant; (vi) women who are breast-feeding; (vii) persons who have or ever had a disease affecting the liver; (viii) persons who had any unexplained abnormal blood tests for liver function; (ix) persons who drink excessive amounts of alcohol; (x) persons having kidney problems; (xi) persons suffering from hypothyroidism; (xii) persons suffering from muscle disorders; (xiii) persons having encountered previous muscular problems during treatment with lipid-lowering medicine; (xiv) persons having serious problems with their breathing; (xv) persons taking one or more of the following medicines: medicines altering the way the immune systems works (e.g. ciclosporin or antihistamines), antibiotics or antifungal medicines (e.g. erythromycin, clarithromycin, ketoconazole, itraconazole, rifampicin, fusidic acid), medicines regulating lipid levels (e.g. gemfibrozil, colestipol), calcium channel blockers (e.g. verapamil, diltiazem), medicines regulating the heart rhythm (digoxin, amiodarone), protease inhibitors used in the treatment of HIV (e.g. nelfinavir), warfarin, oral contraceptives, antacids or St. John's Wort; or (xvi) persons drinking more than 0.1 L of grapefruit juice per day; (xvii) persons having a body mass index (BMI) of more than 40; (xviii) persons having a body mass index (BMI) of less than 18; (xix) persons suffering from type 1 diabetes or type 2 diabetes; (xx) persons positive for hepatitis B or hepatitis C; or (xxi) persons having a known sensitivity to monoclonal antibody therapeutics.

In a fifteenth aspect the present invention is directed to an antibody or an antigen-binding fragment thereof which specifically binds hPCSK9 (human proprotein convertase subtilisin/kexin type 9), wherein the antibody is characterized by one or more of the following features upon administration to a subject, preferably a human or non-human mammal:
1. reduction of low-density lipoprotein (LDL-C) levels of at least about −25% to about −40% relative to a predose level with a sustained reduction over at least a 14 day-period, wherein the sustained reduction is preferably at least −25% and more preferably at least −30% relative to a predose level, particularly if administered in a dose of about 40 to about 60 mg, preferably about 45 to about 55 mg and more preferably about 50 mg in a biweekly administration regime (every other week, E2W),
2. reduction of low-density lipoprotein (LDL-C) of at least about −50% to about −65% relative to a predose level with a sustained reduction over at least a 14 day-period, wherein the sustained reduction is preferably at least −40% and more preferably at least −45% relative to a predose level, particularly if administered in a dose of about 100 mg E2W,
3. reduction of low-density lipoprotein (LDL-C) of at least about −60% to at least about −75% [e.g. at least about −60%, at least about −65%, at least about −70 or at least about −75%] relative to a predose level with a sustained reduction over at least a 14 day-period, wherein the sustained reduction is preferably at least −55% and more preferably at least −60% relative to a predose level, particularly when administered in a dose of about 150 mg E2W,
4. reduction of low-density lipoprotein (LDL-C) of at least about 40% to about 75% relative to a predose level with a sustained reduction over at least a 28 day period, wherein the sustained reduction is preferably at least −35% and more preferably at least −40% relative to a predose level, particularly when administered in a dose of about 200 mg E4W,
5. reduction of low-density lipoprotein (LDL-C) of at least about −50% to about −75% relative to a predose level with a sustained reduction over at least a 28 day-period, wherein the sustained reduction is preferably at least −40% and more preferably at least −45% relative to a predose level, particularly when administered in a dose of about 300 mg E4W,
6. increase of serum HDL cholesterol levels of at least 2%, at least 2.5%, at least, 3%, at least 3.5%, at least 4%, at least 4.5%, at least 5% or at least 5.5% relative to a predose level, particularly when administered in a dose of about 150 mg E2W,
7. reduction of serum total cholesterol at least about 25% to about 35% relative to a predose level with a sustained reduction over at least a 24 day period.
8. reduction of serum total cholesterol at least about 65% to about 80% relative to a predose level with a sustained reduction over at least a 24 day period,
9. reduction of serum triglyceride levels at least about 25% to about 40% relative to a predose level,
10. little or no measurable effect on liver function, as determined by ALT and AST measurements,
11. little or no measurable effect on troponin levels,
12. Increase of one or more of: Total-Cholesterol levels, ApoB levels, non HDL-C levels, Apo-B/ApoA-1 ratio,

In a sixteenth aspect the present invention is directed to an antibody or an antigen-binding fragment thereof which specifically binds hPCSK9 (human proprotein convertase subtilisin/kexin type 9) for use in the treatment of a disease or condition in which PCSK9 expression or activity causes an impact, wherein the antibody or antigen-binding fragment thereof is for administration in a dose of about 50 to 500 mg.

In a seventeenth aspect the present invention relates to an antibody or an antigen-binding fragment thereof which specifically binds hPCSK9 (human proprotein convertase subtilisin/kexin type 9).

for use in the treatment of a disease or condition in which PCSK9 expression or activity causes an impact.

wherein the antibody or antigen-binding fragment thereof is for administration to a subject falling at least into one of the following groups of subjects: (i) subjects having a serum LDL cholesterol (LDL-C) level of at least 100 mg/dL (i.e. at least 2.6 mmol/L) or of at least 115 mg/dL (i.e. at least 3.0 mmol/L); (ii) subjects having a serum HDL-C level of less than 40 mg/dL; (iii) subjects having a serum cholesterol level of at least 200 mg/dL; (iv) subjects having a serum triacylglycerol level of at least 150 mg/dL, wherein said triacylglycerol level is determined after fasting for at least 8 hours; (v) subjects being at least 18, 24 or 35 years old; (vi) subjects being 75 years old or younger; (vii) subjects having a BMI of 25 or more or of 30 or more; (viii) male subjects; (ix) female subjects; (x) subjects in which the administration of said antibody or antigen-binding fragment thereof leads to a reduction in the serum LDL-C level by at least 30 mg/dL at least 40 mg/dL, at least 45 mg/dl or at least 50 mg/dL relative to predose level (especially after 12 weeks of treatment); or (xi) subjects in which the administration of said antibody or antigen-binding fragment thereof leads to a reduction in the serum LDL-C level by at least 20% at least 25%, at least 30%, at least 35%, at least 40%, at least 45%, at least 50%, at least 55%, at least 60%, at least 65% or at least 70% relative to predose level especially after 12 weeks of treatment).

In an eighteenth aspect the present invention relates to an antibody or an antigen-binding fragment thereof which specifically binds hPCSK9 (human proprotein convertase subtilisin/kexin type 9) for use in the treatment of a disease or condition in which PCSK9 expression or activity causes an impact,

wherein the antibody or antigen-binding fragment thereof is for administration to a subject who does not fall into one or more of the following groups of subjects: (i) smokers; (ii) persons being 70 years old or older; (iii) persons suffering from hypertension; (iv) women who are pregnant; (v) women who are trying to become pregnant; (vi) women who are breast-feeding; (vii) persons who have or ever had a disease affecting the liver; (viii) persons who had any unexplained abnormal blood tests for liver function; (ix) persons who drink excessive amounts of alcohol; (x) persons having kidney problems; (xi) persons suffering from hypothyroidism; (xii) persons suffering from muscle disorders; (xiii) persons having encountered previous muscular problems during treatment with lipid-lowering medicine; (xiv) persons having serious problems with their breathing; (xv) persons taking one or more of the following medicines: medicines altering the way the immune systems works (e.g. ciclosporin or antihistamines), antibiotics or antifungal medicines (e.g. erythromycin, clarithromycin, ketoconazole, itraconazole, rifampicin, fusidic acid), medicines regulating lipid levels (e.g. gemfibrozil, colestipol), calcium channel blockers (e.g. verapamil, diltiazem), medicines regulating the heart rhythm (digoxin, amiodarone), protease inhibitors used in the treatment of HIV (e.g. nelfinavir), warfarin, oral contraceptives, antacids or St. John's Wort; or (xvi) persons drinking more than 0.1 L of grapefruit juice per day; (xvii) persons having a body mass index (BMI) of more than 40; (xviii) persons having a body mass index (BMI) of less than 18; (xix) persons suffering from type 1 diabetes or type 2 diabetes; (xx) persons positive for hepatitis B or hepatitis C; or (xxi) persons having a known sensitivity to monoclonal antibody therapeutics.

In a nineteenth aspect the present invention is directed to a pharmaceutical composition comprising the antibody or antigen-binding fragment thereof according to present invention together with a pharmaceutically acceptable excipient or carrier.

In a twentieth aspect, the present invention concerns an injection solution as herein described comprising the antibody or antigen-binding fragment thereof of present invention, and preferably comprising about 40 mg to about 200 mg or about 50 to about 200 mg, e.g. about 40 mg, about 50 mg, about 75 mg, at about 100 mg, about 150 mg or about 200 mg of the antibody or antigen-binding fragment thereof per 1 ml volume.

In a twentyfirst aspect the present invention concerns a dry formulation as herein described comprising the antibody or antigen-binding fragment thereof of present invention, and preferably comprising about 40 mg to about 500 mg, 50 to about 500 mg, about 50 to about 400, about 50 to about 300 e.g. about 40 mg, about 50 mg, about 75 mg, at about 100 mg, about 150 mg, about 200 mg, about 250 mg, about 300 mg, about 350 mg, about 400 mg, about 450 mg or about 500 mg and more preferably about 50 mg, about 100 mg, about 150 mg, about 200 mg, about 250 mg, about 300 mg and even more preferably about 150 mg, about 200 mg or about 300 mg of the antibody or antigen-binding fragment thereof per dose.

In a twentysecond aspect, present invention concerns an antibody or antigen binding fragment thereof as comprised in one of the pharmaceutical compositions according to the nineteenth aspect.

In a twentythird aspect the present invention is directed to a unit dosage form comprising the antibody, antigen-binding fragment thereof or pharmaceutical composition of present invention.

In a twentyfourth aspect, present invention concerns an article of manufacture comprising, the pharmaceutical composition of present invention, the liquid formulation of present invention or the dry formulation of present invention, the antibody or antigen-binding fragment thereof of present invention or one or more unit dosage forms of present invention and a container or package.

In a twentyfifth aspect the present invention relates to an article of manufacture comprising: (a) a packaging material; (b) an antibody or an antigen-binding fragment thereof which specifically binds hPCSK9; and (c) a label or packaging insert contained within the packaging material indicating that patients receiving treatment with said antibody or antigen-binding fragment can be treated for a disease or condition selected from the group consisting of hypercholesterolemia, hyperlipidemia, dyslipidemia, atherosclerosis and cardiovascular diseases and further indicating that subjects falling into one or more groups of subjects as recited in the thirteenth aspect can be treated.

In a twentysixth aspect the present invention relates to an article of manufacture comprising: (a) a packaging material; (b) an antibody or an antigen-binding fragment thereof which specifically binds hPCSK9; and (c) a label or packaging insert contained within the packaging material indicating that patients receiving treatment with said antibody or antigen-binding fragment can be treated for a disease or condition selected from the group consisting of hypercholesterolemia, hyperlipidemia, dyslipidemia, atherosclerosis and cardiovascular diseases and further indicating that the treatment of patients with said antibody or antigen-binding fragment thereof is contraindicated for patients belonging to one or more groups of subjects as recited in the fourteenth aspect.

In a twentyseventh aspect, present invention concerns a pharmaceutical composition or antibody or antigen-binding fragment thereof of present invention, such as according to the fifteenth or nineteenth aspect of present invention, for use in the treatment of a disease or condition in which PCSK9 expression or activity causes an impact.

In a twentyeighth aspect, present invention concerns a method for preparing a pharmaceutical composition of present invention, e.g. according to the nineteenth aspect, comprising mixing the antibody or antigen-binding fragment thereof and optionally the HMG-CoA reductase inhibitor with one or more pharmaceutical excipients or carriers.

In a twentynineth aspect, present invention concerns a method for preparing a unit dosage form of present comprising admeasuring an amount of the pharmaceutical composition, of the antibody or antigen-binding fragment thereof, of the liquid formulation or of the dry formulation according to present invention comprising one or more doses of the antibody or antigen fragment thereof and optionally of the HMG-COA reductase inhibitor and tailoring them as physically discrete units suitable as unitary dosages for human and/or animal administration.

In a thirtieth aspect, present invention concerns a method for preparing or assembling an article of manufacture of present invention comprising packaging the pharmaceutical composition, of the antibody according, of the liquid formulation, of the dry formulation according or of or more of the unit dosage forms of present invention in a container, optionally together with one or more of the following: a label, instructions for use, an application device.

In a thirtyfirst aspect the present invention relates to a method of testing the efficacy of an antibody or an antigen-binding fragment thereof which specifically binds hPCSK9 for the treatment of a disease or condition selected from the group consisting of hypercholesterolemia, hyperlipidemia, dyslipidemia, atherosclerosis and cardiovascular diseases, said method comprising:
treating a selected patient population with said antibody or antigen-binding fragment thereof, wherein each patient in said population has an LDL cholesterol (LDL-C) level of more than 100 mg/dL; and
determining the efficacy of said antibody or antigen-binding fragment thereof by determining the LDL-C level in the patient population before and after administration of said antibody or antigen-binding fragment thereof, wherein a reduction of the LDL-C level by at least 25% relative to a predose level in at least 75% of the patient population indicates that said antibody or antigen-binding fragment thereof is efficacious for the treatment of said disease or condition in said patient population;
wherein each patient falls into one or more groups of subjects as recited in the thirteenth aspect.

In a thirtysecond aspect the present invention relates to a method of testing the efficacy of an antibody or an antigen-binding fragment thereof which specifically binds hPCSK9 for the treatment of a disease or condition selected from the group consisting of hypercholesterolemia, hyperlipidemia, dyslipidemia, atherosclerosis and cardiovascular diseases, said method comprising:
determining the efficacy of an antibody or antigen-binding fragment thereof that has been used for the treatment of a selected patient population with said antibody or antigen-binding fragment thereof, wherein each patient in said population has an LDL cholesterol (LDL-C) level of more than 100 mg/dL by determining the LDL-C level in the patient population before and after administration of said antibody or antigen-binding fragment thereof, wherein a reduction of the LDL-C level by at least 25% relative to a predose level in at least 75% of the patient population indicates that said antibody or antigen-binding fragment thereof is efficacious for the treatment of said disease or condition in said patient population;
wherein each patient falls into one or more groups of subjects as recited in the thirteenth aspect.

In a thirtythird aspect the present invention relates to a method for testing the efficacy of a compound in lowering cholesterol levels in a subject, comprising the steps: (a) providing a rodent; (b) administering an antibody or an antigen-binding fragment thereof which specifically binds PCSK9 to the rodent; (c) administering a test compound to said rodent; (d) determining the effect of the test compound in the rodent, wherein a lowering of the cholesterol level in the rodent as compared to the cholesterol level of a control animal indicates that the test compound is efficacious in lowering cholesterol levels in a subject, wherein the control animal is from the same species as said rodent, and wherein the control animal has not been challenged with the test compound.

In a thirtyfourth aspect, present invention concerns a method of enhancing the LDL-C lowering activity in a subject undergoing statin therapy, the method comprising administering to the subject an antibody, or antigen-binding fragment thereof, which specifically binds to human proprotein convertase subtilisin/kexin type 9 (hPCSK9), wherein the antibody or antigen-binding fragment thereof is administered at a dosage amount within the range of about 5 mg to about 500 mg, thereby enhancing LCL-C lowering activity of the statin therapy in the subject.

In a thirtyfifth aspect, present invention concerns a kit for treating elevated low-density lipoprotein cholesterol (LDL-C) levels in a subject, the kit comprising (a) pharmaceutical unit dosage form comprising an antibody, or antigen-binding fragment thereof, which specifically binds to hPCSK9; and pharmaceutically acceptable carrier, wherein the antibody or antigen-binding fragment is present in a dosage amount within the range of about 5 mg to about 500 mg; and (b) a label or packaging insert with instructions for use.

In a thirtysixth aspect, present invention concerns a method of treating a subject suffering from a disease or disorder characterized by elevated low-density lipoprotein cholesterol (LDL-C) levels, the method comprising:
(a) selecting a subject with a blood LDL-C level greater than 100 mg/dL; and
(b) administering to said subject a composition comprising an antibody or antigen binding fragment thereof that specifically binds to human proprotein convertase subtilisin/kexin type 9 (hPCSK9); thereby lowering cholesterol levels in the subject in need thereof.

In a twentysixth aspect, present invention concerns a method of lowering cholesterol levels in a subject in need thereof, comprising:
(a) selecting a subject with a blood low density lipoprotein cholesterol (LDL-C) level greater than 100 mg/dL; and
(b) administering to said subject a composition comprising an antibody or antigen binding fragment thereof that specifically binds to human proprotein convertase subtilisin/kexin type 9 (hPCSK9); thereby lowering cholesterol levels in the subject in need thereof.

This summary of the invention does not necessarily describe all features of the present invention. Other embodiments will become apparent from a review of the ensuing detailed description.

### BRIEF DESCRIPTION OF THE DRAWINGS

FIG. 1 shows the percentage reduction in LDL-cholesterol (LDL-C) levels relative to the baseline for three groups of patients upon treatment with anti-PCSK9 antibody 316P. These patient groups are: (1) patients with familial hypercholesterolemia (HeFH); (2) patients with other forms of primary hypercholesterolemia (non-FH) on diet and on stable atorvastatin therapy; and (3) patients with other forms of primary hypercholesterolemia (non-FH) on diet alone. A dose of 50 mg of the anti-PCSK9 antibody was administered subcutaneously on days 1, 29 and 43. Results from patient groups receiving the antibody (50-mg-FH-no; 50-mg-FH-Yes; 50-mg-combined) are shown in solid lines, while results from patients receiving a placebo (PBO-FH-no; PBO-FH-Yes; PBO-combined) are shown in dashed lines.

FIG. 2 shows the percentage reduction in LDL-cholesterol (LDL-C) levels relative to the baseline for three groups of patients upon treatment with anti-PCSK9 antibody 316P. These patient groups are: (1) patients with familial hypercholesterolemia (HeFH); (2) patients with other forms of primary hypercholesterolemia (non-FH) on diet and on stable atorvastatin therapy; and (3) patients with other forms of primary hypercholesterolemia (non-FH) on diet alone. A dose of 100 mg of the anti-PCSK9 antibody was administered subcutaneously on days 1, 29 and 43. Results from patient groups receiving the antibody (100-mg-FH-no; 100-mg-FH-Yes; 100-mg-combined) are shown in solid lines, while results from patients receiving a placebo (PBO-FH-no; PBO-FH-Yes; PBO-combined) are shown in dashed lines.

FIG. 3 shows the percentage reduction in LDL-cholesterol (LDL-C) levels relative to the baseline for three groups of patients upon treatment with anti-PCSK9 antibody 316P. These patient groups are: (1) patients with familial hypercholesterolemia (HeFH); (2) patients with other forms of primary hypercholesterolemia (non-FH) on diet and on stable atorvastatin therapy; and (3) patients with other forms of primary hypercholesterolemia (non-FH) on diet alone. A dose of 150 mg of the anti-PCSK9 antibody was administered subcutaneously on days 1, 29 and 43. Results from patient groups receiving the antibody (150-mg-FH-no; 150-mg-FH-Yes; 150-mg-combined) are shown in solid lines, while results from patients receiving a placebo (PBO-FH-no; PBO-FH-Yes; PBO-combined) are shown in dashed lines.

FIG. 4 shows the study design of study 2 for the group of patients receiving a lipid lowering treatment other than atorvastatin or not at stable dose of atorvastatin 10 mg for at least 6 weeks prior to screening, or drug naive patients.

FIG. 5 shows the study design of study 2 for the group of patients receiving atorvastatin 10 mg at stable dose for at least 6 weeks prior to screening.

FIG. 6 shows the distribution of the LDL-C mean values of patients of study 1 receiving antibody 316 P at stable atorvastatin treatment over 12 weeks and LOCF (last observation carried forward). The study was designed to assess the efficacy and safety of antibody 316P in hypercholesteremia patients with an elevated LDL-C (≥100 mg/dL or 2.59 mmol/L) treated with stable dose of atorvastatin (10 mg, 20 mg, or 40 mg). During the run-in period, patients were stabilized to atorvastatin treatment (10 mg, 20 mg, or 40 mg) if the were not already. After one additional week, patients were centrally randomized via IVRS/IWRS in a 1:1:1:1:1:1 ratio to one of the 6 treatment groups (placebo, 316P 50 mg E2W, 316P 100 mg E2W, 316P 150 mg E2W, 316P 200 mg E4W, 316P 300 mg E4W) and treated in a double-bind manner for approximately 12 weeks. Thr randomization is stratified by the dose of atorvastatin received prior to randomization. During the double-bind treatment period patients returned to the site every 2 weeks to receive the study treatment (316 P or placebo). The double-bind treatment period was then followed ba an 8-week follow up period. As can be gained from FIG. 6, all treatment groups except for the group of patients receiving placebo had a significant and persistent reduction of LDL-C levels over the whole study period.

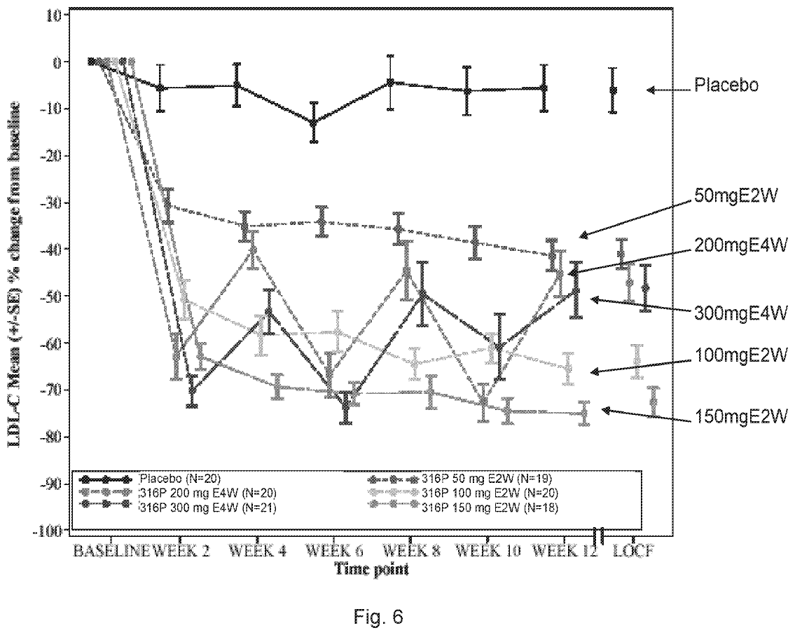

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents (for example: patents, patent applications, scientific publications, manufacturer's specifications, instructions, GenBank Accession Number sequence submissions etc.) are cited throughout the text of this specification. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

Sequences: All sequences referred to herein are disclosed in the attached sequence listing that, with its whole content and disclosure, is a part of this specification.

The term “about” when used in connection with a numerical value is meant to encompass numerical values within a range having a lower limit that is 5% smaller than the indicated numerical value and having an upper limit that is 5% larger than the indicated numerical value.

The term “human proprotein convertase subtilisin/kexin type 9” or “hPCSK9”, as used herein, refers to hPCSK9 having the nucleic acid sequence shown in SEQ ID NO: 754 and the amino acid sequence of SEQ ID NO: 755, or a biologically active fragment thereof.

The terms “specifically binds”, “specific binding” or the like, mean that an antibody or antigen-binding fragment thereof forms a complex with an antigen that is relatively stable under physiologic conditions. Specific binding can be characterized by an equilibrium dissociation constant of at least about 1×10⁻⁶ M or less (e.g., a smaller K_{D} denotes a tighter binding). Methods for determining whether two molecules specifically bind are well known in the art and include, for example, equilibrium dialysis, surface plasmon resonance, and the like. An isolated antibody that specifically binds hPCSK9 may, however, exhibit cross-reactivity to other antigens such as PCSK9 molecules from other species. Moreover, multi-specific antibodies (e.g., bispecifics) that bind to hPCSK9 and one or more additional antigens are nonetheless considered antibodies that “specifically bind” hPCSK9, as used herein.

The term “K_{D}”, as used herein, is intended to refer to the equilibrium dissociation constant of a particular antibody-antigen interaction. The equilibrium dissociation constant is typically measured in “mol/L” (abbreviated as “M”).

By the term “slow off rate”, “Koff” or “kd” is meant an antibody that dissociates from hPCSK9 with a rate constant of 1×10⁻³ s⁻¹ or less, preferably 1×10⁻⁴ s⁻¹ or less, as determined by surface plasmon resonance, e.g., BIACORE™.

The term “high affinity” antibody refers to those mAbs having a binding affinity to hPCSK9 of at least 10⁻¹⁰ M; preferably 10⁻¹¹ M; even more preferably 10⁻¹² M, as measured by surface plasmon resonance, e.g., BIACORE™ or solution-affinity ELISA.

The term “surface plasmon resonance”, as used herein, refers to an optical phenomenon that allows for the analysis of real-time biospecific interactions by detection of alterations in protein concentrations within a biosensor matrix, for example using the BIACORE™ system (Pharmacia Biosensor AB, Uppsala, Sweden and Piscataway, N.J.).

An “epitope”, also known as antigenic determinant, is the region of an antigen that is recognized by the immune system, specifically by antibodies, B cells, or T cells. As used herein, an “epitope” is the part of an antigen capable of binding to an antibody or antigen-binding fragment thereof as described herein. In this context, the term “binding” preferably relates to a “specific binding”, as defined herein. Epitopes usually consist of chemically active surface groupings of molecules such as amino acids, sugar side chains, phosphoryl groups, or sulfonyl groups and may have specific three-dimensional structural characteristics and/or specific charge characteristics. Conformational and non-conformational epitopes can be distinguished in that the binding to the former but not the latter is lost in the presence of denaturing solvents.

A “paratope” is the part of an antibody that specifically binds to the epitope.

The term “antibody”, as used herein, is intended to refer to immunoglobulin molecules comprised of four polypeptide chains, two heavy (H) chains and two light (L) chains inter-connected by disulfide bonds. The term “antibody” also includes all recombinant forms of antibodies, in particular of the antibodies described herein, e.g. antibodies expressed in prokaryotes, unglycosylated antibodies, and any antigen-binding antibody fragments and derivatives as described below. Each heavy chain is comprised of a heavy chain variable region (“HCVR” or “VH”) and a heavy chain constant region (comprised of domains CH1, CH2 and CH3). Each light chain is comprised of a light chain variable region (“LCVR or “VL”) and a light chain constant region (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The term “antigen-binding fragment” of an antibody (or simply “binding portion”), as used herein, refers to one or more fragments of an antibody that retain the ability to specifically bind to hPCSK9. It has been shown that the antigen-binding function of an antibody can be performed by fragments of a full-length antibody. Examples of binding fragments encompassed within the term “antigen-binding fragment” of an antibody include (i) Fab fragments, monovalent fragments consisting of the VL, VH, CL and CH domains; (ii) F(ab′)₂ fragments, bivalent fragments comprising two Fab fragments linked by a disulfide bridge at the hinge region; (iii) Fd fragments consisting of the VH and CH domains; (iv) Fv fragments consisting of the VL and VH domains of a single arm of an antibody, (v) dAb fragments (Ward et al., (1989) Nature 341:544-546), which consist of a VH domain; (vi) isolated complementarity determining regions (CDR), and (vii) combinations of two or more isolated CDRs which may optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. A further example is a binding-domain immunoglobulin fusion protein comprising (i) a binding domain polypeptide that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The binding domain polypeptide can be a heavy chain variable region or a light chain variable region. The binding-domain immunoglobulin fusion proteins are further disclosed in US 2003/0118592 and US 2003/0133939. These antibody fragments are obtained using conventional techniques known to those with skill in the art, and the fragments are screened for utility in the same manner as are intact antibodies. Further examples of “antigen-binding fragments” are so-called microantibodies, which are derived from single CDRs. For example, Heap et al. describe a 17 amino acid residue microantibody derived from the heavy chain CDR3 of an antibody directed against the gp 120 envelope glycoprotein of HIV-1 (Heap C J et al. (2005) J. Gen. Virol. 86:1791-1800). Other examples include small antibody mimetics comprising two or more CDR regions that are fused to each other, preferably by cognate framework regions. Such a small antibody mimetic comprising VH CDR1 and VL CDR3 linked by the cognate VH FR2 has been described by Qiu et al. (Qiu X-Q, et al. (2007) Nature biotechnology 25 (8): 921-929).

Thus, the term “antibody or antigen-binding fragment thereof”, as used herein, refers to immunoglobulin molecules and immunologically active portions of immunoglobulin molecules, i.e. molecules that contain an antigen-binding site that immunospecifically binds an antigen.

Antibodies and antigen-binding fragments thereof usable in the invention may be from any animal origin including birds and mammals. Preferably, the antibodies or fragments are from human, chimpanzee, rodent (e.g. mouse, rat, guinea pig, or rabbit), chicken, turkey, pig, sheep, goat, camel, cow, horse, donkey, cat, or dog origin. It is particularly preferred that the antibodies are of human or murine origin. Antibodies of the invention also include chimeric molecules in which an antibody constant region derived from one species, preferably human, is combined with the antigen binding site derived from another species, e.g. mouse. Moreover antibodies of the invention include humanized molecules in which the antigen binding sites of an antibody derived from a non-human species (e.g. from mouse) are combined with constant and framework regions of human origin.

As exemplified herein, antibodies of the invention can be obtained directly from hybridomas which express the antibody, or can be cloned and recombinantly expressed in a host cell (e.g., a CHO cell, or a lymphocytic cell). Further examples of host cells are microorganisms, such as E. coli, and fungi, such as yeast. Alternatively, they can be produced recombinantly in a transgenic non-human animal or plant.

The term “chimeric antibody” refers to those antibodies wherein one portion of each of the amino acid sequences of heavy and light chains is homologous to corresponding sequences in antibodies derived from a particular species or belonging to a particular class, while the remaining segment of the chain is homologous to corresponding sequences in another species or class. Typically the variable region of both light and heavy chains mimics the variable regions of antibodies derived from one species of mammals, while the constant portions are homologous to sequences of antibodies derived from another. One clear advantage to such chimeric forms is that the variable region can conveniently be derived from presently known sources using readily available B-cells or hybridomas from non-human host organisms in combination with constant regions derived from, for example, human cell preparations. While the variable region has the advantage of case of preparation and the specificity is not affected by the source, the constant region being human is less likely to elicit an immune response from a human subject when the antibodies are injected than would the constant region from a non-human source. However, the definition is not limited to this particular example.

The term “humanized antibody” refers to a molecule having an antigen binding site that is substantially derived from an immunoglobulin from a non-human species, wherein the remaining immunoglobulin structure of the molecule is based upon the structure and/or sequence of a human immunoglobulin. The antigen binding site may either comprise complete variable domains fused onto constant domains or only the complementarity determining regions (CDR) grafted onto appropriate framework regions in the variable domains. Antigen-binding sites may be wild-type or modified by one or more amino acid substitutions, e.g. modified to resemble human immunoglobulins more closely. Some forms of humanized antibodies preserve all CDR sequences (for example a humanized mouse antibody which contains all six CDRs from the mouse antibody). Other forms have one or more CDRs which are altered with respect to the original antibody.

Different methods for humanizing antibodies are known to the skilled person, as reviewed by Almagro & Fransson, the content of which is herein incorporated by reference in its entirety (Almagro J C and Fransson J (2008) Frontiers in Bioscience 13:1619-1633). Almagro & Fransson distinguish between rational approaches and empirical approaches. Rational approaches are characterized by generating few variants of the engineered antibody and assessing their binding or any other property of interest. If the designed variants do not produce the expected results, a new cycle of design and binding assessment is initiated. Rational approaches include CDR grafting, Resurfacing, Superhumanization, and Human String Content Optimization. In contrast, empirical approaches are based on the generation of large libraries of humanized variants and selection of the best clones using enrichment technologies or high-throughput screening. Accordingly, empirical approaches are dependent on a reliable selection and/or screening system that is able to search through a vast space of antibody variants. In vitro display technologies, such as phage display and ribosome display fulfill these requirements and are well-known to the skilled person. Empirical approaches include FR libraries, Guided selection, Framework-shuffling, and Humaneering.

The term “human antibody”, as used herein, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse), have been grafted onto human FR sequences. Human antibodies of the invention include antibodies isolated from human immunoglobulin libraries or from animals transgenic for one or more human immunoglobulin and that do not express endogenous immunoglobulins, as described for example in U.S. Pat. No. 5,939,598 by Kucherlapati & Jakobovits.

The term “monoclonal antibody” as used herein refers to a preparation of antibody molecules of single molecular composition. A monoclonal antibody displays a single binding specificity and affinity for a particular epitope. In one embodiment, the monoclonal antibodies are produced by a hybridoma which includes a B cell obtained from a non-human animal, e.g. mouse, fused to an immortalized cell.

The term “recombinant antibody”, as used herein, includes all antibodies that are prepared, expressed, created or isolated by recombinant means, such as (a) antibodies isolated from an animal (e.g., a mouse) that is transgenic or transchromosomal with respect to the immunoglobulin genes or a hybridoma prepared therefrom, (b) antibodies isolated from a host cell transformed to express the antibody, e.g. from a transfectoma, (c) antibodies isolated from a recombinant, combinatorial antibody library, and (d) antibodies prepared, expressed, created or isolated by any other means that involve splicing of immunoglobulin gene sequences to other DNA sequences.

The term “transfectoma”, as used herein, includes recombinant eukaryotic host cells expressing an antibody, such as CHO cells, NS/0 cells, HEK293 cells, HEK293T cells, plant cells, or fungi, including yeast cells.

As used herein, a “heterologous antibody” is defined in relation to a transgenic organism producing such an antibody. This term refers to an antibody having an amino acid sequence or an encoding nucleic acid sequence corresponding to that found in an organism not consisting of the transgenic organism, and being generally derived from a species other than the transgenic organism.

As used herein, a “heterohybrid antibody” refers to an antibody having light and heavy chains of different organismal origins. For example, an antibody having a human heavy chain associated with a murine light chain is a heterohybrid antibody.

Thus, “antibodies and antigen-binding fragments thereof” suitable for use in the present invention include, but are not limited to, polyclonal, monoclonal, monovalent, bispecific, heteroconjugate, multispecific, recombinant, heterologous, heterohybrid, chimeric, humanized (in particular CDR-grafted), deimmunized, or human antibodies, Fab fragments, Fab′ fragments, F(ab′)₂ fragments, fragments produced by a Fab expression library, Fd, Fv, disulfide-linked Fvs (dsFv), single chain antibodies (e.g. scFv), diabodies or tetrabodies (Holliger P. et al. (1993) Proc. Natl. Acad. Sci. U.S.A. 90 (14), 6444-6448), nanobodies (also known as single domain antibodies), anti-idiotypic (anti-Id) antibodies (including, e.g., anti-Id antibodies to antibodies of the invention), and epitope-binding fragments of any of the above.

The antibodies described herein are preferably isolated. An “isolated antibody”, as used herein, is intended to refer to an antibody that is substantially free of other mAbs having different antigenic specificities (e.g., an isolated antibody that specifically binds hPCSK9 is substantially free of mAbs that specifically bind antigens other than hPCSK9). An isolated antibody that specifically binds hPCSK9 may, however, have cross-reactivity to other antigens, such as PCSK9 molecules from other species.

As used herein, a “PCSK9 antagonist” denotes a compound that inhibits at least one biological activity of PCSK9, preferably the proteinase activity of PCSK9. Preferred PCSK9 antagonists are characterized in that they bind from 10% to 100% (preferably from 50% to 100%) of the PCSK9 present in the blood when used in stoichiometric amounts. Preferred PCSK9 antagonists of the present invention are neutralizing antibodies.

A “neutralizing antibody”, as used herein (or an “antibody that neutralizes PCSK9 activity”), is intended to refer to an antibody whose binding to hPCSK9 results in inhibition of at least one biological activity of PCSK9, preferably inhibition of the proteinase activity of PCSK9. This inhibition of the biological activity of PCSK9 can be assessed by measuring one or more indicators of PCSK9 biological activity by one or more of several standard in vitro or in vivo assays known in the art. Such assays are described for example in US 2010/0166768 A1, the content of which is hereby incorporated by reference in its entirety.

Since PCSK9 increases plasma LDL cholesterol by promoting degradation of the LDL receptor, the activity of PCSK9 has an effect on several diseases associated with increased plasma LDL cholesterol levels. Accordingly, PCSK9 antagonists, such as neutralizing anti-hPCSK9 antibodies or antigen-binding fragments thereof, are useful to reduce elevated total cholesterol, non-HDL cholesterol, LDL cholesterol, and/or apolipoprotein B100 (ApoB100). Consequently, PCSK9 antagonists are useful for ameliorating, improving, inhibiting or preventing several such diseases, including without limitation hypercholesterolemia, hyperlipidemia, dyslipidemia, atherosclerosis and cardiovascular diseases.

In specific embodiments, the anti-PCSK9 antibodies or antigen-binding fragments thereof described herein may be conjugated to a therapeutic moiety (“immunoconjugate”), such as a cytotoxin, a chemotherapeutic drug, an immunosuppressant or a radioisotope.

A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24:307-331. Examples of groups of amino acids that have side chains with similar chemical properties include
1) aliphatic side chains: glycine, alanine, valine, leucine and isoleucine;
2) aliphatic-hydroxyl side chains: serine and threonine;
3) amide-containing side chains: asparagine and glutamine;
4) aromatic side chains: phenylalanine, tyrosine, and tryptophan;
5) basic side chains: lysine, arginine, and histidine;
6) acidic side chains: aspartate and glutamate, and
7) sulfur-containing side chains: cysteine and methionine.

Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-45. A “moderately conservative” replacement is any change having a nonnegative value in the PAM250 log-likelihood matrix. Given the known genetic code, and recombinant and synthetic DNA techniques, the skilled scientist can readily construct DNAs encoding conservative amino acid variants.

As used herein, “non-conservative substitutions” or “non-conservative amino acid exchanges” are defined as exchanges of an amino acid by another amino acid listed in a different group of the seven standard amino acid groups 1) to 7) shown above.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions which are not identical differ by conservative amino acid substitutions.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403 410 and (1997) Nucleic Acids Res. 25:3389 402, each of which is herein incorporated by reference.

When percentages of sequence identity are referred to in the present application, these percentages are calculated in relation to the full length of the longer sequence, if not specifically indicated otherwise. This calculation in relation to the full length of the longer sequence applies both to nucleic acid sequences and to polypeptide sequences.

As used herein, “treat”, “treating” or “treatment” of a disease or disorder means accomplishing one or more of the following: (a) reducing the severity and/or duration of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, “prevent”, “preventing”, “prevention”, or “prophylaxis” of a disease or disorder means preventing that a disorder occurs in subject.

As used herein, the expressions “is for administration” and “is to be administered” have the same meaning as “is prepared to be administered”. In other words, the statement that an active compound “is for administration” has to be understood in that said active compound has been formulated and made up into doses so that said active compound is in a state capable of exerting its therapeutic activity.

The terms “therapeutically effective amount” or “therapeutic amount” are intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. Particularly, the dosage a patient receives can be selected so as to achieve the amount of LDL (low density lipoprotein) cholesterol lowering desired; the dosage a patient receives may also be titrated over time in order to reach a target LDL level. The dosage regimen utilizing an antibody or an antigen-binding fragment thereof as described herein is selected in accordance with a variety of factors including type, species, age, weight, body mass index, sex and medical condition of the patient; the severity of the condition to be treated; the potency of the compound chosen to be administered; the route of administration; the purpose of the administration; and the renal and hepatic function of the patient.

As used herein, a “patient” means any human or non-human animal, such as mammal, reptile or bird who may benefit from a treatment with the antibodies and antigen-biding fragments thereof described herein. Preferably, a “patient” is selected from the group consisting of laboratory animals (e.g. mouse or rat), domestic animals (including e.g. guinea pig, rabbit, chicken, turkey, pig, sheep, goat, camel, cow, horse, donkey, cat, or dog), rodent or primates including chimpanzee, gorilla, bonobo and human beings. It is particularly preferred that the “patient” is a human being.

The terms “subject” or “individual” are used interchangeably herein. As used herein, a “subject” refers to a human or a non-human animal (e.g. a mammal, avian, reptile, fish, amphibian or invertebrate; preferably an individual that can either benefit from one of the different aspects of present invention (e.g. a method of treatment or a drug identified by present methods) or that can be used as laboratory animal for the identification or characterisation of a drug or a method of treatment. The individual can e.g. be a human, a wild-animal, domestic animal or laboratory animal; examples comprise: mammal, e.g. human, non-human primate (chimpanzee, bonobo, gorilla), dog, cat, rodent (e.g. mouse, guinea pig, rat, hamster or rabbit, horse, donkey, cow, sheep, goat, pig, camel; avian, such as duck, dove, turkey, goose or chick; reptile such as: turtle, tortoise, snake, lizard, amphibian such as frog (e.g. Xenopus laevis); fish such as koy or zebrafish; invertebrate such as a worm (e.g. c. elegans) or an insect (such as a fly, e.g. Drosophila melanogaster). The term individual also comprises the different morphological developmental stages of avian, fish, reptile or insects, such as egg, pupa, larva or imago. It is further preferred if the subject is a “patient”.

As used herein, “unit dosage form” refers to physically discrete units suitable as unitary dosages for human and/or animal subjects, each unit containing a predetermined quantity of active material (e.g., about 50 to about 500 mg of PCSK5 antibody and/or of e.g. 0.05 mg to 100 mg HMG-COA reductase inhibitor) calculated to produce the desired therapeutic effect in association with the required pharmaceutical diluent, carrier or vehicle. The specifications for the novel unit dosage forms of this invention are dictated by and are directly dependent on (a) the unique characteristics of the active material and the particular therapeutic effect to be achieved, and (b) the limitation inherent in the art of compounding such an active material for therapeutic use in animals or humans, as disclosed in this specification, these being features of the present invention. Examples of suitable unit dosage forms in accord with this invention are vials, tablets, capsules, troches, suppositories, powder packets, wafers, cachets, ampules, segregated multiples of any of the foregoing, and other forms as herein described or generally known in the art. One or more such unit dosage forms of the antibody can be comprised in an article of manufacture of present invention, optionally further comprising one or more unit dosage forms of an HMG-CoA reductase inhibitor (e.g. a blister of tablets comprising as active ingredient the HMG-COA reductase inhibitor).

The term “active material” refers to any material with therapeutic activity, such as one or more active ingredients. The active ingredients to be employed as therapeutic agents can be easily prepared in such unit dosage form with the employment of pharmaceutical materials which themselves are available in the art and can be prepared by established procedures.

The following preparations are illustrative of the preparation of the unit dosage forms of the present invention, and not as a limitation thereof. Several dosage forms may be prepared embodying the present invention. For example, a unit dosage per vial may contain 0.5 ml, 1 ml, 2 ml, 3 ml, 4 ml, 5 ml, 6 ml, 7 ml, 8 ml, 9 ml, 10 ml, 15 ml, or 20 ml of PCSK5 antibody or a fragment thereof ranging from about 40 to about 500 mg of PCSK5 antibody. If necessary, these preparations can be adjusted to a desired concentration by adding a sterile diluent to each vial. In one embodiment, the ingredients of formulation of the invention are supplied either separately or mixed together in unit dosage form, for example, as a dry lyophilized powder or water free concentrate in a hermetically sealed container such as a vial, an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The formulations of the invention include bulk drug compositions useful in the manufacture of pharmaceutical compositions (e.g., compositions that are suitable for administration to a subject or patient) which can be used in the preparation of unit dosage forms. In a preferred embodiment, a composition of the invention is a pharmaceutical composition. Such compositions comprise a prophylactically or therapeutically effective amount of one or more prophylactic or therapeutic agents (e.g., an antibody of the invention or other prophylactic or therapeutic agent), and a pharmaceutically acceptable carrier. Preferably, the pharmaceutical compositions are formulated to be suitable for the route of administration to a subject.

The active materials or ingredients (e.g. antibodies or fragments thereof and HMG-COA reductase inhibitors) can be formulated as various dosage forms including solid dosage forms for oral administration such as capsules, tablets, pills, powders and granules, liquid dosage forms for oral administration such as pharmaceutically acceptable emulsions, microemulsions, solutions, suspensions, syrups and elixirs, injectable preparations, for example, sterile injectable aqueous or oleaginous suspensions, compositions for rectal or vaginal administration, preferably suppositories, and dosage forms for topical or transdermal administration such as ointments, pastes, creams, lotions, gels, powders, solutions, sprays, inhalants or patches.

In a specific embodiment, the term “pharmaceutically acceptable” means approved by a regulatory agency of the U.S. Federal or a state government or the EMA (European Medicines Agency) or listed in the U.S. Pharmacopeia Pharmacopeia (United States Pharmacopcia-33/National Formulary-28 Reissue, published by the United States Pharmacopeial Convention, Inc., Rockville Md., publication date: April 2010) or other generally recognized pharmacopcia for use in animals, and more particularly in humans. The term “carrier” refers to a diluent, adjuvant {e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. For the use of (further) excipients and their use see also “Handbook of Pharmaceutical Excipients”, fifth edition, R. C. Rowe, P. J. Seskey and S. C. Owen, Pharmaceutical Press, London, Chicago. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of the antibody, preferably in purified form, together with a suitable amount of carrier so as to provide the form for proper administration to the patient. The formulation should suit the mode of administration.

Generally, the ingredients of compositions of the invention are supplied either separately or mixed together in unit dosage form, for example, as a dry formulation for dissolution such as a lyophilized powder, freeze-dried powder or water free concentrate in a hermetically sealed container such as an ampoule or sachette indicating the quantity of active agent. The ingredients of compositions of the invention can also be supplied as admixed liquid formulation (i.e. injection or infusion solution) in a hermetically sealed container such as an ampoule, sachette, a pre-filled syringe or autoinjector, or a cartridge for a reusable syringe or applicator (e.g. pen or autoinjector). Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides that the formulation is packaged in a hermetically sealed container such as an ampoule or sachette indicating the quantity of antibody. In one embodiment, the formulation of the invention comprising an antibody is supplied as a dry formulation, such as a sterilized lyophilized powder, freeze-dried powder, spray-dried powder or water free concentrate in a hermetically sealed container and can be reconstituted, e.g., with water or saline to the appropriate concentration for administration to a subject. In another embodiment the antibody or antigen binding fragment thereof is supplied as a liquid formulation such as an injection or infusion solution. In one embodiment, the formulation of the invention comprising an antibody is supplied as a dry formulation or as a liquid formulation in a hermetically sealed container at a unit dosage of at least 40 mg, at least 50 mg, more preferably at least 75 mg, at least 100 mg, at least 150 mg, at least 200 mg, at least 250 mg, at least 300 mg, at least 350 mg, at least 400 mg, at least 450 mg, or at least 500 mg, of antibody or antigen-binding fragment thereof. The lyophilized formulation of the invention comprising an antibody should be stored at between 2 and 8° C. in its original container and the antibody should be administered within 12 hours, preferably within 6 hours, within 5 hours, within 3 hours, or within 1 hour after being reconstituted. The formulation of the invention comprising antibodies can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Adult subjects are characterized as having “hypertension” or a high blood pressure when they have a systolic blood pressure of more than 140 mmHg and/or a diastolic blood pressure of more than 90 mmHg.

Specific populations treatable by the therapeutic methods of the invention include subjects with one or more of the following conditions: subjects indicated for LDL apheresis, subjects with PCSK9-activating mutations (gain of function mutations, “GOF”), subjects with elevated total cholesterol levels, subjects with elevated low-density lipoprotein cholesterol (LDL-C) levels, subjects with primary hypercholesterolemia, such as subjects primary with Familial or Non-Familial Hypercholesterolemia, subjects with heterozygous Familial Hypercholesterolemia (heFH); subjects with hypercholesterolemia, especially primary hypercholesterolemia, who are statin intolerant or statin uncontrolled; and subjects at risk for developing hypercholesterolemia who may be preventably treated. Other indications include hyperlipidemia and dyslipidemia, especially if associated with secondary causes such as Type 2 diabetes mellitus, cholestatic liver diseases (primary biliary cirrhosis), nephrotic syndrome, hypothyroidism, obesity; and the prevention and treatment of atherosclerosis and cardiovascular diseases, such as coronary heart disease (CHD). The conditions or disorders as listed for the above populations or subjects are conditions or disorders, for which treatment with the antibody of the invention is especially suitable.

However, depending on the severity of the afore-mentioned diseases and conditions, the treatment of subjects with the antibodies and antigen-binding fragments of the invention may be contraindicated for certain diseases and conditions.

The term “adverse effect” (or side-effect) refers to a harmful and undesired effect resulting from a medication. An adverse effect may be termed a “side effect”, when judged to be secondary to a main or therapeutic effect. Some adverse effects occur only when starting, increasing or discontinuing a treatment. Adverse effects may cause medical complications of a disease and negatively affect its prognosis. Examples of side effects are allergic reactions, vomiting, headache, or dizziness or any other effect herein described.

As used herein, “treat”, “treating” or “treatment” of a disease or disorder means accomplishing one or more of the following: (a) reducing the severity and/or duration of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, “prevent”, “preventing”, “prevention”, or “prophylaxis” of a disease, condition or disorder means preventing that a disorder, disease or condition occurs in subject.

Elevated total cholesterol levels are understood in the context of present invention to preferably be total cholesterol levels of 200 mg/dL or more, especially 240 mg/dL or more. International treatment guidelines recommend lowering LDL-C to <2.0-2.6 mmol/L (<77-100 mg/dL) in patients with established cardiovascular diseases (CVDs) and to <1.8-2.0 mmol/L (<70-77 mg/dL) in high-risk groups such as those with CVDs plus diabetes, smoking, poorly controlled hypertension, metabolic syndrome, or previous myocardial infarction. Elevated LDL-C levels are thus understood in the context of present invention to be LDL-C levels of 77 mg/dL or more (especially for patients with one or more of the following characteristics: established CVDs and one or more of [diabetes, with smoking, poorly controlled hypertension, metabolic syndrome or previous myocardial infarction]) and 100 mg/dL or more (especially for patients with established CVDs), 130 mg/dL or more, or 160 mg/dL or 190 mg/dL or more. Low High-density lipoprotein levels (HDL-levels) in the context of present invention are understood to be preferably less than about 40 mg/dL.

The terms “uncontrolled by statins” or “statin-resistant”, especially in the context of hyperlipidemia, hypercholesterolemia etc., are used synonymously herein and refer to conditions such as hyperlipidemia, wherein treatment with a statin (i.e. regular administration of a statin such as atorvastatin to a patient) does not significantly lower total cholesterol or LDL-C or does not suffice to establish normolipidemic levels for the patient or to establish a lipidemic (e.g. total cholesterol or LDL-C) level that is not a significant risk factor for developing cardiovascular diseases. This means for example that statin-treatment does not suffice to establish levels of less than 130 mg/dL in general, or of less than 100 mg/dL (e.g. about ≥77 mg/dL to about 100 mg/dL), especially in patients with established cardiovascular diseases, or to establish levels of about less than 77 mg/dL (e.g. about ≥70-77 mg/dL), especially in high-risk groups such as those with CVDs plus diabetes, smoking, poorly controlled hypertension, metabolic syndrome, or previous myocardial infarction. In the context of present invention, statin resistance preferably relates to atorvastatin resistance.

### Embodiments of the Invention

The present invention will now be further described. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous, unless clearly indicated to the contrary.

In a first aspect the present invention is directed to a method for treating a disease or condition in which PCSK9 expression or activity causes an, comprising:
administering a therapeutic amount of an antibody or an antigen-binding fragment thereof which specifically binds hPCSK9 (human proprotein convertase subtilisin/kexin type 9) to a subject in need thereof, wherein the antibody or antigen-binding fragment thereof is administered in a dosage amount ranging from 5 mg to 500 mg, and
administering a therapeutic amount of an HMG-COA reductase inhibitor to said subject, wherein the HMG-COA reductase inhibitor is preferably administered in a dosage amount ranging from 0.05 mg to 100 mg.

In the context of present application, the term “a disease or condition in which PCSK9 expression or activity causes an impact” is understood to comprise any disease or condition in which the application of a PCSK-9 antibody causes an impact.

In preferred embodiments of the first and the other aspects of present invention, the disease or condition in which PCSK9 expression or activity causes an impact is ameliorated, improved, inhibited or prevented with a PCSK9 antagonist.

In further preferred embodiments of the first and the other aspects of present invention, the disease or condition is selected from the group consisting of: elevated total cholesterol levels, elevated low-density lipoprotein cholesterol (LDL-C) levels, hypercholesterolemia, particularly hypercholesteremia uncontrolled by statins, hyperlipidemia, dyslipidemia, atherosclerosis, cardiovascular diseases, primary hypercholesterolemia, such as primary familial hypercholesterolemia or primary non-familial hypercholesterolemia, hypercholesterolemia (especially primary hypercholesterolemia) uncontrolled by statins (particularly uncontrolled by atorvastatin).

In preferred embodiments of the first and the other aspects of present invention, the subject in need thereof is a subject indicated for LDL apheresis, a subject with PCSK9-activating mutations, a subject with heterozygous Familial Hypercholesterolemia, a subject with primary hypercholesterolemia, a subject with primary hypercholesterolemia who is statin uncontrolled, a subject at risk for developing hypercholesterolemia, a subject with hypercholesterolemia, a subject with hyperlipidemia, a subject with dyslipidemia, a subject with atherosclerosis or a subject with cardiovascular diseases. Most preferably, the subject in need thereof is a human subject.

In some embodiments of the first and other aspects of the invention, the HMG-COA reductase inhibitor is administered three times per day, twice per day, or once per day. In some embodiments of the first and the other aspects of present invention, the HMG-COA reductase inhibitor is administered every day, every other day, every third day, every fourth day, every fifth day, or every sixth day. In some embodiments of the first and the other aspects of present invention, the HMG-COA reductase inhibitor is administered every week, every other week, every third week, or every fourth week. In some embodiments of the first and the other aspects of present invention, the HMG-COA reductase inhibitor is administered in the morning, at noon or in the evening. In preferred embodiments, the HMG-COA reductase inhibitor is administered once per day, preferably orally, preferably in the evening.

Preferably, the HMG-COA reductase inhibitor is a statin. More preferably, the statin is selected from the group consisting of cerivastatin, atorvastatin, simvastatin, pitavastatin, rosuvastatin, fluvastatin, lovastatin, and pravastatin.

In more preferred embodiments of the first and the other aspects of present invention, the statin is
cerivastatin administered in a daily dosage of between 0.05 mg and 2 mg, preferably in a daily dosage of 0.2 mg, 0.4 mg, or 0.8 mg;
atorvastatin administered in a daily dosage of between 2 mg and 100 mg, preferably in a daily dosage of 10 mg, 20 mg, 40 mg, or 80 mg;
simvastatin administered in a daily dosage of between 2 mg and 100 mg, preferably in a daily dosage of 5 mg, 10 mg, 20 mg, 40 mg, or 80 mg;
pitavastatin administered in a daily dosage of between 0.2 mg and 100 mg, preferably in a daily dosage of 1 mg, 2 mg, 5 mg, 10 mg, or 20 mg;
rosuvastatin administered in a daily dosage of between 2 mg and 100 mg, preferably in a daily dosage of 5 mg, 10 mg, 20 mg, or 40 mg;
fluvastatin administered in a daily dosage of between 2 mg and 100 mg, preferably in a daily dosage of 20 mg, 40 mg, or 80 mg;
lovastatin administered in a daily dosage of between 2 mg and 100 mg, preferably in a daily dosage of 10 mg, 20 mg, 40 mg, or 80 mg; or
pravastatin administered in a daily dosage of between 2 mg and 100 mg, preferably in a daily dosage of 10 mg, 20 mg, 40 mg, or 80 mg.

In preferred embodiments of the first and the other aspects of present invention, the antibody or antigen-binding fragment thereof is administered to the subject every other week, every fourth week or once a month. Administration every fourth week or administration once a month (i.e. once per calendar month, e.g. every first, second etc. day of the month or every first, second third Monday, Tuesday etc. each month, in contrast to administration every fourth week) is preferred in view of patient compliance. Administration every other week is preferred in view of a very low variation of blood cholesterol levels. Other suitable time schedules for administration of the antibody or antigen-binding fragment thereof include without limitation an administration once per day, every other day, every third day, every fourth day, every fifth day, every sixth day, every week, every third week, every fifth week, every sixth week, every eighth week, every tenth week, and every twelfth week.

In preferred embodiments of the first and the other aspects of present invention, the antibody or antigen-binding fragment thereof is administered in a dosage amount ranging e.g. from about 40 mg to about 500 mg, from about 50 mg to about 500 mg, from about 50 mg to 300 mg or from about 100 mg to 200 mg. In more preferred embodiments, the antibody or antigen-binding fragment thereof is administered in a dosage amount of about 50 mg, of about 100 mg, of about 150 mg, of about 200 mg, of about 250 mg, of about 300 mg, of about 350 mg, of about 400 mg, of about 450 mg or of about 500 mg. Doses of about 50 to about 200 mg, e.g. of about 50 mg, about 100 mg, about 150 mg or about 200 mg are especially suitable for a biweekly dosage regimen (i.e. the application every other week), doses of about 150 mg to about 400 mg, e.g. about 150 mg, about 200 mg, about 250 mg, about 300 mg about 350 mg or about 400 mg are especially suitable for an administration regime with longer intervals, e.g. an administration every third or every fourth week or once a month.

Antibodies and antigen-binding fragments thereof that can be used for practicing the first and the other aspects of the present invention are described in the section “Preferred Antibodies for Practicing the Present Invention”.

In a second aspect the present invention is directed to an antibody or an antigen-binding fragment thereof which specifically binds hPCSK9 (human proprotein convertase subtilisin/kexin type 9) for use in the treatment of a disease or condition in which PCSK9 expression or activity causes an impact,
wherein the antibody or antigen-binding fragment thereof is for administration in a dosage amount ranging from 5 mg to 500 mg,
wherein the antibody or antigen-binding fragment thereof is further for administration in combination with an HMG-COA reductase inhibitor at a dosage amount ranging from 0.05 mg to 100 mg.

In preferred embodiments of the second and the other aspects of present invention, the disease or condition in which PCSK9 expression or activity causes an impact is ameliorated, improved, inhibited or prevented with a PCSK9 antagonist.

In further preferred embodiments of the second and the other aspects of present invention, the disease or condition is selected from the group consisting of: elevated low-density lipoprotein cholesterol (LDL-C) levels, hypercholesterolemia, particularly hypercholesterolemia uncontrolled by statins, hyperlipidemia, dyslipidemia, atherosclerosis and cardiovascular diseases, particularly primary hypercholesterolemia such as primary familial hypercholesterolemia or primary non-familial hypercholesterolemia.

In preferred embodiments of the second and the other aspects of present invention, the antibody or antigen-binding fragment thereof is for administration to a subject indicated for LDL apheresis, a subject with PCSK9-activating mutations, a subject with heterozygous Familial Hypercholesterolemia, a subject with primary hypercholesterolemia, a subject with primary hypercholesterolemia who is statin uncontrolled, a subject at risk for developing hypercholesterolemia, a subject with hypercholesterolemia, a subject with hyperlipidemia, a subject with dyslipidemia, a subject with atherosclerosis or a subject with cardiovascular diseases. Most preferably, the subject is a human subject.

In some embodiments of the second and the other aspects of present invention, the antibody or antigen-binding fragment thereof is for administration in combination with an HMG-CoA reductase inhibitor, which is to be administered three times per day, twice per day, or once per day. In some embodiments of the second and the other aspects of present invention, the HMG-COA reductase inhibitor is to be administered every day, every other day, every third day, every fourth day, every fifth day, or every sixth day. In some embodiments of the second and the other aspects of present invention, the HMG-CoA reductase inhibitor is to be administered every week, every other week, every third week, or every fourth week. In some embodiments of the second and the other aspects of present invention, the HMG-COA reductase inhibitor is to be administered in the morning, at noon or in the evening. In preferred embodiments, the HMG-CoA reductase inhibitor is to be administered once per day, preferably orally, preferably in the evening.

In preferred embodiments of the second and the other aspects of present invention, the HMG-COA reductase inhibitor is a statin. More preferably, the statin is selected from the group consisting of cerivastatin, atorvastatin, simvastatin, pitavastatin, rosuvastatin, fluvastatin, lovastatin, and pravastatin.

In more preferred embodiments of the second and the other aspects of present invention, the statin is
cerivastatin which is to be administered in a daily dosage of between 0.05 mg and 2 mg, preferably in a daily dosage of 0.2 mg, 0.4 mg, or 0.8 mg;
atorvastatin which is to be administered in a daily dosage of between 2 mg and 100 mg, preferably in a daily dosage of 10 mg, 20 mg, 40 mg, or 80 mg;
simvastatin which is to be administered in a daily dosage of between 2 mg and 100 mg, preferably in a daily dosage of 5 mg, 10 mg, 20 mg, 40 mg, or 80 mg;
pitavastatin which is to be administered in a daily dosage of between 0.2 mg and 100 mg, preferably in a daily dosage of 1 mg, 2 mg, 5 mg, 10 mg, or 20 mg;
rosuvastatin which is to be administered in a daily dosage of between 2 mg and 100 mg, preferably in a daily dosage of 5 mg, 10 mg, 20 mg, or 40 mg;
fluvastatin which is to be administered in a daily dosage of between 2 mg and 100 mg, preferably in a daily dosage of 20 mg, 40 mg, or 80 mg;
lovastatin which is to be administered in a daily dosage of between 2 mg and 100 mg, preferably in a daily dosage of 10 mg, 20 mg, 40 mg, or 80 mg; or
pravastatin which is to be administered in a daily dosage of between 2 mg and 100 mg, preferably in a daily dosage of 10 mg, 20 mg, 40 mg, or 80 mg.

In preferred embodiments of the second and the other aspects of present invention, the antibody or antigen-binding fragment thereof is for administration to the subject every other week, every fourth week or once a month. Administration every fourth week or administration once a month is preferred in view of patient compliance. Administration every other week is preferred in view of a very low variation of blood cholesterol levels. Other suitable time schedules for administration of the antibody or antigen-binding fragment thereof include without limitation an administration once per day, every other day, every third day, every fourth day, every fifth day, every sixth day, every week, every third week, every fifth week, every sixth week, every eighth week, every tenth week, and every twelfth week.

In preferred embodiments of the second and the other aspects of present invention, the antibody or antigen-binding fragment thereof is for administration in a dosage amount ranging from about 40 mg to about 500 mg or from about 50 mg to about 500 mg or from about 50 mg to about 400 mg or from about 50 mg to about 300 mg, or from about 100 mg to about 300 mg or from about 100 mg to about 200 mg. In more preferred embodiments, the antibody or antigen-binding fragment thereof is for administration in a dosage amount of about 50 mg, of about 100 mg, of about 150 mg, of about 200 mg, of about 250 mg, of about 300 mg, of about 350 or of about 400 mg.

In preferred embodiments of the second and the other aspects of present invention the antibody or antigen-binding fragment thereof is for administration in a dosage amount (i.e. a dosage regimen) ranging from about 50 mg to about 200 mg every other week (E2W), preferably about 50 mg E2W, about 100 mg E2W, about 150 mg E2W, about 200 mg E2W, about 250 mg E2W or about 300 mg E2W, with about 50 mg E2W, about 100 mg E2W, about 150 mg E2W, about 200 mg E2W, being even more preferred. According to an especially advantageous embodiment of the second and the other aspects of present invention of present invention the antibody or antigen-binding fragment thereof is for administration in a dosage amount (i.e. a dosage regimen) E2W from about 50 mg to about 200 mg from about 100 mg to about 180 mg, from about 130 mg to about 170 mg, from about 140 to about 160 mg or about 90, about 100, about 110, about 120, about 130, about 140, about 145, about 150, about 155, about 160, about 170, about 180, about 190 or about 200 mg E2W, with dosage regimens of about 145 mg to about 155 mg E2W and particularly about 150 mg E2W belonging to the particularly preferred embodiments.

In other preferred embodiments of the second and the other aspects of present invention, the antibody or antigen-binding fragment thereof is for administration in a dosage amount ranging from about 100 mg to about 400 mg every fourth week (E4W), preferably about 100 mg E4W, about 150 mg E4W, about 200 mg E4W, about 250 mg E4W, about 300 mg E4W, about 350 mg E4W or about 400 mg E4W, with dosage amounts of about 190 to about 310 E4W, of about 200 to about 300 mg E4W, about 190 to about 210 E4W, about 195 to about 205 E4W, about 290 to about 310 E4W, about 295 to about 305 E4W, about 200 mg E4W or about 300 mg E4W belonging to the particularly preferred embodiments. These dosage amounts indicated for administration E4W are also suitable for administration once a month.

Antibodies and antigen-binding fragments thereof that can be used for practicing the second and other aspects of present invention of the present invention are described in the section “Preferred Antibodies for Practicing the Present Invention”.

In a third aspect the present invention is directed to an article of manufacture comprising: (a) a packaging material; (b) an antibody or an antigen-binding fragment thereof which specifically binds hPCSK9; and (c) a label or packaging insert contained within the packaging material indicating that patients receiving treatment with said antibody or antigen-binding fragment can be treated for a disease or condition selected from the group consisting of hypercholesterolemia, hyperlipidemia, dyslipidemia, atherosclerosis and cardiovascular diseases.

In a fourth aspect the present invention is directed to an article of manufacture comprising: (a) a packaging material; (b) an antibody or an antigen-binding fragment thereof which specifically binds hPCSK9; and (c) a label or packaging insert contained within the packaging material indicating the treatment of patients with said antibody or antigen-binding fragment thereof together with the application of an HMG Co A inhibitor such as a statin.

In a fifth aspect the present invention is directed to an article of manufacture comprising (a) a packaging material; (b) an antibody or an antigen-binding fragment thereof which specifically binds hPCSK9; and (c) a label or packaging insert indicating that the treatment of patients with said antibody or antigen-binding fragment thereof together with an HMG-Co A inhibitor such as a statin is contraindicated for patients belonging to one or more of the following groups: (i) smokers; (ii) persons being 70 years old or older; (iii) persons suffering from hypertension; (iv) women who are pregnant; (v) women who are trying to become pregnant; (vi) women who are breast-feeding; (vii) persons who have or ever had a disease affecting the liver; (viii) persons who had any unexplained abnormal blood tests for liver function; (ix) persons who drink excessive amounts of alcohol; (x) persons having kidney problems; (xi) persons suffering from hypothyroidism; (xii) persons suffering from muscle disorders; (xiii) persons having encountered previous muscular problems during treatment with lipid-lowering medicine; (xiv) persons having serious problems with their breathing; (xv) persons taking one or more of the following medicines: medicines altering the way the immune systems works (e.g. cyclosporin or antihistamines), antibiotics or antifungal medicines (e.g. erythromycin, clarithromycin, ketoconazole, itraconazole, rifampicin, fusidic acid), medicines regulating lipid levels (e.g. gemfibrozil, colestipol), calcium channel blockers (e.g. verapamil, diltiazem), medicines regulating the heart rhythm (digoxin, amiodarone), protease inhibitors used in the treatment of HIV (e.g. nelfinavir), warfarin, oral contraceptives, antacids or St. John's Wort; or (xvi) persons drinking more than 0.1 L of grapefruit juice per day or eating more than half a grapefruit per day; (xvii) persons having a body mass index (BMI) of more than 40; (xviii) persons having a body mass index (BMI) of less than 18; (xix) persons suffering from type 1 diabetes or type 2 diabetes; (xx) persons positive for hepatitis B or hepatitis C; (xxi) persons having a known sensitivity to monoclonal antibody therapeutics; (xxii) persons having a neutrophil concentration of less than 1500/mm³; (xxiii) persons having a platelet concentration of less than 100000/mm³; (xxiv) men having a serum creatinine level larger than 1.5×ULN (upper limit of normal); (xxv) women having a serum creatinine level larger than 1.4×ULN (upper limit of normal); (xxvi) persons having an alanine transaminase (ALT) level or aspartate transaminase (AST) level larger than 2×ULN; or (xxvii) persons having a CPK level larger than 3×ULN.

In preferred embodiments of the third, fourth and fifth aspect, the antibody or antigen-binding fragment is an antibody or antigen-binding fragment as specified below in the section “Preferred Antibodies for Practicing the Present Invention”.

The label or packaging insert according to the different aspects and embodiments of the invention, particularly in respect to the different articles of manufacture of the invention, can be any kind of data carrier suitable to be arranged within the package or container or on the outside of the package or container. Preferably, the data carrier (i.e. label or, chip, bar code or leaflet or label comprising a bar code etc.) comprises information such as
(i) composition, formulation, concentration and total amount, identity of active ingredient(s) contained in the article of manufacture, i.e. of the antibody or antigen-fragments, HMG-CoA reductase inhibitor, pharmaceutical composition, unit dosage form or formulation of present invention
(ii) number and composition of unit dosage form contained in the article of manufacture
(iii) indications, contra-indications of the antibody or antigen-fragments, pharmaceutical composition, unit dosage form or formulation of present invention
(iv) (ii) subjects/patients or subject/patient populations indicated or contra-indicated for treatment with the antibody or antigen-fragments, pharmaceutical composition, unit dosage form or formulation of present invention
(v) instructions for use, dosage regimens and/or administration regimes
(vi) quality information such as information about the lot/batch number of the of the antibody or antigen-fragments, pharmaceutical composition, unit dosage form or formulation of present invention, the manufacturing or assembly site or the expiry or sell-by date,
(vii) information concerning the correct storage or handling of the article of manufacture, of the device for application, or of the antibody or antigen-fragments, pharmaceutical composition, unit dosage form or formulation of present invention,
(iv) information concerning the composition of the buffer(s), diluent(s), reagent(s), excipients, carriers, formulations of of the antibody or antigen-fragments, pharmaceutical composition, unit dosage form or formulation of present invention,
(vi) a warning concerning possible consequences when applying unsuitable dosage or administration regimens and/or use in contraindicated indications of patient populations.

In preferred embodiments of the third, fourth and fifth aspect, the label or packaging insert contains reference to a method of treatment or medical use according to the first or second aspect and the embodiments of the first or second aspect as described herein.

A further preferred embodiment of the present invention combines the features of the third aspect and the fourth aspect as described herein.

A further preferred embodiment of the present invention combines the features of the third aspect and the fifth aspect as described herein.

A further preferred embodiment of the present invention combines the features of the fourth aspect and the fifth aspect as described herein.

A further preferred embodiment of the present invention combines the features of the third aspect, the fourth aspect, and the fifth aspect as described herein.

In a sixth aspect the present invention is directed to a method of testing the efficacy of an antibody or an antigen-binding fragment thereof which specifically binds hPCSK9 for the treatment of a disease or condition selected from the group consisting of hypercholesterolemia, hyperlipidemia, dyslipidemia, atherosclerosis and cardiovascular diseases or any of the other conditions or diseases according to the first or second aspect of present invention, said method comprising:
treating a selected patient population with said antibody or antigen-binding fragment thereof, wherein each patient in said population has an LDL cholesterol (LDL-C) level of more than 100 mg/dL; and
determining the efficacy of said antibody or antigen-binding fragment thereof by determining the LDL-C level in the patient population before and after administration of said antibody or antigen-binding fragment thereof, wherein a reduction of the LDL-C level by at least 25% relative to a predose level in at least 75% of the patient population indicates that said antibody or antigen-binding fragment thereof is efficacious for the treatment of said disease or condition in said patient population.

In a seventh aspect the present invention is directed to a method of testing the efficacy of an antibody or an antigen-binding fragment thereof which specifically binds hPCSK9 for the treatment of a disease or condition selected from the group consisting of hypercholesterolemia, hyperlipidemia, dyslipidemia, atherosclerosis and cardiovascular diseases or any of the other conditions or diseases according to the first or second aspect of present invention, said method comprising:

determining the efficacy of an antibody or antigen-binding fragment thereof that has been used for the treatment of a selected patient population with said antibody or antigen-binding fragment thereof, wherein each patient in said population has an LDL cholesterol (LDL-C) level of more than 100 mg/dL by determining the LDL-C level in the patient population before and after administration of said antibody or antigen-binding fragment thereof, wherein a reduction of the LDL-C level by at least 25% relative to a predose level in at least 75% of the patient population indicates that said antibody or antigen-binding fragment thereof is efficacious for the treatment of said disease or condition in said patient population.

In preferred embodiments of the sixth and seventh aspect, each patient in said population has received a lipid lowering treatment by administration of an HMG CoA-Inhibitor, such as a statin for at least 6 weeks prior to treatment with said antibody or antigen-binding fragment thereof.

In preferred embodiments of the sixth and seventh aspect, the antibody or antigen-binding fragment is an antibody or antigen-binding fragment as specified below in the section “Preferred Antibodies for Practicing the Present Invention”.

In preferred embodiments of the sixth and seventh aspect, the selected patient population is treated with a method of treatment according to the first aspect and the embodiments of the first or second aspect as described herein.

In an eighth aspect the present invention is directed to a package comprising an antibody or antigen-binding fragment thereof which specifically binds hPCSK9 and a label, said label comprising a printed statement which informs the patient that the treatment of the antibody together with an HMG-COA reductase inhibitor such as a statin is indicated in one or more of the indications selected from the group consisting of hypercholesterolemia, hyperlipidemia, dyslipidemia, atherosclerosis and cardiovascular diseases or any of the other conditions or diseases according to the first or second aspect of present invention. Antibodies and antigen-binding fragments thereof that can be used for practicing the eighth aspect of the present invention are described in the section “Preferred Antibodies for Practicing the Present Invention”.

In a ninth aspect the present invention is directed to a package comprising an antibody or antigen-binding fragment thereof which specifically binds hPCSK9 and a label, said label comprising a printed statement which informs the patient that the treatment of the antibody together with a statin is contraindicated for patients belonging to one or more of the following groups: (i) smokers; (ii) persons being 70 years old or older; (iii) persons suffering from hypertension; (iv) women who are pregnant; (v) women who are trying to become pregnant; (vi) women who are breast-feeding; (vii) persons who have or ever had a disease affecting the liver; (viii) persons who had any unexplained abnormal blood tests for liver function; (ix) persons who drink excessive amounts of alcohol; (x) persons having kidney problems; (xi) persons suffering from hypothyroidism; (xii) persons suffering from muscle disorders; (xiii) persons having encountered previous muscular problems during treatment with lipid-lowering medicine; (xiv) persons having serious problems with their breathing; (xv) persons taking one or more of the following medicines: medicines altering the way the immune systems works (e.g. ciclosporin or antihistamines), antibiotics or antifungal medicines (e.g. erythromycin, levels (e.g. gemfibrozil, colestipol), calcium channel blockers (e.g. verapamil, diltiazem), medicines regulating the heart rhythm (digoxin, amiodarone), protease inhibitors used in the treatment of HIV (e.g. nelfinavir), warfarin, oral contraceptives, antacids or St. John's Wort; or (xvi) persons drinking more than 0.1 L of grapefruit juice per day or eating more than half a grapefruit per day; (xvii) persons having a body mass index (BMI) of more than 40; (xviii) persons having a body mass index (BMI) of less than 18; (xix) persons suffering from type 1 diabetes or type 2 diabetes; (xx) persons positive for hepatitis B or hepatitis C; (xxi) persons having a known sensitivity to monoclonal antibody therapeutics; (xxii) persons having a neutrophil concentration of less than 1500/mm³; (xxiii) persons having a platelet concentration of less than 100000/mm³; (xxiv) men having a serum creatinine level larger than 1.5×ULN (upper limit of normal); (xxv) women having a serum creatinine level larger than 1.4×ULN (upper limit of normal); (xxvi) persons having an alanine transaminase (ALT) level or aspartate transaminase (AST) level larger than 2×ULN; or (xxvii) persons having a CPK level larger than 3×ULN. Antibodies and antigen-binding fragments thereof that can be used for practicing the ninth aspect of the present invention are described in the section “Preferred Antibodies for Practicing the Present Invention”.

A further preferred embodiment of the present invention combines the features of the eighth aspect and the ninth aspect as described herein.

In a tenth aspect the present invention is directed to a method of regulating the LDL level in the blood comprising:
administering a therapeutic amount of an antibody or an antigen-binding fragment thereof which specifically binds hPCSK9 (human proprotein convertase subtilisin/kexin type 9) to a subject in need thereof, wherein the antibody or antigen-binding fragment thereof is administered in a dosage amount ranging from 5 mg to 500 mg, and
administering a therapeutic amount of an HMG-COA reductase inhibitor to said subject, wherein the HMG-COA reductase inhibitor is preferably administered in a dosage amount ranging from 0.05 mg to 100 mg.

In an eleventh aspect the present invention is directed to a method of preventing effects of a (persistently) increased LDL level in the blood comprising:
administering a therapeutic amount of an antibody or an antigen-binding fragment thereof which specifically binds hPCSK9 (human proprotein convertase subtilisin/kexin type 9) to a subject in need thereof, wherein the antibody or antigen-binding fragment thereof is administered in a dosage amount ranging from 5 mg to 500 mg, and
administering a therapeutic amount of an HMG-COA reductase inhibitor to said subject, wherein the HMG-COA reductase inhibitor is preferably administered in a dosage amount ranging from 0.05 mg to 100 mg.

In preferred embodiments of the tenth and eleventh aspect, the disease or condition in which PCSK9 expression or activity causes an impact is ameliorated, improved, inhibited or prevented with a PCSK9 antagonist. In further preferred embodiments of the tenth and eleventh aspect, the disease or condition in which PCSK9 expression or activity causes an impact is selected from the group consisting of: elevated LDL-C levels, hypercholesterolemia, hyperlipidemia, dyslipidemia, atherosclerosis and cardiovascular diseases or any of the other conditions or diseases according to the first or second aspect of present invention.

In preferred embodiments of the tenth and eleventh aspect, the subject in need thereof is a subject indicated for LDL apheresis, a subject with PCSK9-activating mutations, a subject with heterozygous Familial Hypercholesterolemia, a subject with primary hypercholesterolemia who is statin uncontrolled, a subject at risk for developing hypercholesterolemia, a subject with hypercholesterolemia, a subject with hyperlipidemia, a subject with dyslipidemia, a subject with atherosclerosis or a subject with cardiovascular diseases or any of the subjects as described in the first or second aspect of present invention. Most preferably, the subject in need thereof is a human subject.

In some embodiments of the tenth and eleventh aspect, the HMG-CoA reductase inhibitor is administered three times per day, twice per day, or once per day. In some embodiments, the HMG-COA reductase inhibitor is administered every day, every other day, every third day, every fourth day, every fifth day, or every sixth day. In some embodiments, the HMG-COA reductase inhibitor is administered every week, every other week, every third week, every fourth week, or every month. In some embodiments, the HMG-COA reductase inhibitor is administered in the morning, at noon or in the evening. In preferred embodiments, the HMG-COA reductase inhibitor is administered once per day, preferably orally, preferably in the evening. Further suitable administration regimes are described in the first or second aspect.

Preferably, the HMG-COA reductase inhibitor is a statin. More preferably, the statin is selected from the group consisting of cerivastatin, atorvastatin, simvastatin, pitavastatin, rosuvastatin, fluvastatin, lovastatin, and pravastatin.

In more preferred embodiments of the tenth and eleventh aspect, the statin is
cerivastatin administered in a daily dosage of between 0.05 mg and 2 mg, preferably in a daily dosage of 0.2 mg, 0.4 mg, or 0.8 mg;
atorvastatin administered in a daily dosage of between 2 mg and 100 mg, preferably in a daily dosage of 10 mg, 20 mg, 40 mg, or 80 mg;
simvastatin administered in a daily dosage of between 2 mg and 100 mg, preferably in a daily dosage of 5 mg, 10 mg, 20 mg, 40 mg, or 80 mg;
pitavastatin administered in a daily dosage of between 0.2 mg and 100 mg, preferably in a daily dosage of 1 mg, 2 mg, 5 mg, 10 mg, or 20 mg;
rosuvastatin administered in a daily dosage of between 2 mg and 100 mg, preferably in a daily dosage of 5 mg, 10 mg, 20 mg, or 40 mg;
fluvastatin administered in a daily dosage of between 2 mg and 100 mg, preferably in a daily dosage of 20 mg, 40 mg, or 80 mg;
lovastatin administered in a daily dosage of between 2 mg and 100 mg, preferably in a daily dosage of 10 mg, 20 mg, 40 mg, or 80 mg; or
pravastatin administered in a daily dosage of between 2 mg and 100 mg, preferably in a daily dosage of 10 mg, 20 mg, 40 mg, or 80 mg.

In preferred embodiments of the tenth and eleventh aspect, the antibody or antigen-binding fragment thereof is administered to the subject every other week, every fourth week or once a month. Administration every fourth week or every month is preferred in view of patient compliance. Administration every other week is preferred in view of a very low variation of blood cholesterol levels. Other suitable time schedules for administration of the antibody or antigen-binding fragment thereof include without limitation an administration once per day, every other day, every third day, every fourth day, every fifth day, every sixth day, every week, every third week, every fifth week, every sixth week, every eighth week, every tenth week, and every twelfth week.

In preferred embodiments of the tenth and eleventh aspect, the antibody or antigen-binding fragment thereof is administered in a dosage amount ranging from 50 mg to 300 mg, e.g. from 100 mg to 200 mg. In more preferred embodiments, the antibody or antigen-binding fragment thereof is administered in a dosage amount of about 50 mg, of about 100 mg, of about 150 mg, of about 200 mg, or of about 300 mg. Further suitable and preferred dosage regimens are described in the first or second aspect.

Antibodies and antigen-binding fragments thereof that can be used for practicing the tenth and eleventh aspect of the present invention are described in the section “Preferred Antibodies for Practicing the Present Invention”.

In a twelfth aspect the present invention is directed to a method of determining whether a pharmaceutical compound is utilizable for ameliorating, improving, inhibiting or preventing a disease or condition in which PCSK9 activity or expression has an impact comprising:
(a) administering to a subject a compound that specifically binds to PCSK9, preferably an antibody or antigen-binding fragment thereof specifically binding to PCSK9, and
(b) determining what fraction of PCSK9 in the blood is attached to the compound from (a).

Typically, compounds that specifically bind from 10% to 100% (preferably from 20% to 100%, more preferably from 30% to 100%, more preferably from 40% to 100%, more preferably from 50% to 100%) of the PCSK9 present in the blood when used in stoichiometric amounts, will be utilizable for ameliorating, improving, inhibiting or preventing a disease or condition in which PCSK9 activity or expression has an impact.

Preferably, the disease or condition in which PCSK9 expression or activity has an impact is selected from the group consisting of: elevated LDL-C levels, hypercholesterolemia, hyperlipidemia, dyslipidemia, atherosclerosis and cardiovascular diseases or any of the other diseases described in the first or second aspect.

Antibodies and antigen-binding fragments thereof that can be used for practicing the twelfth aspect of the present invention are described in the section “Preferred Antibodies for Practicing the Present Invention”.

In a thirteenth aspect the present invention is directed to a method for treating a disease or condition in which PCSK9 expression or activity causes an impact comprising
administering a therapeutic amount of an antibody or an antigen-binding fragment thereof which specifically binds hPCSK9 (human proprotein convertase subtilisin/kexin type 9) to a subject in need thereof,
wherein the subject in need thereof falls into one or more of the following groups of subjects: (i) subjects having a serum LDL cholesterol (LDL-C) level of at least 100 mg/dL, preferably at least 130 mg/dL, more preferably at least 160 mg/dL, even more preferably at least 200 mg/dL; (ii) subjects having a serum HDL-C level of less than 40 mg/dL; (iii) subjects having a serum cholesterol level of at least 200 mg/dL, preferably at least 240 mg/dL; (iv) subjects having a serum triacylglycerol level of at least 150 mg/dL, e.g. at least 200 mg/dL or at least 500 mg/dL, wherein said triacylglycerol level is determined after fasting for at least 8 hours; (v) subjects being at least 35 years old, e.g. at least 40 years old, at least 45 years old, at least 50 years old, at least 55 years old, at least 60 years old, at least 65 years old, or at least 75 years old; (vi) subjects younger than 75 years, e.g. younger than 70 years, younger than 65 years, younger than 60 years, younger than 55 years, younger than 50 years, younger than 45 years, or younger than 40 years; (vii) subjects having a BMI of 25 or more (e.g. 26 or more, 27 or more, 28 or more, 29 or more, 30 or more, 31 or more, 32 or more, 33 or more, 34 or more, 35 or more, 36 or more, 37 or more, 38 or more, or 39 or more); (viii) male subjects; (ix) female subjects; (x) subjects in which the administration of said antibody or antigen-binding fragment thereof leads to a reduction in the serum LDL-C level by at least 30 mg/dL, preferably by at least 40 mg/dL, more preferably by at least 50 mg/dL, more preferably by at least 60 mg/dL, more preferably by at least 70 mg/dL, relative to predose level; or (xi) subjects in which the administration of said antibody or antigen-binding fragment thereof leads to a reduction in the serum LDL-C level by at least 20%, preferably by at least 30%, more preferably by at least 40%, more preferably by at least 50%, more preferably by at least 60%, relative to predose level.

In a fourteenth aspect the present invention is directed to a method for treating a disease or condition in which PCSK9 expression or activity causes an impact comprising
administering a therapeutic amount of an antibody or an antigen-binding fragment thereof which specifically binds hPCSK9 (human proprotein convertase subtilisin/kexin type 9) to a subject in need thereof,
wherein the subject in need thereof does not fall into one or more of the following groups of subjects: (i) smokers; (ii) persons being 70 years old or older; (iii) persons suffering from hypertension; (iv) women who are pregnant; (v) women who are trying to become pregnant; (vi) women who are breast-feeding; (vii) persons who have or ever had a disease affecting the liver; (viii) persons who had any unexplained abnormal blood tests for liver function; (ix) persons who drink excessive amounts of alcohol; (x) persons having kidney problems; (xi) persons suffering from hypothyroidism; (xii) persons suffering from muscle disorders; (xiii) persons having encountered previous muscular problems during treatment with lipid-lowering medicine; (xiv) persons having serious problems with their breathing; (xv) persons taking one or more of the following medicines: medicines altering the way the immune systems works (e.g. ciclosporin or antihistamines), antibiotics or antifungal medicines (e.g. erythromycin, levels (e.g. gemfibrozil, colestipol), calcium channel blockers (e.g. verapamil, diltiazem), medicines regulating the heart rhythm (digoxin, amiodarone), protease inhibitors used in the treatment of HIV (e.g. nelfinavir), warfarin, oral contraceptives, antacids or St. John's Wort; or (xvi) persons drinking more than 0.1 L of grapefruit juice per day or eating more than half a grapefruit per day; (xvii) persons having a body mass index (BMI) of more than 40; (xviii) persons having a body mass index (BMI) of less than 18; (xix) persons suffering from type 1 diabetes or type 2 diabetes; (xx) persons positive for hepatitis B or hepatitis C; (xxi) persons having a known sensitivity to monoclonal antibody therapeutics; (xxii) persons having a neutrophil concentration of less than 1500/mm³; (xxiii) persons having a platelet concentration of less than 100000/mm³; (xxiv) men having a serum creatinine level larger than 1.5×ULN (upper limit of normal); (xxv) women having a serum creatinine level larger than 1.4×ULN (upper limit of normal); (xxvi) persons having an alanine transaminase (ALT) level or aspartate transaminase (AST) level larger than 2×ULN; or (xxvii) persons having a CPK level larger than 3×ULN.

In preferred embodiments of the thirteenth and the fourteenth aspect, the disease or condition in which PCSK9 expression or activity causes an impact is ameliorated, improved, inhibited or prevented with a PCSK9 antagonist.

In preferred embodiments of the thirteenth and the fourteenth aspect, the disease or condition in which PCSK9 expression or activity causes an impact is selected from the group consisting of: elevated LDL-C levels, hypercholesterolemia, hyperlipidemia, dyslipidemia, atherosclerosis and cardiovascular diseases or any of the other diseases or conditions described in the other aspects of present invention, such as the first or second aspect.

In preferred embodiments of the thirteenth and the fourteenth aspect, the subject in need thereof is a subject indicated for LDL apheresis, a subject with PCSK9-activating mutations, a subject with heterozygous Familial Hypercholesterolemia, a subject with primary hypercholesterolemia, e.g. a subject with primary Familial or primary non-Familial Hypercholesterolemia, a subject with hypercholesterolemia such as primary hypercholesterolemia who is statin uncontrolled, a subject at risk for developing hypercholesterolemia, a subject with hypercholesterolemia, a subject with hyperlipidemia, a subject with dyslipidemia, a subject with atherosclerosis or a subject with cardiovascular diseases, or any of the other subjects described in the first or second aspects. Most preferably, the subject in need thereof is a human subject. Further preferred or suitable subjects are described at the other aspects of present invention.

Antibodies and antigen-binding fragments thereof that can be used for practicing the thirteenth and fourteenth aspect and the other aspects of the present invention are described in the section “Preferred Antibodies for Practicing the Present Invention”.

In preferred embodiments of the thirteenth and the fourteenth aspect, the method further comprises: administering a therapeutic amount of an HMG-COA reductase inhibitor to the subject in a dosage of between 0.05 mg to 100 mg. In some embodiments, the HMG-COA reductase inhibitor is administered three times per day, twice per day, or once per day. In some embodiments, the HMG-COA reductase inhibitor is administered every day, every other day, every third day, every fourth day, every fifth day, or every sixth day. In some embodiments, the HMG-COA reductase inhibitor is administered every week, every other week, every third week, or every fourth week. In some embodiments, the HMG-COA reductase inhibitor is administered in the morning, at noon or in the evening. In preferred embodiments, the HMG-COA reductase inhibitor is administered once per day, preferably orally, preferably in the evening. Preferably, the HMG-COA reductase inhibitor is a statin. More preferably, the statin is selected from the group consisting of cerivastatin, atorvastatin, simvastatin, pitavastatin, rosuvastatin, fluvastatin, lovastatin, and pravastatin. In further preferred embodiment of the thirteenth and the fourteenth aspect, the method comprises administering a therapeutic amount of a statin to the subject, wherein the statin is:
cerivastatin administered in a daily dosage of between 0.05 mg and 2 mg, preferably in a daily dosage of 0.2 mg, 0.4 mg, or 0.8 mg;
atorvastatin administered in a daily dosage of between 2 mg and 100 mg, preferably in a daily dosage of 10 mg, 20 mg, 40 mg, or 80 mg;
simvastatin administered in a daily dosage of between 2 mg and 100 mg, preferably in a daily dosage of 5 mg, 10 mg, 20 mg, 40 mg, or 80 mg;
pitavastatin administered in a daily dosage of between 0.2 mg and 100 mg, preferably in a daily dosage of 1 mg, 2 mg, 5 mg, 10 mg, or 20 mg;
rosuvastatin administered in a daily dosage of between 2 mg and 100 mg, preferably in a daily dosage of 5 mg, 10 mg, 20 mg, or 40 mg;
fluvastatin administered in a daily dosage of between 2 mg and 100 mg, preferably in a daily dosage of 20 mg, 40 mg, or 80 mg;
lovastatin administered in a daily dosage of between 2 mg and 100 mg, preferably in a daily dosage of 10 mg, 20 mg, 40 mg, or 80 mg; or
pravastatin administered in a daily dosage of between 2 mg and 100 mg, preferably in a daily dosage of 10 mg, 20 mg, 40 mg, or 80 mg.

A further preferred embodiment of the present invention combines the features of the thirteenth aspect and the fourteenth aspect as described herein.

In a fifteenth aspect the present invention is directed to an antibody or an antigen-binding fragment thereof which specifically binds hPCSK9 (human proprotein convertase subtilisin/kexin type 9), wherein the antibody is characterized by one or more of the following features upon administration to a subject, preferably a human or non-human mammal:
13. reduction of low-density lipoprotein (LDL-C) levels of at least about −25% to about −40% relative to a predose level with a sustained reduction over at least a 14 day-period, wherein the sustained reduction is preferably at least −25% and more preferably at least −30% relative to a predose level, particularly if administered in a dose of about 40 to about 60 mg, preferably about 45 to about 55 mg and more preferably about 50 mg in a biweekly administration regime (every other week, E2W),
14. reduction of low-density lipoprotein (LDL-C) of at least about −50% to about −65% relative to a predose level with a sustained reduction over at least a 14 day-period, wherein the sustained reduction is preferably at least −40% and more preferably at least −45% relative to a predose level, particularly if administered in a dose of about 100 mg E2W,
15. reduction of low-density lipoprotein (LDL-C) of at least about −60% to at least about −75% [e.g. at least about −60%, at least about −65%, at least about −70 or at least about −75%] relative to a predose level with a sustained reduction over at least a 14 day-period, wherein the sustained reduction is preferably at least −55% and more preferably at least −60% relative to a predose level, particularly when administered in a dose of about 150 mg E2W,
16. reduction of low-density lipoprotein (LDL-C) of at least about 40% to about 75% relative to a predose level with a sustained reduction over at least a 28 day period, wherein the sustained reduction is preferably at least −35% and more preferably at least −40% relative to a predose level, particularly when administered in a dose of about 200 mg E4W,
17. reduction of low-density lipoprotein (LDL-C) of at least about −50% to about −75% relative to a predose level with a sustained reduction over at least a 28 day-period, wherein the sustained reduction is preferably at least −40% and more preferably at least −45% relative to a predose level, particularly when administered in a dose of about 300 mg E4W,
18. increase of serum HDL cholesterol levels of at least 2%, at least 2.5%, at least, 3%, at least 3.5%, at least 4%, at least 4.5%, at least 5% or at least 5.5% relative to a predose level, particularly when administered in a dose of about 150 mg E2W,
19. reduction of serum total cholesterol at least about 25% to about 35% relative to a predose level with a sustained reduction over at least a 24 day period.
20. reduction of serum total cholesterol at least about 65% to about 80% relative to a predose level with a sustained reduction over at least a 24 day period,
21. reduction of serum triglyceride levels at least about 25% to about 40% relative to a predose level,
22. little or no measurable effect on liver function, as determined by ALT and AST measurements,
23. little or no measurable effect on troponin levels,
24. Increase of one or more of: Total-Cholesterol levels, ApoB levels, non HDL-C levels, Apo-B/ApoA-1 ratio,

The antibody of the fifteenth aspect of present invention exhibits the above properties preferably if administered in combination with an HMG-COA reductase inhibitor treatment. Preferred embodiments of HMG-COA reductase inhibitors to be used in conjunction with the antibody of the invention and dosage and administration regimes thereof can be found throughout the specification, particularly as described in aspects 1, 2 or 19.

According to a preferred embodiment of the antibodies and antigen-binding fragments thereof of present invention, particularly of the antibody or antigen-binding fragment according to the fifteenth aspect, the antibody or antigen binding fragment thereof has one or more of the following characteristics:
(i) The antibody or the antigen-binding fragment comprises the heavy and light chain CDRs of a HCVR/LCVR amino acid sequence pair as shown in SEQ ID NOs: 90/92.
(ii) The antibody or antigen-binding fragment thereof comprises a HCVR/LCVR amino acid sequence pair as shown in SEQ ID NOs: 90/92.
(iii) The antibody or antigen-binding fragment thereof competes for binding to hPCSK9 with an antibody or antigen-binding fragment comprising a HCVR/LCVR amino acid sequence pair as shown in SEQ ID NOs: 90/92.

According to another preferred embodiment of the antibodies and antigen-binding fragments thereof of present invention, particularly of the antibody or antigen-binding fragment according to the fifteenth aspect, the antibody or antigen binding fragment thereof has one or more of the following characteristics:
(i) overcomes statin resistance in mammals, especially in rodents such as hamster
(ii) increase in LDLR expression in mammals, particularly in rodents such as hamster
(iii) decrease of serum LDL-C in rodents such as hamster
(iv) synergistic decrease of LDL-C in conjunction with HMG-COA reductase inhibitor administration, particularly in rodents such as hamster, wherein the HMG-COA reductase inhibitor is preferably Atorvastatin.

Further suitable characteristics and structural features of the antibody of present invention and particularly of the antibody of the fifteenth aspect, as well as antibodies and antigen-binding fragments thereof that can be used for practicing the fifteenth aspect and the other aspects of the present invention are described in the section “Preferred Antibodies for Practicing the Present Invention”.

The antibody of present invention, such as the antibody according to the fifteenth aspect, is preferably formulated as a pharmaceutically applicable formulation as known in the art, and specifically as herein described, such as dry formulation for dissolution or liquid formulation, e.g. as described at the twentyfirst or twentysecond aspect.

In a sixteenth aspect the present invention is directed to an antibody or an antigen-binding fragment thereof which specifically binds hPCSK9 (human proprotein convertase subtilisin/kexin type 9) for use in the treatment of a disease or condition in which PCSK9 expression or activity causes an impact, wherein the antibody or antigen-binding fragment thereof is for administration in a dose of about 50 to 500 mg.

Preferred embodiments of the sixteenth aspect of present invention are described in the fifteenth aspect. Antibodies and antigen-binding fragments thereof that can be used for practicing the sixteenth aspect of the present invention are described in the section “Preferred Antibodies for Practicing the Present Invention”.

According to another preferred embodiment of the sixteenth aspect, the antibody or antigen-binding fragment thereof is for administration in a dose of about 50, 100, 150, 200, 250, 300, 350, 400, 450 or 500 mg and preferably of about 150, 200 or 300 mg.

According to another preferred embodiment of the sixteenth aspect, the disease or condition is selected from the group consisting of: elevated total cholesterol levels, elevated low-density lipoprotein (LDL-C) levels, hypercholesterolemia, hyperlipidemia, dyslipidemia, and atherosclerosis, particularly primary hypercholesterolemia, familial hypercholesterolemia. or hypercholesteremia which is uncontrolled by statins.

According to another preferred embodiment of the sixteenth aspect, the antibody or antigen-binding fragment thereof is administered to the subject every other week (E2W), every fourth week (E4W) or once a month.

According to another preferred embodiment of the sixteenth aspect, the antibody or antigen-binding fragment thereof has one or more of the following characteristics:
(i) is for use in the reduction of low-density lipoprotein (LDL-C) levels of at least about −25% to about −40% relative to a predose level with a sustained reduction over at least a 14 day-period, wherein the sustained reduction is preferably at least −25% and more preferably at least −30% relative to a predose level, wherein the antibody or antigen-binding fragment thereof is preferably administered in a dose of about 40 to about 60 mg, about 45 to about 55 mg or about 50 mg E2W.
(ii) is for use in the reduction of low-density lipoprotein (LDL-C) of at least about −50% to about −65% relative to a predose level with a sustained reduction over at least a 14 day-period, wherein the sustained reduction is preferably at least −40% and more preferably at least −45% relative to a predose level, wherein the antibody or fragment thereof is preferably administered in a dose of about 100 mg E2W.
(iii) is for use in the reduction of low-density lipoprotein (LDL-C) of at least about −60% to at least about −75% [e.g. at least about −60%, at least about −65%, at least about −70 or at least about −75%] relative to a predose level with a sustained reduction over at least a 14 day-period, wherein the sustained reduction is preferably at least −55% and more preferably at least −60% relative to a predose level, wherein the antibody or fragment thereof is preferably administered in a dose of about 150 mg E2W.
(iv) is for use in the reduction of low-density lipoprotein (LDL-C) of at least about 40% to about 75% relative to a predose level with a sustained reduction over at least a 28 day period, wherein the sustained reduction is preferably at least −35% and more preferably at least −40% relative to a predose level, wherein the antibody or fragment thereof is preferably administered in a dose of about 200 mg E4W.
(v) is for use in the reduction of low-density lipoprotein (LDL-C) of at least about −50% to about −75% relative to a predose level with a sustained reduction over at least a 28 day-period, wherein the sustained reduction is preferably at least −40% and more preferably at least −45% relative to a predose level, wherein the antibody or fragment thereof is preferably administered in a dose of about 300 mg E4W.
(vi) is for use in the increase of serum HDL cholesterol levels of at least 2%, at least 2.5%, at least, 3%, at least 3.5%, at least 4%, at least 4.5%, at least 5% or at least 5.5% relative to a predose level.
(vii) Is for use in the reduction of serum total cholesterol at least about 25% to about 35% relative to a predose level with sustained reduction over at least a 24 day period.
(viii) Is for use in the reduction of serum total cholesterol at least about 65% to about 80% relative to a predose level with sustained reduction over at least a 24 day period.
25. Is for use in the reduction of serum triglyceride levels at least about 25% to about 40% relative to a predose level.
26. has little or no measurable effect on liver function, as determined by ALT and AST measurements, or on troponin levels.
27. Is for use in the increase of one or more of: Total-Cholesterol levels, ApoB levels, non HDL-C levels, Apo-B/ApoA-1 ratio.

According to another preferred embodiment of the sixteenth aspect, the antibody or antigen-binding fragment thereof is for use together with an HMG-COA reductase inhibitor, wherein the HMG-COA reductase inhibitor is preferably administered in a dosage amount in the range of about 0.05 mg to about 100 mg and is preferably a statin, wherein the statin is preferably selected from the group consisting of: cerivastatin, atorvastatin, simvastatin, pitavastatin, rosuvastatin, fluvastatin, lovastatin or pravastatin.

According to another preferred embodiment of the sixteenth aspect the statin is administered according to one or more of the following dosage or administration regimes:
(i) the statin is administered once per day,
(ii) the statin administered at a dosage of about 0.5 to about 100 mg, about 5 to about 90 mg, of about 10, 20, 40 or 80 mg and is preferably atorvastatin.
Further dosage and administration regimes of the antibody, antigen fragment thereof or the HMG-COA reductase inhibitor are described at the other aspects of present invention and preferably at the first, second or nineteenth aspect.

In a seventeenth aspect the present invention is directed to an antibody or an antigen-binding fragment thereof which specifically binds hPCSK9 (human proprotein convertase subtilisin/kexin type 9) for use in the treatment of a disease or condition in which PCSK9 expression or activity causes an impact,

wherein the antibody or antigen-binding fragment thereof is for administration to a subject falling at least into one of the following groups of subjects: (i) subjects having a serum LDL cholesterol (LDL-C) level of at least 100 mg/dL, preferably at least 130 mg/dL, more preferably at least 160 mg/dL, even more preferably at least 200 mg/dL; (ii) subjects having a serum HDL-C level of less than 40 mg/dL; (iii) subjects having a serum cholesterol level of at least 200 mg/dL, preferably at least 240 mg/dL; (iv) subjects having a serum triacylglycerol level of at least 150 mg/dL, e.g. at least 200 mg/dL or at least 500 mg/dL, wherein said triacylglycerol level is determined after fasting for at least 8 hours; (v) subjects being at least 35 years old, e.g. at least 40 years old, at least 45 years old, at least 50 years old, at least 55 years old, at least 60 years old, at least 65 years old, or at least 75 years old; (vi) subjects younger than 75 years, e.g. younger than 70 years, younger than 65 years, younger than 60 years, younger than 55 years, younger than 50 years, younger than 45 years, or younger than 40 years; (vii) subjects having a BMI of 25 or more (e.g. 26 or more, 27 or more, 28 or more, 29 or more, 30 or more, 31 or more, 32 or more, 33 or more, 34 or more, 35 or more, 36 or more, 37 or more, 38 or more, or 39 or more); (viii) male subjects; (ix) female subjects; (x) subjects in which the administration of said antibody or antigen-binding fragment thereof leads to a reduction in the serum LDL-C level by at least 30 mg/dL, preferably by at least 40 mg/dL, more preferably by at least 50 mg/dL, more preferably by at least 60 mg/dL, more preferably by at least 70 mg/dL, relative to predose level; or (xi) subjects in which the administration of said antibody or antigen-binding fragment thereof leads to a reduction in the serum LDL-C level by at least 20%, preferably by at least 30%, more preferably by at least 40%, more preferably by at least 50%, more preferably by at least 60%, relative to predose level.

In an eighteenth aspect the present invention is directed to an antibody or an antigen-binding fragment thereof which specifically binds hPCSK9 (human proprotein convertase subtilisin/kexin type 9) for use in the treatment of a disease or condition in which PCSK9 expression or activity causes an impact,

wherein the antibody or antigen-binding fragment thereof is for administration to a subject who does not fall into one or more of the following groups of subjects: (i) smokers; (ii) persons being 70 years old or older; (iii) persons suffering from hypertension; (iv) women who are pregnant; (v) women who are trying to become pregnant; (vi) women who are breast-feeding; (vii) persons who have or ever had a disease affecting the liver; (viii) persons who had any unexplained abnormal blood tests for liver function; (ix) persons who drink excessive amounts of alcohol; (x) persons having kidney problems; (xi) persons suffering from hypothyroidism; (xii) persons suffering from muscle disorders; (xiii) persons having encountered previous muscular problems during treatment with lipid-lowering medicine; (xiv) persons having serious problems with their breathing; (xv) persons taking one or more of the following medicines: medicines altering the way the immune systems works (e.g. ciclosporin or antihistamines), antibiotics or antifungal medicines (e.g. erythromycin, clarithromycin, ketoconazole, itraconazole, rifampicin, fusidic acid), medicines regulating lipid levels (e.g. gemfibrozil, colestipol), calcium channel blockers (e.g. verapamil, diltiazem), medicines regulating the heart rhythm (digoxin, amiodarone), protease inhibitors used in the treatment of HIV (e.g. nelfinavir), warfarin, oral contraceptives, antacids or St. John's Wort; or (xvi) persons drinking more than 0.1 L of grapefruit juice per day or eating more than half a grapefruit per day; (xvii) persons having a body mass index (BMI) of more than 40; (xviii) persons having a body mass index (BMI) of less than 18; (xix) persons suffering from type 1 diabetes or type 2 diabetes; (xx) persons positive for hepatitis B or hepatitis C; (xxi) persons having a known sensitivity to monoclonal antibody therapeutics; (xxii) persons having a neutrophil concentration of less than 1500/mm³; (xxiii) persons having a platelet concentration of less than 100000/mm³; (xxiv) men having a serum creatinine level larger than 1.5×ULN (upper limit of normal); (xxv) women having a serum creatinine level larger than 1.4×ULN (upper limit of normal); (xxvi) persons having an alanine transaminase (ALT) level or aspartate transaminase (AST) level larger than 2×ULN; or (xxvii) persons having a CPK level larger than 3×ULN.

In preferred embodiments of the fifteenth to eighteenth aspect, the disease or condition in which PCSK9 expression or activity causes an impact is ameliorated, improved, inhibited or prevented with a PCSK9 antagonist.

In preferred embodiments of the fifteenth to eighteenth aspect, the disease or condition in which PCSK9 expression or activity causes an impact is selected from the group consisting of: elevated LDL-C levels, hypercholesterolemia, hyperlipidemia, dyslipidemia, atherosclerosis and cardiovascular diseases, or any other of the diseases and conditions described in the first or second aspect.

In preferred embodiments of the fifteenth to eighteenth aspect, the antibody or antigen-binding fragment thereof is for administration to a subject indicated for LDL apheresis, a subject with PCSK9-activating mutations, a subject with heterozygous Familial Hypercholesterolemia, a subject with primary hypercholesterolemia, e.g. a subject with primary Familial or primary non-Familial Hypercholesterolemia, a subject with hypercholesterolemia such as primary hypercholesterolemia who is statin uncontrolled, a subject at risk for developing hypercholesterolemia, a subject with hypercholesterolemia, a subject with hyperlipidemia, a subject with dyslipidemia, a subject with atherosclerosis or a subject with cardiovascular diseases or any other of the subjects as described in the first or second aspect. Most preferably, the subject is a human subject.

Antibodies and antigen-binding fragments thereof that can be used for practicing the fifteenth to eighteenth aspect of the present invention are described in the section “Preferred Antibodies for Practicing the Present Invention”.

In preferred embodiments of the sixteenth to eighteenth aspect, the antibody or antigen-binding fragment thereof is for administration in combination with a dosage of between 0.05 mg to 100 mg of an HMG-CoA reductase inhibitor. In some embodiments, the HMG-COA reductase inhibitor is to be administered three times per day, twice per day, or once per day. In some embodiments, the HMG-COA reductase inhibitor is to be administered every day, every other day, every third day, every fourth day, every fifth day, or every sixth day. In some embodiments, the HMG-COA reductase inhibitor is to be administered every week, every other week, every third week, or every fourth week. In some embodiments, the HMG-COA reductase inhibitor is to be administered in the morning, at noon or in the evening. In preferred embodiments, the HMG-CoA reductase inhibitor is to be administered once per day, preferably orally, preferably in the evening. Preferably, the HMG-COA reductase inhibitor is a statin. More preferably, the statin is selected from the group consisting of cerivastatin, atorvastatin, simvastatin, pitavastatin, rosuvastatin, fluvastatin, lovastatin, and pravastatin. In further preferred embodiment of the sixteenth to eighteenth aspect, the antibody or antigen-binding fragment thereof is for administration in combination with a statin, wherein the statin is
cerivastatin which is to be administered in a daily dosage of between 0.05 mg and 2 mg, preferably in a daily dosage of 0.2 mg, 0.4 mg, or 0.8 mg;
atorvastatin which is to be administered in a daily dosage of between 2 mg and 100 mg, preferably in a daily dosage of 10 mg, 20 mg, 40 mg, or 80 mg;
simvastatin which is to be administered in a daily dosage of between 2 mg and 100 mg, preferably in a daily dosage of 5 mg, 10 mg, 20 mg, 40 mg, or 80 mg;
pitavastatin which is to be administered in a daily dosage of between 0.2 mg and 100 mg, preferably in a daily dosage of 1 mg, 2 mg, 5 mg, 10 mg, or 20 mg;
rosuvastatin which is to be administered in a daily dosage of between 2 mg and 100 mg, preferably in a daily dosage of 5 mg, 10 mg, 20 mg, or 40 mg;
fluvastatin which is to be administered in a daily dosage of between 2 mg and 100 mg, preferably in a daily dosage of 20 mg, 40 mg, or 80 mg;
lovastatin which is to be administered in a daily dosage of between 2 mg and 100 mg, preferably in a daily dosage of 10 mg, 20 mg, 40 mg, or 80 mg; or
pravastatin which is to be administered in a daily dosage of between 2 mg and 100 mg, preferably in a daily dosage of 10 mg, 20 mg, 40 mg, or 80 mg.

A further preferred embodiment of the present invention combines the features of the sixteenth and seventeenth or the sixteenth and eighteenth or the seventeenth and eighteenth or the sixteenth and seventeenth and eighteenth aspect as described herein.

In a nineteenth aspect the present invention is directed to a pharmaceutical composition comprising the antibody or antigen-binding fragment thereof according to present invention together with a pharmaceutically acceptable excipient or carrier.

According to a preferred embodiment of the nineteenth aspect, the antibody or fragment thereof is as described in the fifteenth, first or second aspect; further suitable features of the antibody are described in the section “Preferred Antibodies for Practicing the Present Invention”.

According to another preferred embodiment, the pharmaceutical composition comprises about 40 to about 500 mg of the antibody or antigen-binding fragment per dose.

According to another preferred embodiment, the pharmaceutical composition comprises about 50 mg to about 500 mg, about 50 mg to about 300 mg, about 50 mg, about 100 mg, about 150 mg, about 200 mg, about 250 mg, about 300 mg, about 350 mg, of about 400 mg, about 450 mg or about 500 mg of the antibody or antigen-binding fragment thereof.

According to another preferred embodiment, the pharmaceutical composition comprises about 150, 200 or 300 mg of the antibody or antigen-binding fragment thereof.

According to another preferred embodiment, the pharmaceutical composition comprises an effective dose of an antibody or an antigen-binding fragment thereof which specifically binds hPCSK9 (human proprotein convertase subtilisin/kexin type 9), wherein the dose is sufficient for sustained reduction of low-density lipoprotein (LDL-C) levels over a period of at least 14, at least 15, at least 16, at least 17, at least 18, at least 19, at least 20, at least 21, at least 22, at least 23 or at least 28 days after administration, together with a pharmaceutically acceptable excipient or carrier. According to another preferred embodiment, the dose is sufficient for sustained reduction of LDL-C levels over a period of at least 14 days, 28 days or 1 month.

According to another preferred embodiment, the pharmaceutical composition comprises an effective amount of an HMG-COA reductase inhibitor.

According to another preferred embodiment the pharmaceutical composition is arranged together an effective amount of an HMG-COA reductase inhibitor.

According to another preferred embodiment, the HMG-COA reductase inhibitor is a statin, preferably selected from the list consisting or: cerivastatin, atorvastatin, simvastatin, pitavastatin, rosuvastatin, fluvastatin, lovastatin or pravastatin and is preferably atorvastatin.

According to another preferred embodiment, the pharmaceutical composition comprises about 0.05 mg to about 100 mg, about 0.5 mg to about 100 mg, about 5 mg to about 90 mg, about 10 mg, about 20 mg, about 40 mg or about 80 mg of HMG-COA reductase inhibitor and preferably about 10, about 20, about 40 or about 80 mg.

According to another preferred embodiment, the pharmaceutical composition comprises an effective dose of HMG-COA reductase inhibitor for lowering LDL-D levels by administration once per day.

The antibody or antigen-binding fragment thereof to be used for the pharmaceutical composition according to present invention can be any antibody as described herein, such as in the section “Preferred Antibodies for Practicing the Present Invention” or in the fifteenth and further aspects.

According to a preferred embodiment, the antibody or antigen-binding fragment thereof has one or more of the following features when administered to a subject, such as a human or non-human mammal:
a. reduction of low-density lipoprotein (LDL-C) levels of at least about −25% to about −40% relative to a predose level with a sustained reduction over at least a 14 day-period upon administration to a subject, wherein the sustained reduction is preferably at least −25% and more preferably at least −30% relative to a predose level, particularly if administered in a dose of about 40 to about 60 mg, preferably about 45 to about 55 mg and more preferably about 50 mg in a biweekly administration regime (every other week, E2W);
b. reduction of low-density lipoprotein (LDL-C) of at least about −50% to about −65% relative to a predose level with a sustained reduction over at least a 14 day-period upon administration to a subject, wherein the sustained reduction is preferably at least −40% and more preferably at least −45% relative to a predose level, particularly if administered in a dose of about 100 mg E2W.
c. reduction of low-density lipoprotein (LDL-C) of at least about −60% to at least about −75% [e.g. at least about −60%, at least about −65%, at least about −70 or at least about −75%] relative to a predose level with a sustained reduction over at least a 14 day-period upon administration to a subject, wherein the sustained reduction is preferably at least −55% and more preferably at least −60% relative to a predose level, particularly when administered in a dose of about 150 mg E2W,
d. reduction of low-density lipoprotein (LDL-C) of at least about 40% to about 75% relative to a predose level with a sustained reduction over at least a 28 day period, wherein the sustained reduction is preferably at least −35% and more preferably at least −40% relative to a predose level, particularly when administered in a dose of about 200 mg E4W
e. reduction of low-density lipoprotein (LDL-C) of at least about −50% to about −75% relative to a predose level with a sustained reduction over at least a 28 day-period upon administration to a subject, wherein the sustained reduction is preferably at least −40% and more preferably at least −45% relative to a predose level, particularly when administered in a dose of about 300 mg E4W,
f. increase of serum HDL cholesterol levels of at least 2%, at least 2.5%, at least, 3%, at least 3.5%, at least 4%, at least 4.5%, at least 5% or at least 5.5% relative to a predose level upon administration to a subject, particularly when administered in a dose of about 150 mg E2W,
g. little or no measurable effect on troponin levels upon administration to a subject,
h. increase of one or more of: Total-Cholesterol levels, ApoB levels, non HDL-C levels, Apo-B/ApoA-1 ratio, upon administration to a subject.
According to another preferred embodiment, the antibody or antigen-binding fragment thereof is capable of overcoming statin resistance when administered to a subject with statin-resistant hypercholesterolemia.
According to another preferred embodiment, the antibody or antigen-binding fragment thereof comprises the heavy and light chain CDRs of a HCVR/LCVR amino acid sequence pair as shown in SEQ ID NOs: 90/92 substantially identical sequences having at least 98% or 99% identity therewith.
According to another preferred embodiment, the antibody or antigen-binding fragment thereof comprises a HCVR/LCVR amino acid sequence pair as shown in SEQ ID NOs: 90/92 or a pair of substantially identical sequences having at least 98% or 99% identity therewith.
According to another preferred embodiment, the antibody or antigen-binding fragment thereof competes for binding to hPCSK9 with an antibody or antigen-binding fragment comprising a HCVR/LCVR amino acid sequence pair as shown in SEQ ID NOs: 90/92.
According to another preferred embodiment, the antibody or antigen-binding fragment thereof binds an epitope comprising amino acid residue 238 of hPCSK9 (SEQ ID NO:755).
According to another preferred embodiment, the antibody or antigen-binding fragment thereof binds an epitope comprising one or more of amino acid residues at positions 238, 153, 159 and 343 of hPCSK9 (SEQ ID NO:755).
According to another preferred embodiment, the antibody or antigen-binding fragment thereof binds an epitope which does not comprise an amino acid residue at positions 192, 194, 197 and/or 237 of hPCSK9 (SEQ ID NO:755).

The pharmaceutical composition can be formulated according to any pharmaceutically applicable formulation as known in the art, and specifically as herein described, such as dry formulation for dissolution or liquid formulation. Suitable formulations of antibodies are known in the art and comprise dry formulations (e.g. freeze-dried, spray-dried or lyophilized, water-free concentrate) as well as liquid formulations (e.g. solutions). Suitable formulations of statins are as well known in the art and comprise dry formulations as well as liquid formulations, e.g suspensions, dispersions and solutions (for a reference, see e.g. “Statins therapy: a review on conventional and novel formulation approaches” R. Tiwari and K. Pathak, Journal of Pharmacy and Pharmacology, 2011, that is hereby incorporated in entirety).

According to a preferred embodiment, the pharmaceutical composition comprises the antibody or antigen-binding fragment thereof as dry formulation for dissolution such as a lyophilized powder, freeze-dried or spray-dried powder or water free concentrate.

According to another preferred embodiment, the pharmaceutical composition comprises the antibody or antigen-binding fragment thereof as liquid formulation, e.g. injection or infusion solution.

According to another preferred embodiment, the pharmaceutical composition comprises the HMG-COA reductase inhibitor as oral or peroral formulation, e.g. capsule or tabled, or as liquid formulation, e.g. suspension, dispersion or solution, e.g. for peroral administration, injection or infusion.

According to another preferred embodiment the pharmaceutical composition is for use in the treatment of a disease or disorder for use in the treatment of a disease or condition in which PCSK9 expression or activity causes an impact or for lowering elevated total cholesterol or elevated LDL-C levels. Further preferred uses, dosage regimens, administration regimens of the antibody or fragment thereof or of the HMG-COA reductase inhibitor, or populations to be treated with the pharmaceutical composition described in present application, for example at the other aspects of present invention such as the first or second aspect.

In a twentieth aspect, the present invention concerns an injection solution as herein described comprising the antibody or antigen-binding fragment thereof of present invention, and preferably comprising about 40 mg to about 200 mg or about 50 to about 200 mg, e.g. about 40 mg, about 50 mg, about 75 mg, at about 100 mg, about 150 mg or about 200 mg of the antibody or antigen-binding fragment thereof per 1 ml volume.

In a twentyfirst aspect the present invention concerns a dry formulation as herein described comprising the antibody or antigen-binding fragment thereof of present invention, and preferably comprising about 40 mg to about 500 mg, 50 to about 500 mg, about 50 to about 400, about 50 to about 300 e.g. about 40 mg, about 50 mg, about 75 mg, at about 100 mg, about 150 mg, about 200 mg, about 250 mg, about 300 mg, about 350 mg, about 400 mg, about 450 mg or about 500 mg and more preferably about 50 mg, about 100 mg, about 150 mg, about 200 mg, about 250 mg, about 300 mg and even more preferably about 150 mg, about 200 mg or about 300 mg of the antibody or antigen-binding fragment thereof per dose.

The formulations of present invention can comprise further active ingredients such as an HMG-CoA reductase inhibitor as herein described. Preferred embodiments of the 20^{th} or 21^{st} aspect are described in other sections of present application, e.g. in the other aspects of present invention such as the fifteenth, nineteenth or twentieth aspect.

Suitable formulations of antibodies in general are known in the art and comprise dry formulations (e.g. freeze-dried, spray-dried or lyophilized, water-free concentrate) as well as liquid formulations (e.g. solutions). Suitable formulations of statins are as well known in the art and comprise dry formulations as well as liquid formulations, e.g suspensions, dispersions and solutions (for a reference, see e.g. “Statins therapy: a review on conventional and novel formulation approaches” R. Tiwari and K. Pathak, Journal of Pharmacy and Pharmacology, 2011, that is hereby incorporated in entirety).

According to a twentysecond aspect, present invention concerns an antibody or antigen binding fragment thereof as comprised in one of the pharmaceutical compositions according to the nineteenth aspect.

In a twentythird aspect the present invention is directed to a unit dosage form comprising the antibody, antigen-binding fragment thereof or pharmaceutical composition of present invention. Suitable embodiments of the antibody, pharmaceutical composition or formulation to be used for practicing the twentythird aspect of present invention can be gained from the respective sections of present application, such as the first, second, nineteenth, twentieth, twentyfirst or twentysecond aspects or from the section “Preferred Antibodies for Practicing the Present Invention”.

According to a preferred embodiment, the unit dosage form comprises about 40 mg, about 50 mg, about 75 mg, at about 100 mg, about 150 mg, about 200 mg, about 250 mg, about 300 mg, about 350 mg, about 400 mg, about 450 mg, or about 500 mg of the antibody or antigen-binding fragment thereof.

According to another preferred embodiment, the unit dosage form comprises the antibody or fragment thereof as dry formulation for dissolution in a hermetically sealed container such as a vial, an ampoule or sachette.

According to another preferred embodiment, the unit dosage form comprises the antibody or fragment thereof as liquid formulation in a hermetically sealed container such as a vial, a sachette, a pre-filled syringe, a pre-filled autoinjector or a cartridge for a reusable syringe or applicator.

According to another preferred embodiment, the quantity of active ingredient is indicated on the hermetically-sealed container.

As used in the different aspects and embodiments of present invention and in particularly of the twentythird aspect, the term “unit dosage form” refers to physically discrete units suitable as unitary dosages for human and animal subjects, each unit containing a predetermined quantity of active material (e.g., about 40 mg or about 50 mg to about 500 mg of PCSK5 antibody and/or of e.g. 0.05 mg to 100 mg HMG-CoA reductase inhibitor) calculated to produce the desired therapeutic effect in association with the required pharmaceutical diluent, carrier or vehicle. The specifications for the novel unit dosage forms of this invention are dictated by and are directly dependent on (a) the unique characteristics of the active material and the particular therapeutic effect to be achieved, and (b) the limitation inherent in the art of compounding such an active material for therapeutic use in animals or humans, as disclosed in this specification, these being features of the present invention. Examples of suitable unit dosage forms in accord with this invention are vials, tablets, capsules, troches, suppositories, powder packets, wafers, cachets, ampules, segregated multiples of any of the foregoing, and other forms as herein described or generally known in the art.

One or more such unit dosage forms of the antibody can be comprised in an article of manufacture of present invention, optionally further comprising one or more unit dosage forms of an HMG-CoA reductase inhibitor (e.g. a blister of tablets comprising as active ingredient the HMG-COA reductase inhibitor).

The term “active material” refers to any material with therapeutic activity, such as one or more active ingredients. The active ingredients to be employed as therapeutic agents can be easily prepared in such unit dosage form with the employment of pharmaceutical materials which themselves are available in the art and can be prepared by established procedures. Preferred active ingredients of present invention are the antibody or fragment thereof or an HMG-COA reductase inhibitor such as a statin.

In a preferred embodiment, the unit dosage form comprises 40-about 500 mg of the antibody or an antigen-binding fragment of present invention. According to another preferred embodiment, the unit dosage form comprises about 40 mg, about 50 mg, about 75 mg, at about 100 mg, about 150 mg, about 200 mg, about 250 mg, about 300 mg, about 350 mg, about 400 mg, about 450 mg, or about 500 mg and more preferably about 150 mg, about 200 mg or about 300 mg of the antibody or antigen-binding fragment thereof. Further preferred dosages, and dosage regimens are as described elsewhere in the application, such as at the first, second or fifteenth to nineteenth aspect.

According to another preferred aspect, the unit dosage form comprises the antibody, antigen-binding fragment thereof or pharmaceutical composition as dry formulation for dissolution such as a lyophilized powder, freeze-dried powder or water free concentrate. According to another preferred embodiment the dry formulation is comprised in a hermetically sealed container such as a vial, an ampoule or sachette.

According to another preferred embodiment, the unit dosage form comprises the antibody, antigen-binding fragment thereof or pharmaceutical composition as liquid formulation, e.g. injection or infusion solution. According to another preferred embodiment the liquid formulation is comprised in a hermetically sealed container such as a vial, a sachette, a pre-filled syringe, a pre-filled autoinjector or a cartridge for a reusable syringe or applicator.

It is further preferred, if the quantity of active ingredient (e.g. antibody) is indicated on the hermetically-sealed container of the unit dosage form.

The following preparations are illustrative of the preparation of the unit dosage forms of the present invention, and not as a limitation thereof. Several dosage forms may be prepared embodying the present invention. For example, a unit dosage per vial may contain 0.5 ml, 1 ml, 2 ml, 3 ml, 4 ml, 5 ml, 6 ml, 7 ml, 8 ml, 9 ml, 10 ml, 15 ml, or 20 ml of PCSK5 antibody or a fragment thereof ranging from about 40 to about 500 mg of PCSK5 antibody. If necessary, these preparations can be adjusted to a desired concentration by adding a sterile diluent to each vial.

In one embodiment, the ingredients of formulation of the invention are supplied either separately or mixed together in unit dosage form, for example, as a dry formulation for dissolution or a liquid formulation. The preparation of pharmaceutically acceptable formulations of proteinaceous biomolecules such as antibodies or antigen-binding fragments thereof or of small molecule compounds such as statins is generally known in the art. According to a preferred embodiment, the active ingredients, active material or pharmaceutical composition according to present invention is a dry formulation for liquid dissolution, such as a lyophilized powder, freeze-dried powder or water free concentrate, preferably comprised in a hermetically sealed container such as a vial, an ampoule or sachette, and preferably indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The formulations of the invention include bulk drug compositions useful in the manufacture of pharmaceutical compositions (e.g., compositions that are suitable for administration to a subject or patient) which can be used in the preparation of unit dosage forms. In a preferred embodiment, a composition of the invention is a pharmaceutical composition. Such compositions comprise a prophylactically or therapeutically effective amount of one or more prophylactic or therapeutic agents (e.g., an antibody of the invention or other prophylactic or therapeutic agent), and a pharmaceutically acceptable carrier. Preferably, the pharmaceutical compositions are formulated to be suitable for the route of administration to a subject.

In a specific embodiment, the term “pharmaceutically acceptable” means approved by a regulatory agency of the U.S. Federal or a state government or the EMA (European Medicines Agency) or listed in the U.S. Pharmacopeia Pharmacopeia (United States Pharmacopcia-33/National Formulary-28 Reissue, published by the United States Pharmacopeial Convention, Inc., Rockville Md., publication date: April 2010) or other generally recognized pharmacopcia for use in animals, and more particularly in humans.

The term “carrier” refers to a diluent, adjuvant {e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of the antibody, preferably in purified form, together with a suitable amount of carrier so as to provide the form for proper administration to the patient. The composition may further contain one or more other active ingredients such as an HMG-COA reductase inhibitor. The formulation should suit the mode of administration.

Generally, the ingredients of compositions of the invention are supplied either separately or mixed together in unit dosage form, for example, as a dry formulation for dissolution such as a lyophilized powder, freeze-dried powder or water free concentrate in a hermetically sealed container such as an ampoule or sachette indicating the quantity of active agent. The ingredients of compositions of the invention can also be supplied as admixed liquid formulation (i.e. injection or infusion solution) in a hermetically sealed container such as an ampoule, sachette, a pre-filled syringe or autoinjector, or a cartridge for a reusable syringe or applicator (e.g. pen or autoinjector). Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. The composition can also comprise two or more active ingredients that are each formulated in a different or the same manner, e.g. a combination of an antibody of present invention together with an HMG-COA reductase inhibitor or present invention.

The invention also provides that the formulation is packaged in a hermetically sealed container such as an ampoule or sachette indicating the quantity of antibody. In one embodiment, the formulation of the invention comprising an antibody is supplied as a dry sterilized lyophilized powder, freeze-dried powder or water free concentrate in a hermetically sealed container and can be reconstituted, e.g., with water or saline to the appropriate concentration for administration to a subject. In one embodiment, the formulation of the invention comprising an antibody is supplied as a dry sterile lyophilized powder in a hermetically sealed container at a unit dosage of at least 40 mg, at least 50 mg, more preferably at least 75 mg, at least 100 mg, at least 150 mg, at least 200 mg, at least 250 mg, at least 300 mg, at least 350 mg, at least 400 mg, at least 450 mg, or at least 500 mg, of antibody or antigen-binding fragment thereof. The lyophilized formulation of the invention comprising an antibody should be stored at between 2 and 8° C. in its original container and the antibody should be administered within 12 hours, preferably within 6 hours, within 5 hours, within 3 hours, or within 1 hour after being reconstituted. The formulation of the invention comprising antibodies can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In a twentyfourth aspect, present invention concerns an article of manufacture comprising, the pharmaceutical composition of present invention, the liquid formulation of present invention or the dry formulation of present invention, the antibody or antigen-binding fragment thereof of present invention or one or more unit dosage forms of present invention and a container or package.

According to another preferred embodiment, the article of manufacture comprises sufficient unit dosage forms of antibody for a two-week (14 day), four-week (28 day) or one month period, with either E2W, E4W or once-a-month administration regime.

The article of manufacture can comprise one or more unit dosage form that contain(s) both, the antibody and the HMG CoA-inhibitor, e.g. a unit dosage form comprising a liquid formulation for injection or infusion comprising both active ingredients. The article of manufacture can also comprise the antibody (or antigen-binding fragment thereof) and the HMG-COA reductase inhibitor in two or more separate unit dosage forms.

According one embodiment, the article of manufacture comprises one or more separate unit dosage forms of the and the HMG-COA reductase inhibitor according to present invention.

According to a preferred embodiment, each unit dosage form of HMG-COA reductase inhibitor comprises about 0.05 mg to about 100 mg HMG-CoA reductase inhibitor.

According to another preferred embodiment the HMG-COA reductase inhibitor is a statin, preferably selected from the list containing: cerivastatin, atorvastatin, simvastatin, pitavastatin, rosuvastatin, fluvastatin, lovastatin or pravastatin and preferably atorvastatin.

According to another preferred embodiment the HMG-COA reductase inhibitor, e.g. the statin, is in an effective dose for administration once per day.

According to another preferred embodiment, the unit dosage form of HMG-COA reductase inhibitor comprises about 0.5 to about 100 mg, about 5 to about 90 mg, of about 10, 20, 40 or 80 mg HMG-COA reductase inhibitor.

According to another preferred embodiment, the article of manufacture comprises sufficient unit dosage forms of HMG-COA reductase inhibitor for a daily administration regime.

According to another preferred embodiment, the unit dosage form comprising the antibody is a sachette, a pre-filled syringe, a pre-filled autoinjector or a cartridge for a reusable syringe or applicator, especially comprising 1 ml or 2 ml of injection solution.

According to another embodiment, the article of manufacture comprises one or more of the following components:
i. One or more unit dosage forms comprising the antibody of present invention
j. One or more unit dosage forms comprising the HMG-COA reductase inhibitor of present invention;
k. Instructions for use;
l. A device for application of the antibody such as a syringe.

According to another preferred embodiment, the article of manufacture comprises sufficient unit dosage forms of the antibody and preferably also of the HMG-COA reductase inhibitor . . .
(a) for one single administration of antibody and HMG-COA reductase inhibitor, e.g. comprising an ampoule, sachette, vial, cartridge or pre-filled syringe comprising about 50 mg, about 100 mg, about 150 mg, about 200 mg, about 250 mg, about 300 mg, about 350 mg, about 400 mg, about 450 mg or about 500 mg antibody and preferably about 150 mg antibody, about 200 mg antibody or about 300 mg antibody, together with tablet or capsule, e.g. for oral or peroral administration comprising the HMG, CoA-inhibitor, e.g. comprising about 10 mg, about 20 mg, about 40 mg or about 80 mg of the HMG CoA-inhibitor such as atorvastatin.
(b) for a two-week (i.e. 14-day) treatment with antibody and HMG-COA reductase inhibitor, e.g. comprising an ampoule, sachette, vial, cartridge or pre-filled syringe comprising about 50 mg, about 100 mg, about 150 mg, about 200 mg, about 250 mg, about 300 mg, about 350 mg, about 400 mg, about 450 mg or about 500 mg antibody and preferably about 150 mg antibody, about 200 mg antibody or about 300 mg antibody; together with sufficient units comprising of HMG-COA reductase inhibitor (e.g. tablets or capsules, e.g. for oral or peroral administration) for a 14-day treatment, e.g. 14 units for a once-a day administration regime of HMG-COA reductase inhibitor or 28 units for a twice-a day administration regime etc, wherein the units per day of HMG CoA-inhibitor preferably comprise about 10 mg, about 20 mg, about 40 mg or about 80 mg of the HMG CoA-inhibitor such as atorvastatin. In the case the antibody is to be administered in a dosage of more than 200 mg, two unit dosage forms of antibody together comprising the total dose may be preferable (e.g. two pre-filled syringes comprising about 150 mg of antibody in 1 ml of liquid formulation each for a total administration (e.g. subcutaneous injection) of about 300 mg antibody in two shots) may be preferable (or two units with about 100 mg each for a total administration of about 200 mg antibody, two units with about 175 mg for a total administration of about 350 antibody, etc.,).
(c) for a four week (i.e, 28-day) treatment with antibody and HMG-COA reductase inhibitor, e.g.
   1. for a E2W administration regimen of the antibody with about 50 to about 200 mg antibody per two weeks: comprising two unit dosage forms (e.g. as exemplified above) with each about 50 mg, about 100 mg, about 150 mg or about 200 mg antibody or antigen-binding fragment thereof together with 28 unit dosage forms of HMG-COA reductase inhibitor (as exemplified above) for a daily once a day administration regime or together with 56 unit dosage forms of HMG-COA reductase inhibitor for a daily twice-a day administration regime, preferably 28 unit dosage forms (e.g. capsules or tablets) of about 10 mg, about 20 mg, about 40 mg or about 80 mg atorvastatin
   2. for an E4W administration regime of the antibody or fragment thereof with an administration of about 200 mg per four weeks (28 days): e.g. comprising one unit dosage form of the antibody with about 200 mg antibody (e.g. as exemplified above) together with 28 or 56, and preferably 28 unit dosage forms of HMG-COA reductase inhibitor (e.g. as exemplified above)
   3. for an E4W administration regime of the antibody with more than 200 mg per four weeks (28 days): comprising two unit dosage forms that together comprise the total dose of antibody (e.g. two pre-filled syringes each comprising 1 ml of liquid antibody formulation with 150 mg antibody each) or comprising one unit dosage form that comprises the total amount of antibody to be administered (e.g. a vial comprising about 300 mg antibody for dissolution or a vial, cartridge or pre-filled syringe comprising about 300 mg of the antibody in liquid formulation (i.e. about 2 ml of liquid formulation, wherein 1 ml of liquid formulation comprises about 150 mg of the antibody); together with 28 or 56, and preferably 28 unit dosage forms of HMG-COA reductase inhibitor (e.g. as exemplified above)
(d) for a one-month treatment with antibody and HMG-COA reductase inhibitor: comprising the same numbers of unit dosage forms of antibody as exemplified under (c) for an administration once or twice per month, e.g. every first day of the month or every first Monday etc. of the month for a once a month administration, or e.g. every first and 14th or 15th day of the month for a twice-a-month administration regime; in addition the article of manufacture comprises 31 unit dosage forms of HMG-COA reductase inhibitor, preferably tablets or capsules arranged in a blister containing a consecutive numbering from 1-31 for the days of the month (wherein the superfluous tablets or capsules for excess days are to be discarded).

In a twentyfifth aspect the present invention is directed to an article of manufacture comprising: (a) a packaging material; (b) an antibody or an antigen-binding fragment thereof which specifically binds hPCSK9; and (c) a label or packaging insert contained within the packaging material indicating that patients receiving treatment with said antibody or antigen-binding fragment can be treated for a disease or condition selected from the group consisting of hypercholesterolemia, hyperlipidemia, dyslipidemia, atherosclerosis and cardiovascular diseases and further indicating that subjects falling into one or more groups of subjects as recited in the thirteenth aspect can be treated.

In a twentysixth aspect the present invention is directed to an article of manufacture comprising: (a) a packaging material; (b) an antibody or an antigen-binding fragment thereof which specifically binds hPCSK9; and (c) a label or packaging insert contained within the packaging material indicating that patients receiving treatment with said antibody or antigen-binding fragment can be treated for a disease or condition selected from the group consisting of hypercholesterolemia, hyperlipidemia, dyslipidemia, atherosclerosis and cardiovascular diseases and further indicating that the treatment of patients with said antibody or antigen-binding fragment thereof is contraindicated for patients belonging to one or more groups of subjects as recited in the fourteenth aspect.

Antibodies and antigen-binding fragments thereof that can be used for practicing the different articles of manufacture of the present invention are described in the section “Preferred Antibodies for Practicing the Present Invention”.

In preferred embodiments of the twentyfourth, the twentyfifth or twentysixth aspect, the label or packaging insert contains a reference to a method of treatment according to the first or the medical uses according to the second aspect and the embodiments of the first and second aspect as described herein.

The articles of manufacture described in the 24^{th}, the 25^{th} or the 26^{th} aspect can further comprise one or more of the features or components of the article of manufacture as comprised in the 3^{rd}, 4^{th} or 5^{th} aspect of present invention and vice versa. A further preferred embodiment of the present invention combines one or more of the features of the 24^{th} and the 25^{th}, of the 25^{th} and the 26^{th} or of the 24^{th} and the 25^{th} and the 26^{th} aspect as described herein.

According to a 27^{th} aspect, present invention concerns a pharmaceutical composition or antibody or antigen-binding fragment thereof of present invention, such as according to the nineteenth aspect of present invention, for use in the treatment of a disease or condition in which PCSK9 expression or activity causes an impact, preferably for use in the lowering of elevated LDL-C (low density lipoprotein C) levels

According to a preferred embodiment, the disease or condition is selected from the group consisting of: elevated total cholesterol levels, elevated low-density lipoprotein (LDL-C) levels, hypercholesterolemia, hyperlipidemia, dyslipidemia, and atherosclerosis, particularly primary hypercholesterolemia, familial hypercholesterolemia, or hypercholesteremia which is uncontrolled by statins

According to another preferred embodiment, the composition, the antibody or antigen-binding fragment thereof is administered to the subject every other week (E2W), every fourth week (E4W) or once a month.

According to another preferred embodiment an HMG-COA reductase inhibitor is co-administered with the pharmaceutical composition, the antibody or antigen-binding fragment thereof, preferably an HMG-COA reductase inhibitor according to one of the different aspects of present invention, such as according to the first or second aspect.

According to another preferred embodiment the HMG-COA reductase inhibitor is administered once a day and preferably every day.

In a twentyeighth aspect, present invention concerns a method for preparing a pharmaceutical composition of present invention, e.g. according to the nineteenth aspect, comprising mixing the antibody or antigen-binding fragment thereof and optionally the HMG-CoA reductase inhibitor with one or more pharmaceutical excipients or carriers.

In a twentynineth aspect, present invention concerns a method for preparing a unit dosage form of present comprising admeasuring an amount of the pharmaceutical composition, of the antibody or antigen-binding fragment thereof, of the liquid formulation or of the dry formulation according to present invention comprising one or more doses of the antibody or antigen fragment thereof and optionally of the HMG-COA reductase inhibitor and tailoring them as physically discrete units suitable as unitary dosages for human and/or animal administration.

In a thirtieth aspect, present invention concerns a method for preparing or assembling an article of manufacture of present invention comprising packaging the pharmaceutical composition, of the antibody according, of the liquid formulation, of the dry formulation according or of or more of the unit dosage forms of present invention in a container, optionally together with one or more of the following: a label, instructions for use, an application device.

In a thirtyfirst aspect the present invention is directed to a method of testing the efficacy of an antibody or an antigen-binding fragment thereof which specifically binds hPCSK9 for the treatment of a disease or condition selected from the group consisting of elevated LDL-C levels, hypercholesterolemia, hyperlipidemia, dyslipidemia, atherosclerosis and cardiovascular diseases, or any other disease or condition described in the first or second aspect, said method comprising:
treating a selected patient population with said antibody or antigen-binding fragment thereof, wherein each patient in said population has an LDL cholesterol (LDL-C) level of more than 100 mg/dL; and
determining the efficacy of said antibody or antigen-binding fragment thereof by determining the LDL-C level in the patient population before and after administration of said antibody or antigen-binding fragment thereof, wherein a reduction of the LDL-C level by at least 25% relative to a predose level in at least 75% of the patient population indicates that said antibody or antigen-binding fragment thereof is efficacious for the treatment of said disease or condition in said patient population;
wherein each patient falls into one or more groups of subjects as recited in the thirteenth aspect.

In a thirtysecond aspect the present invention is directed to a method of testing the efficacy of an antibody or an antigen-binding fragment thereof which specifically binds hPCSK9 for the treatment of a disease or condition selected from the group consisting of elevated LDL-C levels, hypercholesterolemia, hyperlipidemia, dyslipidemia, atherosclerosis and cardiovascular diseases (or any other method as described in the first or second aspect), said method comprising:
determining the efficacy of an antibody or antigen-binding fragment thereof that has been used for the treatment of a selected patient population with said antibody or antigen-binding fragment thereof, wherein each patient in said population has an LDL cholesterol (LDL-C) level of more than 100 mg/dL by determining the LDL-C level in the patient population before and after administration of said antibody or antigen-binding fragment thereof, wherein a reduction of the LDL-C level by at least 25% relative to a predose level in at least 75% of the patient population indicates that said antibody or antigen-binding fragment thereof is efficacious for the treatment of said disease or condition in said patient population;
wherein each patient falls into one or more groups of subjects as recited in the thirteenth aspect.

In preferred embodiments of the 31^{th} or 32^{nd} aspect, each patient in said population has received a lipid lowering treatment by administration of an HMG-COA reductase inhibitor such as a statin for at least 6 weeks prior to treatment with said antibody or antigen-binding fragment thereof.

Antibodies and antigen-binding fragments thereof that can be used for practicing the nineteenth and twentieth aspect of the present invention are described in the section “Preferred Antibodies for Practicing the Present Invention” or the other section of present application describing antibodies of present invention, such as e.g. the fifteenth aspect.

In preferred embodiments of the 31^{th} or 32^{nd} aspect, the selected patient population is or has been treated with a method of treatment according to the first or second aspect and the embodiments of the first or second aspect as described herein.

In further preferred embodiments of the 31^{th} or 32^{nd} aspect, the efficacy of said antibody or said antigen-binding fragment thereof is determined for sub-groups of said selected patient population, wherein said sub-groups have been stratified by at least one stratification factor selected from the group consisting of: population with heterozygous familial hypercholesterolemia (heFH); prior history of myocardial infarction (MI); prior history of stroke; receiving high-intensity statin therapy; and geographical region of the patient (e.g. North America, Western Europe, Eastern Europe, and rest of the world).

In hamsters and other rodents statins are not effective on LDL clearance from blood. More specifically, the administration of statins alone (e.g. atorvastatin) has no effect on the expression of the LDL receptor (LDLR) in hamsters or other rodents, presumably due to the activity of the endogenous PCSK9. The experiments contained in the present application (see study 4) show that inhibition of PCSK9 by administration of an anti-PCSK9 antibody renders rodents (e.g. hamsters) sensitive to statin treatment. Accordingly, the present application provides a new animal model for testing the efficacy of statins or other drugs that lower cholesterol levels.

Thus, in a thirtythird aspect the present invention is directed to a method for testing the efficacy of a compound in lowering cholesterol levels in a subject, comprising the steps:
(a) providing a rodent;
(b) administering an antibody or an antigen-binding fragment thereof which specifically binds PCSK9 to the rodent;
(c) administering a test compound to said rodent;
(d) determining the effect of the test compound in the rodent, wherein a lowering of the cholesterol level in the rodent as compared to the cholesterol level of a control animal indicates that the test compound is efficacious in lowering cholesterol levels in a subject, wherein the control animal is from the same species as said rodent, and wherein the control animal has not been challenged with the test compound.

In preferred embodiments of the thirtythird aspect, the rodent is selected from the group consisting of hamster, mouse, rat, guinea pig, and rabbit.

Antibodies and antigen-binding fragments thereof that can be used for practicing the twenty-first aspect of the present invention are described the other sections of present application such as the fifteenth aspect of in the section “Preferred Antibodies for Practicing the Present Invention”. Preferably, the antibody or antigen-binding fragment thereof is administered to the rodent in a concentration of 1 mg/kg body weight, 3 mg/kg body weight, or 10 mg/kg body weight.

In preferred embodiments of the 33^{rd} aspect, the lowering of the cholesterol level is determined by measuring the level of total cholesterol in the serum. In more preferred embodiments, the lowering of the cholesterol level is determined by measuring the level of LDL cholesterol (LDL-C) in the serum.

In preferred embodiments of the 33^{rd} aspect, the control animal is from the same strain as the rodent. Preferably, the same antibody or antigen-binding fragment thereof is administered to the rodent and to the control animal. Preferably, the same concentration (measured in mg/kg body weight) of the antibody or antigen-binding fragment thereof is administered to the rodent and to the control animal.

In one embodiment of the 33^{rd} aspect, the control animal is a different animal, i.e. a different individual, than the rodent. It is also possible to determine the cholesterol level in two or more control animals and to calculate the mean value of the cholesterol level in these two or more control animals. Likewise, it is possible to challenge two or more rodents with the antibody or antigen-binding fragment thereof, to determine the cholesterol level in these two or more rodents and to calculate the mean value of the cholesterol level in these two or more rodents.

In an alternative embodiment of the 33^{rd} aspect, the control animal is the very same animal as the rodent but it is examined at a different time-point. More specifically, the cholesterol level in the rodent after administration of the test compound can be compared to a pre-dose cholesterol level in the same animal. Preferably, said pre-dose cholesterol level is determined between steps (b) and (c) recited above.

According to thirtyfourth aspect, present invention concerns a method of enhancing the LDL-C lowering activity in a subject undergoing statin therapy, the method comprising administering to the subject an antibody, or antigen-binding fragment thereof, which specifically binds to human proprotein convertase subtilisin/kexin type 9 (hPCSK9), wherein the antibody or antigen-binding fragment thereof is administered at a dosage amount within the range of about 5 mg to about 500 mg, thereby enhancing LCL-C lowering activity of the statin therapy in the subject.

According to a preferred embodiment of the 34^{th} aspect, the subject is resistant to the statin therapy prior to administration of the antibody.

According to another preferred embodiment, the subject suffers from a disease or condition selected from the group consisting of hypercholesterolemia, hyperlipidemia, dyslipidemia, and atherosclerosis.

According to another preferred embodiment, the disease condition is primary hypercholesterolemia or familial hypercholesterolemia.

According to another preferred embodiment, the antibody or antigen-binding fragment is administered in a dosage amount within the range of about 50 mg to about 300 mg.

According to another preferred embodiment, the antibody or antigen-binding fragment is administered in a dosage amount of about 150 mg.

According to another preferred embodiment, the antibody or antigen-binding fragment thereof is administered to the subject every other week (E2W).

According to another preferred embodiment, the antibody or antigen-binding fragment thereof is administered to the subject every fourth week (E4W).

According to another preferred embodiment, the treatment reduces serum total cholesterol at least about 25% to about 35% relative to a predose level and sustains the reduction over at least a 24 day period.

According to another preferred embodiment, the treatment reduces serum total cholesterol at least about 65% to about 80% relative to a predose level and sustains the reduction over at least a 24 day period.

According to another preferred embodiment, the treatment reduces serum triglyceride levels at least about 25% to about 40% relative to a predose level.

According to another preferred embodiment, the treatment reduced serum HDL cholesterol no more than 5% relative to a predose level.

According to another preferred embodiment, the treatment has little or no measurable effect on liver function, as determined by ALT and AST measurements.

According to another preferred embodiment, the antibody or the antigen-binding fragment comprises 1the heavy and light chain CDRs of a HCVR/LCVR amino acid sequence pair as shown in SEQ ID NOs: 90/92.

According to another preferred embodiment, the antibody or antigen-binding fragment comprises a HCVR/LCVR amino acid sequence pair as shown in SEQ ID NOs: 90/92.

According to another preferred embodiment, the antibody or antigen-binding fragment thereof competes for binding to hPCSK9 with an antibody or antigen-binding fragment comprising a HCVR/LCVR amino acid sequence pair as shown in SEQ ID NOs: 90/92.

According to another preferred embodiment, the statin is selected from the group consisting of cerivastatin, atorvastatin, simvastatin, pitavastatin, rosuvastatin, fluvastatin, lovastatin, and pravastatin.

According to another preferred embodiment, the statin is atorvastatin administered at a dosage of 10 mg, 20 mg, 40 mg or 80 mg.

In a thirtyfifth aspect, present invention concerns a kit for treating elevated low-density lipoprotein cholesterol (LDL-C) levels in a subject, the kit comprising (a) pharmaceutical unit dosage form comprising an antibody, or antigen-binding fragment thereof, which specifically binds to hPCSK9; and pharmaceutically acceptable carrier, wherein the antibody or antigen-binding fragment is present in a dosage amount within the range of about 5 mg to about 500 mg; and (b) a label or packaging insert with instructions for use.

According to a preferred embodiment of the 35^{th} aspect, the label indicates that patients receiving treatment with said antibody or antigen-binding fragment can be treated for a disease or condition selected from the group consisting of hypercholesterolemia, hyperlipidemia, dyslipidemia, and atherosclerosis and cardiovascular diseases.

According to another preferred embodiment, the disease or condition is primary hypercholesterolemia or familial hypercholesterolemia. According to another preferred embodiment, the disease or condition is hypercholesterolemia which is uncontrolled by statins.

According to another preferred embodiment, the antibody or antigen-binding fragment is present in dosage amount within the range of about 50 mg to about 300 mg. According to another preferred embodiment, the antibody or antigen-binding fragment is present in a dosage amount of about 150 mg.

According to another preferred embodiment, the label or packaging insert indicates that the antibody or antigen-binding fragment thereof is administered to the subject every other week (E2W).

According to another preferred embodiment, the label or packaging insert indicates that the antibody or antigen-binding fragment thereof is administered to the subject every fourth week (E4W).

According to another preferred embodiment, the antibody or the antigen-binding fragment comprises the heavy and light chain CDRs of a HCVR/LCVR amino acid sequence pair as shown in SEQ ID NOs: 90/92

According to another preferred embodiment, the antibody or antigen-binding fragment comprises a HCVR/LCVR amino acid sequence pair as shown in SEQ ID NOs: 90/92.

According to another preferred embodiment, the antibody or antigen-binding fragment thereof competes for binding to hPCSK9 with an antibody or antigen-binding fragment comprising a HCVR/LCVR amino acid sequence pair as shown in SEQ ID NOs: 90/92.

According to another preferred embodiment, the kit further comprises an HMG-COA reductase inhibitor. According to another preferred embodiment, the inhibitor is in a dosage amount in the range of about 0.05 mg to 100 mg. According to another preferred embodiment, the HMG-COA reductase inhibitor is a statin. According to another preferred embodiment, the statin is selected from the group consisting of cerivastatin, atorvastatin, simvastatin, pitavastatin, rosuvastatin, fluvastatin, lovastatin, and pravastatin.

According to another preferred embodiment, the instructions indicate that the statin is atorvastatin administered at a dosage of 10 mg, 20 mg, 40 mg or 80 mg.

According to another preferred embodiment, the instructions indicate that treatment with the antibody or an is contraindicated for patients belonging to one or more of the following groups:
(i) smokers;
(ii) persons being 70 years old or older;
(iii) persons suffering from hypertension;
(iv) women who are pregnant;
(v) women who are trying to become pregnant;
(vi) women who are breast-feeding;
(vii) persons who have or ever had a disease affecting the liver;
(viii) persons who had any unexplained abnormal blood tests for liver function;
(ix) persons who drink excessive amounts of alcohol;
(x) persons having kidney problems;
(xi) persons suffering from hypothyroidism;
(xii) persons suffering from muscle disorders;
(xiii) persons having encountered previous muscular problems during treatment with lipid-lowering medicine;
(xiv) persons having serious problems with their breathing;
(xv) persons taking one or more of the following medicines: medicines altering the way the immune systems works (e.g. ciclosporin or antihistamines), antibiotics or antifungal medicines (e.g. erythromycin, clarithromycin, ketoconazole, itraconazole, rifampicin, fusidic acid), medicines regulating lipid levels (e.g. gemfibrozil, colestipol), calcium channel blockers (e.g. verapamil, diltiazem), medicines regulating the heart rhythm (digoxin, amiodarone), protease inhibitors used in the treatment of HIV (e.g. nelfinavir), warfarin, oral contraceptives, antacids or St. John's Wort; or
(xvi) persons drinking more than 0.1 L of grapefruit juice per day;
(xvii) persons having a body mass index (BMI) of more than 40;
(xviii) persons having a body mass index (BMI) of less than 18;
(xix) persons suffering from type 1 diabetes or type 2 diabetes;
(xx) persons positive for hepatitis B or hepatitis C; or
(xxi) persons having a known sensitivity to monoclonal antibody therapeutics.

In a thirtysixth aspect, present invention concerns a method of treating a subject suffering from a disease or disorder characterized by elevated low-density lipoprotein cholesterol (LDL-C) levels, the method comprising:
(a) selecting a subject with a blood LDL-C level greater than 100 mg/dL; and
(b) administering to said subject a composition comprising an antibody or antigen binding fragment thereof that specifically binds to human proprotein convertase subtilisin/kexin type 9 (hPCSK9); thereby lowering cholesterol levels in the subject in need thereof.

According to a preferred embodiment, the disease or condition is selected from the group consisting of: hypercholesterolemia, hyperlipidemia, dyslipidemia, and atherosclerosis.

According to another preferred embodiment, the disease condition is primary hypercholesterolemia or familial hypercholesterolemia.

According to another preferred embodiment, the disease or condition is hypercholesterolemia which is uncontrolled by statins.

According to another preferred embodiment, the subject has a body mass index (BMI) of less than 18 kg/m² or greater than 40 kg/m².

According to another preferred embodiment, subject was not previously instructed to partake in a cholesterol-lowering diet.

According to another preferred embodiment, the subject has not previously taken a cholesterol-lowering drug except for atorvastatin.

According to another preferred embodiment, the atorvastatin was administered at about 10 mg per day.

According to another preferred embodiment, cholesterol-lowering drug is selected from the group consisting of fibrates, bile acid resins, niacin, intestinal cholesterol absorption (ICA) blockers, and omega-3 fatty acids. According to another preferred embodiment, the niacin is administered at greater than 500 mg per day. According to another preferred embodiment, the omega-3 fatty acids are administered at greater than 1000 mg per day.

According to another preferred embodiment, the subject does not suffer from diabetes. According to another preferred embodiment, the diabetes is type 1 diabetes. According to another preferred embodiment, the diabetes is type 2 diabetes. According to another preferred embodiment, the type 2 diabetes is treated with insulin.

According to another preferred embodiment, the subject has a blood glycated hemoglobin concentration greater than or equal to 8.5%.

According to another preferred embodiment, the subject is negative for hepatitis B and C surface antigen.

According to another preferred embodiment, the subject has a blood triglycerides concentration of greater than 350 mg/dL.

According to another preferred embodiment, the subject has fewer than 1500 neutrophils per cubic mm of blood.

According to another preferred embodiment, the subject has fewer than 100,000 platelets per cubic mm of blood.

According to another preferred embodiment, the subject is female.

According to another preferred embodiment, the subject is not pregnant.

According to another preferred embodiment, the subject has a blood thyroid stimulating hormone concentration that is above the lower limit of normal and below the upper limit of normal.

According to another preferred embodiment, the subject has serum creatine of less than 1.4 of the upper limit of normal.

According to another preferred embodiment, the subject is a male.

According to another preferred embodiment, the subject has serum creatine of less than 1.5 of the upper limit of normal.

According to another preferred embodiment, the subject has an amount of aspartate transaminase that is less than two times the upper limit of normal.

According to another preferred embodiment, the subject has an amount of alanine transaminase that is less than two times the upper limit of normal.

According to another preferred embodiment, the antibody or antigen-binding fragment is administered in a dosage amount within the range of about 5 mg to about 500 mg.

According to another preferred embodiment, the antibody or antigen-binding fragment is administered in a dosage amount within the range of about 50 mg to about 300 mg.

According to another preferred embodiment, the antibody is administered at between 200 and 300 mg every four weeks.

According to another preferred embodiment, the antibody or antigen-binding fragment is administered in a dosage amount of about 150 mg.

According to another preferred embodiment, the antibody or antigen-binding fragment thereof is administered to the subject every other week (E2W).

According to another preferred embodiment, the antibody or antigen-binding fragment thereof is administered to the subject every fourth week (E4W).

According to another preferred embodiment, the antibody or the antigen-binding fragment comprises the heavy and light chain CDRs of a HCVR/LCVR amino acid sequence pair as shown in SEQ ID NOs: 90/92.

According to another preferred embodiment, the antibody or antigen-binding fragment comprises a HCVR/LCVR amino acid sequence pair as shown in SEQ ID NOs: 90/92.

According to another preferred embodiment, the antibody or antigen-binding fragment thereof competes for binding to hPCSK9 with an antibody or antigen-binding fragment comprising a HCVR/LCVR amino acid sequence pair as shown in SEQ ID NOs: 90/92.

According to another preferred embodiment, the antibody is administered subcutaneously.

According to another preferred embodiment, the antibody is administered in the abdomen.

According to another preferred embodiment, an HMG-COA reductase inhibitor is administered to the subject.

According to another preferred embodiment, the HMG-COA reductase inhibitor is administered in a dosage amount in the range of about 0.05 mg to 100 mg.

According to another preferred embodiment, the HMG-COA reductase inhibitor is a statin.

According to another preferred embodiment, the statin is selected from the group consisting of cerivastatin, atorvastatin, simvastatin, pitavastatin, rosuvastatin, fluvastatin, lovastatin, and pravastatin.

According to another preferred embodiment, the statin is atorvastatin administered at a dosage of 10 mg or 80 mg.

According to another preferred embodiment, the atorvastatin is administered at about 10 mg per day and at 80 mg one day in an 8 week period.

In a thirtyseventh aspect, present invention concerns a method of lowering cholesterol levels in a subject in need thereof, comprising:
(a) selecting a subject with a blood low density lipoprotein cholesterol (LDL-C) level greater than 100 mg/dL; and
(b) administering to said subject a composition comprising an antibody or antigen binding fragment thereof that specifically binds to human proprotein convertase subtilisin/kexin type 9 (hPCSK9); thereby lowering cholesterol levels in the subject in need thereof.

According to a preferred embodiment of the 37^{th} aspect, the the disease or condition is selected from the group consisting of: hypercholesterolemia, hyperlipidemia, dyslipidemia, and atherosclerosis.

According to another preferred embodiment, the disease condition is primary hypercholesterolemia or familial hypercholesterolemia.

According to another preferred embodiment, the disease or condition is hypercholesterolemia which is uncontrolled by statins.

According to another preferred embodiment, the subject has a body mass index (BMI) of less than 18 kg/m² or greater than 40 kg/m².

According to another preferred embodiment, the subject was not previously instructed to partake in a cholesterol-lowering diet.

According to another preferred embodiment, the subject has not previously taken a cholesterol-lowering drug except for atorvastatin.

According to another preferred embodiment, the atorvastatin was administered at about 10 mg per day.

According to another preferred embodiment, the cholesterol-lowering drug is selected from the group consisting of fibrates, bile acid resins, niacin, intestinal cholesterol absorption (ICA) blockers, and omega-3 fatty acids.

According to another preferred embodiment, the niacin is administered at greater than 500 mg per day.

According to another preferred embodiment, the omega-3 fatty acids are administered at greater than 1000 mg per day.

According to another preferred embodiment, the subject does not suffer from diabetes.

According to another preferred embodiment, the diabetes is type 1 diabetes.

According to another preferred embodiment, the diabetes is type 2 diabetes.

According to another preferred embodiment, the type 2 diabetes is treated with insulin.

According to another preferred embodiment, the subject has a blood glycated hemoglobin concentration greater than or equal to 8.5%.

According to another preferred embodiment, the subject is negative for hepatitis B and C surface antigen.

According to another preferred embodiment, the subject has a blood triglycerides concentration of greater than 350 mg/dL.

According to another preferred embodiment, the subject has fewer than 1500 neutrophils per cubic mm of blood.

According to another preferred embodiment, the subject has fewer than 100,000 platelets per cubic mm of blood.

According to another preferred embodiment, the subject is female.

According to another preferred embodiment, the subject is not pregnant.

According to another preferred embodiment, the subject has a blood thyroid stimulating hormone concentration that is above the lower limit of normal and below the upper limit of normal.

According to another preferred embodiment, the subject has serum creatine of less than 1.4 of the upper limit of normal.

According to another preferred embodiment, the subject is a male.

According to another preferred embodiment, the subject has serum creatine of less than 1.5 of the upper limit of normal.

According to another preferred embodiment, the subject has an amount of aspartate transaminase that is less than two times the upper limit of normal.

According to another preferred embodiment, the subject has an amount of alanine transaminase that is less than two times the upper limit of normal.

According to another preferred embodiment, the antibody or antigen-binding fragment is administered in a dosage amount within the range of about 5 mg to about 500 mg.

According to another preferred embodiment, the antibody or antigen-binding fragment is administered in a dosage amount within the range of about 50 mg to about 300 mg.

According to another preferred embodiment, the antibody is administered at between 200 and 300 mg every four weeks.

According to another preferred embodiment, the antibody or antigen-binding fragment is administered in a dosage amount of about 150 mg.

According to another preferred embodiment, the antibody or antigen-binding fragment thereof is administered to the subject every other week (E2W).

According to another preferred embodiment, the antibody or antigen-binding fragment thereof is administered to the subject every fourth week (E4W).

According to another preferred embodiment, the antibody or the antigen-binding fragment comprises the heavy and light chain CDRs of a HCVR/LCVR amino acid sequence pair as shown in SEQ ID NOs: 90/92. According to another preferred embodiment, the antibody or antigen-binding fragment comprises a HCVR/LCVR amino acid sequence pair as shown in SEQ ID NOs: 90/92. According to another preferred embodiment, the antibody or antigen-binding fragment thereof competes for binding to hPCSK9 with an antibody or antigen-binding fragment comprising a HCVR/LCVR amino acid sequence pair as shown in SEQ ID NOs: 90/92. According to another preferred embodiment, the antibody is administered subcutaneously. According to another preferred embodiment, the antibody is administered in the abdomen.

According to another preferred embodiment, the method further comprises administering a HMG-COA reductase inhibitor to the subject. According to another preferred embodiment, the HMG-COA reductase inhibitor is administered in a dosage amount in the range of about 0.05 mg to 100 mg. According to another preferred embodiment, the HMG-COA reductase inhibitor is a statin. According to another preferred embodiment, the statin is selected from the group consisting of cerivastatin, atorvastatin, simvastatin, pitavastatin, rosuvastatin, fluvastatin, lovastatin, and pravastatin. According to another preferred embodiment, the statin is atorvastatin administered at a dosage of 10 mg, 20 mg, 40 mg or 80 mg. According to another preferred embodiment, the atorvastatin is administered at about 10 mg per day and at 80 mg one day in an 8 week period.

Several aspects of the invention can be combined with each other. For example, the method for treating a disease or condition according to the first aspect and the method for treating a disease or condition according to the thirteenth aspect can be combined. As a result of this combination the present invention relates to a method for treating a disease or condition which features the treatment of certain groups of subjects by certain dosage regimens. In an analogous manner, the antibody or antigen-binding fragment for use in the treatment of a disease or condition according to the second aspect can be combined with the antibody or antigen-binding fragment for use in the treatment of a disease or condition according to the fifteenth aspect. As a result of this combination the present invention relates to an antibody or antigen-binding fragment thereof for use in the treatment of certain groups of subjects by certain dosage regimens.

According to another example, the method for treating a disease or condition according to the first aspect and the method for treating a disease or condition according to the fourteenth aspect can be combined. As a result of this combination the present invention relates to a method for treating a disease or condition which excludes certain groups of subjects from a treatment by a certain dosage regimen. In an analogous manner, the antibody or antigen-binding fragment for use in the treatment of a disease or condition according to the second aspect can be combined with the antibody or antigen-binding fragment for use in the treatment of a disease or condition according to the sixteenth aspect. As a result of this combination the present invention relates to an antibody or antigen-binding fragment thereof for use in the treatment by a certain dosage regimen, wherein certain groups of subjects are excluded from the treatment.

The skilled artisan will recognize other preferred embodiments resulting of suitable combinations of different aspects and embodiments of present invention.

The pharmaceutical uses of present invention as herein described also relate to uses of the given antibody or antigen-binding fragment thereof, of the given pharmaceutical composition, etc for the manufacture of a medicament for the treatment of one or more of the diseases or conditions as herein described.

### Preferred Antibodies for Practicing the Present Invention

The following section describes functional and structural features of antibodies and antigen-binding fragments thereof that can be used for practicing all twenty-one aspects of the present invention. Thus, expressions such as “in preferred embodiments”, “in some embodiments”, “in another preferred embodiment” and similar expressions should be understood as referring to embodiments of the first aspect of the present invention, the second aspect of the present invention, the third aspect of the present invention, the fourth aspect of the present invention, the fifth aspect of the present invention, the sixth aspect of the present invention, the seventh aspect of the present invention, the eighth aspect of the present invention, the ninth aspect of the present invention, the tenth aspect of the present invention, the eleventh aspect of the present invention, the twelfth aspect of the present invention, the thirteenth aspect of the present invention, the fourteenth aspect of the present invention, the fifteenth aspect of the present invention, the sixteenth aspect of the present invention, the seventeenth aspect of the present invention, the eighteenth aspect of the present invention, the nineteenth aspect of the present invention, the twentieth aspect, and the twenty-first aspect of the present invention, the twentysecond aspect of the present invention, the twentythird aspect of the present invention, the twentyfourth aspect of the present invention, the twentyfifth aspect of the present invention, the twentysixth aspect of the present invention, the twentyseventh aspect of the present invention, the twentyeighth aspect of the present invention, the twentyninth aspect of the present invention, the thirtieth aspect of the present invention, the thirtyfirst aspect of the present invention, the thirtysecond aspect of the present invention, the thirtythird aspect of the present invention, the thirtyfourth aspect of present invention, the thirtyfifth aspect of present invention, the thirtysixth aspect of present invention, the thirtyseventh aspect of present invention.

All antibodies or antigen-binding fragments thereof suitable for practicing the present invention specifically bind hPCSK9. In preferred embodiments of any aspect of the present invention, the antibody or antigen-binding fragment thereof is a recombinant human antibody or fragment thereof. In more specific embodiments, the antibody or antigen-binding fragment thereof is a fully human monoclonal antibody or antigen-binding fragment thereof that specifically binds hPCSK9 and neutralizes PCSK9 activity.

The mAbs usable in the present invention can be full-length (e.g., an IgG1 or IgG4 antibody) or may comprise only an antigen-binding portion (e.g., a Fab, F(ab′)₂ or scFv fragment), and may be modified to affect functionality, e.g., to eliminate residual effector functions (Reddy et al. (2000) J. Immunol. 164:1925-1933).

In preferred embodiments, the antibodies of present invention are characterized by one or more of the following features upon administration to a subject, preferably a human or non-human mammal and more preferably a human:
reduction of low density lipoprotein-C (LDL-C) levels of at least about −25% to about −40% relative to a predose level with a sustained reduction over at least a 14 day-period, wherein the sustained reduction is preferably at least −25% and more preferably at least −30% relative to a predose level, particularly if administered in a dose of about 40 to about 60 mg, preferably about 45 to about 55 mg and more preferably about 50 mg in a biweekly administration regime (every other week, E2W),
reduction of low density lipoprotein-C (LDL-C) of at least about −50% to about −65% relative to a predose level with a sustained reduction over at least a 14 day-period, wherein the sustained reduction is preferably at least −40% and more preferably at least −45% relative to a predose level, particularly if administered in a dose of about 100 mg E2W,
reduction of low-density lipoprotein-C (LDL-C) of at least about −60% to at least about −75% [e.g. at least about −60%, at least about −65%, at least about −70 or at least about −75%] relative to a predose level with a sustained reduction over at least a 14 day-period, wherein the sustained reduction is preferably at least −55% and more preferably at least −60% relative to a predose level, particularly when administered in a dose of about 150 mg E2W,
reduction of low density lipoprotein-C (LDL-C) of at least about 40% to about 75% relative to a predose level with a sustained reduction over at least a 28 day period, wherein the sustained reduction is preferably at least −35% and more preferably at least −40% relative to a predose level, particularly when administered in a dose of about 200 mg E4W,
reduction of low density lipoprotein-C (LDL-C) of at least about −50% to about −75% relative to a predose level with a sustained reduction over at least a 28 day-period, wherein the sustained reduction is preferably at least −40% and more preferably at least −45% relative to a predose level, particularly when administered in a dose of about 300 mg E4W,
increase of serum HDL cholesterol levels of at least 2%, at least 2.5%, at least, 3%, at least 3.5%, at least 4%, at least 4.5%, at least 5% or at least 5.5% relative to a predose level, particularly when administered in a dose of about 150 mg E2W,
little or no detectable effect on troponin levels,
Increase of one or more of: Total-Cholesterol levels, ApoB levels, non HDL-C levels, Apo-B/ApoA-1 ratio,

The antibodies according to present invention exhibit the above properties preferably if administered in combination with an HMG-COA reductase inhibitor treatment. Preferred embodiments of HMG-COA reductase inhibitors to be used in conjunction with the antibody of the invention and dosage and administration regimes thereof can be found throughout the specification, particularly as described in the aspects related to medical uses and methods of treatment.

According to another preferred embodiment of the antibodies and antigen-binding fragments thereof of present invention, the antibody or antigen binding fragment thereof has one or more of the following characteristics:
The antibody or the antigen-binding fragment comprises the heavy and light chain CDRs of a HCVR/LCVR amino acid sequence pair as shown in SEQ ID NOs: 90/92.
The antibody or antigen-binding fragment thereof comprises a HCVR/LCVR amino acid sequence pair as shown in SEQ ID NOs: 90/92.
The antibody or antigen-binding fragment thereof competes for binding to hPCSK9 with an antibody or antigen-binding fragment comprising a HCVR/LCVR amino acid sequence pair as shown in SEQ ID NOs: 90/92.

According to another preferred embodiment of the antibodies and antigen-binding fragments thereof of present invention, the antibody or antigen binding fragment thereof has one or more of the following characteristics:
overcomes statin resistance in mammals, especially in rodents such as hamster
increase in LDLR expression in mammals, particularly in rodents such as hamster
decreases serum LDL-C in rodents such as hamster
synergistic decrease of LDL-C in conjunction with HMG-COA reductase inhibitor administration, particularly in rodents such as hamster, wherein the HMG-COA reductase inhibitor is preferably Atorvastatin.

In preferred embodiments, the antibody or the antigen-binding fragment thereof is characterized by one or more of the following:
(i) capable of reducing serum total cholesterol at least about 25 to about 35% and sustaining the reduction over at least a 24 day period relative to a predose level, preferably the reduction in serum total cholesterol is at least about 30-40%;
(ii) capable of reducing serum LDL cholesterol at least about 65-80% and sustaining the reduction over at least a 24 day period relative to a predose level;
(iii) capable of reducing serum triglyceride at least about 25-40% relative to predose level;
(iv) achieves one or more of (i)-(iii) without reducing serum HDL cholesterol or reducing serum HDL cholesterol no more than 5% relative to predose level;
(v) achieves one or more of (i)-(iii) with little or no measurable effect on liver function, as determined by ALT and AST measurements.

In preferred embodiments, the antibody or the antigen-binding fragment thereof is characterized by one or more of the following:
(i) capable of reducing serum LDL cholesterol at least about 40-70% and sustaining the reduction over at least a 60 or 90 day period relative to a predose level;
(ii) capable of reducing serum triglyceride at least about 25-40% relative to predose level;
(iii) does not reduce serum HDL cholesterol or reduces serum HDL cholesterol no more than 5% relative to predose level.

In one embodiment, the antibody or the antigen-binding fragment thereof is characterized as binding an epitope comprising amino acid residue 238 of hPCSK9 (SEQ ID NO:755). In a more specific embodiment, the antibody or antigen-binding fragment binds an epitope comprising one or more of amino acid residues at positions 238, 153, 159 and 343 of hPCSK9 (SEQ ID NO:755). In a more specific embodiment, the antibody or fragment thereof is characterized as binding an epitope which does not comprise an amino acid residue at positions 192, 194, 197 and/or 237 of SEQ ID NO:755.

In one embodiment, the antibody or the antigen-binding fragment thereof is characterized as binding an epitope comprising amino acid residue 366 of hPCSK9 (SEQ ID NO:755). In a more specific embodiment, the antibody or antigen-binding fragment binds an epitope comprising one or more of amino acid residues at positions 147, 366 and 380 of hPCSK9 (SEQ ID NO: 755). In a more specific embodiment, the antibody or antigen-binding fragment of an antibody is characterized as binding an epitope which does not comprise an amino acid residue at position 215 or 238 of SEQ ID NO:755.

In one embodiment, the antibody or the antigen-binding fragment thereof comprises a heavy chain variable region (HCVR) selected from the group consisting of SEQ ID NO:2, 18, 22, 26, 42, 46, 50, 66, 70, 74, 90, 94, 98, 114, 118, 122, 138, 142, 146, 162, 166, 170, 186, 190, 194, 210, 214, 218, 234, 238, 242, 258, 262, 266, 282, 286, 290, 306, 310, 314, 330, 334, 338, 354, 358, 362, 378, 382, 386, 402, 406, 410, 426, 430, 434, 450, 454, 458, 474, 478, 482, 498, 502, 506, 522, 526, 530, 546, 550, 554, 570, 574, 578, 594, 598, 602, 618, 622, 626, 642, 646, 650, 666, 670, 674, 690, 694, 698, 714, 718, 722, 738 and 742, or a substantially similar sequence thereof having at least 90%, at least 95%, at least 98% or at least 99% sequence identity. In one embodiment, the HCVR comprises an amino acid sequence selected from the group consisting of SEQ ID NO:50, 66, 70, 74, 90, 94, 122, 138, 142, 218, 234, 238, 242, 258, 262, 314, 330 and 334. In a more specific embodiment, the HCVR comprises SEQ ID NO:90 or 218.

In one embodiment, the antibody or the antigen-binding fragment thereof further comprises a light chain variable region (LCVR) selected from the group consisting of SEQ ID NO: 10, 20, 24, 34, 44, 48, 58, 68, 72, 82, 92, 96, 106, 116, 120, 130, 140, 144, 154, 164, 168, 178, 188, 192, 202, 212, 216, 226, 236, 240, 250, 260, 264, 274, 284, 288, 298, 308, 312, 322, 332, 336, 346, 356, 360, 370, 380, 384, 394, 404, 408, 418, 428, 432, 442, 452, 456, 466, 476, 480, 490, 500, 504, 514, 524, 528, 538, 548, 552, 562, 572, 576, 586, 596, 600, 610, 620, 624, 634, 644, 648, 658, 668, 672, 682, 692, 696, 706, 716, 720, 730, 740 and 744, or a substantially similar sequence thereof having at least 90%, at least 95%, at least 98% or at least 99% sequence identity. In one embodiment, the LCVR comprises an amino acid sequence selected from the group consisting of SEQ ID NO: 58, 68, 72, 82, 92, 96, 130, 140, 144, 226, 236, 240, 250, 260, 264, 322, 332 and 336. In a more specific embodiment, the LCVR comprises SEQ ID NO:92 or 226.

In specific embodiments, the antibody or the antigen-binding fragment thereof comprises a HCVR and LCVR (HCVR/LCVR) sequence pair selected from the group consisting of SEQ ID NO: 2/10, 18/20, 22/24, 26/34, 42/44, 46/48, 50/58, 66/68, 70/72, 74/82, 90/92, 94/96, 98/106, 114/116, 118/120, 122/130, 138/140, 142/144, 146/154, 162/164, 166/168, 170/178, 186/188, 190/192, 194/202, 210/212, 214/216, 218/226, 234/236, 238/240, 242/250, 258/260, 262/264, 266/274, 282/284, 286/288, 290/298, 306/308, 310/312, 314/322, 330/332, 334/336, 338/346, 354/356, 358/360, 362/370, 378/380, 382/384, 386/394, 402/404, 406/408, 410/418, 426/428, 430/432, 434/442, 450/452, 454/456, 458/466, 474/476, 478/480, 482/490, 498/500, 502/504, 506/514, 522/524, 526/528, 530/538, 546/548, 550/552, 554/562, 570/572, 574/576, 578/586, 594/596, 598/600, 602/610, 618/620, 622/624, 626/634, 642/644, 646/648, 650/658, 666/668, 670/672, 674/682, 690/692, 694/696, 698/706, 714/716, 718/720, 722/730, 738/740 and 742/744. In one embodiment, the HCVR and LCVR sequence pair comprises one of SEQ ID NO: 50/58, 66/68, 70/72, 74/82, 90/92, 94/96, 122/130, 138/140, 142/144, 218/226, 234/236, 238/240, 242/250, 258/260, 262/264, 314/322, 330/332 and 334/336. In preferred embodiments, the antibody or antigen-binding fragment thereof comprises an HCVR amino acid sequence as shown in SEQ ID NO: 90 and an LCVR amino acid sequence as shown in SEQ ID NO: 92. In another preferred embodiment, the antibody or antigen-binding fragment thereof comprises an HCVR amino acid sequence as shown in SEQ ID NO: 218 and an LCVR amino acid sequence as shown in SEQ ID NO: 226.

In preferred embodiments, the antibody or the antigen-binding fragment thereof comprises a heavy chain CDR3 (HCDR3) domain selected from the group consisting of SEQ ID NO: 8, 32, 56, 80, 104, 128, 152, 176, 200, 224, 248, 272, 296, 320, 344, 368, 392, 416, 440, 464, 488, 512, 536, 560, 584, 608, 632, 656, 680, 704 and 728, or a substantially similar sequence thereof having at least 90%, at least 95%, at least 98% or at least 99% sequence identity; and a light chain CDR3 (LCDR3) domain selected from the group consisting of SEQ ID NO:16, 40, 64, 88, 112, 136, 160, 184, 208, 232, 256, 280, 304, 328, 352, 376, 400, 424, 448, 472, 496, 520, 544, 568, 592, 616, 640, 664, 688, 712 and 736, or substantially similar sequences thereof having at least 90%, at least 95%, at least 98% or at least 99% sequence identity. In one embodiment, the HCDR3/LCDR3 sequence pair is selected from the group consisting of SEQ ID NO:56/64, 80/88, 128/136, 224/232, 248/256 and 320/328. In more preferred embodiments, the antibody or the antigen-binding fragment thereof comprises a HCDR3 domain as shown in SEQ ID NO: 80 and a LCDR3 domain as shown in SEQ ID NO: 88. In another preferred embodiment, the antibody or the antigen-binding fragment thereof comprises a HCDR3 domain as shown in SEQ ID NO: 224 and a LCDR3 domain as shown in SEQ ID NO: 232.

In a further embodiment, the antibody or the antigen-binding fragment thereof further comprises a heavy chain CDR1 (HCDR1) domain selected from the group consisting of SEQ ID NO: 4, 28, 52, 76, 100, 124, 148, 172, 196, 220, 244, 268, 292, 316, 340, 364, 388, 412, 436, 460, 484, 508, 532, 556, 580, 604, 628, 652, 676, 700 and 724, or a substantially similar sequence thereof having at least 90%, at least 95%, at least 98% or at least 99% sequence identity; a heavy chain CDR2 (HCDR2) domain selected from the group consisting of SEQ ID NO:6, 30, 54, 78, 102, 126, 150, 174, 198, 222, 246, 270, 294, 318, 342, 366, 390, 414, 438, 462, 486, 510, 534, 558, 582, 606, 630, 654, 678, 702 and 726, or a substantially similar sequence thereof having at least 90%, at least 95%, at least 98% or at least 99% sequence identity; a light chain CDR1 (LCDR1) domain selected from the group consisting of SEQ ID NO:12, 36, 60, 84, 108, 132, 156, 180, 204, 228, 252, 276, 300, 324, 348, 372, 396, 420, 444, 468, 492, 516, 540, 564, 588, 612, 636, 660, 684, 708 and 732, or a substantially similar sequence thereof having at least 90%, at least 95%, at least 98% or at least 99% sequence identity; and a light chain CDR2 (LCDR2) domain selected from the group consisting of SEQ ID NO:14, 38, 62, 86, 110, 134, 158, 182, 206, 230, 254, 278, 302, 326, 350, 374, 398, 422, 446, 470, 494, 518, 542, 566, 590, 614, 638, 662, 686, 710 and 734, or a substantially similar sequence thereof having at least 90%, at least 95%, at least 98% or at least 99% sequence identity. In one embodiment, the heavy and light chain CDR sequences comprise a sequence selected from the group consisting of SEQ ID NO: 52, 54, 56, 60, 62, 64; 76, 78, 80, 84, 86, 88; 124, 126, 128, 132, 134, 136; 220, 222, 224, 228, 230, 232; 244, 246, 248, 252, 254, 256; and 316, 318, 320, 324, 326, 328. In more specific embodiments, the CDR sequences comprise SEQ ID NO: 76, 78, 80, 84, 86, 88; or 220, 222, 224, 228, 230, 232. In preferred embodiments, the antibody or antigen-binding fragment thereof comprises heavy and light chain CDR amino acid sequences as shown in SEQ ID NOs: 76, 78, 80, 84, 86 and 88. In another preferred embodiment, the antibody or antigen-binding fragment thereof comprises heavy and light chain CDR amino acid sequences as shown in SEQ ID NOS: 220, 222, 224, 228, 230 and 232.

In a related embodiment, the antibody or antigen-binding fragment thereof comprises heavy and light chain CDR domains contained within heavy and light chain sequence pairs selected from the group consisting of SEQ ID NO: 2/10, 18/20, 22/24, 26/34, 42/44, 46/48, 50/58, 66/68, 70/72, 74/82, 90/92, 94/96, 98/106, 114/116, 118/120, 122/130, 138/140, 142/144, 146/154, 162/164, 166/168, 170/178, 186/188, 190/192, 194/202, 210/212, 214/216, 218/226, 234/236, 238/240, 242/250, 258/260, 262/264, 266/274, 282/284, 286/288, 290/298, 306/308, 310/312, 314/322, 330/332, 334/336, 338/346, 354/356, 358/360, 362/370, 378/380, 382/384, 386/394, 402/404, 406/408, 410/418, 426/428, 430/432, 434/442, 450/452, 454/456, 458/466, 474/476, 478/480, 482/490, 498/500, 502/504, 506/514, 522/524, 526/528, 530/538, 546/548, 550/552, 554/562, 570/572, 574/576, 578/586, 594/596, 598/600, 602/610, 618/620, 622/624, 626/634, 642/644, 646/648, 650/658, 666/668, 670/672, 674/682, 690/692, 694/696, 698/706, 714/716, 718/720, 722/730, 738/740 and 742/744. In one embodiment, the CDR sequences are contained within HCVR and LCVR selected from the amino acid sequence pairs of SEQ ID NO: 50/58, 66/68, 70/72, 74/82, 90/92, 94/96, 122/130, 138/140, 142/144, 218/226, 234/236, 238/240, 242/250, 258/260, 262/264, 314/322, 330/332 and 334/336. In more specific embodiments, the CDR sequences are comprised within HCVR/LCVR sequences selected from SEQ ID NO: 90/92 or 218/226. In preferred embodiments, the antibody or the antigen-binding fragment thereof comprises the heavy and light chain CDRs of an HCVR/LCVR amino acid sequence pair as shown in SEQ ID NOs: 90/92. In another preferred embodiment, the antibody or the antigen-binding fragment thereof comprises the heavy and light chain CDRs of an HCVR/LCVR amino acid sequence pair as shown in SEQ ID NOs: 218/226.

In one specific embodiment, the antibody or the antigen-binding fragment thereof comprises the heavy chain variable region (HCVR), of SEQ ID NO:90 or a substantially similar sequence thereof having at least 90%, at least 95%, at least 98% or at least 99% sequence identity.

In one specific embodiment, the antibody or the antigen-binding fragment thereof further comprises the light chain variable region (LCVR) of SEQ Id NO 92 or a substantially similar sequence thereof having at least 90%, at least 95%, at least 98% or at least 99% sequence identity.

In specific embodiments, the antibody or the antigen-binding fragment thereof comprises HCVR amino acid sequence as shown in SEQ ID NO: 90 and an LCVR amino acid sequence as shown in SEQ ID NO: 92.

In specific embodiments, the antibody or the antigen-binding fragment thereof comprises a heavy chain CDR3 (HCDR3) domain of SEQ ID NO: 80 or a substantially similar sequence thereof having at least 90%, at least 95%, at least 98% or at least 99% sequence identity; and/or a light chain CDR3 (LCDR3) domain of SEQ ID NO: 88, or substantially similar sequences thereof having at least 90%, at least 95%, at least 98% or at least 99% sequence identity. In one embodiment, the HCDR3/LCDR3 sequence pair is SEQ ID NO:80/88. In more preferred embodiments, the antibody or the antigen-binding fragment thereof comprises a HCDR3 domain as shown in SEQ ID NO: 80 and a LCDR3 domain as shown in SEQ ID NO: 88.

In a further specific embodiment, the antibody or the antigen-binding fragment thereof further comprises the heavy chain CDR1 (HCDR1) domain of SEQ ID NO: 76, or a substantially similar sequence thereof having at least 90%, at least 95%, at least 98% or at least 99% sequence identity; and/or the heavy chain CDR2 (HCDR2) domain of SEQ ID NO: 78 or a substantially similar sequence thereof having at least 90%, at least 95%, at least 98% or at least 99% sequence identity; and/or a light chain CDR1 (LCDR1) domain of SEQ ID NO: 84 or a substantially similar sequence thereof having at least 90%, at least 95%, at least 98% or at least 99% sequence identity; and/or a light chain CDR2 (LCDR2) domain of SEQ ID NO: 86, or a substantially similar sequence thereof having at least 90%, at least 95%, at least 98% or at least 99% sequence identity. In one embodiment, the heavy and light chain CDR sequences comprise a sequence selected from the group consisting of SEQ ID NO: 76, 78, 80, 84, 86, 88. In preferred embodiments, the antibody or antigen-binding fragment thereof comprises heavy and light chain CDR amino acid sequences as shown in SEQ ID NOs: 76, 78, 80, 84, 86 and 88.

In another specific embodiment, the antibody or antigen-binding fragment thereof comprises heavy and light chain CDR domains contained within the heavy and light chain sequence pair of SEQ ID NO: 90/92.

A particularly preferred embodiment concerns an antibody comprising HCVR/LCVR sequences SEQ ID Nos: 90/92 and/or CDR sequences SEQ ID Nos: 76, 78, 80 and/or CDR sequences SEQ ID NO:s 84, 86, 88. Another particularly preferred embodiment concerns an antibody comprising the HCVR/LCVR sequences SEQ ID Nos: 90/92 and the CDR sequences SEQ ID Nos: 76, 78, 80 and the CDR sequences SEQ ID NO:s 84, 86, 88 (“316P”).

In one embodiment, the antibody or antigen-binding fragment thereof comprises an HCVR encoded by a nucleic acid sequence selected from the group consisting of SEQ ID NO: 1, 17, 21, 25, 41, 45, 49, 65, 69, 73, 89, 93, 97, 113, 117, 121, 137, 141, 145, 161, 165, 169, 185, 189, 193, 209, 213, 217, 233, 237, 241, 257, 261, 265, 281, 285, 289, 305, 309, 313, 329, 333, 337, 353, 357, 361, 377, 381, 385, 401, 405, 409, 425, 429, 433, 449, 453, 457, 473, 477, 481, 497, 501, 505, 521, 525, 529, 545, 549, 553, 569, 573, 577, 593, 597, 601, 617, 621, 625, 641, 645, 649, 665, 669, 673, 689, 693, 697, 713, 717, 721, 737 and 741, or a substantially identical sequence having at least 90%, at least 95%, at least 98%, or at least 99% sequence identity thereof. In one embodiment, the HCVR is encoded by a nucleic acid sequence selected from the group consisting of SEQ ID NO: 49, 65, 69, 73, 89, 93, 121, 137, 141, 217, 233, 237, 241, 257, 261, 313, 329 and 333. In more specific embodiments, the HCVR is encoded by a nucleic acid sequence selected from the group consisting of SEQ ID NO: 89 and 217.

In one embodiment, the antibody or fragment thereof further comprises an LCVR encoded by a nucleic acid sequence selected from the group consisting of SEQ ID NO: 9, 19, 23, 33, 43, 47, 57, 67, 71, 81, 91, 95, 105, 115, 119, 129, 139, 143, 153, 163, 167, 177, 187, 191, 201, 211, 215, 225, 235, 239, 249, 259, 263, 273, 283, 287, 297, 307, 311, 321, 331, 335, 345, 355, 359, 369, 379, 383, 393, 403, 407, 417, 427, 431, 441, 451, 455, 465, 475, 479, 489, 499, 503, 513, 523, 527, 537, 547, 551, 561, 571, 575, 585, 595, 599, 609, 619, 623, 633, 643, 647, 657, 667, 671, 681, 691, 695, 705, 715, 719, 729, 739 and 743, or a substantially identical sequence having at least 90%, at least 95%, at least 98%, or at least 99% sequence identity thereof. In one embodiment, the LCVR is encoded by a nucleic acid sequence selected from the group consisting of SEQ ID NO: 57, 67, 71, 81, 91, 95, 129, 139, 143, 225, 235, 239, 249, 259, 263, 321, 331 and 335. In more specific embodiments, the LCVR is encoded by a nucleic acid sequence selected from the group consisting of SEQ ID NO: 91 and 225.

In one embodiment, the antibody or antigen-binding fragment thereof comprises an HCDR3 domain encoded by a nucleotide sequence selected from the group consisting of SEQ ID NO: 7, 31, 55, 79, 103, 127, 151, 175, 199, 223, 247, 271, 295, 319, 343, 367, 391, 415, 439, 463, 487, 511, 535, 559, 583, 607, 631, 655, 679, 703 and 727, or a substantially identical sequence having at least 90%, at least 95%, at least 98%, or at least 99% sequence identity thereof; and a LCDR3 domain encoded by a nucleotide sequence selected from the group consisting of SEQ ID NO: 15, 39, 63, 87, 111, 135, 159, 183, 207, 231, 255, 279, 303, 327, 351, 375, 399, 423, 447, 471, 495, 519, 543, 567, 591, 615, 639, 663, 687, 711 and 735, or a substantially identical sequence having at least 90%, at least 95%, at least 98%, or at least 99% sequence identity thereof. In one embodiment, the HCDR3 and LCDR3 comprise a sequence pair encoded by the nucleic acid sequence of SEQ ID NO: 55/63, 79/87, 127/135, 223/231, 247/255 and 319/327, respectively. In more specific embodiments, the HCDR3 and LCDR3 comprise a sequence pair encoded by the nucleic acid sequence of SEQ ID NO: 79/87 and 223/231.

In a further embodiment, the antibody or antigen-binding fragment thereof further comprises: an HCDR1 domain encoded by a nucleotide sequence selected from the group consisting of SEQ ID NO: 3, 27, 51, 75, 99, 123, 147, 171, 195, 219, 243, 267, 291, 315, 339, 363, 387, 411, 435, 459, 483, 507, 531, 555, 579, 603, 627, 651, 675, 699 and 723, or a substantially identical sequence having at least 90%, at least 95%, at least 98%, or at least 99% sequence identity thereof; an HCDR2 domain encoded by a nucleotide sequence selected from the group consisting of SEQ ID NO:5, 29, 53, 77, 101, 125, 149, 173, 197, 221, 245, 269, 293, 317, 341, 365, 389, 413, 437, 461, 485, 509, 533, 557, 581, 605, 629, 653, 677, 701 and 725, or a substantially identical sequence having at least 90%, at least 95%, at least 98%, or at least 99% sequence identity thereof; an LCDR1 domain encoded by a nucleotide sequence selected from the group consisting of SEQ ID NO: 11, 35, 59, 83, 107, 131, 155, 179, 203, 227, 251, 275, 299, 323, 347, 371, 395, 419, 443, 467, 491, 515, 539, 563, 587, 611, 635, 659, 683, 707 and 731, or a substantially identical sequence having at least 90%, at least 95%, at least 98%, or at least 99% sequence identity thereof; and an LCDR2 domain encoded by a nucleotide sequence selected from the group consisting of SEQ ID NO: 13, 37, 61, 85, 109, 133, 157, 181, 205, 229, 253, 277, 301, 325, 349, 373, 397, 421, 445, 469, 493, 517, 541, 565, 589, 613, 637, 661, 685, 709 and 733, or a substantially identical sequence having at least 90%, at least 95%, at least 98%, or at least 99% sequence identity thereof. In one embodiment, the heavy and light chain CDR sequences are encoded by the nucleic acid sequences of SEQ ID NO: 51, 53, 55, 59, 61, 63; 75, 77, 79, 83, 85, 87; 123, 125, 127, 131, 133, 135; 219, 221, 223, 227, 229, 231; 243, 245, 247, 251, 253, 255; and 315, 317, 319, 323, 325, 327. In more specific embodiments, the heavy and light chain CDR sequences are encoded by the nucleic acid sequences of SEQ ID NO: 75, 77, 79, 83, 85, 87; and 219, 221, 223, 227, 229, 231.

In a further embodiment, the antibody or antigen-binding fragment thereof comprises an HCDR3 and an LCDR3, wherein HCDR3 comprises an amino acid sequence of the formula X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-X¹⁹-X²⁰ (SEQ ID NO:747), wherein X¹ is Ala, X² is Arg or Lys, X³ is Asp, X⁴ is Ser or Ile, X⁵ is Asn or Val, X⁶ is Leu or Trp, X⁷ is Gly or Met, X⁸ is Asn or Val, X⁹ is Phe or Tyr, X¹⁰ is Asp, X¹¹ is Leu or Met, X¹² is Asp or absent, X¹³ is Tyr or absent, X¹⁴ is Tyr or absent, X¹⁵ is Tyr or absent, X¹⁶ is Tyr or absent, X¹⁷ is Gly or absent, X¹⁸ is Met or absent, X¹⁹ is Asp or absent, and X²⁰ is Val or absent; and LCDR3 comprises an amino acid sequence of the formula X¹-X²-X³-X₄-X⁵-X⁶-X⁷-X⁸-X⁹ (SEQ ID NO:750), wherein X¹ is Gln or Met, X² is Gln, X³ is Tyr or Thr, X⁴ is Tyr or Leu, X⁵ is Thr or Gln, X⁶ is Thr, X⁷ is Pro, X⁸ is Tyr or Leu, and X⁹ is Thr.

In a further embodiment, the antibody or antigen-binding fragment thereof further comprises an HCDR1 sequence of the formula X¹-X²-X³-X⁴-X⁵-X⁶-X⁷-X⁸ (SEQ ID NO: 745), wherein X¹ is Gly, X² is Phe, X³ is Thr, X⁴ is Phe, X⁵ is Ser or Asn, X⁶ is Ser or Asn, X⁷ is Tyr or His, and X⁸ is Ala or Trp; a HCDR2 sequence of the formula X¹-X²-X³-X⁴-X⁵-X⁶-X⁷-X⁸ (SEQ ID NO:746), wherein X¹ is Ile, X² is Ser or Asn, X³ is Gly or Gln, X⁴ is Asp or Ser, X⁵ is Gly, X⁶ is Ser or Gly, X⁷ is Thr or Glu, and X⁸ is Thr or Lys; a LCDR1 sequence of the formula X¹-X²-X³-X⁴-X⁵-X⁶-X⁷-X⁸-X⁹-X¹⁰-X¹¹-X¹² (SEQ ID NO:748) wherein X¹ is Gln, X² is Ser, X³ is Val or Leu, X⁴ is Leu, X⁵ is His or Tyr, X⁶ is Arg or Ser, X⁷ is Ser or Asn, X⁸ is Asn or Gly, X⁹ is Asn, X¹⁰ is Arg or Asn, X¹¹ is Asn or Tyr, and X¹² is Phe or absent; an LCDR2 sequence of the formula X¹-X²-X³ (SEQ ID NO:749) wherein X¹ is Trp or Leu, X² is Ala or Gly, and X³ is Ser.

In a further embodiment, the antibody or antigen-binding fragment thereof is a human anti-PCSK9 antibody or antigen-binding fragment thereof comprising a heavy chain variable region (HCVR) encoded by nucleotide sequence segments derived from VH, DH and JH germline sequences, and a light chain variable region (LCVR) encoded by nucleotide sequence segments derived from V_{K} and J_{K} germline sequences, wherein the germline sequences are (a) VH gene segment 3-23, DH gene segment 7-27, JH gene segment 2, V_{K} gene segment 4-1 and J_{K} gene segment 2; or (b) VH gene segment 3-7, DH gene segment 2-8, JH gene segment 6, V_{K} gene segment 2-28 and J_{K} gene segment 4.

In preferred embodiments, the antibody or antigen-binding fragment thereof binds to the same epitope on hPCSK9 as an antibody comprising heavy and light chain CDR amino acid sequences as shown in SEQ ID NOs: 76, 78, 80, 84, 86, and 88 or as shown in SEQ ID NOs: 220, 222, 224, 228, 230 and 232.

In preferred embodiments, the antibody or antigen-binding fragment thereof competes for binding to hPCSK9 with an antibody comprising heavy and light chain CDR amino acid sequences as shown in SEQ ID NOs: 76, 78, 80, 84, 86, and 88 or as shown in SEQ ID NOs: 220, 222, 224, 228, 230 and 232.

The invention encompasses anti-PCSK9 antibodies having a modified glycosylation pattern. In some applications, modification to remove undesirable glycosylation sites may be useful, or e.g., removal of a fucose moiety to increase antibody dependent cellular cytotoxicity (ADCC) function (see Shield et al. (2002) JBC 277:26733). In other applications, modification of galactosylation can be made in order to modify complement dependent cytotoxicity (CDC).

Some preferred sequences related to preferred antibodies for practicing present invention:

```
SEQ ID NO: 76:
Gly Phe Thr Phe Asn Asn Tyr Ala
SEQ ID NO: 78:
Ile Ser Gly Ser Gly Gly Thr Thr
SEQ ID NO: 80:
Ala Lys Asp Ser Asn Trp Gly Asn Phe Asp Leu
SEQ ID NO: 84:
Gln Ser Val Leu Tyr Arg Ser Asn Asn Arg Asn Phe
SEQ ID NO: 86:
Trp Ala Ser
SEQ ID NO: 88:
Gln Gln Tyr Tyr Thr Thr Pro Tyr Thr
SEQ ID NO: 90:
Glu Val Gln Leu Val Glu Ser Gly Gly Gly Leu Val Gln Pro Gly Gly
  1 5 10 15
Ser Leu Arg Leu Ser Cys Ala Ala Ser Gly Phe Thr Phe Asn Asn Tyr
                        20 25 30
Ala Met Asn Trp Val Arg Gln Ala Pro Gly Lys Gly Leu Asp Trp Val
                35 40 45
Ser Thr Ile Ser Gly Ser Gly Gly Thr Thr Asn Tyr Ala Asp Ser Val
        50 55 60
Lys Gly Arg Phe Ile Ile Ser Arg Asp Ser Ser Lys His Thr Leu Tyr
65 70 75 80
Leu Gln Met Asn Ser Leu Arg Ala Glu Asp Thr Ala Val Tyr Tyr Cys
                                85 90 95
Ala Lys Asp Ser Asn Trp Gly Asn Phe Asp Leu Trp Gly Arg Gly Thr
                        100 105 110
Leu Val Thr Val Ser Ser
                115
SEQ ID NO: 92:
Asp Ile Val Met Thr Gln Ser Pro Asp Ser Leu Ala Val Ser Leu Gly
  1 5 10 15
Glu Arg Ala Thr Ile Asn Cys Lys Ser Ser Gln Ser Val Leu Tyr Arg
                        20 25 30
Ser Asn Asn Arg Asn Phe Leu Gly Trp Tyr Gln Gln Lys Pro Gly Gln
                35 40 45
Pro Pro Asn Leu Leu Ile Tyr Trp Ala Ser Thr Arg Glu Ser Gly Val
        50 55 60
Pro Asp Arg Phe Ser Gly Ser Gly Ser Gly Thr Asp Phe Thr Leu Thr
65 70 75 80
Ile Ser Ser Leu Gln Ala Glu Asp Val Ala Val Tyr Tyr Cys Gln Gln
                                85 90 95
Tyr Tyr Thr Thr Pro Tyr Thr Phe Gly Gln Gly Thr Lys Leu Glu Ile
                        100 105 110
Lys
SEQ ID NO: 755 (hPCSK9):
Met Gly Thr Val Ser Ser Arg Arg Ser Trp Trp Pro Leu Pro Leu Leu
  1 5 10 15
Leu Leu Leu Leu Leu Leu Leu Gly Pro Ala Gly Ala Arg Ala Gln Glu
                        20 25 30
Asp Glu Asp Gly Asp Tyr Glu Glu Leu Val Leu Ala Leu Arg Ser Glu
                35 40 45
Glu Asp Gly Leu Ala Glu Ala Pro Glu His Gly Thr Thr Ala Thr Phe
        50 55 60
His Arg Cys Ala Lys Asp Pro Trp Arg Leu Pro Gly Thr Tyr Val Val
65 70 75 80
Val Leu Lys Glu Glu Thr His Leu Ser Gln Ser Glu Arg Thr Ala Arg
                                85 90 95
Arg Leu Gln Ala Gln Ala Ala Arg Arg Gly Tyr Leu Thr Lys Ile Leu
                        100 105 110
His Val Phe His Gly Leu Leu Pro Gly Phe Leu Val Lys Met Ser Gly
                115 120 125
Asp Leu Leu Glu Leu Ala Leu Lys Leu Pro His Val Asp Tyr Ile Glu
        130 135 140
Glu Asp Ser Ser Val Phe Ala Gln Ser Ile Pro Trp Asn Leu Glu Arg
145 150 155 160
Ile Thr Pro Pro Arg Tyr Arg Ala Asp Glu Tyr Gln Pro Pro Asp Gly
                                165 170 175
Gly Ser Leu Val Glu Val Tyr Leu Leu Asp Thr Ser Ile Gln Ser Asp
                        180 185 190
His Arg Glu Ile Glu Gly Arg Val Met Val Thr Asp Phe Glu Asn Val
                195 200 205
Pro Glu Glu Asp Gly Thr Arg Phe His Arg Gln Ala Ser Lys Cys Asp
        210 215 220
Ser His Gly Thr His Leu Ala Gly Val Val Ser Gly Arg Asp Ala Gly
225 230 235 240
Val Ala Lys Gly Ala Ser Met Arg Ser Leu Arg Val Leu Asn Cys Gln
                                245 250 255
Gly Lys Gly Thr Val Ser Gly Thr Leu Ile Gly Leu Glu Phe Ile Arg
                        260 265 270
Lys Ser Gln Leu Val Gln Pro Val Gly Pro Leu Val Val Leu Leu Pro
                275 280 285
Leu Ala Gly Gly Tyr Ser Arg Val Leu Asn Ala Ala Cys Gln Arg Leu
        290 295 300
Ala Arg Ala Gly Val Val Leu Val Thr Ala Ala Gly Asn Phe Arg Asp
305 310 315 320
Asp Ala Cys Leu Tyr Ser Pro Ala Ser Ala Pro Glu Val Ile Thr Val
                                325 330 335
Gly Ala Thr Asn Ala Gln Asp Gln Pro Val Thr Leu Gly Thr Leu Gly
                        340 345 350
Thr Asn Phe Gly Arg Cys Val Asp Leu Phe Ala Pro Gly Glu Asp Ile
                355 360 365
Ile Gly Ala Ser Ser Asp Cys Ser Thr Cys Phe Val Ser Gln Ser Gly
        370 375 380
Thr Ser Gln Ala Ala Ala His Val Ala Gly Ile Ala Ala Met Met Leu
385 390 395 400
Ser Ala Glu Pro Glu Leu Thr Leu Ala Glu Leu Arg Gln Arg Leu Ile
                                405 410 415
His Phe Ser Ala Lys Asp Val Ile Asn Glu Ala Trp Phe Pro Glu Asp
                        420 425 430
Gln Arg Val Leu Thr Pro Asn Leu Val Ala Ala Leu Pro Pro Ser Thr
                435 440 445
His Gly Ala Gly Trp Gln Leu Phe Cys Arg Thr Val Trp Ser Ala His
        450 455 460
Ser Gly Pro Thr Arg Met Ala Thr Ala Val Ala Arg Cys Ala Pro Asp
465 470 475 480
Glu Glu Leu Leu Ser Cys Ser Ser Phe Ser Arg Ser Gly Lys Arg Arg
                                485 490 495
Gly Glu Arg Met Glu Ala Gln Gly Gly Lys Leu Val Cys Arg Ala His
                        500 505 510
Asn Ala Phe Gly Gly Glu Gly Val Tyr Ala Ile Ala Arg Cys Cys Leu
                515 520 525
Leu Pro Gln Ala Asn Cys Ser Val His Thr Ala Pro Pro Ala Glu Ala
        530 535 540
Ser Met Gly Thr Arg Val His Cys His Gln Gln Gly His Val Leu Thr
545 550 555 560
Gly Cys Ser Ser His Trp Glu Val Glu Asp Leu Gly Thr His Lys Pro
                                565 570 575
Pro Val Leu Arg Pro Arg Gly Gln Pro Asn Gln Cys Val Gly His Arg
                        580 585 590
Glu Ala Ser Ile His Ala Ser Cys Cys His Ala Pro Gly Leu Glu Cys
                595 600 605
Lys Val Lys Glu His Gly Ile Pro Ala Pro Gln Glu Gln Val Thr Val
        610 615 620
Ala Cys Glu Glu Gly Trp Thr Leu Thr Gly Cys Ser Ala Leu Pro Gly
625 630 635 640
Thr Ser His Val Leu Gly Ala Tyr Ala Val Asp Asn Thr Cys Val Val
                                645 650 655
Arg Ser Arg Asp Val Ser Thr Thr Gly Ser Thr Ser Glu Gly Ala Val
                        660 665 670
Thr Ala Val Ala Ile Cys Cys Arg Ser Arg His Leu Ala Gln Ala Ser
                675 680 685
Gln Glu Leu Gln
        690
```

### Preparation of Human Antibodies

Methods for generating human antibodies in transgenic mice are known (see for example, U.S. Pat. No. 6,596,541, Regeneron Pharmaceuticals, VELOCIMMUNE™). The VELOCIMMUNE™ technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody. In specific embodiment, the cell is a CHO cell.

Antibodies may be therapeutically useful in blocking a ligand-receptor interaction or inhibiting receptor component interaction, rather than by killing cells through fixation of complement and participation in complement-dependent cytotoxicity (CDC), or killing cells through antibody-dependent cell-mediated cytotoxicity (ADCC). The constant region of an antibody is thus important in the ability of an antibody to fix complement and mediate cell-dependent cytotoxicity. Thus, the isotype of an antibody may be selected on the basis of whether it is desirable for the antibody to mediate cytotoxicity.

Human antibodies can exist in two forms that are associated with hinge heterogeneity. In one form, an antibody molecule comprises a stable four-chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses antibodies having one or more mutations in the hinge, CH2 or CH3 region which may be desirable, for example, in production, to improve the yield of the desired antibody form.

Generally, a VELOCIMMUNE™ mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. As described below, the antibodies are characterized and selected for desirable characteristics, including affinity, selectivity, epitope, etc. The mouse constant regions are replaced with a desired human constant region to generate the fully human antibody of the invention, for example wild-type or modified IgG1 or IgG4 (for example, SEQ ID NO: 751, 752, 753). While the constant region selected may vary according to specific use, high affinity antigen-binding and target specificity characteristics reside in the variable region.

### Epitope Mapping and Related Technologies

To screen for antibodies that bind to a particular epitope (e.g., those which block binding of IgE to its high affinity receptor), a routine cross-blocking assay such as that described Antibodies, Harlow and Lane (Cold Spring Harbor Press, Cold Spring Harb., NY) can be performed. Other methods include alanine scanning mutants, peptide blots (Reineke (2004) Methods Mol Biol 248:443-63) (herein specifically incorporated by reference in its entirety), or peptide cleavage analysis. In addition, methods such as epitope excision, epitope extraction and chemical modification of antigens can be employed (Tomer (2000) Protein Science 9:487-496) (herein specifically incorporated by reference in its entirety).

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (US 2004/0101920, herein specifically incorporated by reference in its entirety). Each category may reflect a unique epitope either distinctly different from or partially overlapping with epitope represented by another category. This technology allows rapid filtering of genetically identical mAbs, such that characterization can be focused on genetically distinct mAbs. When applied to hybridoma screening, MAP may facilitate identification of rare hybridoma clones that produce mAbs having the desired characteristics. MAP may be used to sort the anti-PCSK9 mAbs of the invention into groups of mAbs binding different epitopes.

In various embodiments, the anti-hPCSK9 antibody or antigen-binding fragment of an antibody binds an epitope within the catalytic domain, which is about 153 to 425 of SEQ ID NO: 755); more specifically, an epitope from about 153 to about 250 or from about 250 to about 425; more specifically, the antibody or antibody fragment of the invention binds an epitope within the fragment from about 153 to about 208, from about 200 to about 260, from about 250 to about 300, from about 275 to about 325, from about 300 to about 360, from about 350 to about 400, and/or from about 375 to about 425.

In various embodiments, the anti-hPCSK9 antibody or antigen-binding fragment of an antibody binds an epitope within the propeptide domain (residues 31 to 152 of SEQ ID NO:755); more specifically, an epitope from about residue 31 to about residue 90 or from about residue 90 to about residue 152; more specifically, the antibody or antibody fragment of the invention binds an epitope within the fragment from about residue 31 to about residue 60, from about residue 60 to about residue 90, from about residue 85 to about residue 110, from about residue 100 to about residue 130, from about residue 125 to about residue 150, from about residue 135 to about residue 152, and/or from about residue 140 to about residue 152.

In some embodiments, the anti-hPCSK9 antibody or antigen-binding fragment of an antibody binds an epitope within the C-terminal domain, (residues 426 to 692 of SEQ ID NO: 755); more specifically, an epitope from about residue 426 to about residue 570 or from about residue 570 to about residue 692; more specifically, the antibody or antibody fragment of the invention binds an epitope within the fragment from about residue 450 to about residue 500, from about residue 500 to about residue 550, from about residue 550 to about residue 600, and/or from about residue 600 to about residue 692.

In some embodiments, the antibody or antibody fragment binds an epitope which includes more than one of the enumerated epitopes within the catalytic, propeptide or C-terminal domain, and/or within two or three different domains (for example, epitopes within the catalytic and C-terminal domains, or within the propeptide and catalytic domains, or within the propeptide, catalytic and C-terminal domains.

In some embodiments, the antibody or antigen-binding fragment binds an epitope on hPCSK9 comprising amino acid residue 238 of hPCSK9 (SEQ ID NO:755). Experimental results (see US 2010/0166768) showed that when D238 was mutated, the K_{D} of mAb 316P exhibited >400-fold reduction in binding affinity (˜1×10⁻⁹ M to ˜410×10⁻⁹ M) and T_{1/2} decreased >30-fold (from ˜37 to ˜1 min). In a specific embodiment, the mutation was D238R. In specific embodiments, the antibody or antigen-binding fragment of the invention binds an epitope of hPCSK9 comprising two or more of amino acid residues at positions 153, 159, 238 and 343.

As shown before (see US 2010/0166768), a mutation in amino acid residue 153, 159 or 343 resulted in about a 5- to 10-fold decrease in affinity or similar shortening in T_{1/2}. In specific embodiments, the mutation was S153R, E159R and/or D343R.

In some embodiments, the antibody or antigen-binding fragment binds an epitope on hPCSK9 comprising amino acid residue 366 of hPCSK9 (SEQ ID NO:755). Experimental results (see US 2010/0166768) showed that when E366 was mutated, the affinity of mAb 300N exhibited about 50-fold decrease (˜0.7×10⁻⁹ M to ˜36×10⁻⁹ M) and a similar shortening in T_{1/2} (from ˜120 to ˜2 min). In a specific embodiment, the mutation is E366K.

The present invention includes anti-PCSK9 antibodies that bind to the same epitope as any of the specific exemplary antibodies described herein. Likewise, the present invention also includes anti-PCSK9 antibodies that compete for binding to PCSK9 or a PCSK9 fragment with any of the specific exemplary antibodies described herein.

One can easily determine whether an antibody binds to the same epitope as, or competes for binding with, a reference anti-PCSK9 antibody by using routine methods known in the art. For example, to determine if a test antibody binds to the same epitope as a reference anti-PCSK9 antibody of the invention, the reference antibody is allowed to bind to a PCSK9 protein or peptide under saturating conditions. Next, the ability of a test antibody to bind to the PCSK9 molecule is assessed. If the test antibody is able to bind to PCSK9 following saturation binding with the reference anti-PCSK9 antibody, it can be concluded that the test antibody binds to a different epitope than the reference anti-PCSK9 antibody. On the other hand, if the test antibody is not able to bind to the PCSK9 molecule following saturation binding with the reference anti-PCSK9 antibody, then the test antibody may bind to the same epitope as the epitope bound by the reference anti-PCSK9 antibody of the invention.

To determine if an antibody competes for binding with a reference anti-PCSK9 antibody, the above-described binding methodology is performed in two orientations: In a first orientation, the reference antibody is allowed to bind to a PCSK9 molecule under saturating conditions followed by assessment of binding of the test antibody to the PCSK9 molecule. In a second orientation, the test antibody is allowed to bind to a PCSK9 molecule under saturating conditions followed by assessment of binding of the reference antibody to the PCSK9 molecule. If, in both orientations, only the first (saturating) antibody is capable of binding to the PCSK9 molecule, then it is concluded that the test antibody and the reference antibody compete for binding to PCSK9. As will be appreciated by a person of ordinary skill in the art, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

In a specific embodiment, the invention comprises an anti-PCSK9 antibody or antigen binding fragment of an antibody that binds an PCSK9 protein of SEQ ID NO:755, wherein the binding between the antibody or fragment thereof to PCSK9 and a variant PCSK9 protein is less than 50% of the binding between the antibody or fragment and the PCSK9 protein of SEQ ID NO: 755. In one specific embodiment, the variant PCSK9 protein comprises at least one mutation of a residue at a position selected from the group consisting of 153, 159, 238 and 343. In a more specific embodiment, the at least one mutation is S153R, E159R, D238R, and/or D343R. In another specific embodiment, the variant PCSK9 protein comprises at least one mutation of a residue at a position selected from the group consisting of 366. In one specific embodiment, the variant PCSK9 protein comprises at least one mutation of a residue at a position selected from the group consisting of 147, 366 and 380. In a more specific embodiment, the mutation is S147F, E366K and V380M.

### Immunoconjugates

The invention encompasses a human anti-PCSK9 monoclonal antibody conjugated to a therapeutic moiety (“immunoconjugate”), such as a cytotoxin, a chemotherapeutic drug, an immunosuppressant or a radioisotope. Cytotoxin agents include any agent that is detrimental to cells. Examples of suitable cytotoxin agents and chemotherapeutic agents for forming immunoconjugates are known in the art, see for example, WO 05/103081.

### Bispecifics

The antibodies of the present invention may be monospecific, bispecific, or multispecific. Multispecific mAbs may be specific for different epitopes of one target polypeptide or may contain antigen-binding domains specific for more than one target polypeptide. See, e.g., Tutt et al. (1991) J. Immunol. 147:60-69. The human anti-PCSK9 mAbs can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment, to produce a bispecific or a multispecific antibody with a second binding specificity.

An exemplary bi-specific antibody format that can be used in the context of the present invention involves the use of a first immunoglobulin (Ig) CH3 domain and a second Ig CH3 domain, wherein the first and second Ig CH3 domains differ from one another by at least one amino acid, and wherein at least one amino acid difference reduces binding of the bispecific antibody to Protein A as compared to a bi-specific antibody lacking the amino acid difference. In one embodiment, the first Ig CH3 domain binds Protein A and the second Ig CH3 domain contains a mutation that reduces or abolishes Protein A binding such as an H95R modification (by IMGT exon numbering; H435R by EU numbering). The second CH3 may further comprise a Y96F modification (by IMGT; Y436F by EU). Further modifications that may be found within the second CH3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies. Variations on the bi-specific antibody format described above are contemplated within the scope of the present invention.

### Bioequivalents

The anti-PCSK9 antibodies and antibody fragments of the present invention encompass proteins having amino acid sequences that vary from those of the described mAbs, but that retain the ability to bind human PCSK9. Such variant mAbs and antibody fragments comprise one or more additions, deletions, or substitutions of amino acids when compared to parent sequence, but exhibit biological activity that is essentially equivalent to that of the described mAbs. Likewise, the anti-PCSK9 antibody-encoding DNA sequences of the present invention encompass sequences that comprise one or more additions, deletions, or substitutions of nucleotides when compared to the disclosed sequence, but that encode an anti-PCSK9 antibody or antibody fragment that is essentially bioequivalent to an anti-PCSK9 antibody or antibody fragment of the invention. Examples of such variant amino acid and DNA sequences are discussed above.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied. In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-PCSK9 antibodies of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation.

### Treatment Population

The invention provides therapeutic methods for treating a human patient in need of a composition of the invention. While modifications in lifestyle and conventional drug treatment are often successful in reducing cholesterol levels, not all patients are able to achieve the recommended target cholesterol levels with such approaches. Various conditions, such as familial hypercholesterolemia (FH), appear to be resistant to lowering of LDL-C levels in spite of aggressive use of conventional therapy. Homozygous and heterozygous familial hypercholesterolemia (hoFH, heFH) is a condition associated with premature atherosclerotic vascular disease. However, patients diagnosed with hoFH are largely unresponsive to conventional drug therapy and have limited treatment options. Specifically, treatment with statins, which reduce LDL-C by inhibiting cholesterol synthesis and upregulating the hepatic LDL receptor, may have little effect in patients whose LDL receptors are non-existent or defective. A mean LDL-C reduction of only less than about 20% has been recently reported in patients with genotype-confirmed hoFH treated with the maximal dose of statins. The addition of ezetimibe 10 mg/day to this regimen resulted in a total reduction of LDL-C levels of 27%, which is still far from optimal. Likewise, many patients are statin non-responsive, poorly controlled with statin therapy, or cannot tolerate statin therapy; in general, these patients are unable to achieve cholesterol control with alternative treatments. There is a large unmet medical need for new treatments that can address the short-comings of current treatment options.

Specific populations treatable by the therapeutic methods of the invention include subjects indicated for LDL apheresis, subjects with PCSK9-activating mutations (gain of function mutations, “GOF”), subjects with heterozygous Familial Hypercholesterolemia (heFH); subjects with primary hypercholesterolemia who are statin intolerant or statin uncontrolled; and subjects at risk for developing hypercholesterolemia who may be preventably treated. Other indications include hyperlipidemia and dyslipidemia associated with secondary causes such as Type 2 diabetes mellitus, cholestatic liver diseases (primary biliary cirrhosis), nephrotic syndrome, hypothyroidism, obesity; and the prevention and treatment of atherosclerosis and cardiovascular diseases. However, depending on the severity of the afore-mentioned diseases and conditions, the treatment of subjects with the antibodies and antigen-binding fragments of the invention may be contraindicated for certain diseases and conditions.

### Therapeutic Administration and Formulations

The invention provides therapeutic compositions comprising the anti-PCSK9 antibodies or antigen-binding fragments thereof of the present invention. The administration of therapeutic compositions in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose may vary depending upon the age and the size of a subject to be administered, target disease, conditions, route of administration, and the like. When the antibody of the present invention is used for treating various conditions and diseases associated with PCSK9, including hypercholesterolemia, disorders associated with LDL and apolipoprotein B, and lipid metabolism disorders, and the like, in an adult patient, it is advantageous to intravenously administer the antibody of the present invention normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted.

Various delivery systems are known and can be used to administer the pharmaceutical compositions of the invention, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, intramuscular, intraperitoneal, intravenous, subcutaneous, intranasal, epidural, and oral or peroral routes. If the antibody of present invention is administered per injection, subcutaneous injection is preferred. Oral or peroral administration is preferred for the HMG-COA inhibitor, e.g. the statin.

The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can be also delivered in a vesicle, in particular a liposome (see Langer (1990) Science 249:1527-1533; Treat et al. (1989) in Liposomes in the Therapy of Infectious Disease and Cancer, Lopez Berestein and Fidler (eds.), Liss, New York, pp. 353-365; Lopez-Berestein, ibid., pp. 317-327; see generally ibid.).

In certain situations, the pharmaceutical composition can be delivered in a controlled release system. In one embodiment, a pump may be used (see Langer, supra; Sefton (1987) CRC Crit. Ref. Biomed. Eng. 14:201). In another embodiment, polymeric materials can be used; see, Medical Applications of Controlled Release, Langer and Wise (eds.), CRC Pres., Boca Raton, Florida (1974). In yet another embodiment, a controlled release system can be placed in proximity of the composition's target, thus requiring only a fraction of the systemic dose (see, e.g., Goodson, in Medical Applications of Controlled Release, supra, vol. 2, pp. 115-138, 1984).

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule. A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but certainly are not limited to the SOLOSTAR™ pen (sanofi-aventis), the FLEXPEN™ (Novo Nordisk), and the KWIKPEN™ (Eli Lilly).

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 4 to about 500 mg or from about, from about 5 to about 500 mg per dosage form in a unit dose; especially in the form of injection, it is preferred that the aforesaid antibody is contained in about 5 to about 100 mg or about 5 to 400 mg (such as from about 50 to about 200 mg per 1 ml injection solution) and in about 10 to about 250 mg or to about 500 mg for the other dosage forms.

The invention provides therapeutic methods in which the antibody or antibody fragment of the invention is useful to treat hypercholesterolemia associated with a variety of conditions involving hPCSK9. The anti-PCSK9 antibodies or antibody fragments of the invention are particularly useful for the treatment of hypercholesterolemia and the like. Combination therapies may include the anti-PCSK9 antibody of the invention with, for example, one or more of any agent that (1) induces a cellular depletion of cholesterol synthesis by inhibiting 3-hydroxy-3-methylglutaryl (HMG)-coenzyme A (CoA) reductase, such as cerivastatin, atorvastatin, simvastatin, pitavastatin, rosuvastatin, fluvastatin, lovastatin, pravastatin; (2) inhibits cholesterol uptake and or bile acid re-absorption; (3) increase lipoprotein catabolismosuch as niacin); and activators of the LXR transcription factor that plays a role in cholesterol elimination such as 22-hydroxycholesterol or fixed combinations such as ezetimibe plus simvastatin; a statin with a bile resin (e.g., cholestyramine, colestipol, colesevelam), a fixed combination of niacin plus a statin (e.g., niacin with lovastatin); or with other lipid lowering agents such as omega-3-fatty acid ethyl esters (for example, omacor).

### Preferred Aspects of Present Invention

In the following, some preferred aspects and embodiments of present invention will be listed:

### Aspects Related to Patient Populations—A)

1. A method for treating a disease or condition in which PCSK9 expression or activity causes an impact comprising
   administering a therapeutic amount of an antibody or an antigen-binding fragment thereof which specifically binds hPCSK9 (human proprotein convertase subtilisin/kexin type 9) to a subject in need thereof,
   wherein the subject in need thereof falls into one or more of the following groups of subjects:
   (i) subjects having a serum LDL cholesterol (LDL-C) level of at least 100 mg/dL, [at least 130 mg/dL, at least 160 mg/dL/at least 200 mg/dL];
   (ii) subjects having a serum HDL-C level of less than 40 mg/dL;
   (iii) subjects having a serum cholesterol level of at least 200 mg/dL [240 mg/dL];
   (iv) subjects having a serum triacylglycerol level of at least 150 mg/dL [at least 200 mg/dL; at least 500 mg/dL], wherein said triacylglycerol level is determined after fasting for at least 8 hours;
   (v) subjects being at least 35 years old [at least 40/50/55/60/65/70 years old];
   (vi) subjects younger than 75 years [65/60/55/50/45/40 years];
   (vii) subjects having a BMI of 25 [26/27/28/29/30/31/32/33/34/35/36/37/38/39] or more;
   (viii) male subjects;
   (ix) female subjects;
   (x) subjects in which the administration of said antibody or antigen-binding fragment thereof leads to a reduction in the serum LDL-C level by at least 30 mg/dL [40 mg/dL; 50 mg/dL; 60 mg/dL; 70 mg/dL] relative to predose level; or
   (xi) subjects in which the administration of said antibody or antigen-binding fragment thereof leads to a reduction in the serum LDL-C level by at least 20% [30%; 40%; 50%; 60%] relative to predose level.
2. A method for treating a disease or condition in which PCSK9 expression or activity causes an impact comprising
   administering a therapeutic amount of an antibody or an antigen-binding fragment thereof which specifically binds hPCSK9 (human proprotein convertase subtilisin/kexin type 9) to a subject in need thereof,
   wherein the subject in need thereof does not fall into one or more of the following groups of subjects:
   (i) smokers;
   (ii) persons being 70 years old or older;
   (iii) persons suffering from hypertension;
   (iv) women who are pregnant;
   (v) women who are trying to become pregnant;
   (vi) women who are breast-feeding;
   (vii) persons who have or ever had a disease affecting the liver;
   (viii) persons who had any unexplained abnormal blood tests for liver function;
   (ix) persons who drink excessive amounts of alcohol;
   (x) persons having kidney problems;
   (xi) persons suffering from hypothyroidism;
   (xii) persons suffering from muscle disorders;
   (xiii) persons having encountered previous muscular problems during treatment with lipid-lowering medicine;
   (xiv) persons having serious problems with their breathing;
   (xv) persons taking one or more of the following medicines: medicines altering the way the immune systems works (e.g. ciclosporin or antihistamines), antibiotics or antifungal medicines (e.g. erythromycin, clarithromycin, ketoconazole, itraconazole, rifampicin, fusidic acid), medicines regulating lipid levels (e.g. gemfibrozil, colestipol), calcium channel blockers (e.g. verapamil, diltiazem), medicines regulating the heart rhythm (digoxin, amiodarone), protease inhibitors used in the treatment of HIV (e.g. nelfinavir), warfarin, oral contraceptives, antacids or St. John's Wort; or
   (xvi) persons drinking more than 0.1 L of grapefruit juice per day;
   (xvii) persons having a body mass index (BMI) of more than 40;
   (xviii) persons having a body mass index (BMI) of less than 18;
   (xix) persons suffering from type 1 diabetes or type 2 diabetes;
   (xx) persons positive for hepatitis B or hepatitis C; or
   (xxi) persons having a known sensitivity to monoclonal antibody therapeutics.
3. An antibody or an antigen-binding fragment thereof which specifically binds hPCSK9 (human proprotein convertase subtilisin/kexin type 9) for use in the treatment of a disease or condition in which PCSK9 expression or activity causes an impact,
   wherein the antibody or antigen-binding fragment thereof is for administration to a subject falling at least into one of the following groups of subjects:
   (i) subjects having a serum LDL-C level of at least 100 mg/dL [at least 130 mg/dL/at least 160 mg/dL/at least 200 mg/dL];
   (ii) subjects having a serum HDL-C level of less than 40 mg/dL;
   (iii) subjects having a serum cholesterol level of at least 200 mg/dL [240 mg/dL];
   (iv) subjects having a serum triacylglycerol level of at least 150 mg/dL [at least 200 mg/dL; at least 500 mg/dL], wherein said triacylglycerol level is determined after fasting for at least 8 hours;
   (v) subjects being at least 35 years old [at least 40/50/55/60/65/70 years old];
   (vi) subjects younger than 75 years [65/60/55/50/45/40 years];
   (vii) subjects having a BMI of 25 [26/27/28/29/30/31/32/33/34/35/36/37/38/39] or more;
   (viii) male subjects;
   (ix) female subjects;
   (x) subjects in which the administration of said antibody or antigen-binding fragment thereof leads to a reduction in the serum LDL-C level by at least 20 mg/dl [30 mg/dL; 40 mg/dL; 50 mg/dL; 60 mg/dL; 70 mg/dL]; or
   (xi) subjects in which the administration of said antibody or antigen-binding fragment thereof leads to a reduction in the serum LDL-C level by at least 10% [20%; 30%; 40%; 50%; 60%].
4. An antibody or an antigen-binding fragment thereof which specifically binds hPCSK9 (human proprotein convertase subtilisin/kexin type 9) for use in the treatment of a disease or condition in which PCSK9 expression or activity causes an impact, wherein the antibody or antigen-binding fragment thereof is for administration to a subject who does not fall into one or more of the following groups of subjects:
   (i) smokers;
   (ii) persons being 70 years old or older;
   (iii) persons suffering from hypertension;
   (iv) women who are pregnant;
   (v) women who are trying to become pregnant;
   (vi) women who are breast-feeding;
   (vii) persons who have or ever had a disease affecting the liver;
   (viii) persons who had any unexplained abnormal blood tests for liver function;
   (ix) persons who drink excessive amounts of alcohol;
   (x) persons having kidney problems;
   (xi) persons suffering from hypothyroidism;
   (xii) persons suffering from muscle disorders;
   (xiii) persons having encountered previous muscular problems during treatment with lipid-lowering medicine;
   (xiv) persons having serious problems with their breathing;
   (xv) persons taking one or more of the following medicines: medicines altering the way the immune systems works (e.g. ciclosporin or antihistamines), antibiotics or antifungal medicines (e.g. erythromycin, clarithromycin, ketoconazole, itraconazole, rifampicin, fusidic acid), medicines regulating lipid levels (e.g. gemfibrozil, colestipol), calcium channel blockers (e.g. verapamil, diltiazem), medicines regulating the heart rhythm (digoxin, amiodarone), protease inhibitors used in the treatment of HIV (e.g. nelfinavir), warfarin, oral contraceptives, antacids or St. John's Wort;
   (xvi) persons drinking more than 0.1 L of grapefruit juice per day;
   (xvii) persons having a body mass index (BMI) of more than 40;
   (xviii) persons having a body mass index (BMI) of less than 18;
   (xix) persons suffering from type 1 diabetes or type 2 diabetes;
   (xx) persons positive for hepatitis B or hepatitis C; or
   (xxi) persons having a known sensitivity to monoclonal antibody therapeutics.
5. The method of aspect 1 or 2 or the antibody of aspect 2 or 3, wherein the disease or condition in which PCSK9 expression or activity causes an impact is ameliorated, improved, inhibited or prevented with a PCSK9 antagonist.
6. The method or the antibody of any one of aspects 1 to 5, wherein the disease or condition in which PCSK9 expression or activity causes an impact is selected from the group consisting of:
   hypercholesterolemia, hyperlipidemia, dyslipidemia, atherosclerosis and cardiovascular diseases.
7. The method or the antibody of any one of aspects 1 to 6, wherein the subject in need thereof is a subject indicated for LDL apheresis, a subject with PCSK9-activating mutations, a subject with heterozygous Familial Hypercholesterolemia, a subject with primary hypercholesterolemia who is statin uncontrolled, a subject at risk for developing hypercholesterolemia, a subject with hypercholesterolemia, hyperlipidemia, dyslipidemia, atherosclerosis or cardiovascular diseases.
8. The method or the antibody of any one of aspects 1 to 7, wherein the antibody or antigen-binding fragment thereof is a recombinant human antibody or fragment thereof.
9. The method or the antibody of any one of aspects 1 to 8, wherein the antibody or the antigen-binding fragment thereof is characterized by one or more of the following:
   (i) capable of reducing serum total cholesterol at least about 25 to about 35% and sustaining the reduction over at least a 24 day period relative to a predose level;
   (ii) capable of reducing serum LDL cholesterol at least about 65-80% and sustaining the reduction over at least a 24 day period relative to a predose level;
   (iii) capable of reducing serum triglyceride at least about 25-40% relative to predose level;
   (iv) achieves one or more of (i)-(iii) without reducing serum HDL cholesterol or reducing serum HDL cholesterol no more than 5% relative to predose level;
   (v) achieves one or more of (i)-(iii) with little or no measurable effect on liver function, as determined by ALT and AST measurements.
10. The method or the antibody of any one of aspects 1 to 9, wherein the antibody or the antigen-binding fragment thereof comprises
   a heavy chain CDR3 (HCDR3) domain selected from the group consisting of SEQ ID NO: 8, 32, 56, 80, 104, 128, 152, 176, 200, 224, 248, 272, 296, 320, 344, 368, 392, 416, 440, 464, 488, 512, 536, 560, 584, 608, 632, 656, 680, 704 and 728; and
   a light chain CDR3 (LCDR3) domain selected from the group consisting of SEQ ID NO: 16, 40, 64, 88, 112, 136, 160, 184, 208, 232, 256, 280, 304, 328, 352, 376, 400, 424, 448, 472, 496, 520, 544, 568, 592, 616, 639, 664, 688, 712 and 736.
11. The method or the antibody of any one of aspects 1 to 9, wherein the antibody or the antigen-binding fragment thereof comprises the heavy and light chain CDRs of a HCVR/LCVR amino acid sequence pair as shown in SEQ ID NOs: 90/92.
12. The method or the antibody of aspect 11, wherein the antibody or antigen-binding fragment thereof comprises heavy and light chain CDR amino acid sequences as shown in SEQ ID NOs: 76, 78, 80, 84, 86, and 88.
13. The method or the antibody of aspect 12, wherein the antibody or antigen-binding fragment thereof comprises an HCVR amino acid sequence as shown in SEQ ID NO: 90 and an LCVR amino acid sequence as shown in SEQ ID NO: 92.
14. The method or the antibody of any one of aspects 1 to 9, wherein the antibody or antigen-binding fragment thereof binds to the same epitope on hPCSK9 as an antibody comprising heavy and light chain CDR amino acid sequences as shown in SEQ ID NOs: 76, 78, 80, 84, 86, and 88.
15. The method or the antibody of any one of aspects 1 to 9, wherein the antibody or antigen-binding fragment thereof competes for binding to hPCSK9 with an antibody comprising heavy and light chain CDR amino acid sequences as shown in SEQ ID NOs: 76, 78, 80, 84, 86, and 88.
16. The method or the antibody of any one of aspects 1 to 15, further comprising:
   administering a therapeutic amount of an HMG-COA reductase inhibitor to the subject in a dosage of between 0.05 mg to 100 mg.
17. The method or the antibody of aspect 16, wherein the HMG-COA reductase inhibitor is a statin.
18. The method or the antibody of aspect 17, wherein the statin is selected from the group consisting of cerivastatin, atorvastatin, simvastatin, pitavastatin, rosuvastatin, fluvastatin, lovastatin, and pravastatin.
19. The method or the antibody of aspect 18, wherein the statin is
   cerivastatin administered in a daily dosage of between 0.05 mg and 2 mg;
   atorvastatin administered in a daily dosage of between 2 mg and 100 mg;
   simvastatin administered in a daily dosage of between 2 mg and 100 mg;
   pitavastatin administered in a daily dosage of between 0.2 mg and 100 mg;
   rosuvastatin administered in a daily dosage of between 2 mg and 100 mg;
   fluvastatin administered in a daily dosage of between 2 mg and 100 mg;
   lovastatin administered in a daily dosage of between 2 mg and 100 mg; or
   pravastatin administered in a daily dosage of between 2 mg and 100 mg.
20. An article of manufacture comprising
   (a) a packaging material;
   (b) an antibody or an antigen-binding fragment thereof which specifically binds hPCSK9; and
   (c) a label or packaging insert contained within the packaging material indicating that patients receiving treatment with said antibody or antigen-binding fragment can be treated for a disease or condition selected from the group consisting of hypercholesterolemia, hyperlipidemia, dyslipidemia, atherosclerosis and cardiovascular diseases and further indicating that subjects falling into one or more groups of subjects as recited in aspect 1 can be treated.
21. An article of manufacture comprising
   (a) a packaging material;
   (b) an antibody or an antigen-binding fragment thereof which specifically binds hPCSK9; and
   (c) a label or packaging insert contained within the packaging material indicating that patients receiving treatment with said antibody or antigen-binding fragment can be treated for a disease or condition selected from the group consisting of hypercholesterolemia, hyperlipidemia, dyslipidemia, atherosclerosis and cardiovascular diseases and further indicating that the treatment of patients with said antibody or antigen-binding fragment thereof is contraindicated for patients belonging to one or more groups of subjects as recited in aspect 2.
22. The article of manufacture according to aspect 20 or 21, wherein the antibody or antigen-binding fragment is an antibody or antigen-binding fragment as specified in any of aspects 3 to 19.
23. The article of manufacture according to any of aspects 20 to 22, wherein the label or packaging insert contains reference to a method of treatment according to any of aspects 1, 2 or 5-19.
24. A method of testing the efficacy of an antibody or an antigen-binding fragment thereof which specifically binds hPCSK9 for the treatment of a disease or condition selected from the group consisting of hypercholesterolemia, hyperlipidemia, dyslipidemia, atherosclerosis and cardiovascular diseases, said method comprising:
   treating a selected patient population with said antibody or antigen-binding fragment thereof, wherein each patient in said population has an LDL cholesterol (LDL-C) level of more than 100 mg/dL; and
   determining the efficacy of said antibody or antigen-binding fragment thereof by determining the LDL-C level in the patient population before and after administration of said antibody or antigen-binding fragment thereof, wherein a reduction of the LDL-C level by at least 25% relative to a predose level in at least 75% of the patient population indicates that said antibody or antigen-binding fragment thereof is efficacious for the treatment of said disease or condition in said patient population;
   wherein each patient falls into one or more groups of subjects as recited in aspect 1.
25. A method of testing the efficacy of an antibody or an antigen-binding fragment thereof which specifically binds hPCSK9 for the treatment of a disease or condition selected from the group consisting of hypercholesterolemia, hyperlipidemia, dyslipidemia, atherosclerosis and cardiovascular diseases, said method comprising:
   determining the efficacy of an antibody or antigen-binding fragment thereof that has been used for the treatment of a selected patient population with said antibody or antigen-binding fragment thereof, wherein each patient in said population has an LDL cholesterol (LDL-C) level of more than 100 mg/dL by determining the LDL-C level in the patient population before and after administration of said antibody or antigen-binding fragment thereof, wherein a reduction of the LDL-C level by at least 25% relative to a predose level in at least 75% of the patient population indicates that said antibody or antigen-binding fragment thereof is efficacious for the treatment of said disease or condition in said patient population;
   wherein each patient falls into one or more groups of subjects as recited in aspect 1.
26. The method of aspect 25, wherein each patient in said population has received a lipid lowering treatment by administration of a statin for at least 6 weeks prior to treatment with said antibody or antigen-binding fragment thereof.
27. The method of any of aspects 24 to 26, wherein the antibody or antigen-binding fragment is an antibody or antigen-binding fragment as specified in any of aspects 3 to 19.
28. The method of any of aspects 24 to 27, wherein the selected patient population is or has been treated with a method of treatment according to any of aspects 1, 2 or 5-19.
29. A method for testing the efficacy of a compound in lowering cholesterol levels in a subject, comprising the steps:
   (a) providing a rodent;
   (b) administering an antibody or an antigen-binding fragment thereof which specifically binds PCSK9 to the rodent;
   (c) administering a test compound to said rodent;
   (d) determining the effect of the test compound in the rodent, wherein a lowering of the cholesterol level in the rodent as compared to the cholesterol level of a control animal indicates that the test compound is efficacious in lowering cholesterol levels in a subject, wherein the control animal is from the same species as said rodent, and wherein the control animal has not been challenged with the test compound.

### Aspects Related to Patient Populations—B)

1. A method of treating a subject suffering from a disease or disorder characterized by elevated low-density lipoprotein cholesterol (LDL-C) levels, the method comprising:
(a) selecting a subject with a blood LDL-C level greater than 100 mg/dL; and
(b) administering to said subject a composition comprising an antibody or antigen binding fragment thereof that specifically binds to human proprotein convertase subtilisin/kexin type 9 (hPCSK9); thereby lowering cholesterol levels in the subject in need thereof.
   2. The method of aspect 1, wherein the disease or condition is selected from the group consisting of: hypercholesterolemia, hyperlipidemia, dyslipidemia, and atherosclerosis.
   3. The method of aspect 1, wherein the disease condition is primary hypercholesterolemia or familial hypercholesterolemia.
   4. The method of aspect 1, wherein the disease or condition is hypercholesterolemia which is uncontrolled by statins.
   5. The method of aspect 1, wherein the subject has a body mass index (BMI) of less than 18 kg/m² or greater than 40 kg/m².
   6. The method of aspect 1, wherein the subject was not previously instructed to partake in a cholesterol-lowering diet.
   7. The method of aspect 1, wherein the subject has not previously taken a cholesterol-lowering drug except for atorvastatin.
   8. The method of aspect 7, wherein the atorvastatin was administered at about 10 mg per day.
   9 The method of aspect 7, wherein the cholesterol-lowering drug is selected from the group consisting of fibrates, bile acid resins, niacin, intestinal cholesterol absorption (ICA) blockers, and omega-3 fatty acids.
   10. The method of aspect 9, wherein the niacin is administered at greater than 500 mg per day.
   11. The method of aspect 9, wherein the omega-3 fatty acids are administered at greater than 1000 mg per day.
   12. The method of aspect 1, wherein the subject does not suffer from diabetes.
   13. The method of aspect 12, wherein the diabetes is type 1 diabetes.
   14. The method of aspect 12, wherein the diabetes is type 2 diabetes.
   15. The method of aspect 12, wherein the type 2 diabetes is treated with insulin.
   16. The method of aspect 12, wherein the subject has a blood glycated hemoglobin concentration greater than or equal to 8.5%.
   17. The method of aspect 1, wherein the subject is negative for hepatitis B and C surface antigen.
   18. The method of aspect 1, wherein the subject has a blood triglycerides concentration of greater than 350 mg/dL.
   19. The method of aspect 1, wherein the subject has fewer than 1500 neutrophils per cubic mm of blood.
   20. The method of aspect 1, wherein the subject has fewer than 100,000 platelets per cubic mm of blood.
   21. The method of aspect 1, wherein the subject is female.
   22. The method of aspect 21, wherein the subject is not pregnant.
   23. The method of aspect 1, wherein the subject has a blood thyroid stimulating hormone concentration that is above the lower limit of normal and below the upper limit of normal.
   24. The method of aspect 23, wherein the subject has serum creatine of less than 1.4 of the upper limit of normal.
   25. The method of aspect 1, wherein the subject is a male.
   26. The method of aspect 25, wherein the subject has serum creatine of less than 1.5 of the upper limit of normal.
   27. The method of aspect 1, wherein the subject has an amount of aspartate transaminase that is less than two times the upper limit of normal.
   28. The method of aspect 1, wherein the subject has an amount of alanine transaminase that is less than two times the upper limit of normal.
   29. The method of aspect 1, wherein the antibody or antigen-binding fragment is administered in a dosage amount within the range of about 5 mg to about 500 mg.
   30. The method of aspect 29, wherein the antibody or antigen-binding fragment is administered in a dosage amount within the range of about 50 mg to about 300 mg.
   31. The method of aspect 29, wherein the antibody is administered at between 200 and 300 mg every four weeks.
   32. The method of aspect 29, wherein the antibody or antigen-binding fragment is administered in a dosage amount of about 150 mg.
   33. The method of aspect 1, wherein the antibody or antigen-binding fragment thereof is administered to the subject every other week (E2W).
   34. The method of aspect 1, wherein the antibody or antigen-binding fragment thereof is administered to the subject every fourth week (E4W).
   35. The method of aspect 1, wherein the antibody or the antigen-binding fragment comprises the heavy and light chain CDRs of a HCVR/LCVR amino acid sequence pair as shown in SEQ ID NOs: 90/92.
   36. The method of aspect 1, wherein the antibody or antigen-binding fragment comprises a HCVR/LCVR amino acid sequence pair as shown in SEQ ID NOs: 90/92.
   37. The method of aspect 1, wherein the antibody or antigen-binding fragment thereof competes for binding to hPCSK9 with an antibody or antigen-binding fragment comprising a HCVR/LCVR amino acid sequence pair as shown in SEQ ID NOs: 90/92.
   38. The method of aspect 1, wherein the antibody is administered subcutaneously.
   39. The method of aspect 38, wherein the antibody is administered in the abdomen.
   40. The method of aspect 1, further comprising administering a HMG-COA reductase inhibitor to the subject.
   41. The method of aspect 40, wherein the HMG-COA reductase inhibitor is administered in a dosage amount in the range of about 0.05 mg to 100 mg.
   42. The method of aspect 41, wherein the HMG-COA reductase inhibitor is a statin.
   43. The method of aspect 42, wherein the statin is selected from the group consisting of cerivastatin, atorvastatin, simvastatin, pitavastatin, rosuvastatin, fluvastatin, lovastatin, and pravastatin.
   44. The method of aspect 42, wherein the statin is atorvastatin administered at a dosage of 10 mg or 80 mg.
   45. The method of aspect 44, wherein the atorvastatin is administered at about 10 mg per day and at 80 mg one day in an 8 week period.
   46. A method of lowering cholesterol levels in a subject in need thereof, comprising:
(a) selecting a subject with a blood low density lipoprotein cholesterol (LDL-C) level greater than 100 mg/dL; and
(b) administering to said subject a composition comprising an antibody or antigen binding fragment thereof that specifically binds to human proprotein convertase subtilisin/kexin type 9 (hPCSK9); thereby lowering cholesterol levels in the subject in need thereof.
   47. The method of aspect 46, wherein the disease or condition is selected from the group consisting of: hypercholesterolemia, hyperlipidemia, dyslipidemia, and atherosclerosis.
   48. The method of aspect 46, wherein the disease condition is primary hypercholesterolemia or familial hypercholesterolemia.
   49. The method of aspect 46, wherein the disease or condition is hypercholesterolemia which is uncontrolled by statins.
   50. The method of aspect 46, wherein the subject has a body mass index (BMI) of less than 18 kg/m² or greater than 40 kg/m².
   51. The method of aspect 46, wherein the subject was not previously instructed to partake in a cholesterol-lowering diet.
   52. The method of aspect 46, wherein the subject has not previously taken a cholesterol-lowering drug except for atorvastatin.
   53. The method of aspect 52, wherein the atorvastatin was administered at about 10 mg per day.
   54. The method of aspect 52, wherein the cholesterol-lowering drug is selected from the group consisting of fibrates, bile acid resins, niacin, intestinal cholesterol absorption (ICA) blockers, and omega-3 fatty acids.
   55. The method of aspect 54, wherein the niacin is administered at greater than 500 mg per day.
   56. The method of aspect 54, wherein the omega-3 fatty acids are administered at greater than 1000 mg per day.
   57. The method of aspect 46, wherein the subject does not suffer from diabetes.
   58. The method of aspect 57, wherein the diabetes is type 1 diabetes.
   59. The method of aspect 57, wherein the diabetes is type 2 diabetes.
   60. The method of aspect 57, wherein the type 2 diabetes is treated with insulin.
   61. The method of aspect 57, wherein the subject has a blood glycated hemoglobin concentration greater than or equal to 8.5%.
   62. The method of aspect 46, wherein the subject is negative for hepatitis B and C surface antigen.
   63. The method of aspect 46, wherein the subject has a blood triglycerides concentration of greater than 350 mg/dL.
   64. The method of aspect 46, wherein the subject has fewer than 1500 neutrophils per cubic mm of blood.
   65. The method of aspect 46, wherein the subject has fewer than 100,000 platelets per cubic mm of blood.
   66. The method of aspect 46, wherein the subject is female.
   67. The method of aspect 66, wherein the subject is not pregnant.
   68. The method of aspect 46, wherein the subject has a blood thyroid stimulating hormone concentration that is above the lower limit of normal and below the upper limit of normal.
   69. The method of aspect 68, wherein the subject has serum creatine of less than 1.4 of the upper limit of normal.
   70. The method of aspect 46, wherein the subject is a male.
   71. The method of aspect 70, wherein the subject has serum creatine of less than 1.5 of the upper limit of normal.
   72. The method of aspect 46, wherein the subject has an amount of aspartate transaminase that is less than two times the upper limit of normal.
   73. The method of aspect 46, wherein the subject has an amount of alanine transaminase that is less than two times the upper limit of normal.
   74. The method of aspect 46, wherein the antibody or antigen-binding fragment is administered in a dosage amount within the range of about 5 mg to about 500 mg.
   75. The method of aspect 74, wherein the antibody or antigen-binding fragment is administered in a dosage amount within the range of about 50 mg to about 300 mg.
   76. The method of aspect 74, wherein the antibody is administered at between 200 and 300 mg every four weeks.
   77. The method of aspect 74, wherein the antibody or antigen-binding fragment is administered in a dosage amount of about 150 mg.
   78. The method of aspect 46 wherein the antibody or antigen-binding fragment thereof is administered to the subject every other week (E2W).
   79. The method of aspect 46, wherein the antibody or antigen-binding fragment thereof is administered to the subject every fourth week (E4W).
   80. The method of aspect 46 wherein the antibody or the antigen-binding fragment comprises the heavy and light chain CDRs of a HCVR/LCVR amino acid sequence pair as shown in SEQ ID NOs: 90/92.
   81. The method of aspect 46, wherein the antibody or antigen-binding fragment comprises a HCVR/LCVR amino acid sequence pair as shown in SEQ ID NOs: 90/92.
   82. The method of aspect 46, wherein the antibody or antigen-binding fragment thereof competes for binding to hPCSK9 with an antibody or antigen-binding fragment comprising a HCVR/LCVR amino acid sequence pair as shown in SEQ ID NOs: 90/92.
   83. The method of aspect 46, wherein the antibody is administered subcutaneously.
   84. The method of aspect 38, wherein the antibody is administered in the abdomen.
   85. The method of aspect 46, further comprising administering a HMG-COA reductase inhibitor to the subject.
   86. The method of aspect 85, wherein the HMG-COA reductase inhibitor is administered in a dosage amount in the range of about 0.05 mg to 100 mg.
   87. The method of aspect 86, wherein the HMG-COA reductase inhibitor is a statin.
   88. The method of aspect 87, wherein the statin is selected from the group consisting of cerivastatin, atorvastatin, simvastatin, pitavastatin, rosuvastatin, fluvastatin, lovastatin, and pravastatin.
   89. The method of aspect 88, wherein the statin is atorvastatin administered at a dosage of 10 mg or 80 mg.
   90. The method of aspect 89, wherein the atorvastatin is administered at about 10 mg per day and at 80 mg one day in an 8 week period.

### Aspects Related to Dosage Regimens—A)

1. A method for treating a disease or condition in which PCSK9 expression or activity causes an impact, comprising:
   administering a therapeutic amount of an antibody or an antigen-binding fragment thereof which specifically binds hPCSK9 (human proprotein convertase subtilisin/kexin type 9) to a subject in need thereof, wherein the antibody or antigen-binding fragment thereof is administered in a dosage amount ranging from 5 mg to 500 mg, and
   administering a therapeutic amount of an HMG-COA reductase inhibitor to said subject, wherein the HMG-COA reductase inhibitor is administered in a dosage amount ranging from 0.05 mg to 100 mg.
2. An antibody or an antigen-binding fragment thereof which specifically binds hPCSK9 (human proprotein convertase subtilisin/kexin type 9) for use in the treatment of a disease or condition in which PCSK9 expression or activity causes an impact,
   wherein the antibody or antigen-binding fragment thereof is for administration in a dosage amount ranging from 5 mg to 500 mg,
   wherein the antibody or antigen-binding fragment thereof is further for administration in combination with an HMG-COA reductase inhibitor at a dosage amount ranging from 0.05 mg to 100 mg.
3. The method of aspect 1 or the antibody of aspect 2, wherein the disease or condition in which PCSK9 expression or activity causes an impact is ameliorated, improved, inhibited or prevented with a PCSK9 antagonist.
4. The method or the antibody of any one of aspects 1-3, wherein the disease or condition in which PCSK9 expression or activity causes an impact is selected from the group consisting of:
   hypercholesterolemia, hyperlipidemia, dyslipidemia, atherosclerosis and cardiovascular diseases.
5. The method or the antibody of anyone of aspects 1 to 4, wherein the subject in need thereof is a subject indicated for LDL apheresis, a subject with PCSK9-activating mutations, a subject with heterozygous Familial Hypercholesterolemia, a subject with primary hypercholesterolemia who is statin uncontrolled, a subject at risk for developing hypercholesterolemia, a subject with hypercholesterolemia, hyperlipidemia, dyslipidemia, atherosclerosis or cardiovascular diseases.
6. The method or the antibody of any one of aspects 1 to 5, wherein the HMG-COA reductase inhibitor is administered three times per day, twice per day, or once per day.
7. The method or the antibody of any one of aspects 1 to 6, wherein the HMG-COA reductase inhibitor is administered every day, every other day, every third day, every fourth day, every fifth day, or every sixth day.
8. The method or the antibody of any one of aspects 1 to 6, wherein the HMG-COA reductase inhibitor is administered every week, every other week, every third week, or every fourth week.
9. The method or the antibody of any one of aspects 1 to 8 wherein the HMG-COA reductase inhibitor is administered in the morning, at noon or in the evening.
10. The method or the antibody of any one of aspects 1 to 9, wherein the HMG-COA reductase inhibitor is a statin.
11. The method or the antibody of aspect 10, wherein the statin is selected from the group consisting of cerivastatin, atorvastatin, simvastatin, pitavastatin, rosuvastatin, fluvastatin, lovastatin, and pravastatin.
12. The method or the antibody of aspect 11, wherein the statin is
   cerivastatin administered in a daily dosage of between 0.05 mg and 2 mg;
   atorvastatin administered in a daily dosage of between 2 mg and 100 mg;
   simvastatin administered in a daily dosage of between 2 mg and 100 mg;
   pitavastatin administered in a daily dosage of between 0.2 mg and 100 mg;
   rosuvastatin administered in a daily dosage of between 2 mg and 100 mg;
   fluvastatin administered in a daily dosage of between 2 mg and 100 mg;
   lovastatin administered in a daily dosage of between 2 mg and 100 mg; or
   pravastatin administered in a daily dosage of between 2 mg and 100 mg;
13. The method or the antibody of any one of aspects 1 to 12, wherein the antibody or antigen-binding fragment thereof is administered to the subject every other week.
14. The method or the antibody of any one of aspects 1 to 13, wherein the antibody or antigen-binding fragment thereof is administered in a dosage amount ranging from 50 mg to 300 mg.
15. The method or the antibody of any one of aspects 1 to 14, wherein the antibody or antigen-binding fragment thereof is a recombinant human antibody or fragment thereof.
16. The method or the antibody of any one of aspects 1 to 15, wherein the antibody or the antigen-binding fragment thereof is characterized by one or more of the following:
   (i) capable of reducing serum total cholesterol at least about 25 to about 35% and sustaining the reduction over at least a 24 day period relative to a predose level;
   (ii) capable of reducing serum LDL cholesterol at least about 65-80% and sustaining the reduction over at least a 24 day period relative to a predose level;
   (iii) capable of reducing serum triglyceride at least about 25-40% relative to predose level;
   (iv) achieves one or more of (i)-(iii) without reducing serum HDL cholesterol or reducing serum HDL cholesterol no more than 5% relative to predose level;
   (v) achieves one or more of (i)-(iii) with little or no measurable effect on liver function, as determined by ALT and AST measurements.
17. The method or the antibody of any one of aspects 1 to 16, wherein the antibody or the antigen-binding fragment thereof comprises
   a heavy chain CDR3 (HCDR3) domain selected from the group consisting of SEQ ID NO: 8, 32, 56, 80, 104, 128, 152, 176, 200, 224, 248, 272, 296, 320, 344, 368, 392, 416, 440, 464, 488, 512, 536, 560, 584, 608, 632, 656, 680, 704 and 728; and
   a light chain CDR3 (LCDR3) domain selected from the group consisting of SEQ ID NO: 16, 40, 64, 88, 112, 136, 160, 184, 208, 232, 256, 280, 304, 328, 352, 376, 400, 424, 448, 472, 496, 520, 544, 568, 592, 616, 639, 664, 688, 712 and 736.
18. The method or the antibody of any one of aspects 1 to 16, wherein the antibody or the antigen-binding fragment thereof comprises the heavy and light chain CDRs of a HCVR/LCVR amino acid sequence pair as shown in SEQ ID NOs: 90/92.
19. The method or the antibody of aspect 18, wherein the antibody or antigen-binding fragment thereof comprises heavy and light chain CDR amino acid sequences as shown in SEQ ID NOs: 76, 78, 80, 84, 86 and 88.
20. The method or the antibody of aspect 19, wherein the antibody or antigen-binding fragment thereof comprises an HCVR amino acid sequence as shown in SEQ ID NO: 90 and an LCVR amino acid sequence as shown in SEQ ID NO: 92.
21. The method or the antibody of any one of aspects 1 to 16, wherein the antibody or antigen-binding fragment thereof binds to the same epitope on hPCSK9 as an antibody comprising heavy and light chain CDR amino acid sequences as shown in SEQ ID NOs: 76, 78, 80, 84, 86, and 88.
22. The method or the antibody of any one of aspects 1 to 16, wherein the antibody or antigen-binding fragment thereof competes for binding to hPCSK9 with an antibody comprising heavy and light chain CDR amino acid sequences as shown in SEQ ID NOs: 76, 78, 80, 84, 86, and 88.
23. An article of manufacture comprising
   (a) a packaging material;
   (b) an antibody or an antigen-binding fragment thereof which specifically binds hPCSK9; and
   (c) a label or packaging insert contained within the packaging material indicating that patients receiving treatment with said antibody or antigen-binding fragment can be treated for a disease or condition selected from the group consisting of hypercholesterolemia, hyperlipidemia, dyslipidemia, atherosclerosis and cardiovascular diseases.
24. An article of manufacture comprising
   (a) a packaging material;
   (b) an antibody or an antigen-binding fragment thereof which specifically binds hPCSK9; and
   (c) a label or packaging insert contained within the packaging material indicating the treatment of patients with said antibody or antigen-binding fragment thereof together with the application of a statin.
25. An article of manufacture comprising
   (a) a packaging material;
   (b) an antibody or an antigen-binding fragment thereof which specifically binds hPCSK9; and
   (c) a label or packaging insert indicating that the treatment of patients with said antibody or antigen-binding fragment thereof together with a statin is contraindicated for patients belonging to one or more of the following groups:
      (i) smokers;
      (ii) persons being 70 years old or older;
      (iii) persons suffering from hypertension;
      (iv) women who are pregnant;
      (v) women who are trying to become pregnant;
      (vi) women who are breast-feeding;
      (vii) persons who have or ever had a disease affecting the liver;
      (viii) persons who had any unexplained abnormal blood tests for liver function;
      (ix) persons who drink excessive amounts of alcohol;
      (x) persons having kidney problems;
      (xi) persons suffering from hypothyroidism;
      (xii) persons suffering from muscle disorders;
      (xiii) persons having encountered previous muscular problems during treatment with lipid-lowering medicine;
      (xiv) persons having serious problems with their breathing;
      (xv) persons taking one or more of the following medicines: medicines altering the way the immune systems works (e.g. ciclosporin or antihistamines), antibiotics or antifungal medicines (e.g. erythromycin, clarithromycin, ketoconazole, itraconazole, rifampicin, fusidic acid), medicines regulating lipid levels (e.g. gemfibrozil, colestipol), calcium channel blockers (e.g. verapamil, diltiazem), medicines regulating the heart rhythm (digoxin, amiodarone), protease inhibitors used in the treatment of HIV (e.g. nelfinavir), warfarin, oral contraceptives, antacids or St. John's Wort; or
      (xvi) persons drinking more than 0.1 L of grapefruit juice per day;
      (xvii) persons having a body mass index (BMI) of more than 40;
      (xviii) persons having a body mass index (BMI) of less than 18;
      (xix) persons suffering from type 1 diabetes or type 2 diabetes;
      (xx) persons positive for hepatitis B or hepatitis C; or
      (xxi) persons having a known sensitivity to monoclonal antibody therapeutics.
26. The article of manufacture according to one of aspects 23 to 25, wherein the antibody or antigen-binding fragment is an antibody or antigen-binding fragment as specified in any of aspects 2 to 22.
27. The article of manufacture according to one of aspects 23 to 26, wherein the label or packaging insert contains reference to a method of treatment according to any of aspects 1, or 3-22.
28. A method of testing the efficacy of an antibody or an antigen-binding fragment thereof which specifically binds hPCSK9 for the treatment of a disease or condition selected from the group consisting of hypercholesterolemia, hyperlipidemia, dyslipidemia, atherosclerosis and cardiovascular diseases, said method comprising:
   treating a selected patient population with said antibody or antigen-binding fragment thereof, wherein each patient in said population has an LDL cholesterol (LDL-C) level of more than 100 mg/dL; and
   determining the efficacy of said antibody or antigen-binding fragment thereof by determining the LDL-C level in the patient population before and after administration of said antibody or antigen-binding fragment thereof, wherein a reduction of the LDL-C level by at least 25% relative to a predose level in at least 75% of the patient population indicates that said antibody or antigen-binding fragment thereof is efficacious for the treatment of said disease or condition in said patient population.
29. A method of testing the efficacy of an antibody or an antigen-binding fragment thereof which specifically binds hPCSK9 for the treatment of a disease or condition selected from the group consisting of hypercholesterolemia, hyperlipidemia, dyslipidemia, atherosclerosis and cardiovascular diseases, said method comprising:
   determining the efficacy of an antibody or antigen-binding fragment thereof that has been used for the treatment of a selected patient population with said antibody or antigen-binding fragment thereof, wherein each patient in said population has an LDL cholesterol (LDL-C) level of more than 100 mg/dL by determining the LDL-C level in the patient population before and after administration of said antibody or antigen-binding fragment thereof, wherein a reduction of the LDL-C level by at least 25% relative to a predose level in at least 75% of the patient population indicates that said antibody or antigen-binding fragment thereof is efficacious for the treatment of said disease or condition in said patient population.
30. The method of aspect 28 or 29, wherein each patient in said population has received a lipid lowering treatment by administration of a statin for at least 6 weeks prior to treatment with said antibody or antigen-binding fragment thereof.
31. The method of any of aspects 28 to 30, wherein the antibody or antigen-binding fragment is an antibody or antigen-binding fragment as specified in any of aspects 2 to 22.
32. The method of any of aspects 28 to 31, wherein the selected patient population is treated with a method of treatment according to any of aspects 1, or 3-22.
33. A package comprising an antibody or antigen-binding fragment thereof of one or more of aspects 2 to 22 and a label, said label comprising a printed statement which informs the patient that the treatment of the antibody together with a statin is indicated in one or more of the indications of aspect 4.
34. A package comprising an antibody or antigen-binding fragment thereof of one or more of aspects 2 to 22 and a label, said label comprising a printed statement which informs the patient that the treatment of the antibody together with a statin is contraindicated for patients belonging to one or more of the following groups:
   (i) smokers;
   (ii) persons being 70 years old or older;
   (iii) persons suffering from hypertension;
   (iv) women who are pregnant;
   (v) women who are trying to become pregnant;
   (vi) women who are breast-feeding;
   (vii) persons who have or ever had a disease affecting the liver;
   (viii) persons who had any unexplained abnormal blood tests for liver function;
   (ix) persons who drink excessive amounts of alcohol;
   (x) persons having kidney problems;
   (xi) persons suffering from hypothyroidism;
   (xii) persons suffering from muscle disorders;
   (xiii) persons having encountered previous muscular problems during treatment with lipid-lowering medicine;
   (xiv) persons having serious problems with their breathing;
   (xv) persons taking one or more of the following medicines: medicines altering the way the immune systems works (e.g. ciclosporin or antihistamines), antibiotics or antifungal medicines (e.g. erythromycin, clarithromycin, ketoconazole, itraconazole, rifampicin, fusidic acid), medicines regulating lipid levels (e.g. gemfibrozil, colestipol), calcium channel blockers (e.g. verapamil, diltiazem), medicines regulating the heart rhythm (digoxin, amiodarone), protease inhibitors used in the treatment of HIV (e.g. nelfinavir), warfarin, oral contraceptives, antacids or St. John's Wort; or
   (xvi) persons drinking more than 0.1 L of grapefruit juice per day;
   (xvii) persons having a body mass index (BMI) of more than 40;
   (xviii) persons having a body mass index (BMI) of less than 18;
   (xix) persons suffering from type 1 diabetes or type 2 diabetes;
   (xx) persons positive for hepatitis B or hepatitis C; or
   (xxi) persons having a known sensitivity to monoclonal antibody therapeutics.
35. A method of regulating the LDL level in the blood comprising:
   administering a therapeutic amount of an antibody or an antigen-binding fragment thereof which specifically binds hPCSK9 (human proprotein convertase subtilisin/kexin type 9) to a subject in need thereof, wherein the antibody or antigen-binding fragment thereof is administered in a dosage amount ranging from 5 mg to 500 mg, and
   administering a therapeutic amount of an HMG-COA reductase inhibitor to said subject, wherein the HMG-COA reductase inhibitor is administered in a dosage amount ranging from 0.05 mg to 100 mg.
36. A method of preventing effects of a (persistently) increased LDL level in the blood comprising:
   administering a therapeutic amount of an antibody or an antigen-binding fragment thereof which specifically binds hPCSK9 (human proprotein convertase subtilisin/kexin type 9) to a subject in need thereof, wherein the antibody or antigen-binding fragment thereof is administered in a dosage amount ranging from 5 mg to 500 mg, and
   administering a therapeutic amount of an HMG-COA reductase inhibitor to said subject, wherein the HMG-COA reductase inhibitor is administered in a dosage amount ranging from 0.05 mg to 100 mg.
37. A method of determining whether a pharmaceutical compound is utilizable for ameliorating, improving, inhibiting or preventing a disease or condition in which PCSK9 activity or expression has an impact comprising
   (a) administering to a subject a compound that specifically binds to PCSK9, preferably an antibody or antigen-binding fragment thereof specifically binding to PCSK9, and
   (b) determining what fraction of PCSK9 in the blood is attached to the compound from (a).

### Aspects Related to Dosage Regimens—B)

1. A method of treating a subject suffering from a disease or disorder characterized by elevated low-density lipoprotein cholesterol (LDL-C) levels, the method comprising administering to the subject: (1) an antibody, or antigen-binding fragment thereof, which specifically binds to human proprotein convertase subtilisin/kexin type 9 (hPCSK9); and (2) an HMG-COA reductase inhibitor, wherein the antibody or antigen-binding fragment thereof is administered at a dosage amount within the range of about 5 mg to about 500 mg, thereby treating the subject.
2. The method of aspect 1, wherein the disease or condition is selected from the group consisting of: hypercholesterolemia, hyperlipidemia, dyslipidemia, and atherosclerosis.
3. The method of aspect 1, wherein the disease condition is primary hypercholesterolemia or familial hypercholesterolemia.
4. The method of aspect 1, wherein the disease or condition is hypercholesterolemia which is uncontrolled by statins.
5. The method of aspect 1, wherein the antibody or antigen-binding fragment is administered in a dosage amount within the range of about 50 mg to about 300 mg.
6. The method of aspect 1, wherein the antibody or antigen-binding fragment is administered in a dosage amount of about 150 mg.
7. The method of aspect 1, wherein the antibody or antigen-binding fragment thereof is administered to the subject every other week (E2W).
8. The method of aspect 1, wherein the antibody or antigen-binding fragment thereof is administered to the subject every fourth week (E4W).
9. The method of aspect 1, wherein the treatment reduces serum total cholesterol at least about 25% to about 35% relative to a predose level and sustains the reduction over at least a 24 day period.
10. The method of aspect 1, wherein the treatment reduces serum total cholesterol at least about 65% to about 80% relative to a predose level and sustains the reduction over at least a 24 day period.
11. The method of aspect 1, wherein the treatment reduces serum triglyceride levels at least about 25% to about 40% relative to a predose level.
12. The method of aspect 1, wherein the treatment reduced serum HDL cholesterol no more than 5% relative to a predose level.
13. The method of aspect 1, wherein the treatment has little or no measurable effect on liver function, as determined by ALT and AST measurements.
14. The method of aspect 1, wherein the antibody or the antigen-binding fragment comprises the heavy and light chain CDRs of a HCVR/LCVR amino acid sequence pair as shown in SEQ ID NOs: 90/92.
15. The method of aspect 1, wherein the antibody or antigen-binding fragment comprises a HCVR/LCVR amino acid sequence pair as shown in SEQ ID NOs: 90/92.
16. The method of aspect 1, wherein the antibody or antigen-binding fragment thereof competes for binding to hPCSK9 with an antibody or antigen-binding fragment comprising a HCVR/LCVR amino acid sequence pair as shown in SEQ ID NOs: 90/92.
17. The method of aspect 1, wherein the HMG-COA reductase inhibitor is administered in a dosage amount in the range of about 0.05 mg to 100 mg.
18. The method of aspect 1, wherein the HMG-COA reductase inhibitor is a statin.
19. The method of aspect 1, wherein the statin is selected from the group consisting of cerivastatin, atorvastatin, simvastatin, pitavastatin, rosuvastatin, fluvastatin, lovastatin, and pravastatin.
20. The method of aspect 1, wherein the statin is atorvastatin administered at a dosage of 10 mg or 80 mg.
21. A method of enhancing the LDL-C lowering activity in a subject undergoing statin therapy, the method comprising administering to the subject an antibody, or antigen-binding fragment thereof, which specifically binds to human proprotein convertase subtilisin/kexin type 9 (hPCSK9), wherein the antibody or antigen-binding fragment thereof is administered at a dosage amount within the range of about 5 mg to about 500 mg, thereby enhancing LCL-C lowering activity of the statin therapy in the subject.
22. The method of aspect 21, wherein the subject is resistant to the statin therapy prior to administration of the antibody.
23. The method of aspect 21, wherein the subject suffers from a disease or condition selected from the group consisting of hypercholesterolemia, hyperlipidemia, dyslipidemia, and atherosclerosis.
24. The method of aspect 21, wherein the disease condition is primary hypercholesterolemia or familial hypercholesterolemia.
25. The method of aspect 21, wherein the antibody or antigen-binding fragment is administered in a dosage amount within the range of about 50 mg to about 300 mg.
26. The method of aspect 21, wherein the antibody or antigen-binding fragment is administered in a dosage amount of about 150 mg.
27. The method of aspect 21, wherein the antibody or antigen-binding fragment thereof is administered to the subject every other week (E2W).
28. The method of aspect 21, wherein the antibody or antigen-binding fragment thereof is administered to the subject every fourth week (E4W).
29. The method of aspect 21, wherein the treatment reduces serum total cholesterol at least about 25% to about 35% relative to a predose level and sustains the reduction over at least a 24 day period.
30. The method of aspect 21, wherein the treatment reduces serum total cholesterol at least about 65% to about 80% relative to a predose level and sustains the reduction over at least a 24 day period.
31. The method of aspect 21, wherein the treatment reduces serum triglyceride levels at least about 25% to about 40% relative to a predose level.
32. The method of aspect 21, wherein the treatment reduced serum HDL cholesterol no more than 5% relative to a predose level.
33. The method of aspect 21, wherein the treatment has little or no measurable effect on liver function, as determined by ALT and AST measurements.
34. The method of aspect 21, wherein the antibody or the antigen-binding fragment comprises 1the heavy and light chain CDRs of a HCVR/LCVR amino acid sequence pair as shown in SEQ ID NOs: 90/92.
35. The method of aspect 21, wherein the antibody or antigen-binding fragment comprises a HCVR/LCVR amino acid sequence pair as shown in SEQ ID NOs: 90/92.
36. The method of aspect 21, wherein the antibody or antigen-binding fragment thereof competes for binding to hPCSK9 with an antibody or antigen-binding fragment comprising a HCVR/LCVR amino acid sequence pair as shown in SEQ ID NOs: 90/92.
37. The method of aspect 21, wherein the statin is selected from the group consisting of cerivastatin, atorvastatin, simvastatin, pitavastatin, rosuvastatin, fluvastatin, lovastatin, and pravastatin.
38. The method of aspect 21, wherein the statin is atorvastatin administered at a dosage of 10 mg or 80 mg.
39. A pharmaceutical unit dosage form comprising an antibody, or antigen-binding fragment thereof, which specifically binds to hPCSK9; and pharmaceutically acceptable carrier, wherein the antibody or antigen-binding fragment is present in a dosage amount within the range of about 5 mg to about 500 mg.
40. The dosage form of aspect 39, wherein the antibody or antigen binding fragment is present in a dosage amount within the range of about 50 mg to about 300 mg.
41. The dosage form of aspect 39, wherein the antibody or antigen binding fragment is present in a dosage amount of about 150 mg.
42. The dosage form of aspect 39, wherein the antibody or the antigen-binding fragment comprises the heavy and light chain CDRs of a HCVR/LCVR amino acid sequence pair as shown in SEQ ID NOs: 90/92.
43. The dosage form of aspect 39, wherein the antibody or antigen-binding fragment comprises a HCVR/LCVR amino acid sequence pair as shown in SEQ ID NOs: 90/92.
44. The dosage form of aspect 39, wherein the antibody or antigen-binding fragment thereof competes for binding to hPCSK9 with an antibody or antigen-binding fragment comprising a HCVR/LCVR amino acid sequence pair as shown in SEQ ID NOs: 90/92.
45. The dosage form of aspect 39, further comprising a HMG-CoA reductase inhibitor.
46. The dosage form of aspect 39, wherein the HMG-COA reductase inhibitor is present in a dosage amount in the range of about 0.05 mg to 100 mg.
47. The dosage form of aspect 39, wherein the HMG-COA reductase inhibitor is a statin.
48. The dosage form of aspect 39, wherein the statin is selected from the group consisting of cerivastatin, atorvastatin, simvastatin, pitavastatin, rosuvastatin, fluvastatin, lovastatin, and pravastatin.
49. The dosage form of aspect 39, wherein the statin is atorvastatin present at dosage amount of 10 mg or 80 mg.
50. A kit for treating elevated low-density lipoprotein cholesterol (LDL-C) levels in a subject, the kit comprising (a) pharmaceutical unit dosage form comprising an antibody, or antigen-binding fragment thereof, which specifically binds to hPCSK9; and pharmaceutically acceptable carrier, wherein the antibody or antigen-binding fragment is present in a dosage amount within the range of about 5 mg to about 500 mg; and (b) a label or packaging insert with instructions for use.
51. The kit of aspect 50, wherein the label indicates that patients receiving treatment with said antibody or antigen-binding fragment can be treated for a disease or condition selected from the group consisting of hypercholesterolemia, hyperlipidemia, dyslipidemia, and atherosclerosis and cardiovascular diseases.
52. The kit of aspect 51, wherein the disease or condition is primary hypercholesterolemia or familial hypercholesterolemia.
53. The kit of aspect 51, wherein the disease or condition is hypercholesterolemia which is uncontrolled by statins.
54. The kit of aspect 50, wherein the antibody or antigen-binding fragment is present in dosage amount within the range of about 50 mg to about 300 mg.
55. The kit of aspect 50, wherein the antibody or antigen-binding fragment is present in a dosage amount of about 150 mg.
56. The kit of aspect 50, wherein the label or packaging insert indicates that the antibody or antigen-binding fragment thereof is administered to the subject every other week (E2W).
57. The kit of aspect 50, wherein the label or packaging insert indicates that the antibody or antigen-binding fragment thereof is administered to the subject every fourth week (E4W).
58. The kit of aspect 50, wherein the antibody or the antigen-binding fragment comprises the heavy and light chain CDRs of a HCVR/LCVR amino acid sequence pair as shown in SEQ ID NOs: 90/92.
59. The kit of aspect 50, wherein the antibody or antigen-binding fragment comprises a HCVR/LCVR amino acid sequence pair as shown in SEQ ID NOs: 90/92.
60. The kit of aspect 50, wherein the antibody or antigen-binding fragment thereof competes for binding to hPCSK9 with an antibody or antigen-binding fragment comprising a HCVR/LCVR amino acid sequence pair as shown in SEQ ID NOs: 90/92.
61. The kit of aspect 50, further comprising an HMG-COA reductase inhibitor.
62. The kit of aspect 61, wherein the inhibitor in a dosage amount in the range of about 0.05 mg to 100 mg.
63. The kit of aspect 50, wherein the HMG-COA reductase inhibitor is a statin.
64. The kit of aspect 50, wherein the statin is selected from the group consisting of cerivastatin, atorvastatin, simvastatin, pitavastatin, rosuvastatin, fluvastatin, lovastatin, and pravastatin.
65. The kit of aspect 50, wherein the instructions indicate that the statin is atorvastatin administered at a dosage of 10 mg or 80 mg.
66. The kit of aspect 50, wherein the instructions indicate that treatment with the antibody or an is contraindicated for patients belonging to one or more of the following groups:
   (xxii) smokers;
   (xxiii) persons being 70 years old or older;
   (xxiv) persons suffering from hypertension;
   (xxv) women who are pregnant;
   (xxvi) women who are trying to become pregnant;
   (xxvii) women who are breast-feeding;
   (xxviii) persons who have or ever had a disease affecting the liver;
   (xxix) persons who had any unexplained abnormal blood tests for liver function; (xxx) persons who drink excessive amounts of alcohol;
   (xxxi) persons having kidney problems;
   (xxxii) persons suffering from hypothyroidism;
   (xxxiii) persons suffering from muscle disorders;
   (xxxiv) persons having encountered previous muscular problems during treatment with lipid-lowering medicine;
   (xxxv) persons having serious problems with their breathing;
   (xxxvi) persons taking one or more of the following medicines: medicines altering the way the immune systems works (e.g. ciclosporin or antihistamines), antibiotics or antifungal medicines (e.g. erythromycin, clarithromycin, ketoconazole, itraconazole, rifampicin, fusidic acid), medicines regulating lipid levels (e.g. gemfibrozil, colestipol), calcium channel blockers (e.g. verapamil, diltiazem), medicines regulating the heart rhythm (digoxin, amiodarone), protease inhibitors used in the treatment of HIV (e.g. nelfinavir), warfarin, oral contraceptives, antacids or St. John's Wort; or
   (xxxvii) persons drinking more than 0.1 L of grapefruit juice per day;
   (xxxviii) persons having a body mass index (BMI) of more than 40;
   (xxxix) persons having a body mass index (BMI) of less than 18;
   (xl) persons suffering from type 1 diabetes or type 2 diabetes;
   (xli) persons positive for hepatitis B or hepatitis C; or
   (xlii) persons having a known sensitivity to monoclonal antibody therapeutics.

### Aspects Related to Compositions

1. Pharmaceutical composition comprising about 40 to about 500 mg per dose of an antibody or an antigen-binding fragment thereof which specifically binds hPCSK9 (human proprotein convertase subtilisin/kexin type 9) together with a pharmaceutically acceptable excipient or carrier.
2. Pharmaceutical composition according to aspect 1, comprising about 50 mg to about 500 mg, about 50 mg to about 300 mg, about 50 mg, about 100 mg, about 150 mg, about 200 mg, about 250 mg, about 300 mg, about 350 mg, of about 400 mg, about 450 mg or about 500 mg of the antibody or antigen-binding fragment thereof.
3. Pharmaceutical composition according to one of the aspects 1 or 2 comprising about 150, 200 or 300 mg of the antibody or antigen-binding fragment thereof.
4. Pharmaceutical composition according to one of the aspects 1-3 comprising an effective dose of an antibody or an antigen-binding fragment thereof which specifically binds hPCSK9 (human proprotein convertase subtilisin/kexin type 9), wherein the dose is sufficient for sustained reduction of low-density lipoprotein (LDL-C) levels over a period of at least 14, at least 15, at least 16, at least 17, at least 18, at least 19, at least 20, at least 21, at least 22, at least 23 or at least 28 days after administration, together with a pharmaceutically acceptable excipient or carrier.
5. Pharmaceutical composition according to one of the aspects 1-4, wherein the dose is sufficient for sustained reduction of LDL-C levels over a period of at least 14 days, 28 days or 1 month.
6. Pharmaceutical composition according to one of the aspects 1-5 further comprising an effective amount of an HMG-COA reductase inhibitor.
7. Pharmaceutical composition according to aspect 6, wherein the HMG-COA reductase inhibitor is a statin, preferably selected from the list consisting or: cerivastatin, atorvastatin, simvastatin, pitavastatin, rosuvastatin, fluvastatin, lovastatin or pravastatin and is preferably atorvastatin.
8. Pharmaceutical composition according to aspect 6 or 7, comprising about 0.05 mg to about 100 mg, about 0.5 mg to about 100 mg, about 5 mg to about 90 mg, about 10 mg, about 20 mg, about 40 mg or about 80 mg of HMG-COA reductase inhibitor and preferably about 10, about 20, about 40 or about 80 mg.
9. Pharmaceutical composition according to one of the aspects 6 to 8, comprising an effective dose of HMG-CoA reductase inhibitor for lowering LDL-D levels by administration once per day.
10. Pharmaceutical composition according to one of the aspects 1 to 9, wherein the antibody or antigen-binding fragment thereof has one or more of the following features:
   a. reduction of low-density lipoprotein (LDL-C) levels of at least about −25% to about −40% relative to a predose level with a sustained reduction over at least a 14 day-period upon administration to a subject, wherein the sustained reduction is preferably at least −25% and more preferably at least −30% relative to a predose level, particularly if administered in a dose of about 40 to about 60 mg, preferably about 45 to about 55 mg and more preferably about 50 mg in a biweekly administration regime (every other week, E2W);
   b. reduction of low-density lipoprotein (LDL-C) of at least about −50% to about −65% relative to a predose level with a sustained reduction over at least a 14 day-period upon administration to a subject, wherein the sustained reduction is preferably at least −40% and more preferably at least −45% relative to a predose level, particularly if administered in a dose of about 100 mg E2W.
   c. reduction of low-density lipoprotein (LDL-C) of at least about −60% to at least about −75% [e.g. at least about −60%, at least about −65%, at least about −70 or at least about −75%] relative to a predose level with a sustained reduction over at least a 14 day-period upon administration to a subject, wherein the sustained reduction is preferably at least −55% and more preferably at least −60% relative to a predose level, particularly when administered in a dose of about 150 mg E2W,
   d. reduction of low-density lipoprotein (LDL-C) of at least about 40% to about 75% relative to a predose level with a sustained reduction over at least a 28 day period, wherein the sustained reduction is preferably at least −35% and more preferably at least −40% relative to a predose level, particularly when administered in a dose of about 200 mg E4W
   e. reduction of low-density lipoprotein (LDL-C) of at least about −50% to about −75% relative to a predose level with a sustained reduction over at least a 28 day-period upon administration to a subject, wherein the sustained reduction is preferably at least −40% and more preferably at least −45% relative to a predose level, particularly when administered in a dose of about 300 mg E4W,
   f. increase of serum HDL cholesterol levels of at least 2%, at least 2.5%, at least, 3%, at least 3.5%, at least 4%, at least 4.5%, at least 5% or at least 5.5% relative to a predose level upon administration to a subject, particularly when administered in a dose of about 150 mg E2W,
   g. little or no measurable effect on troponin levels upon administration to a subject,
   h. increase of one or more of: Total-Cholesterol levels, ApoB levels, non HDL-C levels, Apo-B/ApoA-1 ratio, upon administration to a subject.
11. Pharmaceutical composition according to one of the aspects 1-9, wherein the antibody or antigen-binding fragment thereof is capable of overcoming statin resistance when administered to a subject with statin-resistant hypercholesterolemia.
12. Pharmaceutical composition according to one of the aspects 1-10, wherein the antibody or antigen-binding fragment thereof comprises the heavy and light chain CDRs of a HCVR/LCVR amino acid sequence pair as shown in SEQ ID NOs: 90/92 substantially identical sequences having at least 98% or 99% identity therewith.
13. Pharmaceutical composition according to one of the aspects 1-11, wherein the antibody or antigen-binding fragment thereof comprises a HCVR/LCVR amino acid sequence pair as shown in SEQ ID NOs: 90/92 or a pair of substantially identical sequences having at least 98% or 99% identity therewith.
14. Pharmaceutical composition according to one of the aspects 1-10, wherein the antibody or antigen-binding fragment thereof competes for binding to hPCSK9 with an antibody or antigen-binding fragment comprising a HCVR/LCVR amino acid sequence pair as shown in SEQ ID NOs: 90/92.
15. Pharmaceutical composition according to one of the aspects 1-13, wherein the antibody or antigen-binding fragment thereof binds an epitope comprising amino acid residue 238 of hPCSK9 (SEQ ID NO:755).
16. Pharmaceutical composition according to one of the aspects 1-14, wherein the antibody or antigen-binding fragment thereof binds an epitope comprising one or more of amino acid residues at positions 238, 153, 159 and 343 of hPCSK9 (SEQ ID NO:755).
17. Pharmaceutical composition according to one of the aspects 1-15, wherein the antibody or antigen-binding fragment thereof binds an epitope which does not comprise an amino acid residue at positions 192, 194, 197 and/or 237 of hPCSK9 (SEQ ID NO:755).
18. Pharmaceutical composition according to one of the aspects 1-16 comprising the antibody or antigen-binding fragment thereof as dry formulation for dissolution such as a lyophilized powder, freeze-dried powder or water free concentrate.
19. Pharmaceutical composition according to one of the aspects 1-17 comprising the antibody or fragment thereof as liquid formulation, e.g. injection or infusion solution.
20. Pharmaceutical composition according to one of the aspects 4-18 comprising the HMG-CoA reductase inhibitor as peroral formulation, e.g. capsule or tabled, or as liquid formulation, e.g. suspension, dispersion or solution, e.g. for peroral administration, injection or infusion.
21. Injection solution according to aspect 19, preferably comprising about 40 mg to about 200 mg or about 40 to about 200 mg, e.g. about 40 mg, about 50 mg, about 75 mg, at about 100 mg, about 150 mg or about 200 mg of the antibody or antigen-binding fragment thereof per 1 ml volume.
22. Dry formulation according to aspect 17, preferably comprising about 40 mg to about 500 mg, 50 to about 500 mg, about 50 to about 400, about 50 to about 300 e.g. about 40 mg, about 50 mg, about 75 mg, at about 100 mg, about 150 mg, about 200 mg, about 250 mg, about 300 mg, about 350 mg, about 400 mg, about 450 mg or about 500 mg and preferably about 50 mg, about 100 mg, about 150 mg, about 200 mg, about 250 mg, about 300 mg and even more preferably about 150 mg, about 200 mg or about 300 mg of the antibody or antigen-binding fragment thereof per dose.
23. Antibody or antigen binding fragment thereof as comprised in one of the pharmaceutical compositions according to one of the aspects 1-17.
24. Unit dosage form comprising the pharmaceutical composition according to one of the aspects 1-20, the injection solution according to aspect 21, the dry formulation according to aspect 22, or the antibody according to aspect 23.
25. Unit dosage form according to aspect 24, comprising about 40 mg, about 50 mg, about 75 mg, at about 100 mg, about 150 mg, about 200 mg, about 250 mg, about 300 mg, about 350 mg, about 400 mg, about 450 mg, or about 500 mg of the antibody or antigen-binding fragment thereof.
26. Unit dosage form according to one of the aspects 24 or 25 comprising the antibody or fragment thereof as dry formulation for dissolution in a hermetically sealed container such as a vial, an ampoule or sachette.
27. Unit dosage form according to one of the aspects 24 or 25 comprising the antibody or fragment thereof as liquid formulation in a hermetically sealed container such as a vial, a sachette, a pre-filled syringe, a pre-filled autoinjector or a cartridge for a reusable syringe or applicator.
28. Unit dosage form according to aspect 26 or 27, wherein the quantity of active ingredient is indicated on the hermetically-sealed container.
29. Article of manufacture comprising, the pharmaceutical composition according to one of the aspects 1-20, the liquid formulation according to aspect 21 or the dry formulation according to aspect 22, the antibody or antigen-binding fragment thereof according to aspect 23 or one or more unit dosage forms according to one of the aspects 24-28, and a container.
30. Article of manufacture according to aspect 29 comprising separate unit dosage forms the antibody according to aspect 23 and the HMG-COA reductase inhibitor according to one of the aspects 5-9 or 20.
31. Article of manufacture according to aspect 30 comprising one or more of the following components:
   a. One or more unit dosage forms comprising the antibody according to aspect 23;
   b. One or more unit dosage forms comprising the HMG-COA reductase inhibitor according to one of the aspects 6-9 or 20;
   c. Instructions for use;
   d. A device for application of the antibody such as a syringe.
32. Article of manufacture according to aspect 31, comprising sufficient unit dosage forms of the antibody and preferably also of the HMG-COA reductase inhibitor, for one single administration of antibody and HMG-COA reductase inhibitor, for a two-week (i.e. 14-day) treatment with antibody and HMG-COA reductase inhibitor, for a four week (i.e, 28-day) treatment with antibody and HMG-COA reductase inhibitor or for a one-month treatment with antibody and HMG-COA reductase inhibitor.
33. Article of manufacture according to aspect 32, comprising sufficient unit dosage forms of antibody for a bi-weekly administration regime or a four-weekly administration regime or a monthly administration regime.
34. Article of manufacture according to aspect 32 or 33 comprising sufficient unit dosage forms of HMG-COA reductase inhibitor for a daily administration regime.
35. Pharmaceutical composition according to one of the aspects 1 to 20 or antibody or antigen-binding fragment thereof according to aspect 21 for use in the treatment of a disease or condition in which PCSK9 expression or activity causes an impact, preferably for use in the lowering of elevated LDL-C (low density lipoprotein C) levels
36. Pharmaceutical composition or antibody or antigen-binding fragment thereof according to aspect 35, wherein the disease or condition is selected from the group consisting of: elevated total cholesterol levels, elevated low-density lipoprotein (LDL-C) levels, hypercholesterolemia, hyperlipidemia, dyslipidemia, and atherosclerosis, particularly primary hypercholesterolemia, familial hypercholesterolemia, or hypercholesteremia which is uncontrolled by statins
37. Pharmaceutical composition or antibody or antigen-binding fragment thereof according to aspect 35 or 36, wherein the composition, the antibody or antigen-binding fragment thereof is administered to the subject every other week (E2W), every fourth week (E4W) or once a month.
38. Pharmaceutical composition or antibody or antigen-binding fragment thereof according to one of the aspects 35-37, comprising co-administration of an HMG-COA reductase inhibitor, preferably an HMG-CoA reductase inhibitor according to one of the aspects 7-9 or 20.
39. Pharmaceutical composition or antibody according to aspect 38, wherein the HMG-COA reductase inhibitor is administered once a day and preferably every day.
40. Method for preparing a pharmaceutical composition according to one of the aspects 1-20 comprising mixing the antibody or antigen-binding fragment thereof and optionally the HMG-CoA reductase inhibitor with one or more pharmaceutical excipients or carriers.
41. Method for preparing a unit dosage form according to one of the aspects 24 to 28 comprising admeasuring an amount of the pharmaceutical composition according to one of the aspects 1-20, the antibody according to aspect 21, the liquid formulation according to aspect 22 or the dry formulation according to aspect 23 comprising one or more doses of the antibody and optionally of the HMG-COA reductase inhibitor and tailor them as physically discrete units suitable as unitary dosages for human and/or animal subjects.
42. Method for preparing an article of manufacture according to one of the aspects 29-34 comprising packaging the pharmaceutical composition according to one of the aspects 1-20, the antibody according to aspect 21, the liquid formulation according to aspect 22, the dry formulation according to aspect 23 or one or more of the unit dosage forms of one of aspects 24 to 28 in a container.

### Examples

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used but some experimental errors and deviations should be accounted for. Unless indicated otherwise, molecular weight is average molecular weight, temperature is in degrees Centigrade, and pressure is at or near atmospheric.

### Study 1

This was a multicenter, randomized, double-blind, parallel-group, placebo-controlled, 12-week study to assess the efficacy and safety of antibody 316P in patients with an elevated low-density lipoprotein cholesterol (LDL-C) (>100 mg/dL or 2.59 mmol/L), when treated with atorvastatin (10 mg, 20 mg, or 40 mg) at a stable dose for at least 6 weeks. The randomization was stratified by the dose of atorvastatin received prior to randomization. After the double-blind period patients were followed during an 8-week follow-up period. The primary objective of the study was to evaluate the effect of antibody 316P on LDL-C levels after 12 weeks of treatment in comparison with placebo in patients with LDL-C (≥100 mg/dL or 2.59 mmol/L) on ongoing stable atorvastatin therapy.

The following doses/dose regimens were evaluated: 50 mg, 100 mg and 150 mg every 2 weeks (E2W), 200 mg and 300 mg every 4 weeks (E4W) in comparison with placebo.

Present study comprised a total of 122 patients (20 in placebo, 19 in 50 mg E2W group, 20 in 100 mg E2W group, 20 in 150 mg E2W group, 22 in 200 mg E4W group, and 21 in 300 mg E4W group). Forty six (37.7%) of these patients were randomized in the stratum atorvastatin 10 mg, 43 (35.2%) in the stratum atorvastatin 20 mg and 33 (27.0%) in the stratum atorvastatin 40 mg.

### Patient Selection

### Inclusion Criteria:

Patients (patients receiving a lipid lowering treatment other than atorvastatin/or not at stable dose of atorvastatin 10 mg, 20 mg or 40 mg for at least 6 weeks prior to screening period or drug naïve patients) with primary hypercholesterolemia likely to have low-density lipoprotein cholesterol (LDL-C) ≥100 mg/dl (>2.59 mmol/L) at the end of the run-in period on atorvastatin therapy (Week-1).
OR
Patients with primary hypercholesterolemia treated with atorvastatin at stable dose of 10 mg, 20 mg, or 40 mg for at least 6 weeks prior to screening period and likely to have LDL-C≥100 mg/dl (≥2.59 mmol/L) at the screening visit Week-1.

### Exclusion Criteria:

LDL-C<100 mg/dL (<2.59 mmol/L) at Week-1 (V1):
   After the run-in period on atorvastatin (10 mg, 20 mg, or 40 mg) for patients receiving a lipid lowering treatment other than atorvastatin/or not at stable dose of atorvastatin 10 mg, 20 mg or 40 mg for at least 6 weeks prior to the screening period, or drug naive patients.
   OR
   At the first visit for patients who are being treated with stable dose of atorvastatin (10 mg, 20 mg, or 40 mg) for at least 6 weeks prior to screening visit Week-1.
Use of a statin other than atorvastatin 10 mg, 20 mg, or 40 mg, or use of other lipid lowering drugs including but not limited to fibrates, bile acid resins, niacin >500 mg, intestinal cholesterol absorption (ICA) blockers, or omega-3 fatty acids at doses >1000 mg during the screening period.
Body mass index (BMI)<18 or >40 kg/m² at Week-7 or Week-1.
Patients not previously instructed on a cholesterol-lowering diet.
Patients with type 1 diabetes.
Patients with type 2 diabetes treated with insulin.
Patients with type 2 diabetes and with an HbA1c ≥8.5% at Week-7 or Week-1 (considered poorly controlled).
Laboratory findings measured before randomization:
   Positive test for hepatitis B surface antigen and/or hepatitis C antibody.
   Triglycerides (TG)>350 mg/dL (>3.95 mmol/L) at Week-7 or Week-1.
   Neutrophils <1,500/mm³ and/or platelets <100,000/mm³.
   Positive serum or urine pregnancy test in females of childbearing potential.
   Abnormal sensitive TSH level (>ULN or <LLN) according to the normal values of the Central Laboratory
   Evidence of renal impairment as determined by:
      Men: serum creatinine >1.5×ULN.
      Women: serum creatinine >1.4×ULN.
   ALT or AST >2×ULN.
   CPK>3×ULN (1 repeat lab is allowed).
All contraindications to the protocol mandated background therapy (i.e. atorvastatin) or warning/precaution of use (when appropriate) as displayed in the respective National Product Labeling that was used for defining these exclusion criteria.
Known sensitivity to monoclonal antibody therapeutics.
Pregnant or breast-feeding women.
Women of childbearing potential with no effective contraceptive method.

### Patient Population:

Demographics and baseline characteristics were generally similar across treatment groups. The median age of patients was 58.0 years (28.7% of patients were ≥65 years of age) with patients aged 24-75 years. The mean range for baseline LDL-C and Total-C among treatment groups was similar and ranged between 3.214 mmol/L and 3.500 mmol/L for LDL-C and between 5.284 mmol/L and 5.521 mmol/L for Total-C. The BMI (kg/m²) was between 19.7 to 40.5 with a mean value of 29.04 and a median value of 28.4 (with 63.6% of the patients having a BMI of <30 and 36.4% of the patients having a BMI of >30). 80 (65.6%) of the 122 patients had hyperlipoproteinemia Type IIa (familial hypercholesterinemia) according to Fredrickson classification, 41 (33.6%) type IIb (combined hyperlipidemia) and 1 (0.8%) type IV (endogenous hyperlipidemia). Overall 82% of the patients had received previous treatment with a lipid lowering agent, whereas 22% had not.

### Duration of Study Period Per Subject:

The duration of study participation depended on the status of the patient at screening:
For patients receiving atorvastatin 10 mg, 20 mg or 40 mg at a stable dose for at least 6 weeks prior to screening, the study participation was approximately 21 weeks including a screening period of 1 week, a double-blind treatment period of 12 weeks and a follow-up period of 8 weeks.
For patients receiving a lipid lowering treatment other than atorvastatin/or not at stable dose of atorvastatin 10 mg, 20 mg or 40 mg for at least 6 weeks prior to screening, or drug naive patients, the study participation was approximately 27 weeks including a screening period of 7 weeks (including a run-in period of 6 weeks), a double-blind treatment period of 12 weeks, and a follow-up period of 8 weeks.

### Active Compounds:

### Antibody 316P:

Antibody 316P is a fully human antibody comprising a HCVR as shown in SEQ ID NO:

90 and LCVR as Shown in SEQ ID NO: 92 of the Sequence Listing. The CDR Sequences are shown in SEQ ID NOs: 76, 78, and 80 (CDR1, CDR2, CDR3 of the heavy chain) as well as in SEQ ID NOs: 84, 86, and 88 (CDR1, CDR2, CDR3 of the light chain).

### Antibody 300N:

Antibody 300N is a fully human antibody comprising a HCVR as shown in SEQ ID NO: 218 and LCVR as shown in SEQ ID NO: 226 of the sequence listing. The CDR sequences are shown in SEQ ID NOs: 220, 222, and 224 (CDR1, CDR2, CDR3 of the heavy chain) as well as in SEQ ID NOs: 228, 230, and 232 (CDR1, CDR2, CDR3 of the light chain).

### Study Arms:

Arm 1: The first group of patients received two injections of 1 mL each of antibody 316P, administered subcutaneously in the abdomen, with a dose regimen at 50 mg, every two weeks, for a treatment period of 12 weeks;
   Atorvastatin was administered once per day at a stable dose of 10 mg, 20 mg, or 40 mg as background therapy.
Arm 2: The second group of patients received two injections of 1 mL each of antibody 316P, administered subcutaneously in the abdomen, with a dose regimen at 100 mg, every two weeks, for a treatment period of 12 weeks;
   Atorvastatin was administered once per day at a stable dose of 10 mg, 20 mg, or 40 mg as background therapy.
Arm 3: The third group of patients received two injections of 1 mL each of antibody 316P, administered subcutaneously in the abdomen, with a dose regimen at 150 mg, every two weeks, for a treatment period of 12 weeks;
   Atorvastatin was administered once per day at a stable dose of 10 mg, 20 mg, or 40 mg as background therapy.
Arm 4: The fourth group of patients received two injections of 1 mL each of a placebo solution, administered subcutaneously in the abdomen, every two weeks, for a treatment period of 12 weeks;
   Atorvastatin was administered once per day at stable dose of 10 mg, 20 mg, or 40 mg as background therapy.
Arm 5: The fifth group of patients received two injections of 1 mL each of antibody 316P, administered subcutaneously in the abdomen, with a dose regimen at 200 mg, every four weeks, for a treatment period of 12 weeks;
   a placebo solution was administered alternating with the administration of antibody 316P so that the patient has the same injection scheme as the patients in arms 1 to 4, i.e. the patient received two injections of 1 mL each of a placebo solution in weeks 2, 6, and 10 and two injections of 1 mL each of antibody 316P in weeks 0, 4, 8, and 12;
   Atorvastatin was administered once per day at a stable dose of 10 mg, 20 mg, or 40 mg as background therapy.
Arm 6: The sixth group of patients received two injections of 1 mL each of antibody 316P, administered subcutaneously in the abdomen, with a dose regimen at 300 mg, every four weeks, for a treatment period of 12 weeks;
   a placebo solution was administered alternating with the administration of antibody 316P so that the patient has the same injection scheme as the patients in arms 1 to 4, i.e. the patient received two injections of 1 mL each of a placebo solution in weeks 2, 6, and 10 and two injections of 1 mL each of antibody 316P in weeks 0, 4, 8, and 12;
   Atorvastatin was administered once per day at a stable dose of 10 mg, 20 mg, or 40 mg as background therapy.

### Primary and Key Secondary Endpoints:

The primary efficacy variable is the percent change in calculated LDL-C from baseline to Week 12, which is defined as: 100× (calculated LDL-C value at Week 12-calculated LDL-C value at baseline)/calculated LDL-C value at baseline.

In case of unavailable calculated LDL-C value at Week 12 as defined above, then the last calculated LDL-C value measured during the efficacy period and before the Week 12 time window will be used to impute the missing Week 12 calculated LDL-C value (Last Observation Carried Forward [LOCF] procedure).

### Secondary Efficacy Endpoints are:

The absolute change (mmol/L and mg/dL) from baseline in calculated LDL-C to Week 12, defined as: (calculated LDL-C value at Week 12-calculated LDL-C value at baseline), using same definitions and imputation rules as for the primary endpoint.

The percentage of patients with calculated LDL-C<70 mg/dL (1.81 mmol/L) and <100 mg/dL (2.59 mmol/L) at Week 12.

Percent change in ApoB from baseline to Week 12: same definitions and rules as for LDL-C, except for baseline value that will be the ApoB value measured at randomization visit (Visit2) and before first IP injection, or, if missing, the last unscheduled value obtained from Visit 1 (Week-1) up to before the first IP injection.

Percent and absolute (mmol/L and mg/dL) change in non HDL-C from baseline to Week12: same definitions and rules as for LDL-C.

Percent and absolute (mmol/L and mg/dL) change in fasting Triglycerides from baseline to Week 12: same definitions and rules as for LDL-C, excluding measurements in not fasting patients or measurements with missing fasting status.

Percent change in ApoA-1 from baseline to week 12: same definitions and rules as for AboB.

Absolute change in the ration AboB/ApoA-1 from baseline to Week12: same definitions and rules as for AboB.

Percent change in Lp(a) from baseline to Week 12: same definitions and rules as for ApoB. In case of Lp(a) value below the detection limit, a value halfway between zero and the detection limit will be used for calculation

### Results:

The efficacy of 316P treatment on LDL-C level-lowering

TABLE 1
LDL-C in mmol/L (mg/dL) at Week 12

| | LDL Cholesterol | | | | | |
|---|---|---|---|---|---|---|
| | Placebo | 50 mg | 200 mg | 100 mg | 300 mg | 150 mg |
| | mmol/L (mg/dL) | | | | | |
| | | E2W | E4W | E2W | E4W | E2W |
| | Number of patients | | | | | |
| | N = 20 | N = 19 | N = 20 | N = 20 | N = 21 | N = 18 |
| Baseline | | | | | | |
| Mean | 3.489 (134.7) | 3.214 | 3.318 | 3.422 | 3.500 | 3.238 |
| | | (124.1) | (128.1) | (132.1) | (135.1) | (125.0) |
| Median | 3.134 (121) | 3.121 | 3.225 | 3.225 | 3.250 | 3.121 |
| | | (120.5) | (124.5) | (124.5) | (125.5) | (120.5) |
| Week 12 | | | | | | |
| Mean | 3.173 (122.5) | 1.859 | 1.722 | 1.251 | 1.766 | 0.860 |
| | | (71.8) | (66.5) | (48.3) | (68.2) | (33.2) |
| Median | 3.121 (120.5) | 1.813 | 1.567 | 1.101 | 1.632 | 0.984 |
| | | (70.0) | (60.5) | (42.5) | (63.0) | (38.0) |
| Week 12 - change from | | | | | | |
| baseline | | | | | | |
| Mean | −0.317 (−12.2) | −1.355 | −1.595 | −2.171 | −1.733 | −2.378 |
| | | (−52.3) | (−61.6) | (−83.8) | (−66.9) | (−91.8) |
| Median | −0.265 (−10.3) | −1.295 | −1.593 | −2.117 | −1.904 | −2.363 |
| | | (−50.0) | (−61.5) | (−81.8) | (−73.5) | (−91.3) |
| Week 12 - % change | | | | | | |
| from baseline | | | | | | |
| Mean | −6.08 | −41.06 | −47.23 | −63.90 | −48.29 | −72.68 |
| Median | −6.92 | −37.04 | −49.46 | −64.28 | −51.98 | −74.83 |

Statistically significant decreases in percent change from baseline in LDL-C at 12 weeks were observed in all groups compared to the placebo group. The greatest decrease was seen in the 100 mg E2W (−63.90%) and 150 mg E2W (−72.68%) groups compared with a slight decrease in the placebo group (−6.08%) (LS mean difference vs. placebo of −58.36% and −68.78%, respectively); these decreases observed after the first injection were maintained throughout the study and more particularly throughout the interval period between the injections. Large decreases from baseline in LDL-C at 12 weeks were also observed in the 200 mg and 300 mg E4W groups (−47.23% and 48.29%, respectively with a LS mean difference vs. placebo of

−42.53% and −42.26%) with also significant decreases of at least about −40% during the interval periods. Among 18 patients in the 150 mg E2W group, 17 had a LDL-C reduction from baseline >50% at week 12.

| | 50 mg | 200 mg | 100 mg | 300 mg | 150 mg |
|---|---|---|---|---|---|
| Placebo | E2W | E4W | E2W | E4W | E2W |
| Number of patients | | | | | |
| N = 20 | N = 19 | N = 20 | N = 20 | N = 21 | N = 18 |

Cholesterol mmol/L

| | | | | | | |
|---|---|---|---|---|---|---|
| Baseline | | | | | | |
| Mean | 5.521 | 5.286 | 5.305 | 5.386 | 5.416 | 5.388 |
| Median | 5.458 | 5.232 | 5.394 | 5.199 | 5.180 | 5.361 |
| Week 12 | | | | | | |
| Mean | 5.378 | 3.974 | 3.709 | 3.288 | 3.778 | 2.922 |
| Median | 5.258 | 3.937 | 3.587 | 3.238 | 3.393 | 2.823 |
| Week 12 | | | | | | |
| change from | | | | | | |
| baseline | | | | | | |
| Mean | −0.143 | −1.313 | −1.596 | −2.098 | −1.638 | −2.466 |
| Median | −0.188 | −1.399 | −1.716 | −2.163 | −2.020 | −2.331 |
| Week 12 % | | | | | | |
| change from | | | | | | |
| baseline | | | | | | |
| Mean | −1.47 | −24.21 | −29.54 | −38.97 | −29.61 | −45.21 |
| Median | −3.73 | −23.34 | −29.51 | −40.21 | −33.48 | −45.03 |

Non-HDL Cholesterol (mmol/L)

| | | | | | | |
|---|---|---|---|---|---|---|
| Week 12 % | | | | | | |
| change from | | | | | | |
| baseline | | | | | | |
| Mean | −2-29 | −35.23 | −40.07 | −54.78 | −41.17 | −63.71 |
| Median | −4.71 | −36.62 | −39.91 | −55.91 | −45.55 | −65.94 |

Consistent results (decrease) were seen for Total-C, ApoB, non HDL-C. For HDL-C there was a trend of increase in all groups, similar pattern was seen for ApoA-1. Antibody 316P was well tolerated during the 12 weeks of treatment at all tested doses/dose regimens. Significantly, no change in troponin levels was noted in all treatment groups.

### Conclusion:

The results of this study showed that dosage regimens with E2W or E4W application schemes and different dosages of anti-PCSK 9 antibody 316P as used in this study are efficient and safe therapies for lowering LDL-C levels in patients with hyperlipoproteinemia and or hyperlipidemia and thus for the treatment of hyperlipoproteinemia and/or hyperlipidemia. Best overall results were achieved using the 150 mg E2W dosage regimen. However, taking into consideration the patient comfort in only obtaining antibody treatments once a month, also both E4W dosage regimens tested in present study provided very good results.

### Study 2

This was a randomized, double-blind, 3-parallel-groups, placebo-controlled, fixed dose/dose regimen, multicenter, 8-week study in subjects with primary hypercholesterolemia, aged 18-75 years. One aim of this study was to assess the efficacy and safety of 316P in patients with an elevated LDL-C (≥100 mg/dL or 2.59 mmol/L) treated with a stable dose of atorvastatin 10 mg.

During the screening period, patients had to be stabilized to atorvastatin 10 mg for at least 6 weeks, if they are not already. Then, after 1 additional screening week, patients were centrally randomized via IVRS/IWRS in a 1:1:1 ratio to one of the 3 treatment groups (placebo for 316P+atorvastatin 80 mg, 316P 150 mg E2W+atorvastatin 80 mg, 316P 150 mg E2W+atorvastatin 10 mg) and treated in a double-blind manner for approximately 8 weeks. 316P was administered every 2 weeks on site trough subcutaneous injection and atorvastatin was administered orally once daily in the evening at home. The double-blind treatment period was then followed by an 8-week follow-up period.

Approximately 90 patients (30 patients per treatment group) were recruited and randomized from approximately 20 sites.

### Objectives

### Primary Objective

To evaluate the effect of 316P on low-density lipoprotein cholesterol (LDL-C) levels compared with placebo when co-administered with 80 mg of atorvastatin after 8 weeks of treatment in patients with LDL-C ≥100 mg/dL (≥2.59 mmol/L) on atorvastatin 10 mg.

### Secondary Objective

The key secondary objectives presented in this KRM are:
To evaluate the effects of 316P on other lipid levels in comparison with placebo, when co-administered with 80 mg of atorvastatin after 8 weeks of treatment
To evaluate the efficacy of 316P when co-administered with a high dose of atorvastatin (80 mg) versus atorvastatin 10 mg
To evaluate the safety and tolerability of 316P when co-administered with 2 different doses of atorvastatin
To evaluate the effects of 316P on other exploratory endpoints: fasting plasma glucose, glycated hemoglobin A1c (HbA1c), high-sensitivity C-reactive protein (hs-CRP).

### Patient Selection:

### Inclusion Criteria:

Patients (patients receiving a lipid lowering treatment other than atorvastatin/or not at stable dose of atorvastatin 10 mg for at least 6 weeks prior to screening period, or drug naive patients) with primary hypercholesterolemia likely to have low-density lipoprotein cholesterol (LDL-C) ≥100 mg/dl (≥2.59 mmol/L) at the end of the run-in period on atorvastatin therapy (Week-1).
OR
Patients with primary hypercholesterolemia treated with stable dose of atorvastatin 10 mg for at least 6 weeks prior to screening period and likely to have low-density lipoprotein cholesterol (LDL-C) ≥100 mg/dl (≥2.59 mmol/L) at the screening visit (Week-1).

### Exclusion Criteria:

LDL-C<100 mg/dL (<2.59 mmol/L) at Week-1 (V1):
   After the run-in period on atorvastatin 10 mg for patients receiving a lipid lowering treatment other than atorvastatin/or not at stable dose of atorvastatin 10 mg for at least 6 weeks prior to the screening period, or drug naive patients.
   OR
   At the first visit for patients who are being treated with atorvastatin 10 mg at stable dose for at least 6 weeks prior to screening visit Week-1.
Body mass index (BMI)<18 or >40 kg/m² at Week-7 or Week-1.
Patients not previously instructed on a cholesterol-lowering diet.
Use of a statin other than atorvastatin 10 mg, or use of other lipid lowering drugs including but not limited to fibrates, bile acid resins, niacin >500 mg, intestinal cholesterol absorption (ICA) blockers, or omega-3 fatty acids at doses >1000 mg during the screening period.
Patients with type 1 diabetes.
Patients with type 2 diabetes treated with insulin.
Patients with type 2 diabetes and with an HbA1c ≥8.5% at Week-7 or Week-1 (considered poorly controlled).
Laboratory findings measured before randomization:
   Positive test for hepatitis B surface antigen and/or hepatitis C antibody.
   Triglycerides (TG)>350 mg/dL (>3.95 mmol/L) at Week-7 or Week-1.
   Neutrophils <1,500/mm³ and/or platelets <100,000/mm³.
   Positive serum or urine pregnancy test in females of childbearing potential.
   Abnormal sensitive TSH level (>ULN or <LLN) according to the normal values of the Central Laboratory.
   Evidence of renal impairment as determined by:
      Men: serum creatinine >1.5×ULN.
      Women: serum creatinine >1.4×ULN.
   ALT or AST >2×ULN (1 repeat lab is allowed).
   CPK>3×ULN (1 repeat lab is allowed).
All contraindications to the protocol mandated background therapy (i.e., atorvastatin) or warning/precaution of use (when appropriate) as displayed in the respective National Product Labeling that was used for defining these exclusion criteria.
Known sensitivity to monoclonal antibody therapeutics.
Pregnant or breast-feeding women.
Women of childbearing potential with no effective contraceptive method.

### Duration of Study Period Per Subject:

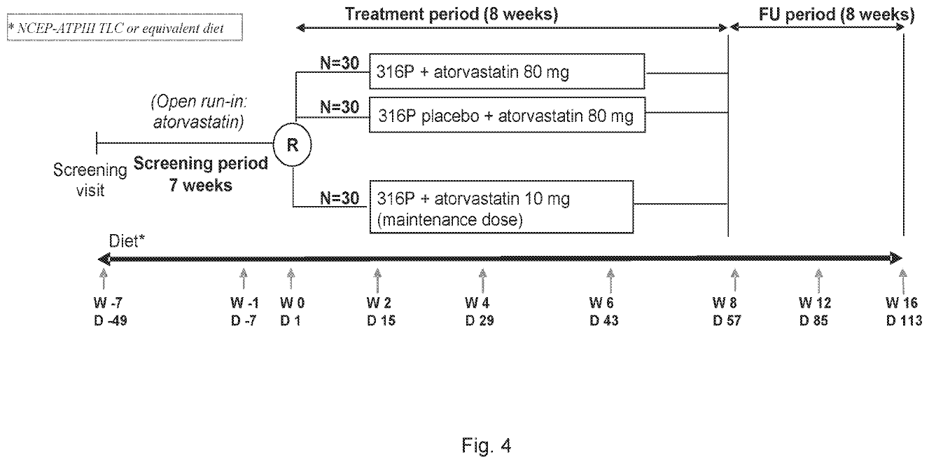

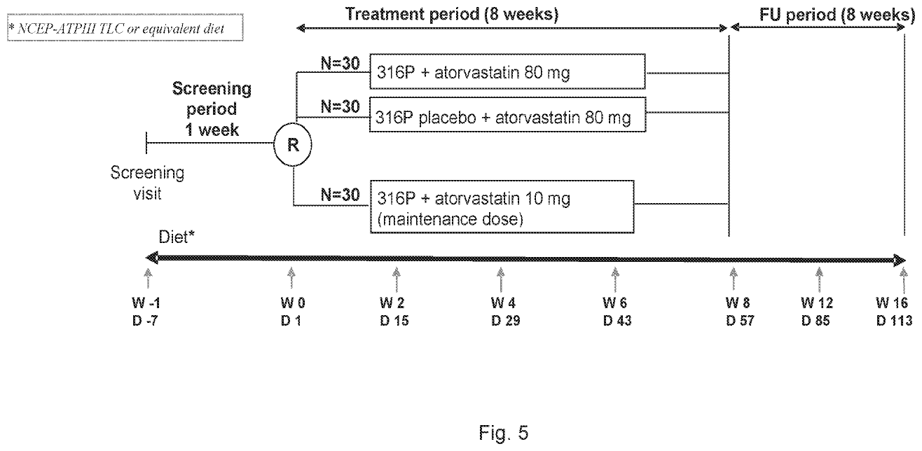

The duration of study participation will depend on the status of the patient at screening:
For patients receiving atorvastatin 10 mg at stable dose for at least 6 weeks prior to screening, the study participation will be approximately 17 weeks including a screening period of 1 week, a double-blind treatment period of 8 weeks and a follow-up period of 8 weeks (see FIG. 5).
For patients receiving a lipid lowering treatment other than atorvastatin/or not at stable dose of atorvastatin 10 mg for at least 6 weeks prior to screening, or drug naive patients the study participation will be approximately 23 weeks with a screening period of 7 weeks (including a run-in period of 6 weeks), a double-blind treatment period of 8 weeks and a follow-up period of 8 weeks (see FIG. 4).

### Active Compounds:

### Antibody 316P

Antibody 316P is a fully human antibody comprising a HCVR as shown in SEQ ID NO: 90 and LCVR as shown in SEQ ID NO: 92 of the sequence listing. The CDR sequences are shown in SEQ ID NOs: 76, 78, and 80 (CDR1, CDR2, CDR3 of the heavy chain) as well as in SEQ ID NOs: 84, 86, and 88 (CDR1, CDR2, CDR3 of the light chain).

### Antibody 300N

Antibody 300N is a fully human antibody comprising a HCVR as shown in SEQ ID NO: 218 and LCVR as shown in SEQ ID NO: 226 of the sequence listing. The CDR sequences are shown in SEQ ID NOs: 220, 222, and 224 (CDR1, CDR2, CDR3 of the heavy chain) as well as in SEQ ID NOs: 228, 230, and 232 (CDR1, CDR2, CDR3 of the light chain).

### Study Arms:

Arm 1: The first group of patients receives one subcutaneous injection of 1 mL of antibody 316P, administered in the abdomen every two weeks, with a dose regimen at 150 mg, for a double-blind treatment period of 8 weeks;
   Atorvastatin is administered once per day at a stable dose of 10 mg as background therapy.
   Atorvastatin is administered at a dose of 80 mg once during the double-blind treatment period of 8 weeks.
Arm 2: The second group of patients receives one subcutaneous injection of 1 mL of a placebo solution, administered in the abdomen every two weeks, with a dose regimen at 150 mg, for a double-blind treatment period of 8 weeks;
   Atorvastatin is administered once per day at a stable dose of 10 mg as background therapy.
   Atorvastatin is administered at a dose of 80 mg (2 over-encapsulated atorvastatin 40 mg tablets) once during the double-blind treatment period of 8 weeks.
Arm 3: The third group of patients receives one subcutaneous injection of 1 mL of antibody 316P, administered in the abdomen every two weeks, with a dose regimen at 150 mg, for a double-blind treatment period of 8 weeks;
   Atorvastatin is administered once per day at a stable dose of 10 mg as background therapy.
   Atorvastatin is administered at a dose of 10 mg (1 over-encapsulated atorvastatin 10 mg tablet+1 matching placebo tablet) once during the double-blind treatment period of 8 weeks.

### Primary and Key Secondary Endpoints

### Primary Endpoints

The primary efficacy variable is the percent change in calculated LDL-C from baseline to Week 8, which is defined as: 100× (calculated LDL-C value at Week 8-calculated LDL-C value at baseline)/calculated LDL-C value at baseline.

In case of unavailable calculated LDL-C value at Week 8 as defined above, then the last calculated LDL-C value measured during the efficacy period and before the Week 8 time window was used to impute the missing Week 8 calculated LDL-C value (Last Observation Carried Forward [LOCF] procedure).

### Key Secondary Endpoints

### The Secondary Efficacy Variables are:

The absolute change (mmol/L and mg/dL) from baseline in calculated LDL-C to Week 8, defined as: (calculated LDL-C value at Week 8-calculated LDL-C value at baseline)
The percentage of patients with calculated LDL-C<70 mg/dL (1.81 mmol/L) and <100 mg/dL (2.59 mmol/L) at Week 8.
Percent change in ApoB from baseline to Week 8
Percent and absolute (mmol/L and mg/dL) change in non HDL-C from baseline to Week8
Percent and absolute (mmol/L and mg/dL) change in total cholesterol from baseline to Week 8
Percent and absolute (mmol/L and mg/dL) change in HDL-C from baseline to Week 8
Percent and absolute (mmol/L and mg/dL) change in fasting Triglycerides from baseline to Week 8
Percent change in ApoA-1 from baseline to Week 8
Absolute change in the ratio ApoB/ApoA-1 from baseline to Week 8
Percent change in Lp (a) from baseline to Week 8.

### Sample Size Calculation Assumptions

The study was expected to enroll approximately 90 patients.

To detect a difference of 20% in LDL-C percent change from baseline to Week 8 between 316P 150 mg+atorvastatin 80 mg group and Placebo for 316P+atorvastatin 80 mg group, assuming a 5% rate of unevaluable primary endpoint, 30 patients by arm were estimated to result in 95% power, with a standard deviation of 20%, and using a two-sided t-test at the 0.05 significance level.

Calculations were made using nQuery Advisor 6.01.

### Statistical Methods

### Analysis Populations

### Efficacy Populations

The primary efficacy analysis population is the modified intent-to-treat (mITT) population.

### Modified Intent-to-Treat Population

Modified ITT (mITT) population: randomized population with an evaluable primary endpoint.

The primary endpoint was evaluable when both of the following conditions are met:
Availability of at least one calculated LDL-C value from the Visit 1 (Week-1) and up to before first IP injection.
Availability of at least one calculated LDL-C value during the efficacy period and, within or before the Week 8 time window.

Patients in the mITT population were analyzed according to the treatment group allocated by randomization.

### Per-Protocol Population

Per-protocol (PP) population is a subset of the mITT population, excluding patients:
with important protocol deviations impacting LDL-C baseline or LDL-C assessment at Week 8,
receiving prohibited therapy potentially impacting lipids levels during the pre-treatment period or during the efficacy period before the primary endpoint assessment
with a poor compliance to 316P IP administrations.
with a poor compliance to atorvastatin non IP during the pre-treatment period or with non compliance to atorvastatin IP during the 3 days preceding primary endpoint assessment.

### Safety Population

Safety population is defined as the randomized population who did actually receive at least one dose or partial dose of 316P IP analyzed according to the treatment actually received. Patients treated without being randomized would not be considered as randomized and would not be included in any populations. The safety experience of patients treated and not randomized would be reported separately.

### Primary Efficacy Analysis

The percent change from baseline in calculated LDL-C at Week 8-LOCF as defined above was analyzed in the mITT population using an analysis of covariance (ANCOVA) model with treatment group as fixed effect and the baseline LDL-C as covariate. The treatment group factor had three levels: placebo+atorvastatin 80 mg, 316P 150 mg E2W+atorvastatin 10 mg and 316P 150 mg E2W+atorvastatin 80 mg.

Throughout the ANCOVA model, the 316P 150 mg E2W+atorvastatin 80 mg group was compared to the placebo+atorvastatin 80 mg group using appropriate contrast and the 95% confidence interval (CI) of the difference was provided.

No formal comparison with the 316P 150 mg E2W+atorvastatin 10 mg group was performed: only 95% CIs for difference versus the other arms was provided.

### Key Secondary Efficacy Analysis

Continuous secondary efficacy variables were analyzed in the mITT population using the same ANCOVA model as for the primary endpoint. For triglycerides and LP (a) known to have non Gaussian distribution, the rank-based ANCOVA method was used.

Binary secondary efficacy variables were analyzed in the mITT population using an exact conditional logistic regression model with treatment group and baseline LDL-C level as effects.

### Safety Analysis

The safety analysis was based on reported adverse events (AEs) (if any) and other safety information, such as clinical laboratory data, vital signs, and ECG.

The TEAE period was defined as the time from first IP injection to last IP injection+70 days (10 weeks).

AEs of interest included the following terms:
Possible injection site reaction (HLT “Injection site reactions”)
Possible allergic events (HLGT “Allergic conditions”)
Patients with LDL-C<25 mg/dL (if any) or LDL-C<15 mg/dL (if any).

### Other Assessments Analysis

Other assessment endpoints defined below are exploratory variables. They include metabolic and inflammatory parameters:
Absolute change in HbA1c (%) from baseline to Week 8
Absolute change from baseline in fasting plasma glucose (mmol/L) to Week 8
Percent change from baseline in hs-CRP to Week 8.

Those endpoints were summarized in the m-ITT population by time points using descriptive statistics. The time profile (including LOCF value) of each parameter was also plotted by treatment group with the corresponding standard errors.

PCSA criterion for hs-CRP was also summarized by treatment group using descriptive statistics.

### Results

Study 2 was a multicenter, randomized, double-blind, parallel-group, placebo-controlled, 8-week study conducted in the United States to assess the efficacy and safety of 316P in patients with an elevated low-density lipoprotein cholesterol (LDL-C) (≥100 mg/dL or 2.59 mmol/L), treated with a stable dose of atorvastatin 10 mg for at least 6 weeks. After the 8-week double-blind period patients were followed during an 8-week follow-up period.

The primary objective of the study was to evaluate the effect of 316P on LDL-C levels compared with placebo when co-administered with 80 mg of atorvastatin after 8 weeks of treatment in patients with LDL-C≥100 mg/dL (≥2.59 mmol/L) previously on atorvastatin 10 mg. Evaluation of the efficacy of the co-administration of 316P with this high dose of atorvastatin (80 mg) compared with that of the co-administration of 316P with atorvastatin 10 mg was one of the secondary objectives. The dose regimen of 150 mg every 2 weeks (E2W) in comparison with placebo was evaluated.

Efficacy analyses were performed on 88 patients (29 in the placebo+atorvastatin 80 mg group, 29 in the 316P 150 mg+atorvastatin 10 mg group, and 30 in the 316P 150 mg+atorvastatin 80 mg group).

Demographics and baseline characteristics were similar across the treatment groups. The median age of patients was 58.0 years (25.0% of patients were ≥65 years of age). The mean baseline LDL-C and Total-C ranged between 3.101 mmol/L and 3.288 mmol/L, and between 5.447 mmol/L and 5.200 mmol/L, respectively.

### Efficacy:

A statistically significant decrease in percent change from baseline in LDL-C at 8 weeks was observed in the 316P 150 mg+atorvastatin 80 mg group compared with the placebo+atorvastatin 80 mg group (LS mean difference of −55.8%; p<0.0001). Because of the non-gaussian distribution and non homogeneity of variance of the primary efficacy endpoint, a sensitivity analysis was also performed using rank-based analysis of covariance which showed similar results: effect size estimate of 316P 150 mg+atorvastatin 80 mg vs placebo+atorvastatin 80 mg of −54.5%, p<0.0001. Large decreases from baseline were seen in both treatments groups where 316P 150 mg was co-administered with atorvastatin, with a median reduction of −70.4% for the 316P 150 mg+atorvastatin 10 mg group, and of −70.6% for the 316P 150 mg+atorvastatin 80 mg group compared with a median reduction of −26.9% in the placebo+atorvastatin 80 mg group.

Consistent results were seen for Total-C, ApoB, non HDL-C and Apo-B/ApoA-1 ratio. For HDL-C, an increase in the percent change from baseline was observed in both treatment groups where 316P 150 mg was co-administered with atorvastatin 10 mg or 80 mg (LS mean+2.6%, and +5.8%, respectively) compared with a decrease in the placebo+atorvastatin 80 mg group (LS mean-3.6%).

### Safety:

316P was well tolerated during the 8 weeks of treatment in all treatment groups. Significantly, no change in troponin levels was noted in all treatment groups.

### Conclusion:

There was a statistically significant decrease in percent change from baseline in LDL-C at 8 weeks in the 316P 150 mg+atorvastatin 80 mg group as compared with the placebo+atorvastatin 80 mg group (LS mean difference of −55.8%; p<0.0001). A similar magnitude of effect observed with 316P was noted regardless of the dose of atorvastatin (10 mg or 80 mg) with a substantial decrease in LDL-C when co-administered to these 2 atorvastatin doses.

Consistent results were seen for Total-C, ApoB, non HDL-C and Apo-B/ApoA-1 ratio. For HDLC, there was a trend of increase in both treatment groups where 316P 150 mg was co-administered with atorvastatin 10 mg or 80 mg.

316P 150 mg E2W was well tolerated during the 8 weeks of treatment in all treatment groups. No particular safety signal was noted.

Efficacy of 316P 150 mg E2W as well its good safety profile were confirmed in this study regardless of the dose of atorvastatin administered (10 mg or 80 mg).

### Study 3

This is a randomized, double-blind, placebo-controlled, multiple ascending dose, multicenter clinical trial in subjects with primary hypercholesterolemia.

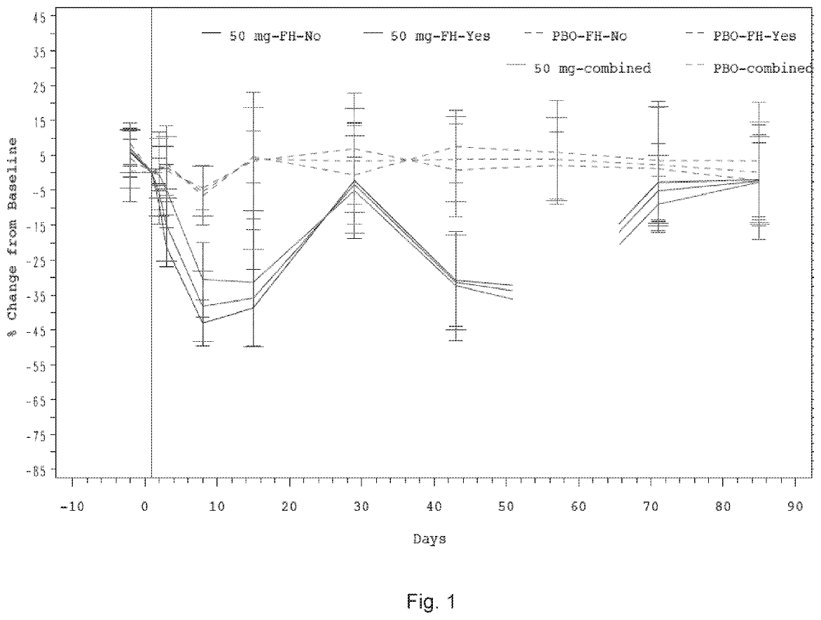

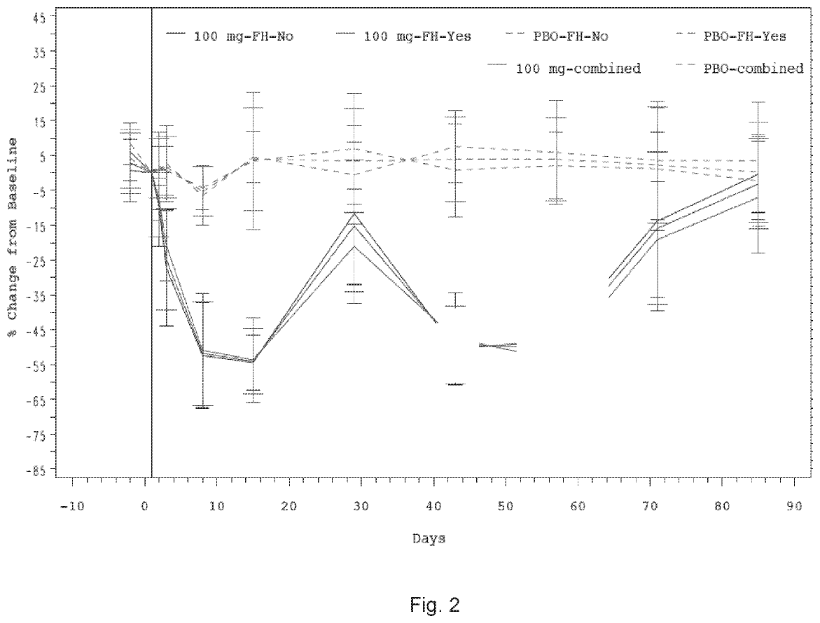

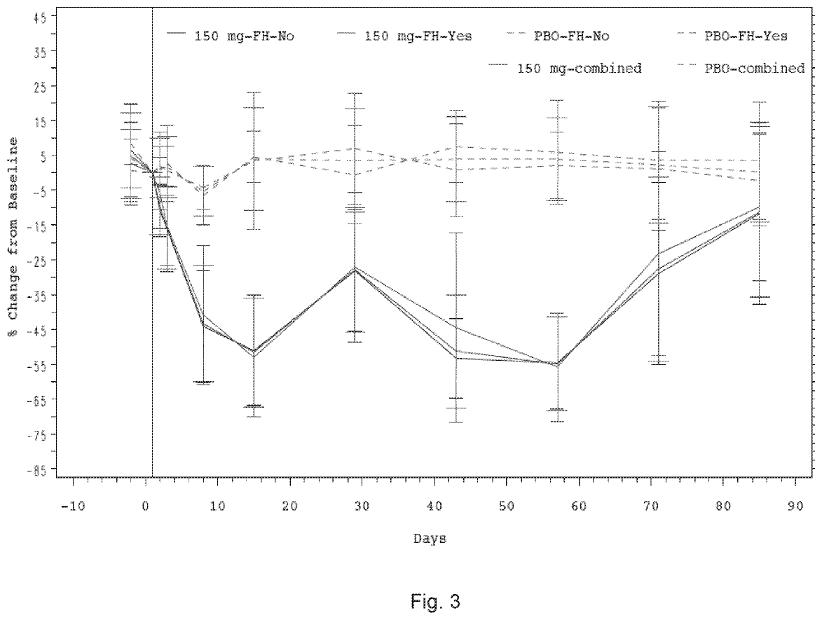

The objective of this study was to determine whether a fully human monoclonal antibody to PCSK9 (316P) is effective and safe as either a primary or adjunctive agent to lower LDL-C in patients with Heterozygous Familial Hypercholesterolemia (HeFH) or other forms of primary hypercholesteremia (nonFH). 61 adults with either documented HeFH (n=21) or nonFH (n=30), on diet plus stable atorvastatin therapy (atorvaRx) or nonFH (n=10) on diet alone enrolled in this clinical trial. Subjects on stable atorvastatin therapy had LDL-C ≥2.6 mmol/L and those on diet alone had LDL-C ≥3.4 mmol/L. 316P at doses of 50, 100 and 150 mg was administered subcutaneously (sc) at 1, 29 and 43 days. The primary endpoint was the incidence and severity of treatment emergent adverse events (TEAE). The primary efficacy endpoint was percent and absolute change in serum LDL-C from baseline to each visit. Additional endpoints included apolipoprotein (apo) B, total cholesterol, HDL-C, VLDL-C, and the ratio of apoB to apoA1. 109 patients were screened, and 61 patients were randomized (14 placebo, 47 316P) with 100% completing 148+/−7 days of treatment and follow up. Compared to the nonFH cohort, the FH group was younger (mean 40 vs. 52 yrs), had more males (81% vs. 57%) and was on higher doses of atorvastatin (52% on 40 mg vs. 3%). Baseline LDL-C was 3.45, 2.88 and 4.46 mmol/L in the FH, nonFH atorvaRx and nonFH diet only groups, respectively. Treatment with 316P resulted in mean % reductions in LDL-C on top of statins on day 57 of 35.6%, 50.2% and 57.5% at the 50, 100 and 150 mg doses, respectively, in the combined FH and nonFH populations. Although no statistical analysis was performed, there did not appear to be differences in response between FH and nonFH or those on or not on statin therapy. Response to 316P is shown in FIGS. 1, 2 and 3. Favourable changes were observed in HDL-C and apoA1. No serious adverse events were seen and treatment was generally well-tolerated. No drug-related adverse effects were seen on liver function testing or other laboratory parameters.

This first multiple-dose, proof-of-concept trial of a PCSK9 inhibitor, in FH and nonFH on stable statin therapy, shows that treatment with an anti-PCSK9 antibody, such as 316P, is a promising therapeutic option for patients with or without HeFH with elevated cholesterol on statin therapy.

### Study 4

This is an animal study on the cholesterol lowering effect of 316P, a fully human PCSK9 blocking monoclonal antibody in male Syrian hamster

### Introduction

The hepatic LDL receptor (LDLR) is the key component for cholesterol homeostasis. PCSK9 regulates hepatic LDLR levels by enhancing its degradation. The transcription of both the LDLR and PCSK9 is up-regulated by statins through SREBP-2, thereby limiting the extent that statins can lower LDL-cholesterol (LDL-C) in humans and even more in rodents where statins are not effective in reducing LDL-C.

### Objective

The aim of this study was to investigate the effect of 316P, a human monoclonal antibody to human PCSK9, alone and in combination with statins on expression of the hepatic LDLR and the resulting effects on serum LDL-C.

### Results

In hamster, a single s.c. injection of 316P (1/3/10 mg/kg) resulted in a dose-dependent decrease in LDL-C lasting more than 2 weeks. The maximal effect on LDL-C (-17/-27/-60%) was seen within 7 days. PK data of 316P are in line with the dose-dependent effect on LDL-C. Atorvastatin treatment up to the maximal tolerated dose has no effect on hepatic LDLR expression and did not decrease LDL-C. 316P on top of Atorvastatin could overcome the statin resistance increased LDLR expression and decreased serum LDL-C. The combination treatment was more effective than single treatment with 316P alone, although Atorvastatin alone has no effect.

### Conclusion

PCSK9 inhibition resulted in dose-related LDL-C-lowering in hamsters. However, when administered in combination with a normally ineffective dose of Atorvastatin, a potentiated reduction in LDL-C was observed. These data suggest that neutralizing PCSK9 is effective in overcoming the statin-resistance observed in the hamster model. This data are in accordance with results in a human phase I study, where LDL-C reduction exceeded 60% and lasted for 30 days following a single i.v. administration. This confirmed that the hamster is a suitable model to investigate drugs targeting PCSK9.

### Study 5

This is a randomized, double-blind, placebo-controlled, unbalanced (2:1, 316P:placebo), parallel-group study with an open-label extension.

### Objective(s)

The primary objective of this study is to evaluate the long-term safety and tolerability of 316P over the main treatment period in hypercholesterolemic patients at risk of cardiovascular disease not adequately controlled with their lipid lowering treatment.
Secondary objectives are
To evaluate the long-term safety and tolerability of 316P over the whole study duration.
To evaluate the effect of 316P on low-density lipoprotein cholesterol (LDL-C) levels after 12 weeks of treatment in comparison with placebo.
To evaluate the long-term efficacy of 316P on low density lipoprotein cholesterol (LDL-C) levels.
To evaluate the effect of 316P on Total-Cholesterol (TC), non-high density lipoprotein cholesterol (non-HDL-C), Apolipoprotein B (ApoB), HDL-C, Triglycerides (TG), Apolipoprotein A-1 (ApoA-1), ratio ApoB/ApoA-1, and Lipoprotein a (Lp (a)) after 12 weeks of treatment in comparison with placebo and after long term treatment
To evaluate the development of anti-316P antibodies.
To evaluate the pharmacokinetics (PK) of 316P.
To explore the effect of 316P on adjudicated cardiovascular events over the main treatment period in comparison with placebo and over the whole study duration.

### Study Design

Patients will be stratified according to heterozygous familial hypercholesterolemia (heFH) population, prior history of myocardial infarction (MI) or stroke, high-intensity statin therapy (ie, atorvastatin 40 to 80 mg daily or rosuvastatin 20 to 40 mg daily) and geographic region. Patients randomized to 316P will receive 150 mg subcutaneous (sc) every 2 weeks. This dose/dose regimen, assessed in the Phase 1 program, is also one of the doses/dose regimens being evaluated in the Phase 2 program. For the present study, the administration of 150 mg subcutaneous every 2 weeks has been selected as the dose/dose regimen providing the highest systemic exposure to 316P in the range of doses/regimens likely to be effective. This dose and regimen may be adjusted, if needed, to a different dose/dosing frequency during the course of the study, through a protocol amendment, when the full data set of dose/regimen finding data become available.

### The Study Consists of:

A screening period of up to 2 weeks, including an intermediate visit during which the patient or another designated person (such as spouse, relative, etc.,) will be trained to selfinject/inject with placebo.
A double-blind period of 18 months study treatment with 316P or placebo for all patients.
   The main treatment period is defined for the purpose of the primary analysis and this period ends 12 months after the last patient in (LPI) is randomized, and includes patients with variable duration of double blind treatment between 12 months and 18 months.
An open-label period (OLP) which consists of study treatment with 316P in patients who have completed the 18-month double-blind period. The OLP will be of variable duration and ends for all patients at 24 months after the LPI or at 39 months after the FPI, whichever comes first.
A follow-up period (off-treatment) of 8 weeks after the end of the open-label period.

Patients will be instructed to be on a stable diet (NCEP-ATPIII TLC diet or equivalent) throughout the entire study duration from screening. Statin dose as well as dose of other lipid-lowering treatment(s) (if applicable) should be stable throughout the whole study duration. During the double-blind period, modification is allowed under certain conditions. During the open-label period, modification is based upon investigator's judgment. Fibrates other than fenofibrate are not allowed during the study. The lipid parameters will be blinded during the double-blind period.

### Study Population:

### Inclusion Criteria

Either A or B below AND not adequately controlled with a maximally tolerated stable dose of statin for at least 6 weeks prior to the screening visit (Week-2) with or without other lipid lowering therapy (LLT).
A) Patients with heterozygous familial hypercholesterolemia (heFH)
   OR
B) Patients with non-familial hypercholesterolemia (non-FH) with established coronary heart disease (CHD) or CHD risk equivalents

### Note:

1 All background LLT, including therapy other than statins, should be at a stable dose for at least 6 weeks prior to the screening visit (week-2).
The only statins which are permissible at study inclusion are simvastatin, atorvastatin, and rosuvastatin taken daily.
Patients are eligible for the study if they are on maximally tolerated statin even if this is not high-intensity statin. Maximally tolerated statin is defined as any daily dose of simvastatin, atorvastatin, and rosuvastatin that is maximally tolerated. High-intensity statin is defined as atorvastatin 40 to 80 mg daily or rosuvastatin 20 to 40 mg daily.
If patient is not on high-intensity statin during screening, then the reason needs to be documented (ie, myalgias, liver enzyme abnormalities, etc.).
If Screening (Week-2 visit) LDL-C is ≥160 mg/dL (4.14 mmol/L), patients should have been offered another LLT in the past in addition to their maximally tolerated statin. In addition, if patients are on maximally tolerated statin therapy only, then reason needs to be documented; such patients are still eligible for the study and are not excluded.
Daily doses above simvastatin 80 mg, atorvastatin 80 mg or rosuvastatin 40 mg are not allowed for study inclusion.
Simvastatin 80 mg should be used only in patients who have been taking this dose for 12 months or more without evidence of muscle injury (myopathy) and should not be started in new patients, including patients already taking lower doses of the drug.
Prescriptions of other LLT should be in accordance with the national product label.

### Key Exclusion Criteria

LDL-C<70 mg/dL (<1.8 mmol/L) at the screening visit (Week-2).
TG >350 mg/dL (>3.95 mmol/L) at the screening visit (Week-2).
Use of fibrates other than fenofibrate within 6 weeks prior to screening visit (Week-2) or plan to receive it.

### Total Expected Number of Patients:

Approximately 2100 randomized (1400:700, 316P:placebo)

### Study Treatment(s)

### Investigational Medicinal Product(s): Antibody 316P and Placebo for 316P

Antibody 316P is a fully human antibody comprising a HCVR as shown in SEQ ID NO: 90 and LCVR as shown in SEQ ID NO: 92 of the sequence listing. The CDR sequences are shown in SEQ ID NOs: 76, 78, and 80 (CDR1, CDR2, CDR3 of the heavy chain) as well as in SEQ ID NOs: 84, 86, and 88 (CDR1, CDR2, CDR3 of the light chain).

Alternatively, the study can be carried out with antibody 300N (=back-up compound) instead of antibody 316P. Antibody 300N is a fully human antibody comprising a HCVR as shown in SEQ ID NO: 218 and LCVR as shown in SEQ ID NO: 226 of the sequence listing. The CDR sequences are shown in SEQ ID NOs: 220, 222, and 224 (CDR1, CDR2, CDR3 of the heavy chain) as well as in SEQ ID NOs: 228, 230, and 232 (CDR1, CDR2, CDR3 of the light chain).

### Formulation

Prefilled syringes: 316P 150 mg/mL, or placebo for 316P.

### Route(s) of Administration:

Subcutaneous (SC)
Injection volume: 1 mL in total for the dose of 150 mg
One injection of 1 mL subcutaneous over the abdomen, thigh, or outer area of upper arm (ie, deltoid region).
   Dose regimen: Dose of 150 mg every 2 weeks

### Primary and Secondary Endpoint(s)

### Primary Endpoint:

Safety parameters (adverse events [including adjudicated cardiovascular events], laboratory data, vital signs, and ECG) assessed throughout the main treatment period.

### Main Secondary Endpoints:

Safety parameters (adverse events [including adjudicated cardiovascular events], laboratory data, vital signs, and ECG) assessed throughout the whole study duration
The percent change in LDL-C from baseline to Week 12 (as main time point).
Anti-316P antibodies
Serum 316P concentrations

### Assessment Schedule

### Patient's Assessments in the Screening Period:

On-site visits: Week-2 (screening visit), Week-1 (Injection training visit).

### Patient's Assessments in the Double-Blind Period:

On-site visits: Week 0 (randomization visit=baseline), Week 4, Week 8, Week 12, Week 16, Week 24, Week 36, Week 52/Month 12, Week 64/Month 15, Week 78/Month 18 (end of double blind period).
Phone calls: Week 2*, Week 20, Week 28, Week 32, Week 40, Week 44, Week 48, Week 56, Week 60, Week 68, Week 72 and Week 76.
   *Note: Week 2 could become an on-site visit for further injection training with the patient's scheduled injection from the double-blind study treatment kit allocated by IVRS, as needed.

### Patient's Assessments in the Open-Label Period:

On-site visits: Every 12 weeks after the end of the double-blind period visit and until the end of open label period visit.
Phone calls: Every 4 weeks between on-site visits.

Note: During the course of the study, through the ongoing safety reviews, the Data Monitoring Committee (DMC) will assess the adequacy of the visit frequency and corresponding procedures for the open-label period and make appropriate recommendations.

### Patient's Assessments in the Follow-Up Period:

On-site visit: 8 weeks after the end of open label period visit.

### Statistical Considerations

For safety assessment, a sample size of 2100 patients (randomization ratio 2:1, ie, 316P: 1400 and placebo: 700) will allow to have long term safety data in a broad database. With this sample size, 1050 and 364 patients are expected to be exposed to 316P for a minimum of 12 months and 18 months, respectively, at the time of the primary analysis (12 months after the last patient in). Moreover, with 1400 patients treated with 316P, adverse events with a rate ≥0.002 will be detected with 95% confidence.

The stratification factors include heFH population, prior history of MI or stroke, high-intensity statin and region (North America, Western Europe, Eastern Europe, Rest of World).

Summary of safety variables will be performed based on the safety population. The safety population consists of the randomized population who did actually receive at least one dose or partial dose of Investigational Medicinal Product (IMP) analyzed according to the treatment actually received.

Descriptive statistics will be used for the summary of safety variables from this study. For adverse events, in addition to summary tables presented with crude rates, the table of all TEAEs will be provided using patient-year adjusted incidence rates. If any clinically significant signal is detected and need further characterization or for adverse event or Potentially Clinically Significant Abnormality (PCSA) of interest, a time-to-event analysis will be performed using Kaplan-Meier methodology. Moreover, the frequency of adverse event or PCSA of interest over time will be provided. The primary safety analysis will be done on the safety events that can be attributed to the administration of double blind treatment during the main treatment period. Secondary safety analyses will be conducted on the safety events observed during the double-blind period and the open-label period.

The efficacy analysis population will be the modified intent to-treat (mITT) population, defined as the ITT population (i.e., randomized population) with an evaluable LDL-C endpoint. This endpoint will be considered as evaluable when both of the following conditions are met:
The baseline LDL-C value is available.
At least one LDL-C value collected in the main efficacy period is available.

The main efficacy period will be defined as:
The time from the first IMP injection (excluding training injection) up to 21 days after the last IMP injection for patients who permanently discontinue the IMP before Week 12.
The time from the first IMP injection (excluding training injection) up to Week 12 for patients who were treated at least 12 weeks.

Patients in the mITT population will be analyzed according to the treatment group allocated by randomization.

The percent change in LDL-C from baseline to Week 12 (main secondary endpoint) and at other time points throughout the study (other secondary endpoints) will be analyzed using an analysis of covariance (ANCOVA) model with treatment group and each stratification factor (heFH population, prior history of MI or stroke, high-intensity statin, region) as fixed effect and the baseline LDL C as covariate. The treatment group factor will have 2 levels: placebo and 316P. Throughout the ANCOVA model, the 316P group will be compared to placebo using appropriate contrast, and the 95% CI of the difference will be provided.

In case of missing Week 12 LDL-C on treatment value, the last-observation-carried-forward (LOCF) principle will be used.

### Duration of Study Period (Per Patient)

The study duration for each patient is variable. The maximum study duration includes up to 2 weeks of screening period, 18 months study treatment during double blind period, up to 21 months of 316P treatment in the open label period (depending on when patient randomized into study and duration of recruitment) and 8 weeks of follow up period. Thus, the maximum study duration is up to ˜ 42 months for the first patient randomized into the study and up to ˜ 27 months for the last patient randomized into the study.

### Study 6

A randomized, double-blind, multi-dose, placebo controlled, 75-patient trial in patients with heterozygous familial hypercholesterolemia (heFH). In this trial, patients must meet the World Health Organization criteria for heFH, be on a stable daily statin regimen for at least 6-weeks before entering the trial, and have serum LDL-C levels ≥100 mg/dL. Patients were permitted to be taking ezetimibe in addition to a daily statin. The primary endpoint of the study is the change in LDL cholesterol from baseline compared to placebo over the 12-week study period.

An interim analysis of study 6 in heterozygous familial hypercholesterolemia patients with elevated cholesterol (LDL-C≥100 mg/dL) on stable doses of statins with or without ezetimibe demonstrated that patients treated with 316P every two or four weeks achieved significantly greater mean LDL-C reductions at 12-weeks compared to patients treated with placebo. Patients treated with different doses of 316P achieved mean LDL-C reductions of approximately 30% to greater than 60% from baseline at 12-weeks compared to a 10% reduction with placebo (p<0.01), which was the primary endpoint of the study. The interim analysis was conducted when all patients completed the primary endpoint at 12-weeks.

1-29. (canceled)

30. A method for treating a disease or condition in which proprotein convertase subtilisin/kexin type 9 (PCSK9) expression or activity causes an impact, the method comprising:
administering to a subject in need thereof a pharmaceutical composition comprising a fixed dose of 75, 150, or 300 mg of an antibody or an antigen-binding fragment thereof which specifically binds human proprotein convertase subtilisin/kexin type 9 (hPCSK9) together with a pharmaceutically acceptable excipient or carrier,
wherein the pharmaceutical composition is administered about every two or four weeks, and wherein the antibody or antigen-binding fragment thereof comprises the three heavy chain complementarity determining regions (CDRs) set forth in SEQ ID NOs: 76, 78, and 80 and the three light chain CDRs set forth in SEQ ID NOs: 84, 86, and 88;
wherein the pharmaceutical composition is administered as an adjunct to diet, alone, or in combination with other low-density lipoprotein cholesterol (LDL-C) lowering therapies.

31. The method of claim 30, wherein the antibody or antigen-binding fragment thereof comprises the heavy chain variable region (HCVR) amino acid sequence and the light chain variable region (LCVR) amino acid sequence set forth in SEQ ID NOs: 90 and 92, respectively.

32. The method of claim 30, wherein the disease or condition in which PCSK9 expression or activity causes an impact is selected from the group consisting of: elevated total cholesterol levels, elevated non-high-density lipoprotein (HDL) cholesterol levels, elevated low-density lipoprotein (LDL-C) levels, elevated apolipoprotein B100 (ApoB100) levels, hyperlipidemia, dyslipidemia, atherosclerosis, cardiovascular diseases, coronary heart disease (CHD), hypercholesterolemia, primary hypercholesterolemia, familial hypercholesterolemia, non-familial hypercholesterolemia, heterozygous familial hypercholesterolemia (heFH), hypercholesteremia that is uncontrolled by statins, and hypercholesterolemia that is resistant to statins.

33. The method of claim 30, wherein the antibody or antigen-binding fragment thereof is for administration to a subject falling into at least one of the following groups of subjects:
(i) subjects having a serum LDL cholesterol (LDL-C) level of at least 100 mg/dL,
(ii) subjects having a serum HDL-C level of less than 40 mg/dL;
(iii) subjects having a serum cholesterol level of at least 200 mg/dL; and
(iv) subjects having a serum triacylglycerol level of at least 150 mg/dL; wherein said triacylglycerol level is determined after fasting for at least 8 hours.

34. The method of claim 30, wherein the pharmaceutical composition comprises a fixed dose of 75 mg of the antibody or antigen-binding fragment thereof, and wherein the pharmaceutical composition is administered about every two weeks.

35. The method of claim 30, wherein the pharmaceutical composition comprises a fixed dose of 150 mg of the antibody or antigen-binding fragment thereof, and wherein the pharmaceutical composition is administered about every two weeks or about every four weeks.

36. The method of claim 30, wherein the pharmaceutical composition comprises a fixed dose of 300 mg of the antibody or antigen-binding fragment thereof, wherein the pharmaceutical composition is administered about every four weeks.

37. The method of claim 30, wherein the pharmaceutical composition is a 1 mL injection solution comprising a fixed dose of 75 mg, 150 mg, or 300 mg of the antibody or antigen-binding fragment thereof.

38. The method of claim 37, wherein the injection solution comprises a fixed dose of 75 mg of the antibody or antigen-binding fragment thereof and wherein the pharmaceutical composition is administered about every two weeks.

39. The method of claim 37, wherein the injection solution comprises a fixed dose of 150 mg of the antibody or antigen-binding fragment thereof and wherein the pharmaceutical composition is administered about every two weeks or about every four weeks.

40. The method of claim 37, wherein the injection solution comprises a fixed dose of 300 mg of the antibody or antigen-binding fragment thereof and wherein the pharmaceutical composition is administered about every four weeks.

41. The method of claim 30, wherein the pharmaceutical composition is comprised in a unit dosage form, and wherein the unit dosage form comprises a fixed dose of 75, 150, or 300 mg of the antibody or antigen-binding fragment thereof.

42. The method of claim 41, wherein the unit dosage form comprises a fixed dose of 75 mg of the antibody or antigen-binding fragment thereof and wherein the pharmaceutical composition is administered about every two weeks.

43. The method of claim 41, wherein the unit dosage form comprises a fixed dose of 150 mg of the antibody or antigen-binding fragment thereof and wherein the pharmaceutical composition is administered about every two weeks or about every four weeks.

44. The method of claim 41, wherein the unit dosage form comprises a fixed dose of 300 mg of the antibody or antigen-binding fragment thereof and wherein the pharmaceutical composition is administered about every four weeks.

45. The method of claim 30, wherein the pharmaceutical composition is in a hermetically sealed container selected from the group consisting of a vial, a sachette, a pre-filled syringe, a pre-filled autoinjector, a cartridge for a reusable syringe, and an applicator.
optionally wherein the pharmaceutical composition is administered by subcutaneous injection.

46. The method of claim 30, wherein the antibody or antigen-binding fragment thereof achieves one or more of the following when administered to a subject:
(a) reduction of LDL-C of at least −60% to at least −75% relative to a predose level with a sustained reduction over at least a 14 day-period upon administering to a subject a dose of 150 mg every two weeks;
(b) reduction of LDL-C of at least −50% to −75% relative to a predose level with a sustained reduction over at least a 28 day-period upon administering to a subject a dose of 300 mg every four weeks;
(c) increase of serum HDL cholesterol levels of at least 2%, at least 2.5%, at least 3%, at least 3.5%, at least 4%, at least 4.5%, at least 5%, or at least 5.5% relative to a predose level upon administering to a subject a dose of 150 mg every two weeks; and
(d) reduction of one or more of: total-cholesterol levels, ApoB levels, non HDL-C levels, and ApoB/ApoA-1 ratio.
